(19) United States
(12) Patent Application Publication
(10) Pub. No.: US 2014/0221374 A1
(43) Pub. Date: Aug. 7, 2014

(54) RAF KINASE INHIBITORS

(71) Applicants: Jean-Michel Vernier, San Diego, CA (US); Stephanie Hopkins, Poway, CA (US); Pierre-Yves Bounaud, San Diego, CA (US); Patrick O'Connor, San Diego, CA (US); David Matthews, Encinitas, CA (US); Steve Bender, Oceanside, CA (US)

(72) Inventors: Jean-Michel Vernier, San Diego, CA (US); Stephanie Hopkins, Poway, CA (US); Pierre-Yves Bounaud, San Diego, CA (US); Patrick O'Connor, San Diego, CA (US); David Matthews, Encinitas, CA (US); Steve Bender, Oceanside, CA (US)

(73) Assignee: Ruga Corporation, Palo Alto, CA (US)

(21) Appl. No.: 14/005,720

(22) PCT Filed: Mar. 16, 2012
(86) PCT No.: PCT/US12/29547

§ 371 Date: Mar. 3, 2014

Related U.S. Application Data
(60) Provisional application No. 61/453,898, filed on Mar. 17, 2011.
(60) Provisional application No. 61/467,930, filed on Mar. 25, 2011.
(60) Provisional application No. 61/467,876, filed on Mar. 25, 2011.

(51) Int. Cl. C07D 417/14; C07D 403/14; C07D 401/14; C07D 401/04; C07D 403/04; C07D 417/04

(52) U.S. Cl. CPC C07D 417/14; C07D 403/04; C07D 417/04; C07D 401/14; C07D 401/04; C07D 403/14
USPC 514/235.8; 514/235.5; 514/236.8; 514/275; 514/341; 514/342; 514/343; 544/122; 544/131; 544/331; 546/270.4; 546/274.4; 546/275.4; 546/276.4; 435/184

(57) ABSTRACT

Described herein are compounds, pharmaceutical compositions and methods for the inhibition of RAF kinase mediated signaling. Said compounds, pharmaceutical compositions and methods have utility in the treatment of human disease and disorders.

### CROSS-REFERENCE

This application claims the benefit of U.S. Provisional Application No. 61/453,898, filed Mar. 17, 2011; U.S. Provisional Application No. 61/467,876, filed. Mar. 25, 2011; and U.S. Provisional Application No. 61/467,930, filed. Mar. 25, 2011, each of which is incorporated herein by reference in its entirety.

### BACKGROUND OF THE INVENTION

Described herein are compounds, pharmaceutical compositions and methods for the inhibition of RAF kinase mediated signaling. Said compounds; pharmaceutical compositions and methods have utility in the treatment of human disease and disorders.

### SUMMARY OF THE INVENTION

One embodiment provides a compound of Formula (I), or a tautomer, steroisomer, geometric isomer, a pharmaceutically acceptable salt, solvate, or hydrate thereof:

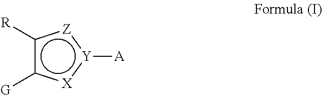

wherein
Z is N, Y is C, and X is NH;
or
Z is CH, Y is N, and X is N;
or
Z is N, Y is N, and X is N;
R is

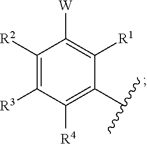

G is selected from:

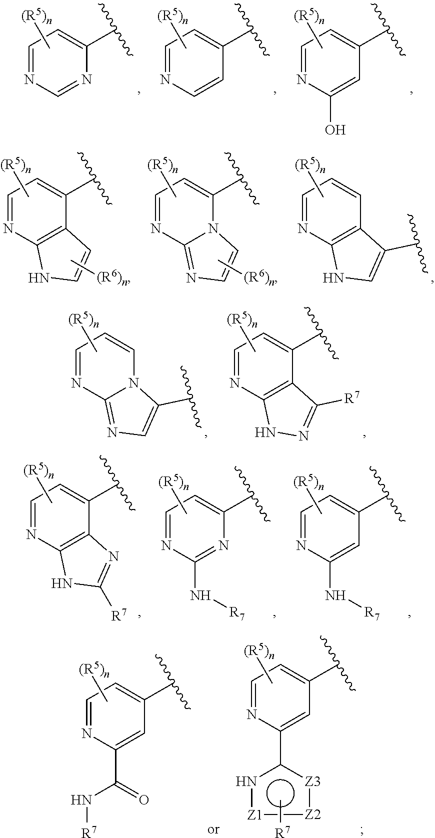

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHSO₂Ar, —NHCOAr, —NHSO₂NHAr, —NHSO₂N(Ar)₂, —NHCONHAr, —N(OH)CONHAr, —NHCON(Ar)₂, —NHCSNHAr, —NHCSN(Ar)₂, —NHCOC(R¹¹)(R¹²)Ar, —C(R¹¹)(R¹²)CONHAr;
Ar is:

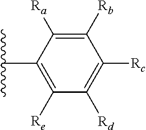

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently selected from H, or C₁-C₆ alkyl; or for the instance wherein R¹¹ and R¹² are attached geminal carbon substituents, R¹¹ and R¹² together with the carbon atom to which they are attached are joined to form a C₃-C₆ cycloalkyl; and
n is 0, 1, or 2.

Another embodiment provides the compound of Formula (r), wherein. Z is CH, Y is N, and X is N.

Another embodiment provides the compound of Formula (I), wherein Z is N, Y is C, and X is NH.

Another embodiment provides the compound of Formula (I), wherein Z is N, Y is N, and X is N.

Another embodiment provides the compound of Formula (I), wherein W is NHSO₂Ar.

Another embodiment provides the compound of Formula (I), wherein W is NHCONHAr.

Another embodiment provides the compound of Formula (I), wherein G is

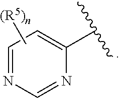

Another embodiment provides the compound of Formula (I), wherein G is

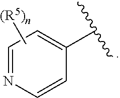

Another embodiment provides the compound of Formula (I), wherein G is

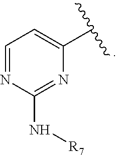

Another embodiment provides the compound of Formula (I), wherein G is

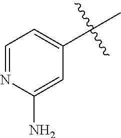 [SMILES: *c1ccnc(N)c1]

Another embodiment provides the compound of Formula (I), wherein A is an optionally substituted alkyl or optionally substituted cycloalkyl. Another embodiment provides the compound of Formula (I), wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (I), wherein A is NR⁹R¹⁰.

Another embodiment provides the compound of Formula (I), wherein A is an optionally substituted C-attached heterocycloalkyl.

Another embodiment provides the compound of Formula (I), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl. Another embodiment provides the compound of Formula (I), wherein R³ and R⁴ are hydrogen. Another embodiment provides the compound of Formula (I), wherein R¹ and R² are each independently selected from hydrogen, F, Cl, CN, OH, CH₂F, CHF₂, CF₃, or C₂F₅.

Another embodiment provides the compound of Formula (I), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

Another embodiment provides the compound of Formula (I) selected from the following:

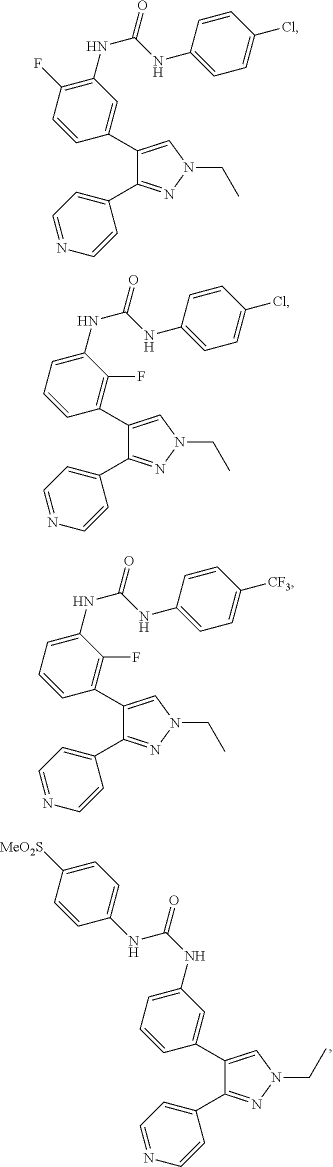

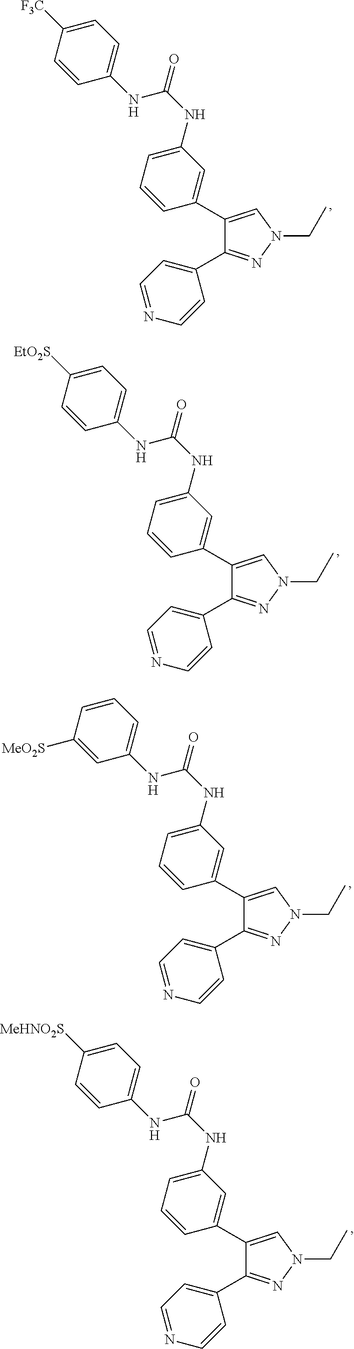

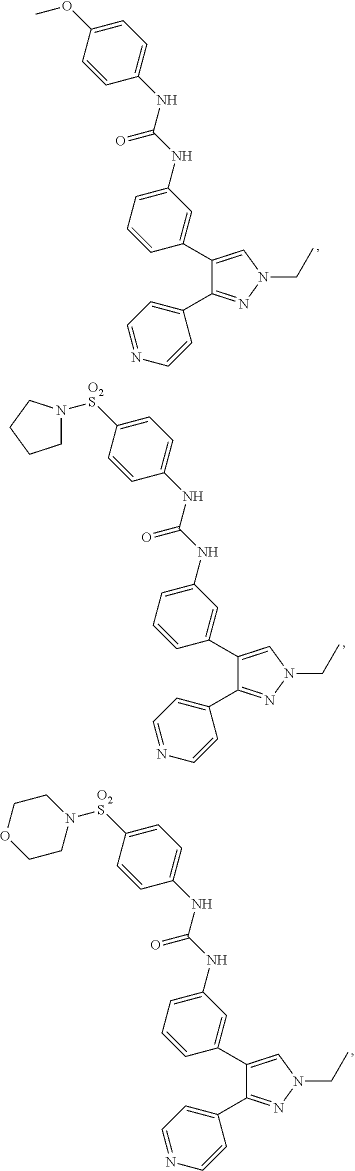 [SMILES: CCn1cc(-c2cccc(NC(=O)Nc3ccc(OC)cc3)c2)c(-c2ccncc2)n1 ; CCn1cc(-c2cccc(NC(=O)Nc3ccc(S(=O)(=O)N4CCCC4)cc3)c2)c(-c2ccncc2)n1 ; CCn1cc(-c2cccc(NC(=O)Nc3ccc(S(=O)(=O)N4CCOCC4)cc3)c2)c(-c2ccncc2)n1]

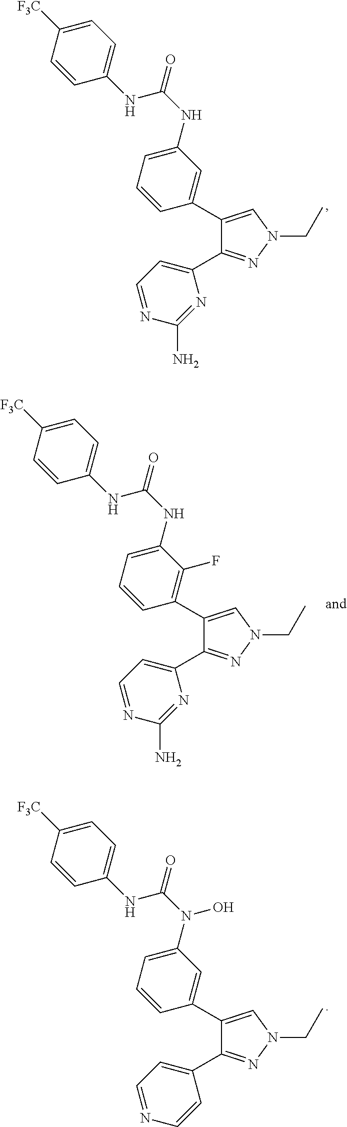 [SMILES: CCn1cc(-c2cccc(NC(=O)Nc3ccc(C(F)(F)F)cc3)c2)c(-c2ccnc(N)n2)n1 ; CCn1cc(-c2cccc(NC(=O)Nc3ccc(C(F)(F)F)cc3)c2F)c(-c2ccnc(N)n2)n1 ; CCn1cc(-c2cccc(N(O)C(=O)Nc3ccc(C(F)(F)F)cc3)c2)c(-c2ccncc2)n1]

One embodiment provides a compound of Formula (II), or a tautomer, steroisomer, geometric isomer, a pharmaceutically acceptable salt, solvate, or hydrate thereof:

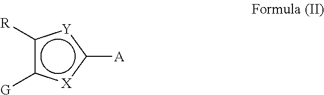

wherein
X is S and Y is N; or
X is N and Y is S; or
X is O and Y is N; or
X is N and Y is O;
R is

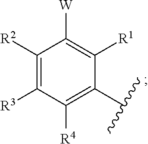

G is selected from:

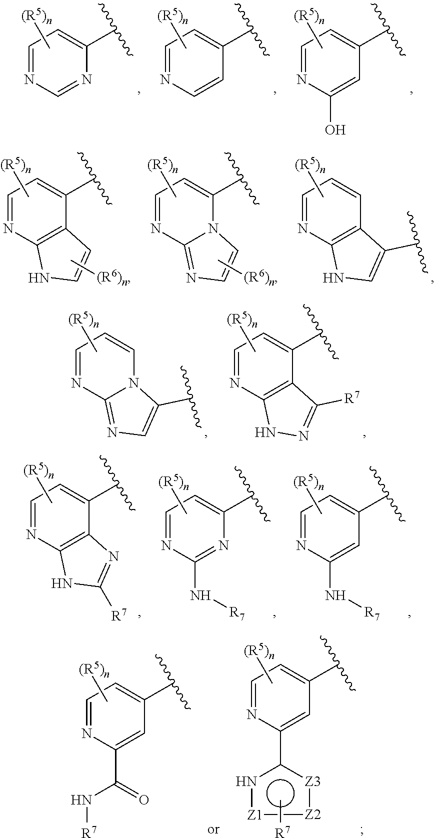

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy, -(optionally substituted alkylene)-(NHCO₂H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHCOAr, —NHSO₂NHAr, —N(OH)CONHAr, —NHSO₂N(Ar)₂, —NHCONHAr, —NHCON(Ar)₂, —NHCOC(R¹¹)(R¹²)Ar, —C(R¹¹)(R¹²)CONHAr, NHCSNHAr, NHCSN(Ar)₂ or —N(R¹¹)CON(R¹²)Ar;
Ar is:

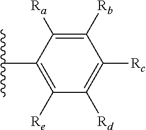

Ra, Rb, Re, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl), NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently selected, from H, or C₁-C₆ alkyl; or for the instance wherein R¹¹ and R¹² are attached germinal carbon substituents, R¹¹ and R¹² together with the carbon atom to which they are attached are joined to form a C₃-C₆ cycloalkyl; and n is 0, 1, or 2.

Another embodiment, provides the compound of Formula (II), wherein X is S and Y is N.

Another embodiment provides the compound of Formula (II), wherein X is N and Y is S.

Another embodiment provides the compound of Formula (II), wherein W is NHSO₂Ar.

Another embodiment provides the compound of Formula (II), wherein W is NHCONHAr.

Another embodiment provides the compound of Formula (II), wherein G is

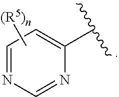

Another embodiment provides the compound of Formula (II), wherein G is

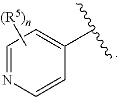

Another embodiment provides the compound of Formula (II), wherein G is

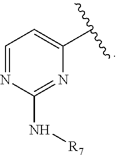

Another embodiment provides the compound of Formula (II), wherein G is

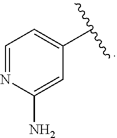 [SMILES: *c1ccnc(N)c1]

Another embodiment provides the compound of Formula (II), wherein A is an optionally substituted alkyl or optionally substituted cycloalkyl. Another embodiment provides the compound of Formula (II), wherein A is an optionally substituted group selected from methyl, ethyl, trifluoromethyl, 2,2,2-trifluoroethyl, n-propyl, i-propyl, n-butyl, s-butyl, t-butyl, cyclopropyl or cyclobutyl.

Another embodiment provides the compound of Formula (II), wherein A is —NR⁹R¹⁰.

Another embodiment provides the compound of Formula (II), wherein A is an optionally C-attached heterocycloallyl.

Another embodiment provides the compound of Formula (II), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl. Another embodiment provides the compound of Formula (II), wherein R³ and R⁴ are hydrogen. Another embodiment provides the compound of Formula (II), wherein R¹ and R² are each independently selected from hydrogen, F, Cl, CN, OH, CH₂F; CHF₂, CF₃, or C₂F₅.

Another embodiment provides the compound of Formula (II), wherein Ra, Rb, Rc, Rd and Re are each independently selected from hydrogen, F, Cl, CN, CF₃, OH, C₂F₅, NO₂, NH₂; —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

Another embodiment provides the compound of Formula (II) selected from the following:

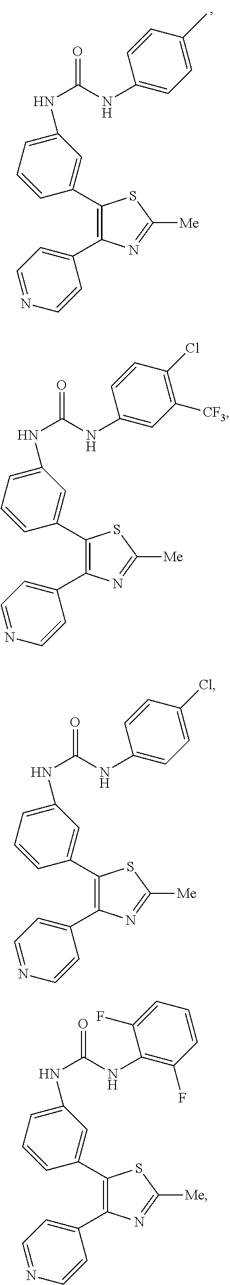 [SMILES: Cc1ccc(NC(=O)Nc2cccc(-c3sc(C)nc3-c3ccncc3)c2)cc1 ; Cc1nc(-c2ccncc2)c(-c2cccc(NC(=O)Nc3ccc(Cl)c(C(F)(F)F)c3)c2)s1 ; Cc1nc(-c2ccncc2)c(-c2cccc(NC(=O)Nc3ccc(Cl)cc3)c2)s1 ; Cc1nc(-c2ccncc2)c(-c2cccc(NC(=O)Nc3c(F)cccc3F)c2)s1]

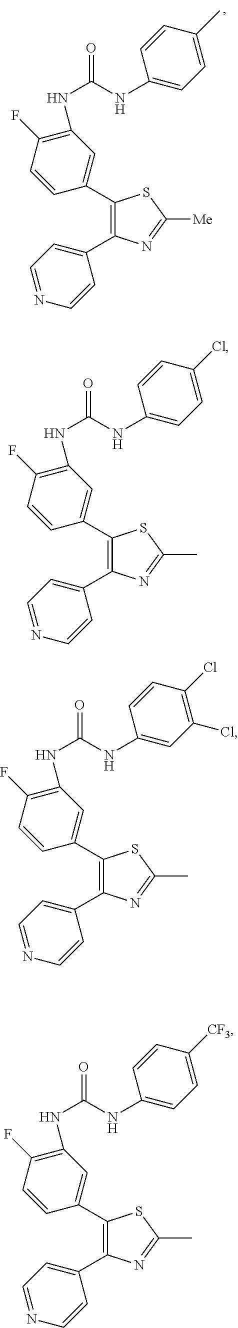 [SMILES: Cc1ccc(NC(=O)Nc2cc(-c3sc(C)nc3-c3ccncc3)ccc2F)cc1 ; Cc1nc(-c2ccncc2)c(-c2ccc(F)c(NC(=O)Nc3ccc(Cl)cc3)c2)s1 ; Cc1nc(-c2ccncc2)c(-c2ccc(F)c(NC(=O)Nc3ccc(Cl)c(Cl)c3)c2)s1 ; Cc1nc(-c2ccncc2)c(-c2ccc(F)c(NC(=O)Nc3ccc(C(F)(F)F)cc3)c2)s1]

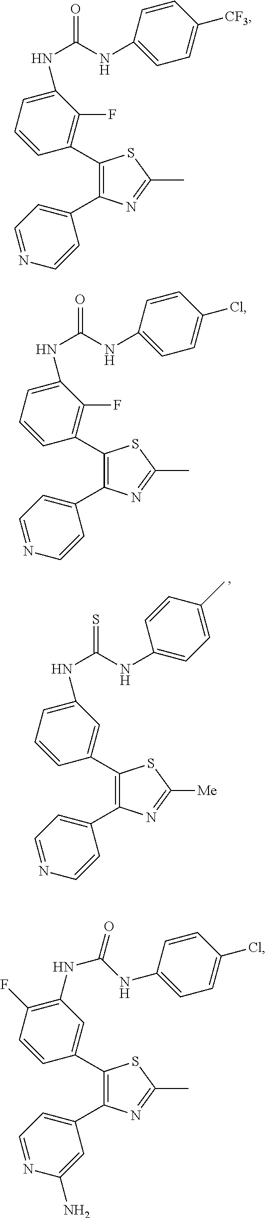

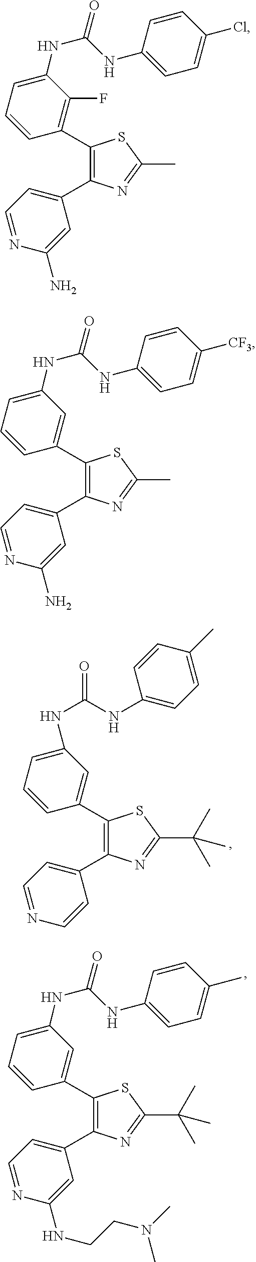

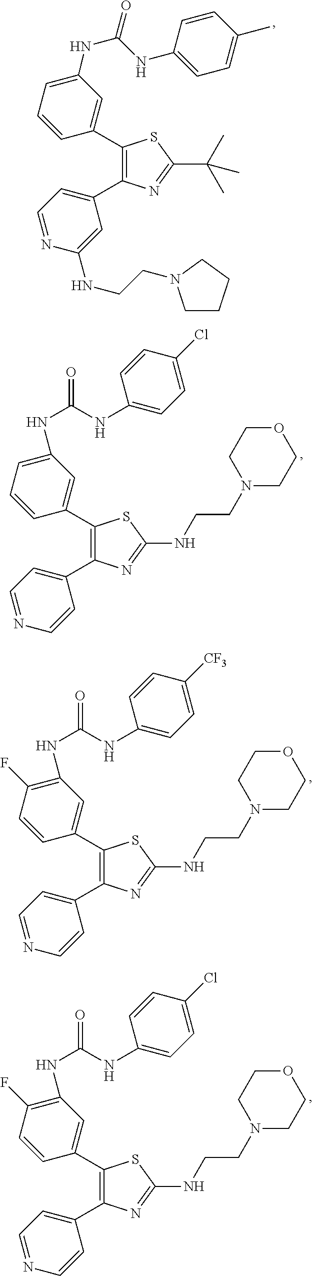

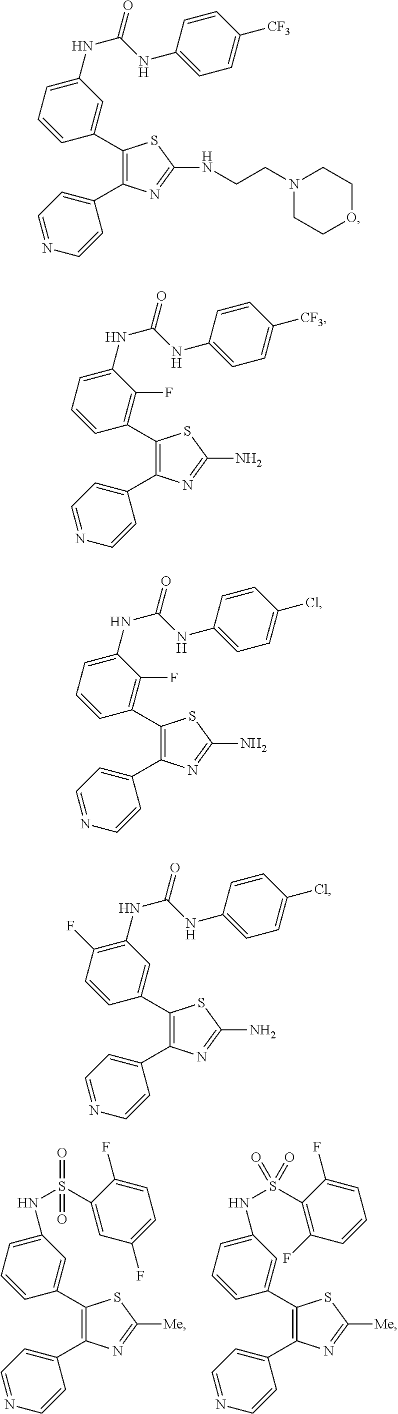

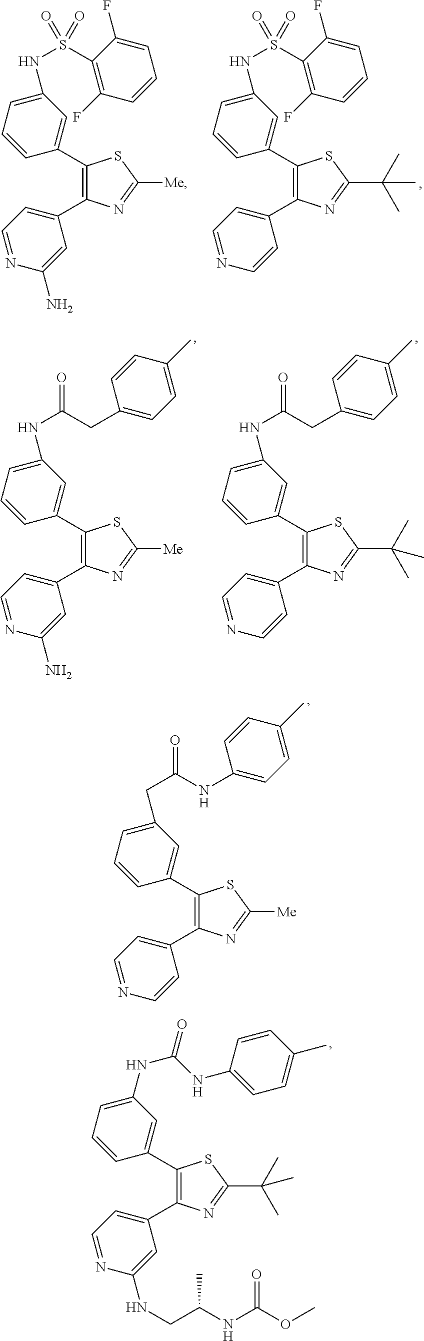

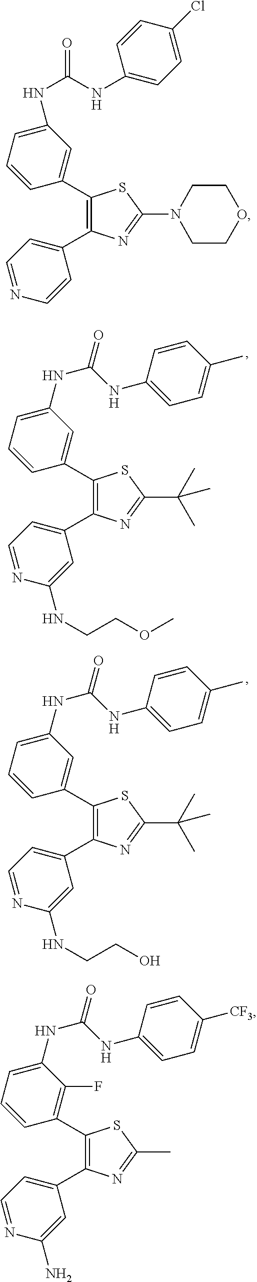

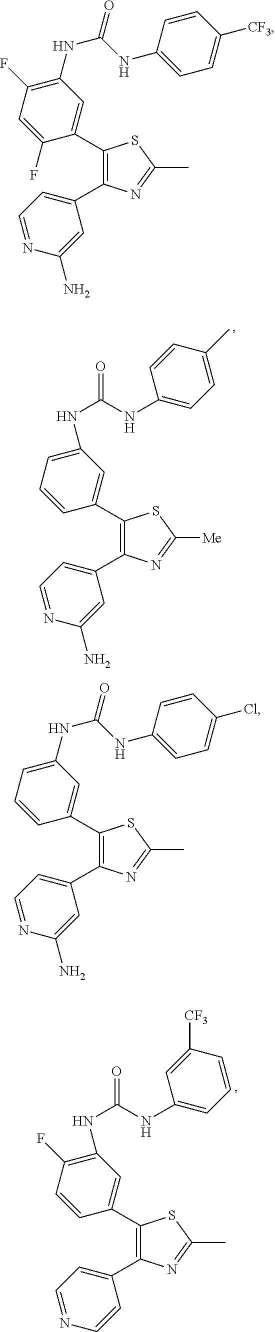

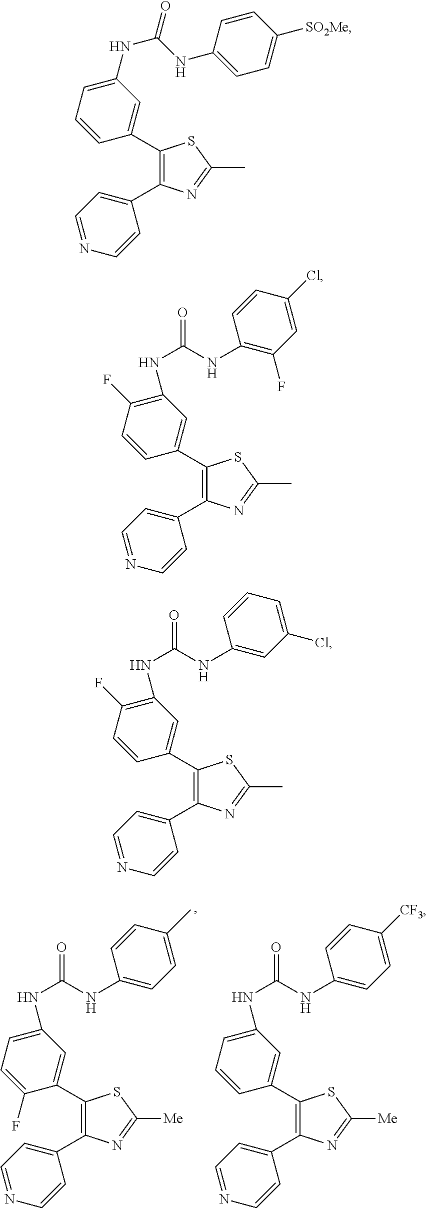

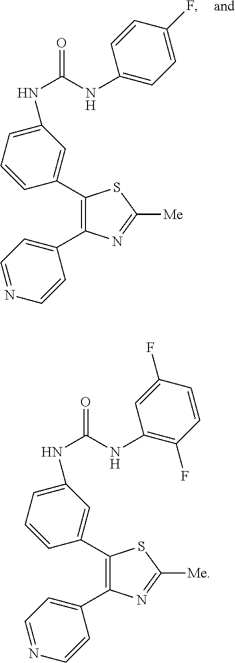 [SMILES: Cc1nc(-c2ccncc2)c(-c2cccc(NC(=O)Nc3ccc(F)cc3)c2)s1 ; "and" ; Cc1nc(-c2ccncc2)c(-c2cccc(NC(=O)Nc3cc(F)ccc3F)c2)s1]

One embodiment provides a compound of Formula (I), or a tautomer, steroisomer, geometric isomer, a pharmaceutically acceptable salt, solvate, or hydrate thereof:

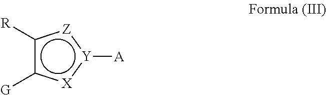

wherein
Z is N, Y is C, and X is NH;
or
Z is N, Y is N, and X is CH;
or
Z is CH, Y is N, and X is N;
or
Z is N, Y is N, and X is N;
R is

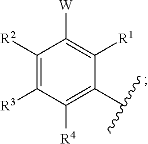

G is selected from:

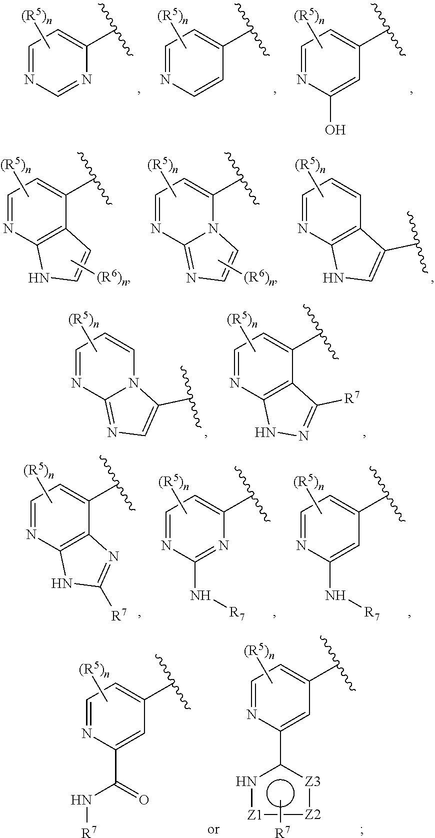

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted allyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

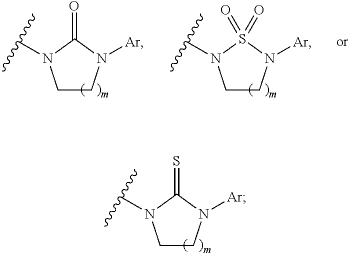

Ar is:

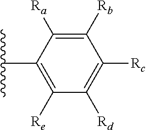

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3; and
n is 0, 1, or 2.

Another embodiment provides the compound of Formula (III), wherein Z is N, Y is C, and X is NH.

Another embodiment provides the compound of Formula (III), wherein Z is N, Y is N, and X is CH.

Another embodiment provides the compound of Formula (III), wherein Z is CH, Y is N, and X is N.

Another embodiment provides the compound of Formula (III), wherein Z is N, Y is N, and X is N.

Another embodiment provides the compound of Formula (III), wherein W is

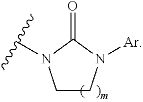

Another embodiment provides the compound of Formula (III), wherein G is

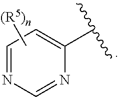

Another embodiment provides the compound of Formula (III), wherein G is

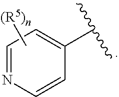

Another embodiment provides the compound of Formula (M), wherein G is

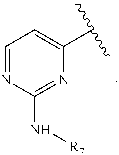

Another embodiment provides the compound of Formula (III), wherein G is

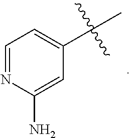 [SMILES: *c1ccnc(N)c1]

Another embodiment provides the compound of Formula (III), wherein A is an optionally substituted alkyl or optionally substituted cycloalkyl.

Another embodiment provides the compound of Formula (III), wherein. A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (III), wherein A is NR⁹R¹⁰.

Another embodiment provides the compound of Formula (III), wherein A is an optionally substituted C-attached heterocycloalkyl.

Another embodiment provides the compound of Formula (III), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl. Another embodiment provides the compound of Formula (III), wherein. R³ and R⁴ are hydrogen. Another embodiment provides the compound of Formula (III), wherein R¹ and R² are each independently selected from hydrogen, F, Cl, CN, OH, CH₂F, CHF₂, CF₃, or C₂F₅.

Another embodiment provides the compound of Formula (III), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

Another embodiment provides the compound of Formula (III) selected from the following:

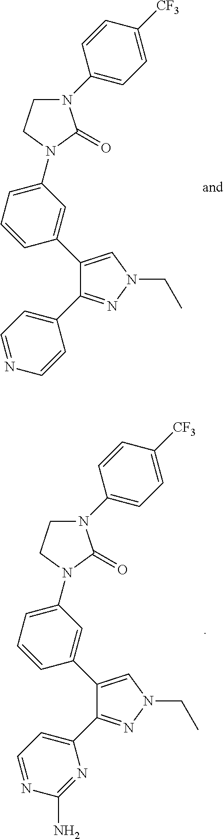 [SMILES: CCn1cc(-c2cccc(N3CCN(c4ccc(C(F)(F)F)cc4)C3=O)c2)c(-c2ccncc2)n1 ; CCn1cc(-c2cccc(N3CCN(c4ccc(C(F)(F)F)cc4)C3=O)c2)c(-c2ccnc(N)n2)n1]

One embodiment provides a compound of Formula (IV), or a tautomer, steroisomer, geometric isomer, a pharmaceutically acceptable salt, solvate, or hydrate thereof:

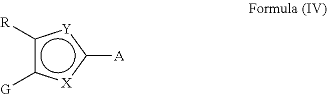

wherein
X is S and Y is N; or
X is N and Y is S; or
X is O and Y is N; or
X is N and Y is O;
R is

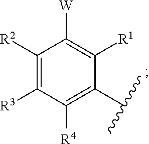

G is selected from:

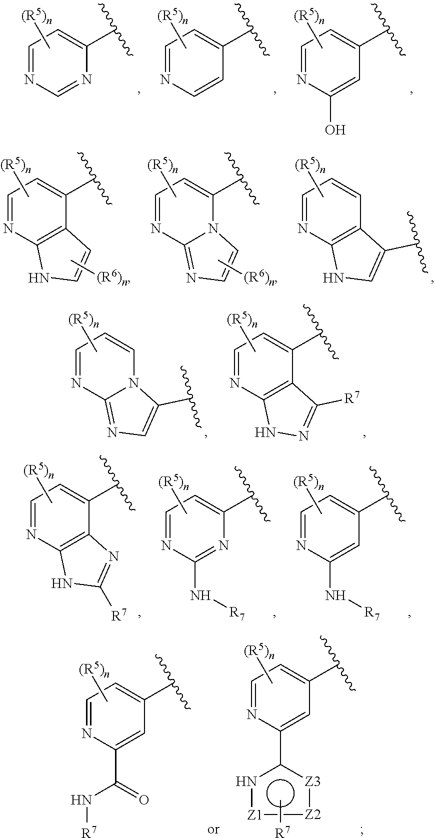

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl; -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally Substituted, alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹R¹⁰, optionally substituted N-attached heterocycloalkyl, optionally substituted. C-attached heterocycloalkyl, optionally substituted cycloalkyl, or optionally substituted heteroalkyl;
R¹, R², R³ and R⁴ are each independently selected from hydrogen, F, Cl, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

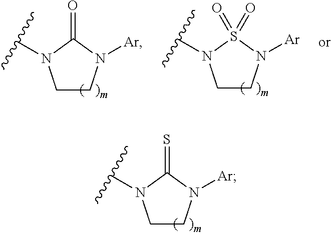

Ar is:

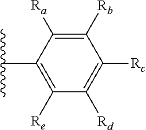

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl), NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3;
n is 0, 1, or 2.

Another embodiment provides the compound of Formula (IV), wherein. X is S and Y is N.

Another embodiment provides the compound of Formula (IV), wherein X is N and Y is S.

Another embodiment provides the compound of Formula (IV), wherein W is

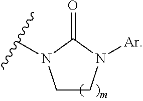

Another embodiment provides the compound of Formula (IV), wherein G is

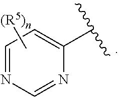

Another embodiment provides the compound of Formula (IV), wherein G is

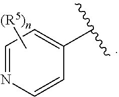

Another embodiment provides the compound of Formula (IV), wherein G is

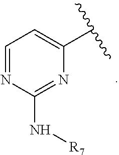

Another embodiment provides the compound of Formula (IV), wherein G is

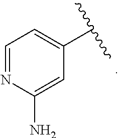 [SMILES: *c1ccnc(N)c1]

Another embodiment provides the compound of Formula (IV), wherein A is an optionally substituted alkyl or optionally substituted cycloalkyl.

Another embodiment provides the compound of Formula (IV), wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (IV); wherein A is NR⁹R¹⁰.

Another embodiment provides the compound of Formula (IV), wherein A is an optionally C-attached heterocycloalkyl.

Another embodiment provides the compound of Formula (IV), wherein R¹, R², R³ and R⁴ are each independently selected from hydrogen, F, Cl, CN, OH, CH₂F, CHF₂, CF₃, C₂F₅, NO₂, —NH(C₁-C₅ optionally substituted alkyl), —N(C₁-C₅ optionally substituted alkyl)₂, or C₁-C₅ optionally substituted alkyl.

Another embodiment provides the compound of Formula (IV), wherein R³ and R⁴ are hydrogen.

Another embodiment provides the compound of Formula (IV), wherein R¹ and R² are each independently selected from hydrogen, F, Cl, CN, OH, CH₂F, CHF₂, CF₃, or C₂F₅.

Another embodiment provides the compound of Formula (IV), wherein Ra, Rb, Rc, Rd and Re are each independently selected from hydrogen, F, Cl; CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

One embodiment provides a pharmaceutical composition comprising a compound of Formula (I), or a stereoisomer; tautomer, hydrate, solvate or pharmaceutically acceptable salt thereof, and at least one pharmaceutically acceptable excipient.

One embodiment provides a pharmaceutical composition comprising a compound of Formula (II), or a stereoisomer, tautomer, hydrate, solvate or pharmaceutically acceptable salt thereof, and at least one pharmaceutically acceptable excipient.

One embodiment provide a pharmaceutical composition comprising a compound of Formula (III), or a stereoisomer, tautomer, hydrate, solvate or pharmaceutically acceptable salt thereof; and at least one pharmaceutically acceptable excipient.

One embodiment provides a pharmaceutical composition comprising a compound of Formula (IV); or a stereoisomer, tautomer, hydrate, solvate or pharmaceutically acceptable salt thereof, and at least one pharmaceutically acceptable excipient.

One embodiment provides a method of inhibiting a protein kinase comprising contacting the protein kinase with an inhibitory concentration of a compound of Formula (I).

One embodiment provides a method of inhibiting a protein kinase comprising contacting the protein kinase with an inhibitory concentration of a compound of Formula (II).

One embodiment provides a method of inhibiting a protein kinase comprising contacting the protein kinase with an inhibitory concentration of a compound of Formula (III).

One embodiment provides a method of inhibiting a protein kinase comprising contacting the protein kinase with an inhibitory concentration of a compound of Formula (IV).

Another embodiment provides a method of inhibiting a protein kinase, wherein the protein kinase is selected from A-RAF, B-RAF and C-RAF. Another embodiment provides a method of inhibiting a protein kinase, wherein the protein kinase is B-RAF. Another embodiment provides a method of inhibiting a protein kinase, wherein the protein kinase is C-RAF. Another embodiment provides a method of inhibiting a protein kinase, wherein the protein kinase is a B-RAF mutant. Another embodiment provides a method of inhibiting a protein kinase, wherein the protein kinase is the B-RAF-V600E mutant.

One embodiment provides a′ method of inhibiting RAF kinase mediated signalling in a cell comprising contacting the cell with an inhibitory concentration of a compound of Formula (I). Another embodiment provides a method of inhibiting RAF kinase mediated signalling in a cell, wherein the cell is characterized by increased activity of the RAS-RAF-pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (II). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (II). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (IV). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of treating a human disease or disorder mediated by RAF kinase signalling comprising administering to a patient a therapeutically effective amount of a composition comprising a compound of Formula (I). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a composition comprising a compound of Formula (II). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a composition comprising a compound of Formula (III). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a composition comprising a compound of Formula (IV). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the structures of 17 additional examples of the compounds of Formula (I);

FIG. 2 illustrates the structures of 12 additional examples of the compounds of Formula (I);

FIG. 3 illustrates the structures of 12 additional examples of the compounds of Formula (I);

FIG. 4 illustrates the structures of 14 additional examples of the compounds of Formula (I);

FIG. 5 illustrates the structures of 12 additional examples of the compounds of Formula (I);

FIG. 6 illustrates the structures of 11 additional examples of the compounds of Formula (I);

FIG. 7 illustrates the structures of 17 additional examples of the compounds Of Formula (I);

FIG. 8 illustrates the structures of 12 additional examples of the compounds of Formula (I);

FIG. 9 illustrates the structures of 12 additional examples of the compounds of Formula (I);

FIG. 10 illustrates the structures of 14 additional examples of the compounds of Formula (I);

FIG. 11 illustrates the structures of 12 additional examples of the compounds of Formula (I);

FIG. 12 illustrates the structures of 11 additional examples of the compounds of Formula (I);

FIG. 13 illustrates the structures of 17 additional examples of the compounds of Formula (II);

FIG. 14 illustrates the structures of 12 additional examples of the compounds of Formula (II);

FIG. 15 illustrates the structures of 12 additional examples of the compounds of Formula (II);

FIG. 16 illustrates the structures of 14 additional examples of the compounds of Formula (II);

FIG. 17 illustrates the structures of 12 additional examples of the compounds of Formula (II); and

FIG. 18 illustrates the structures of 11 additional examples of the compounds of Formula (II).

### DETAILED DESCRIPTION OF THE INVENTION

Growth factor signaling through cell membrane associated receptor tyrosine kinases (RTKs) is commonly defective in human cancers. These RTKs transduce signals to intracellular machinery responsible for a variety of cellular processes including cell proliferation, survival, migration and differentiation (Hunter, T., Cell, 100: 113-127, 2000; Hanahan, D. and Weinberg, R. A., Cell, 100: 57-70, 2000).

An important intracellular signaling conduit is the RAS-RAF-MEK-ERK pathway that relays growth factor mediated RTK signals to responder elements in the cytoplasm and/or nuclear compartments (Robinson, M. J. and Cobb, M. H., Curr. Opin. Cell Biol., 9: 180-186, 1997). Within this pathway both RAS and RAF members were initially discovered as viral oncogenes that transformed mammalian cells and such eventually lead to the identification of human homologs with similar oncogenic transforming activity (Rapp, U. R., et al., Proc. Natl. Acad. Sci., 80: 4218-4222, 1983: Malumbres, M. and Barbacid, M., Nat. Rev. Cancer, 3: 459-465, 2003 and references therein).

RAF activation is normally regulated by an upstream RAS-GTP bound complex that orchestrates RAF binding to the cell membrane. Subsequent conformational changes induce RAF phosphorylation and kinase activity. The active RAF kinase then phosphorylates and activates MEK, that in-turn phosphorylates and activates ERK1/2 in a signaling cascade that is conserved across a wide variety of animal species (Kolch, W. Biochem. J. 351: 289-305, 2000 and references therein). There are 3 recognized human isoforms of RAF: A-RAF, B-RAF and C-RAP (also known as c-RAF-1), and signaling of RAF to MEK normally requires KSR, a RAF homolog lacking intrinsic kinase activity acting as a scaffold in protein-protein interactions.

Aberrant activation of the RAS-RAF-MEK-ERK pathway is common across human cancers, with gain-of-function mutations reported for RAS and B-RAF that lead to constitutive activation of these proteins. For example, B-RAF mutations have been identified in a wide variety of tumors including melanoma (50-70%), colon cancer (10-15%), ovarian cancer (30-40%) and papillary thyroid cancer (45%) (Davies, H., et al., Nature, 417: 949-954, 2002; Yuen, S. T., et al., Cancer Research, 62: 6451-6455, 2002: Singer, G., et al., J. Natl. Cancer Inst., 95: 484-486, 2003; Brose, M. S., et al., Cancer Res., 62: 6997-7000, 2002; Rajagopalan, H., et al., Nature, 418: 934, 2002; Tuveson, D.; et al., Cancer Cell, 4: 95-98, 2003).

The vast majority of B-RAF gain-of-function mutations identified to date (˜90%) involve substitution of a valine for a glutamic acid at position 600. Often referred to as B-RAF (V600E), this single amino acid substitution leads to constitutive kinase activity approximately 500-fold higher than basal wild-type B-RAF kinase activity (Wan, P. T. C., et al., Cell, 116: 855-867, 2004; Garnett, M. J. and Marais, R. Cancer Cell, 6: 313-319, 2004). In addition, B-RAF (V600E) is by itself transforming, and increases tumor cell proliferation, survival and tumor growth in viva (Davies, H., et al., Nature, 417: 949-954, 2002; Wellbrock, C., et al., Cancer Res., 64: 2338-2342, 2004). Furthermore, B-RAF (V600E) mutations have been correlated with decreased response rates in cancer patients undergoing chemotherapy (Samowitz, W. S., et al., Cancer Research, 65: 6063-6069, 2005; Houben R., et al., J. Carcinogenesis, 3: 6-18, 2004). Consistent with a pivotal role of B-RAF (V600E) in tumor growth, siRNA directed to B-RAF (V600E) results in tumor cell growth arrest and/or apoptosis (Karasarides, M., et al., Oncogene, 23: 6292-6298, 2004; Hingorani, S. R., et al., Cancer Res., 63: 5198-5202, 2003; Hoeflich, K. P., et al., Cancer Res., 66: 999-1006, 2006). Selective B-RAF (V600E) inhibition is important to achieve selective killing of tumor cells harboring this gain-of-function mutation while sparing normal cells, thereby reducing or eliminating side-effects in cancer patients on long-term therapy.

### Heterocyclic RAF Kinase Inhibitors

One embodiment provides a compound of Formula (I), or a tautomer, steroisomer, geometric isomer, a pharmaceutically acceptable salt, solvate, or hydrate thereof.

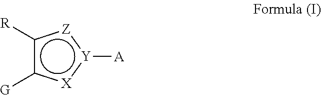

wherein
Z is N, Y is C, and X is NH;
or
Z is CH, Y is N, and X is N;
or
Z is N, Y is N, and X is N;
R is

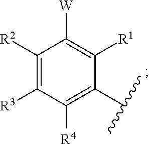

is selected from:

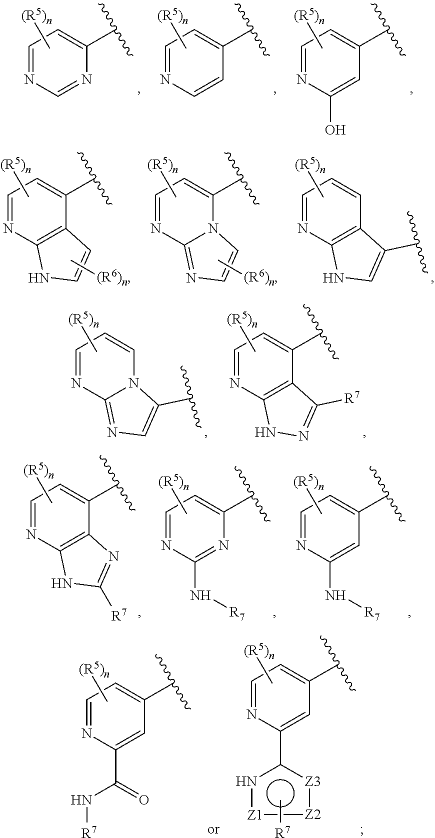

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected, from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHSO₂Ar, —NHCOAr, —NHSO₂NHAr, —NHSO₂N(Ar)₂, —NHCONHAr, —N(OH)CONHAr, —NHCON(Ar)₂, —NHCSNHAr, —NHCSN(Ar)₂, —NHCOC(R¹¹)(R¹²)Ar, —C(R¹¹)(R¹²)CONHAr;
Ar is:

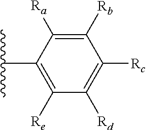

Ra, Rb, Rc, Rd and Re are each independently selected from hydrogen, F, Cl, CN, CF₃, OH, C₂F₅NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently selected from H, or C₁-C₆ alkyl; or for the instance wherein R¹¹ and R¹² are attached geminal carbon substituents, R¹¹ and R¹² together with the carbon atom to which they are attached are joined to form a C₃-C₆ cycloalkyl; and
n is 0, 1, or 2.

Another embodiment provides the compound of Formula (I), wherein Z is CH, Y is N, and X is N.

Another embodiment provides the compound of Formula (I), wherein Z is N, Y is C, and X is NH.

Another embodiment provides the compound of Formula (I), wherein Z is N, Y is N, and X is N.

Another embodiment provides the compound of Formula (I), wherein W is NHSO₂Ar.

Another embodiment provides the compound of Formula (I), wherein W is NHCONHAr.

Another embodiment provides the compound of Formula (I), wherein G is

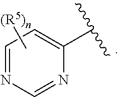

Another embodiment provides the compound of Formula (I), wherein G is

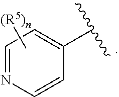

Another embodiment provides the compound of Formula (I), wherein G is

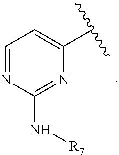

Another embodiment provides the compound of Formula (I), wherein G is

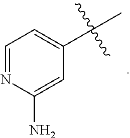 [SMILES: *c1ccnc(N)c1]

Another embodiment provides the compound of Formula (I), wherein A is an optionally substituted alkyl or optionally substituted cycloalkyl. Another embodiment provides the compound of Formula (I), wherein A is an optionally substituted group selected from methyl, ethyl, trifluoromethyl, 2,2,2-trifluoroethyl, n-propyl, i-propyl, n-butyl, s-butyl i-butyl, t-butyl, cyclopropyl or cyclobutyl.

Another embodiment provides the compound of Formula (I), wherein A is NR⁹R¹⁰.

Another embodiment provides the compound of Formula (I), wherein A is an optionally substituted C-attached heterocycloalkyl.

Another embodiment provides the compound of Formula (I), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl. Another embodiment provides the compound of Formula (I), wherein R³ and R⁴ are hydrogen. Another embodiment provides the compound of Formula (I), wherein R¹ and R² are each independently selected from hydrogen, F, Cl, CN, OH, CH₂F, CHF₂, CF₃, or C₂F₅.

Another embodiment provides the compound of Formula (I), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

Another embodiment provides the compound of Formula (I) selected from the following:

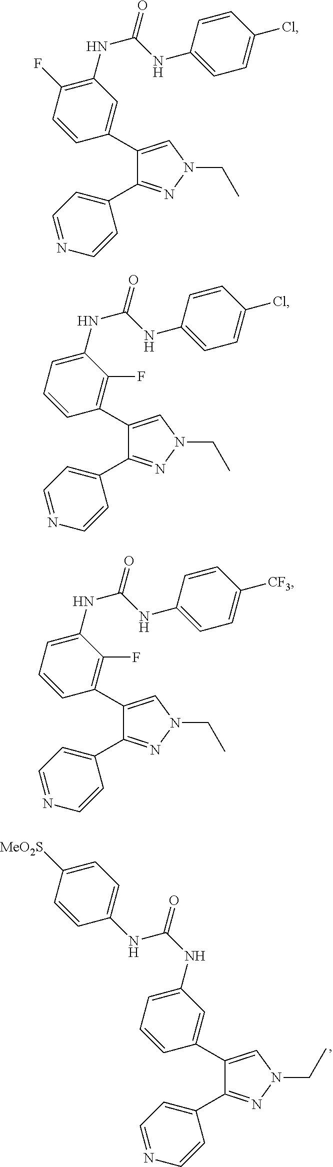

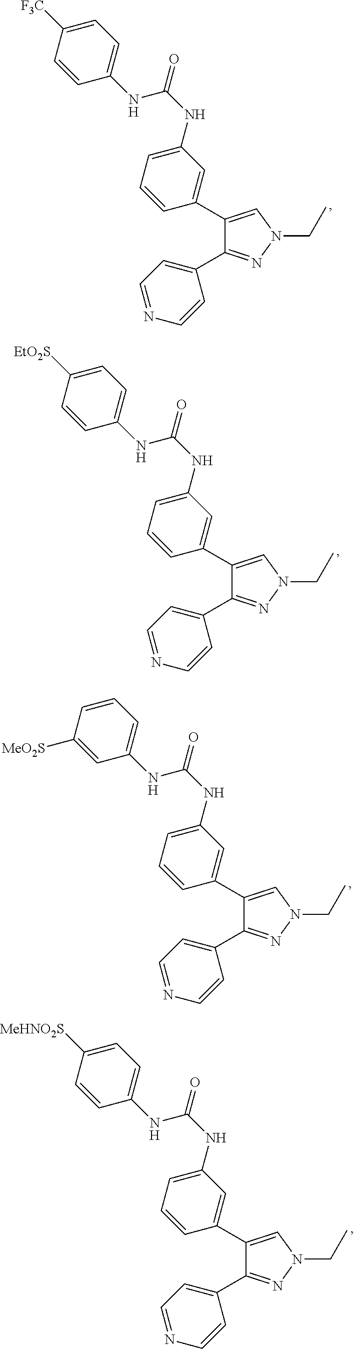

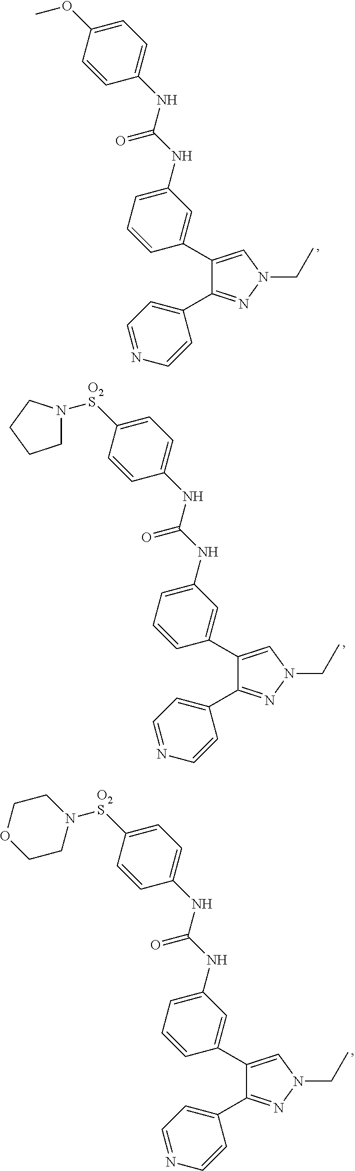 [SMILES: CCn1cc(-c2cccc(NC(=O)Nc3ccc(OC)cc3)c2)c(-c2ccncc2)n1 ; CCn1cc(-c2cccc(NC(=O)Nc3ccc(S(=O)(=O)N4CCCC4)cc3)c2)c(-c2ccncc2)n1 ; CCn1cc(-c2cccc(NC(=O)Nc3ccc(S(=O)(=O)N4CCOCC4)cc3)c2)c(-c2ccncc2)n1]

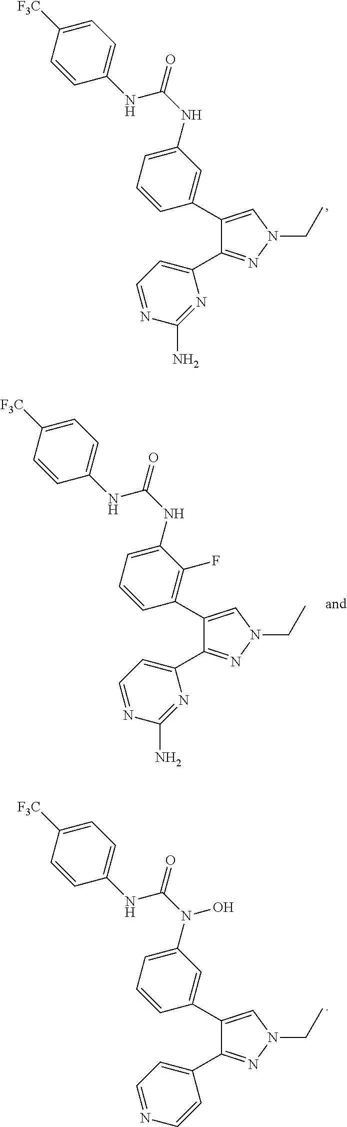 [SMILES: CCn1cc(-c2cccc(NC(=O)Nc3ccc(C(F)(F)F)cc3)c2)c(-c2ccnc(N)n2)n1 ; CCn1cc(-c2cccc(NC(=O)Nc3ccc(C(F)(F)F)cc3)c2F)c(-c2ccnc(N)n2)n1 ; CCn1cc(-c2cccc(N(O)C(=O)Nc3ccc(C(F)(F)F)cc3)c2)c(-c2ccncc2)n1]

One embodiment provides a compound of Formula (II), or a tautomer, steroisomer, geometric isomer, a pharmaceutically acceptable salt, solvate, or hydrate thereof:

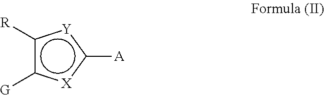

wherein
X is S and Y is N; or
X is N and Y is S; or
X is O and Y is N; or
X is N and Y is O;
R is

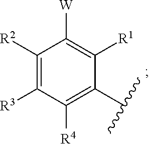

G is selected from:

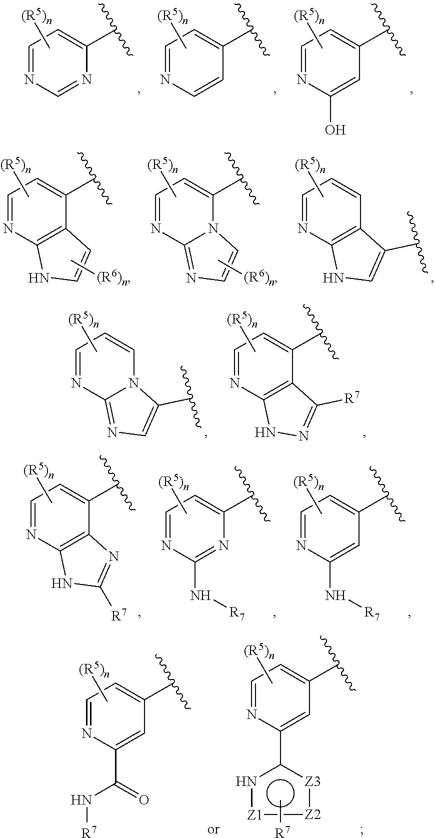

R⁵ and R⁶ are each independently selected from H, optionally substituted alkyl, -optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂₁H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹, R¹⁰, optionally substituted N-attached heterocycloalkyl, optionally substituted C-attached heterocycloalkyl, optionally substituted cycloalkyl, or optionally substituted heteroalkyl;

R¹, R², R³ and R⁴ are each independently selected from hydrogen, F, Cl, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHCOAr, —NHSO₂NHAr, —N(OH)CONHAr, —NHSO₂N(Ar)₂, —NHCONHAr, —NHCON(Ar)₂, —NHCOC(R¹¹)(R¹²)Ar, —C(R¹¹)(R¹²)CONHAr, NHCSNHAr, NHCSN(Ar)₂ or —N(R¹¹)CON(R¹²)Ar;
Ar is:

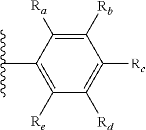

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl), NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently selected from H, or C₁-C₆, alkyl; or for the instance wherein R¹¹ and R¹² are attached germinal carbon substituents, R¹¹ and R¹² together with the carbon atom to which they are attached are joined to form a C₃-C₆ cycloalkyl; and n is 0, 1, or 2.

Another embodiment provides the compound of Formula (II), wherein X is S and Y is N.

Another embodiment provides the compound of Formula (II), wherein X is N and Y is S,

Another embodiment provides the compound of Formula (II) wherein W is NHSO₂Ar.

Another embodiment provides the compound of Formula (II), wherein W is NHCONHAr.

Another embodiment provides the compound of Formula (II) wherein G is

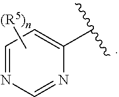

Another embodiment provides the compound of Formula (II), wherein G is

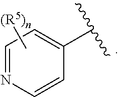

Another embodiment provides the compound of Formula (II), wherein G is

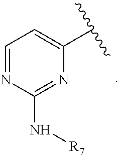

Another embodiment provides the compound of Formula (II), wherein G is

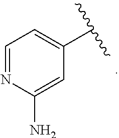 [SMILES: *c1ccnc(N)c1]

Another embodiment provides the compound of Formula (II), wherein A is an optionally substituted alkyl or optionally substituted cycloalkyl. Another embodiment provides the compound of Formula (II), wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (II), wherein A is —NR⁹R¹⁰.

Another embodiment provides the compound of Formula (II), wherein A is an optionally C-attached heterocycloalkyl.

Another embodiment provides the compound of Formula (II), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or —C₁-C₅ optionally substituted alkyl. Another embodiment provides the compound of Formula (II), wherein R³ and R⁴ are hydrogen. Another embodiment provides the compound of Formula (II), wherein R¹ and R² are each independently selected from hydrogen; F, Cl, CN; OH, CH₂F, CHF₂, CF₃, or C₂F₅.

Another embodiment provides the compound of Formula (II), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

Another embodiment provides the compound of Formula (II) selected from the following:

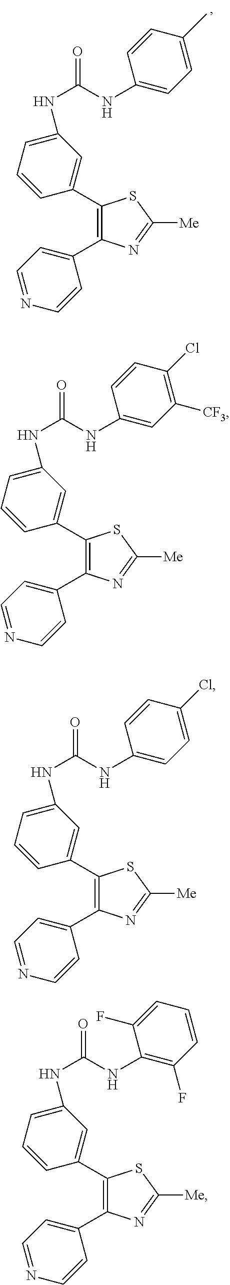 [SMILES: Cc1ccc(NC(=O)Nc2cccc(-c3sc(C)nc3-c3ccncc3)c2)cc1 ; Cc1nc(-c2ccncc2)c(-c2cccc(NC(=O)Nc3ccc(Cl)c(C(F)(F)F)c3)c2)s1 ; Cc1nc(-c2ccncc2)c(-c2cccc(NC(=O)Nc3ccc(Cl)cc3)c2)s1 ; Cc1nc(-c2ccncc2)c(-c2cccc(NC(=O)Nc3c(F)cccc3F)c2)s1]

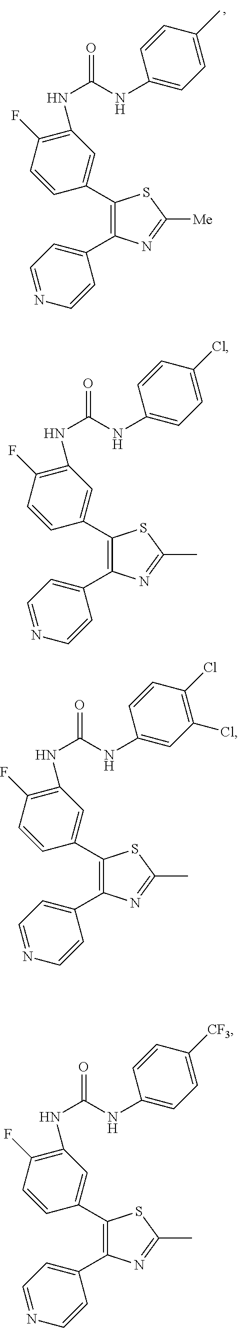 [SMILES: Cc1ccc(NC(=O)Nc2cc(-c3sc(C)nc3-c3ccncc3)ccc2F)cc1 ; Cc1nc(-c2ccncc2)c(-c2ccc(F)c(NC(=O)Nc3ccc(Cl)cc3)c2)s1 ; Cc1nc(-c2ccncc2)c(-c2ccc(F)c(NC(=O)Nc3ccc(Cl)c(Cl)c3)c2)s1 ; Cc1nc(-c2ccncc2)c(-c2ccc(F)c(NC(=O)Nc3ccc(C(F)(F)F)cc3)c2)s1]

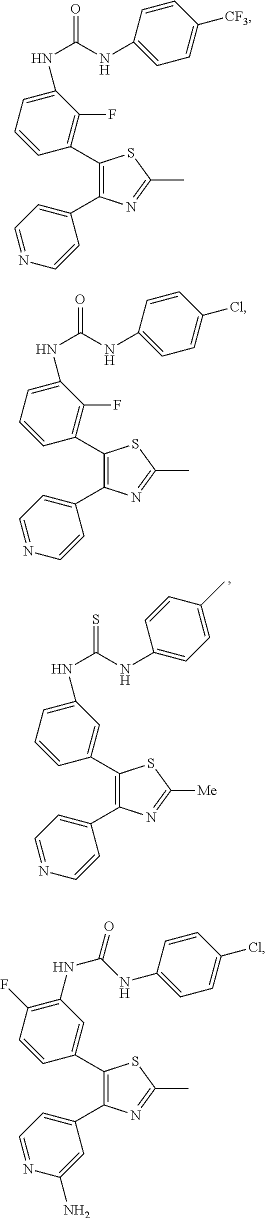

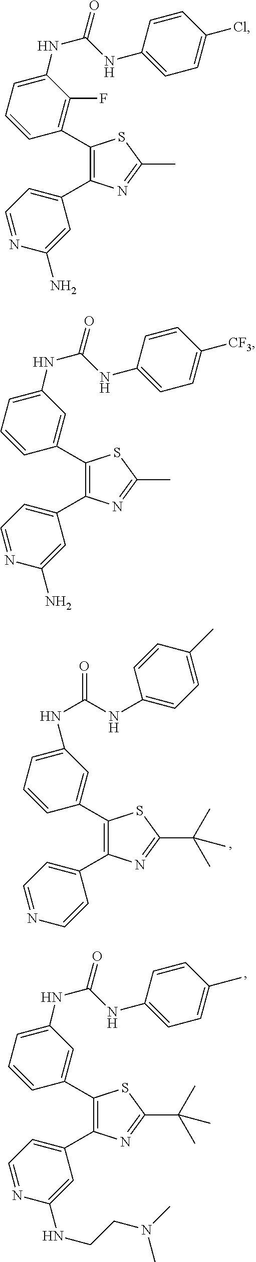

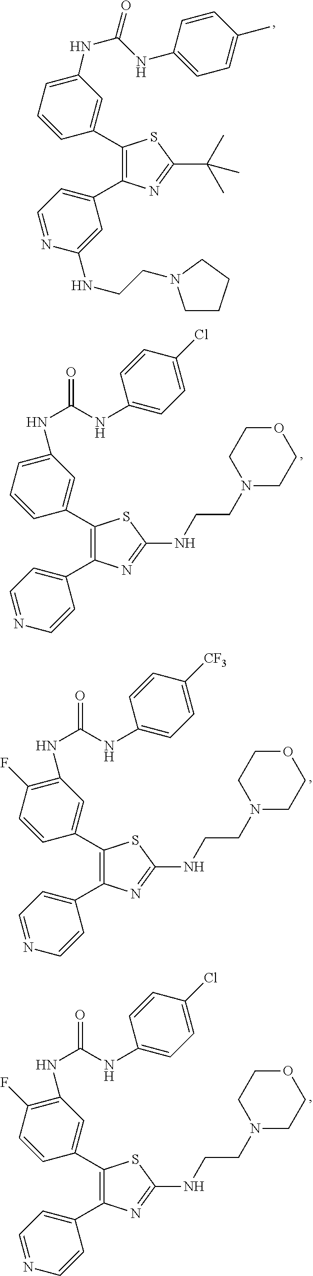

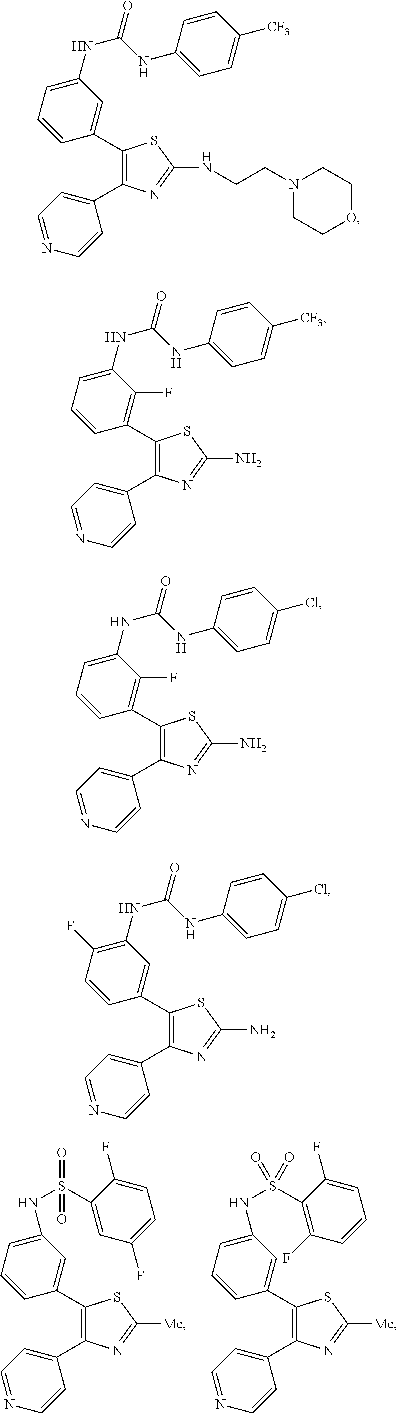

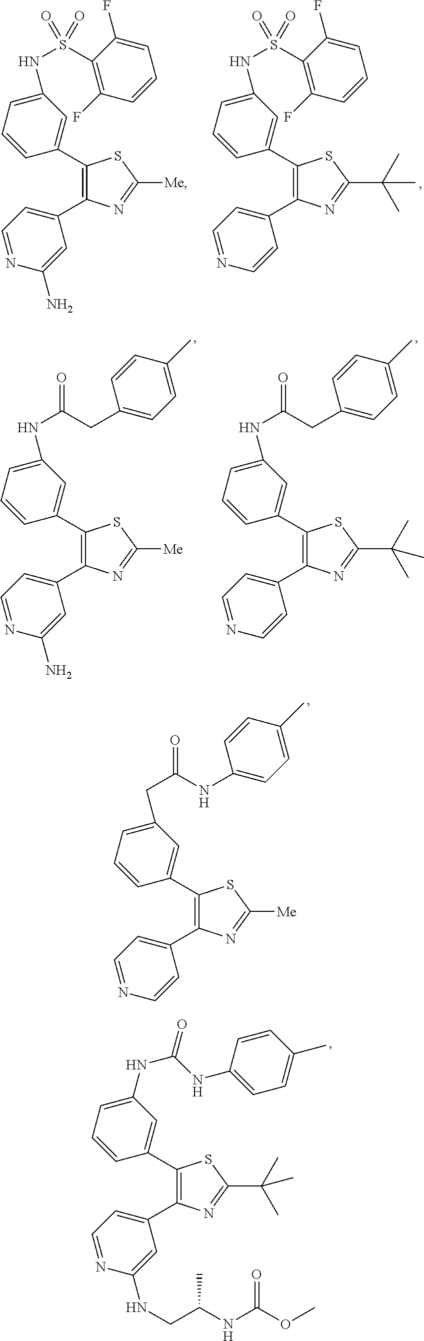

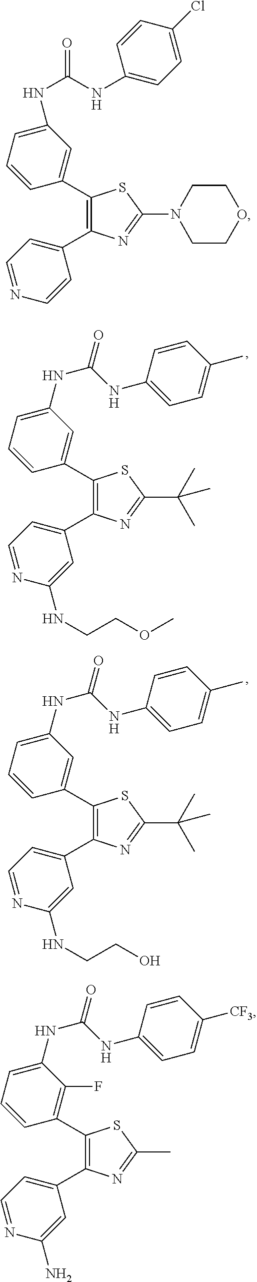

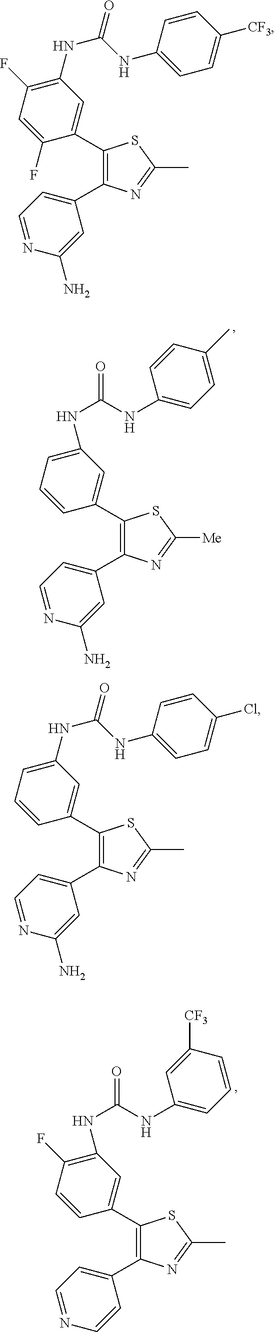

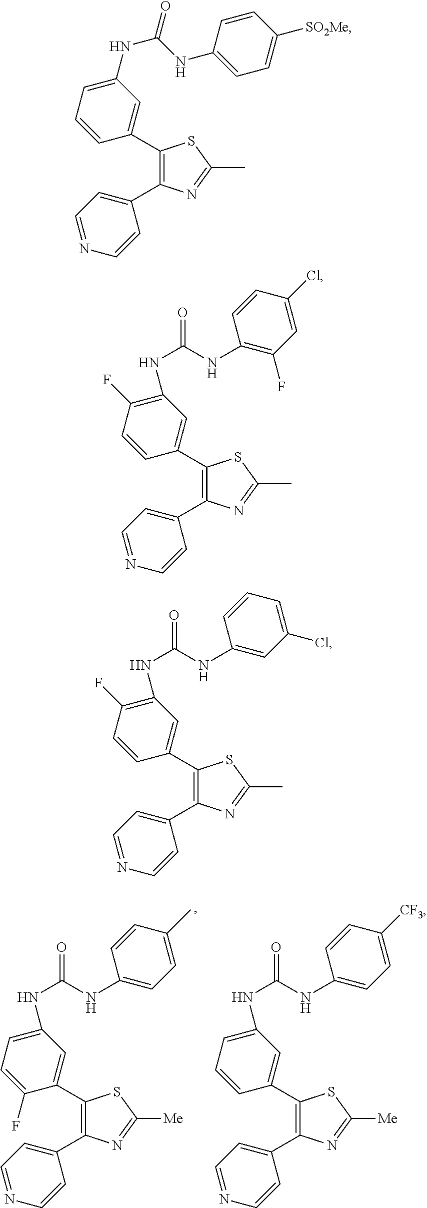

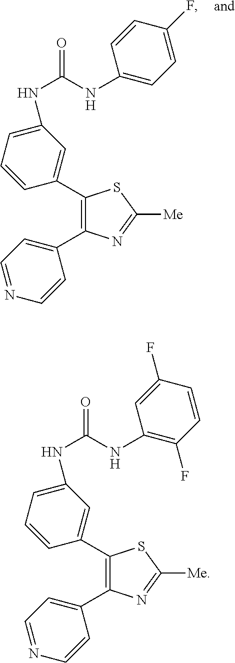 [SMILES: Cc1nc(-c2ccncc2)c(-c2cccc(NC(=O)Nc3ccc(F)cc3)c2)s1 ; "and" ; Cc1nc(-c2ccncc2)c(-c2cccc(NC(=O)Nc3cc(F)ccc3F)c2)s1]

One embodiment provides a compound of Formula (III), or a tautomer, steroisomer, geometric isomer, a pharmaceutically acceptable salt, solvate, or hydrate thereof:

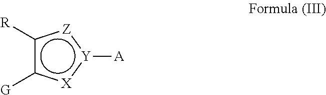

wherein
Z is N, Y is C, and X is NH;
or
Z is N, Y is N, and X is CH;
or
Z is CH, Y is N, and X is N;
or
Z is N, Y is N, and X is N;
R is

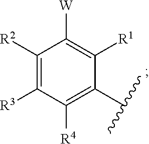

G is selected from:

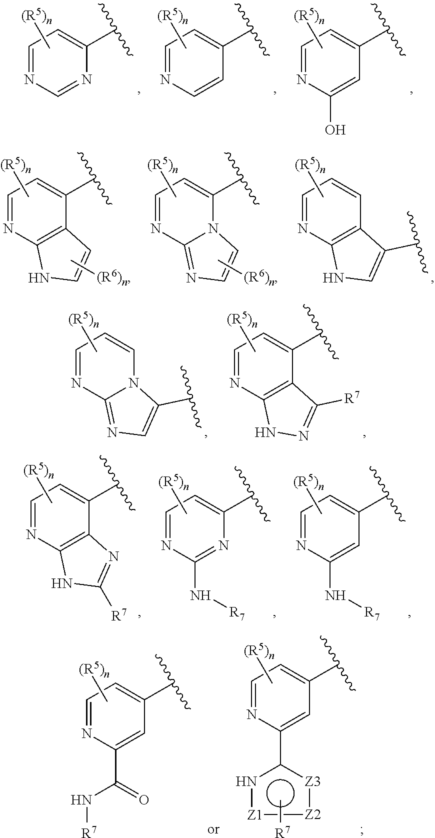

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(Optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is Nor C(R⁵);
Z2 is N or C(R⁵);
Z3 is N or C(R⁵);

A is selected from H, alkyl, optionally substituted alkyl, —NR⁹R¹⁰, optionally substituted N-attached heterocycloalkyl, optionally substituted, C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

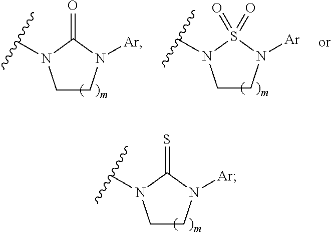

Ar is:

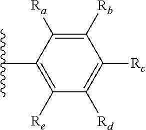

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3; and
n is 0, 1, or 2.

Another embodiment provides the compound of Formula (III), wherein Z is N, Y is C, and X is NH.

Another embodiment provides the compound of Formula (III), wherein Z is N, Y is N, and X is CH.

Another embodiment provides the compound of Formula (III), wherein Z is CH, Y is N, and X is N.

Another embodiment provides the compound of Formula (III), wherein Z is N, Y is N, and X is N.

Another embodiment provides the compound of Formula (III), wherein W is

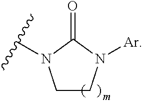

Another embodiment provides the compound of Formula (III), wherein G is

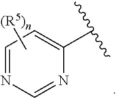

Another embodiment provides the compound of Formula (III), wherein G is

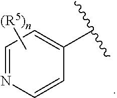

Another embodiment provides the compound of Formula (III), wherein G is

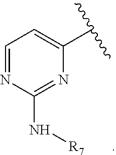

Another embodiment provides the compound of Formula (III), wherein G is

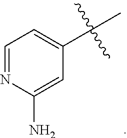 [SMILES: *c1ccnc(N)c1]

Another embodiment provides the compound of Formula (III), wherein A is an optionally substituted alkyl or optionally substituted cycloalkyl.

Another embodiment provides the compound of Formula (III), wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (III), wherein A is NR⁹R¹⁰.

Another embodiment provides the compound of Formula (III), wherein A is an optionally substituted. C-attached heterocycloalkyl.

Another embodiment provides the compound of Formula (III), wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl. Another embodiment provides the compound of Formula (III), wherein R³ and R⁴ are hydrogen. Another embodiment provides the compound of Formula (III), wherein R¹ and R² are each independently selected from hydrogen, F, Cl, CN, OH, CH₂F, CHF₂, CF₃, or C₂F₅.

Another embodiment provides the compound of Formula (III), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂ (C₁-C₅ optionally substituted alkyl).

Another embodiment provides the compound of Formula (III) selected from the following:

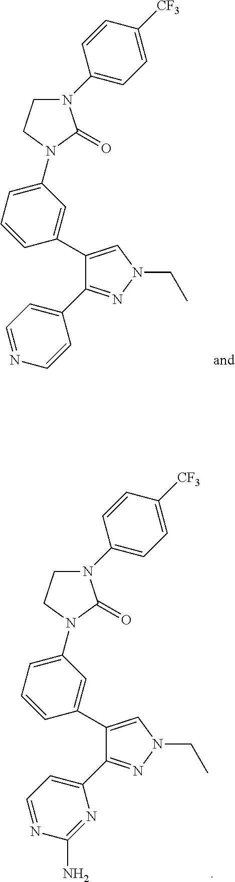 [SMILES: CCn1cc(-c2cccc(N3CCN(c4ccc(C(F)(F)F)cc4)C3=O)c2)c(-c2ccncc2)n1 ; CCn1cc(-c2cccc(N3CCN(c4ccc(C(F)(F)F)cc4)C3=O)c2)c(-c2ccnc(N)n2)n1]

One embodiment provides a compound of Formula (IV), or a tautomer, steroisomer, geometric isomer, a pharmaceutically acceptable salt, solvate, or hydrate thereof:

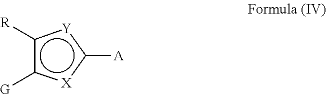

wherein
X is S and Y is N; or
X is N and Y is S; or
X is O and Y is N; or
X is N and Y is O;
R is

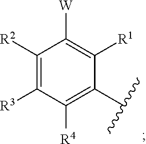

G is selected from:

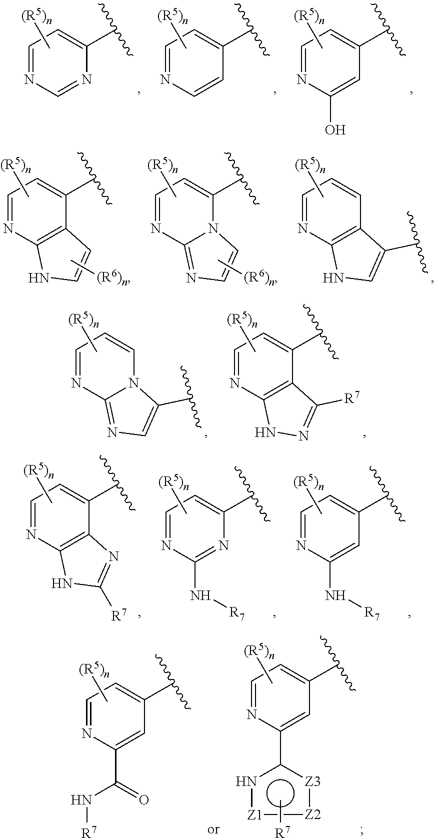

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted-alkylene)-(optionally substituted alkoxy), -(optionally substituted alkylene)-(NHCO₂H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;

R¹, R², R³ and R⁴ are each independently selected from hydrogen, F, Cl, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

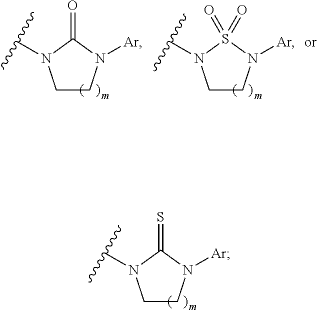

Ar is:

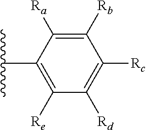

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl); NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3;
n is 0, 1, or 2.

Another embodiment provides the compound of Formula (IV), wherein X is S and Y is N.

Another embodiment provides the compound of Formula (IV), wherein X is N and —Y is S.

Another embodiment provides the compound of Formula (IV), wherein W is

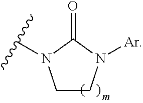

Another embodiment provides the compound of Formula (IV), wherein G is

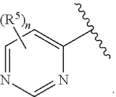

Another embodiment provides the compound of Formula (IV), wherein G is

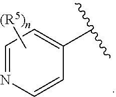

Another embodiment provides the compound of Formula (IV), wherein G is

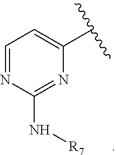

Another embodiment provides the compound of Formula (IV), wherein G is

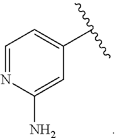 [SMILES: *c1ccnc(N)c1]

Another embodiment provides the compound of Formula (IV), wherein A is an optionally substituted alkyl or optionally substituted cycloalkyl.

Another embodiment provides the compound of Formula (IV), wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.

Another embodiment provides the compound of Formula (IV), wherein A is —NR⁹R¹⁰.

Another embodiment provides the compound of Formula (IV), wherein A is an optionally C-attached heterocycloalkyl.

Another embodiment provides the compound of Formula (IV), wherein R¹, R², R³ and R⁴ are each independently selected from hydrogen, F, Cl, CN, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl.

Another embodiment provides the compound of Formula (IV), wherein R³ and R⁴ are hydrogen.

Another embodiment provides the compound of Formula (IV), wherein R¹ and R² are each independently selected from hydrogen, F, Cl, CN, OH, CH₂F, CHF₂, CF₃, or C₂F₅.

Another embodiment provides the compound of Formula (IV), wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

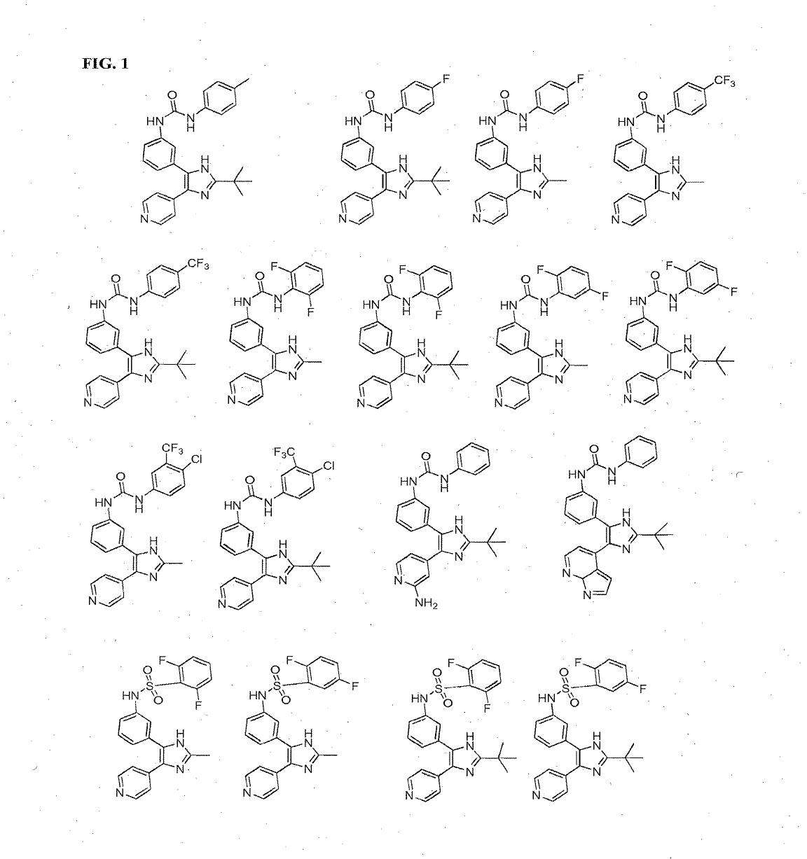

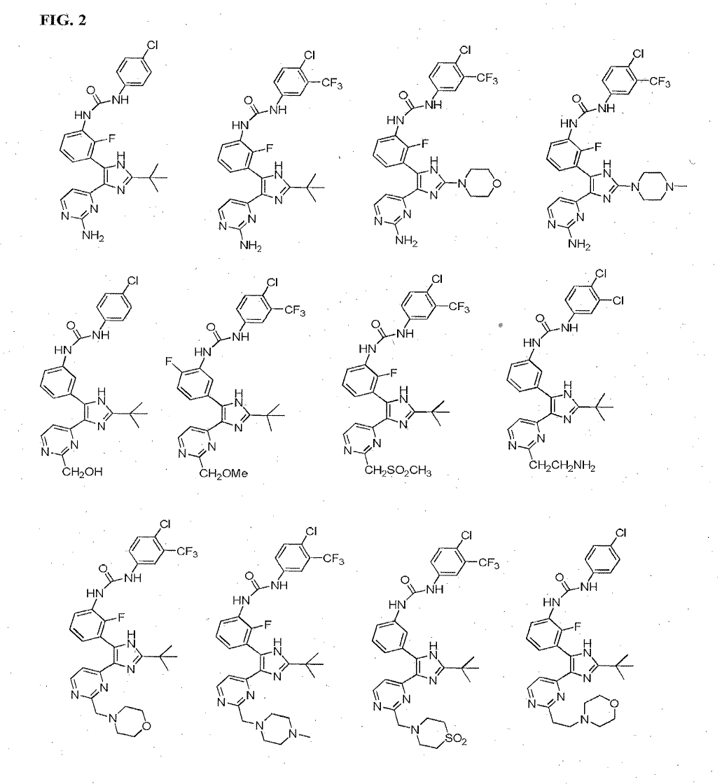

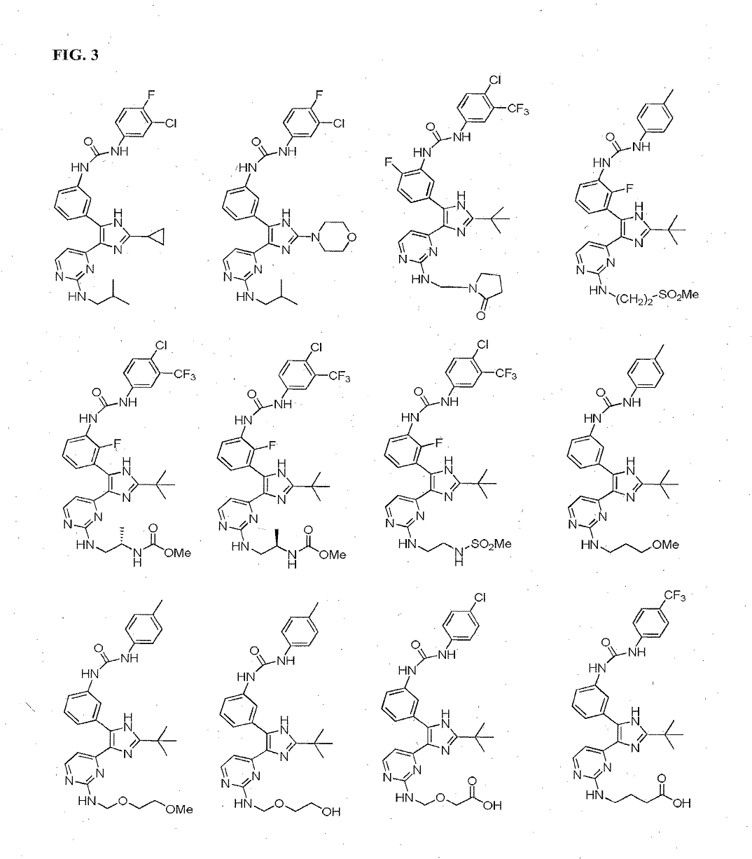

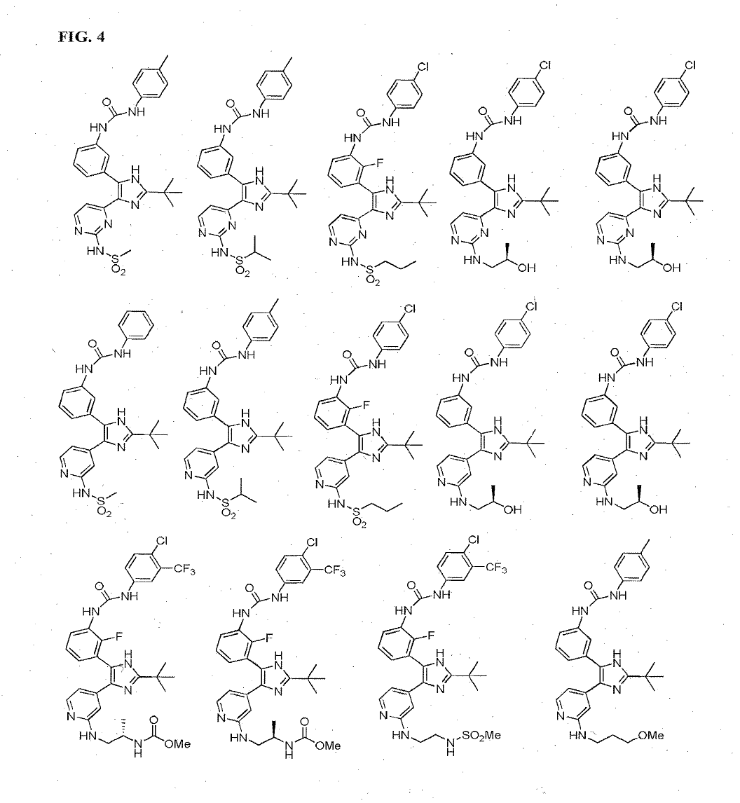

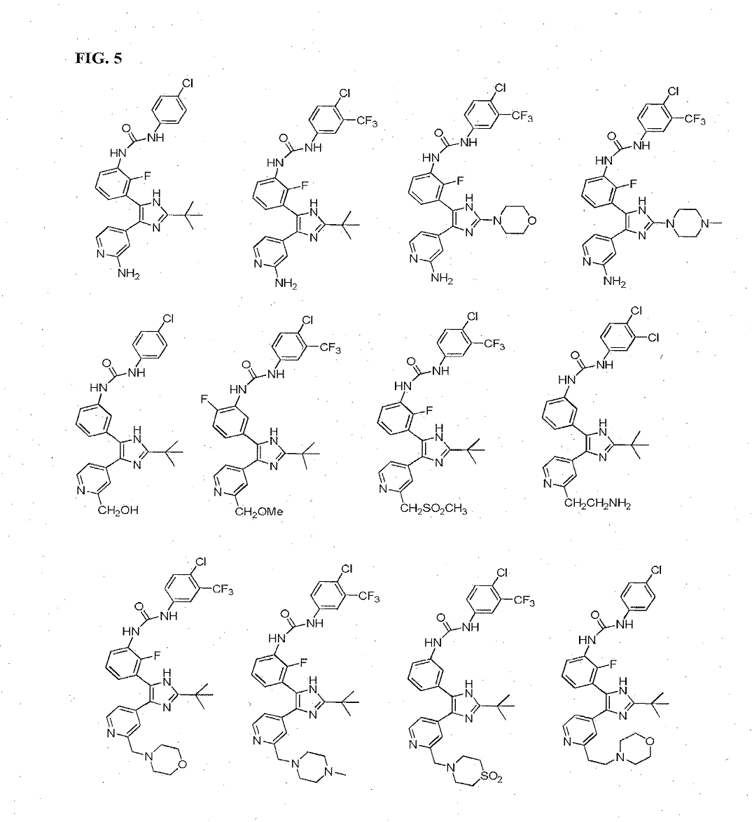

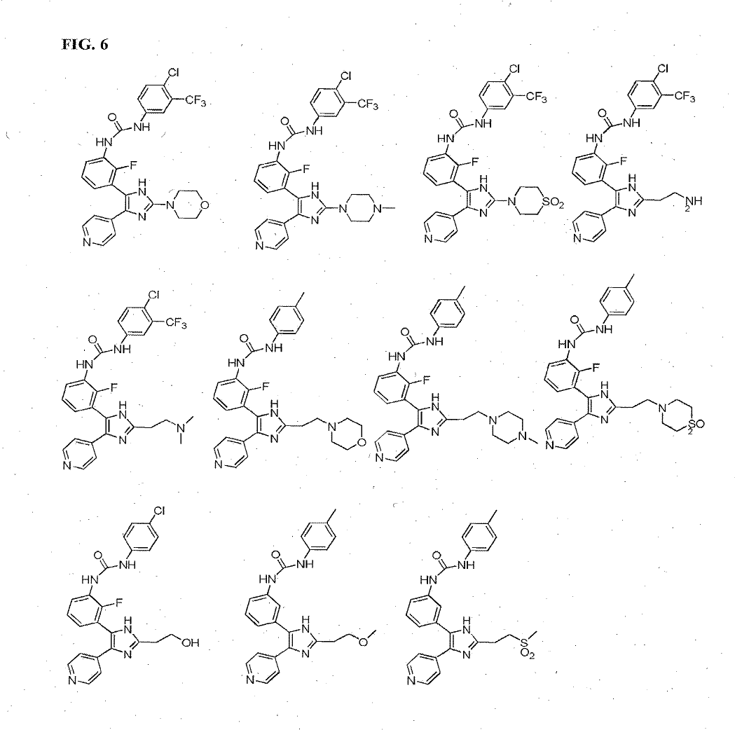

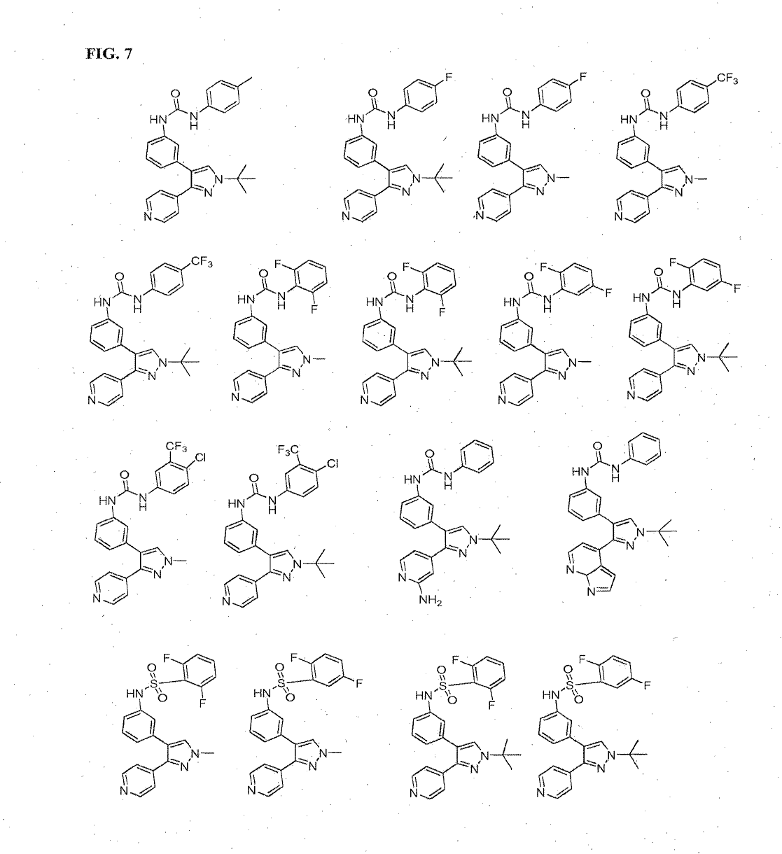

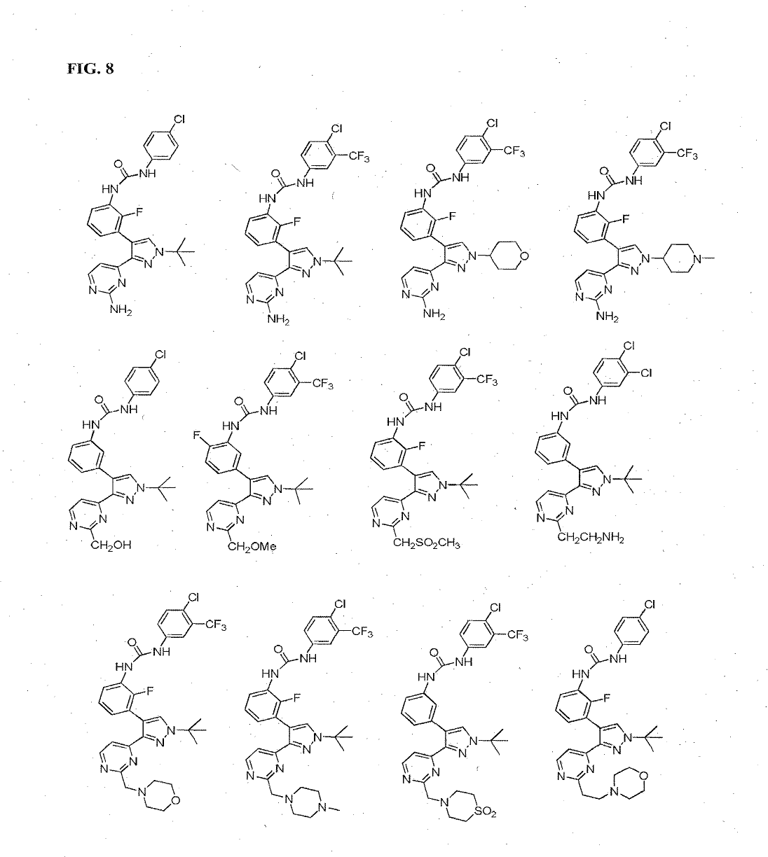

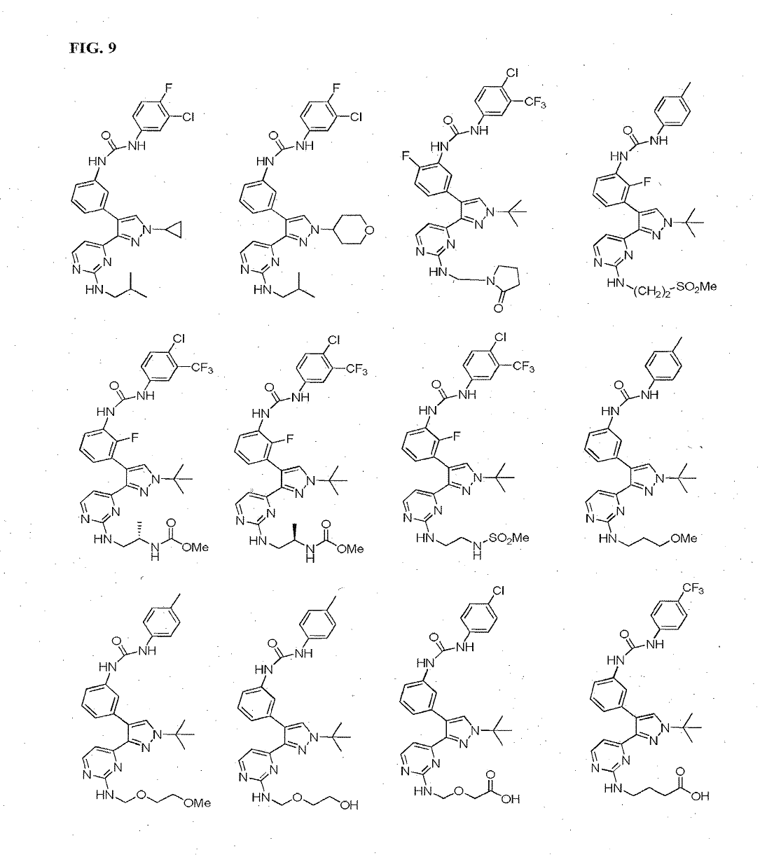

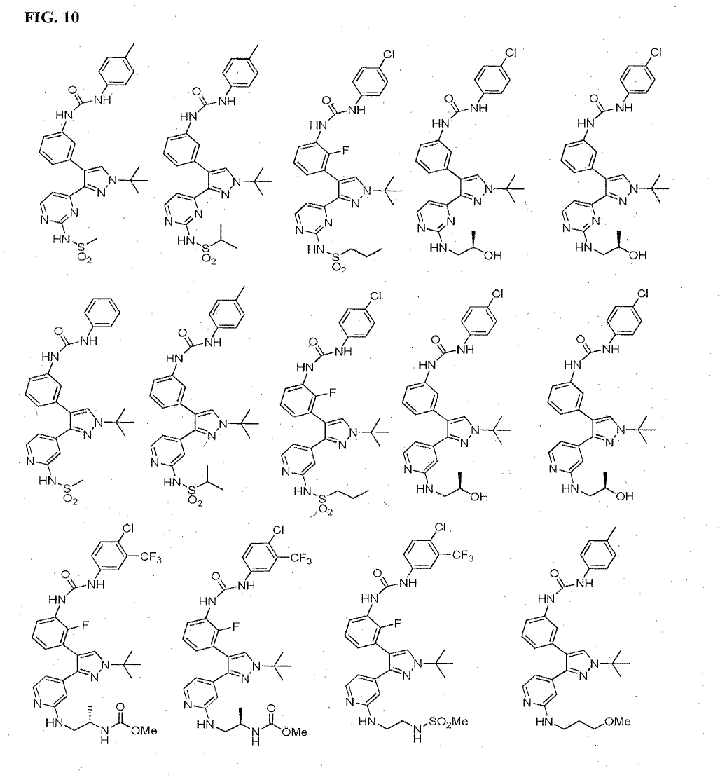

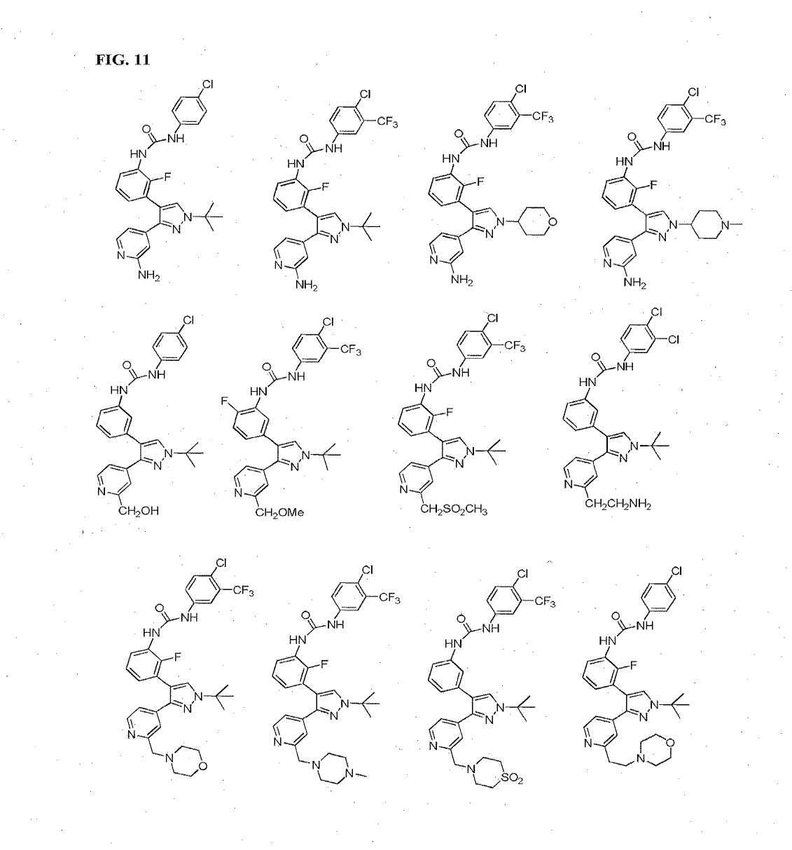

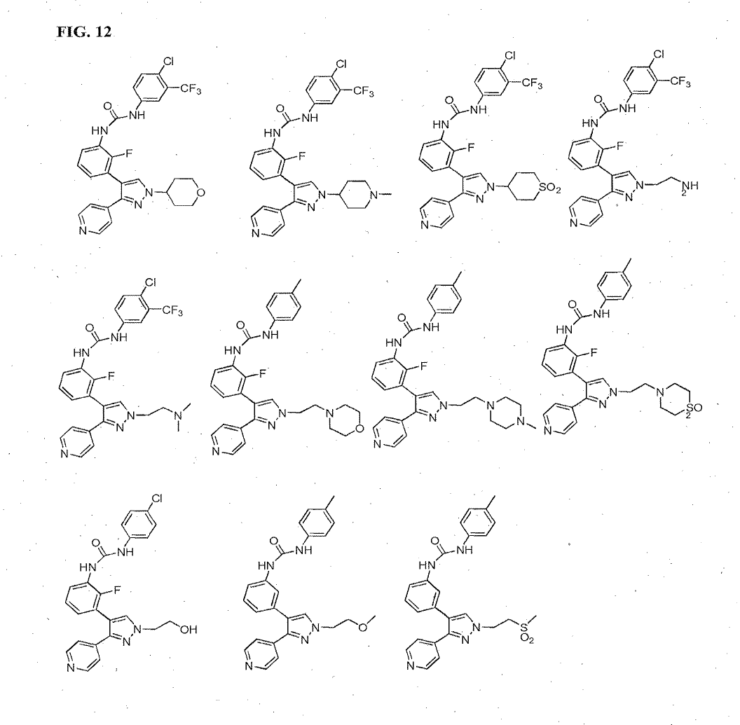

In certain specific embodiments, the compounds of Formula (I) have the structures shown in FIGS. 1 to 12.

In certain specific embodiments, the compounds of Formula (I)-(IV) have the structures shown in Table 1.

TABLE 1

| Example | Structure | Name |
|---|---|---|
| 1 | 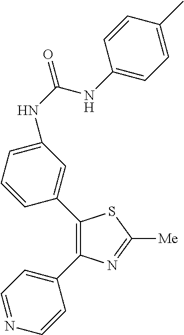 [SMILES: Cc1ccc(NC(=O)Nc2cccc(-c3sc(C)nc3-c3ccncc3)c2)cc1] | 1-(3-(2-methyl-4-(pyridin-4- yl)thiazol-5-yl)phenyl)-3-p- tolylurea |
| 2 | 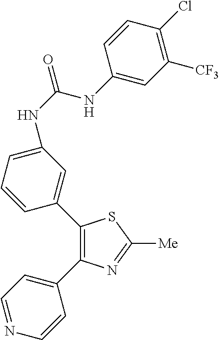 [SMILES: Cc1nc(-c2ccncc2)c(-c2cccc(NC(=O)Nc3ccc(Cl)c(C(F)(F)F)c3)c2)s1] | 1-(4-chloro-3- (trifluoromethyl)phenyl)-3-(3- (2-methyl-4-(pyridin-4- yl)thiazol-5-yl)phenyl)urea |
| 3 | 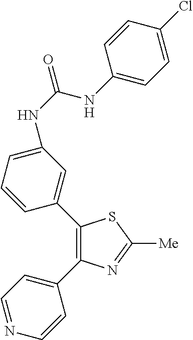 [SMILES: Cc1nc(-c2ccncc2)c(-c2cccc(NC(=O)Nc3ccc(Cl)cc3)c2)s1] | 1-(4-chlorophenyl)-3-(3-(2- methyl-4-(pyridin-4-yl)thiazol- 5-yl)phenyl)urea |
| 4 | 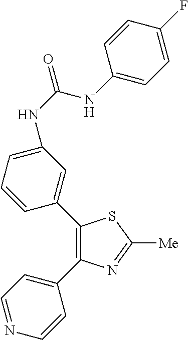 [SMILES: Cc1nc(-c2ccncc2)c(-c2cccc(NC(=O)Nc3ccc(F)cc3)c2)s1] | 1-(4-fluorophenyl)-3-(3-(2- methyl-4-(pyridin-4-yl)thiazol- 5-yl)phenyl)urea |
| 5 | 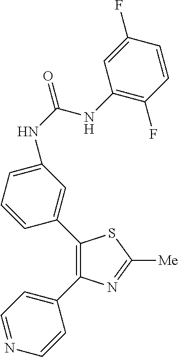 [SMILES: Cc1nc(-c2ccncc2)c(-c2cccc(NC(=O)Nc3cc(F)ccc3F)c2)s1] | 1-(2,5-difluorophenyl)-3-(3-(2- methyl-4-(51yridine-4- yl)thiazol-5-yl)phenyl)urea |
| 6 | 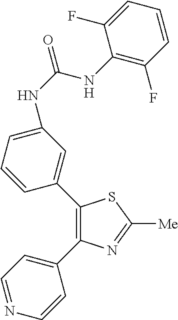 [SMILES: Cc1nc(-c2ccncc2)c(-c2cccc(NC(=O)Nc3c(F)cccc3F)c2)s1] | 1-(2,6-difluorophenyl)-3-(3-(2- methyl-4-(pyridin-4-yl)thiazol- 5-yl)phenyl)urea |
| 7 | 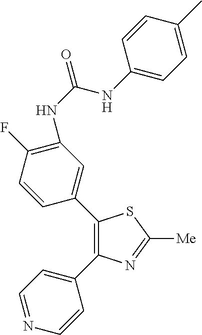 [SMILES: Cc1ccc(NC(=O)Nc2cc(-c3sc(C)nc3-c3ccncc3)ccc2F)cc1] | 1-(2-fluoro-5-(2-methyl-4- (pyridin-4-yl)thiazol-5- yl)phenyl)-3-p-tolylurea |
| 8 | 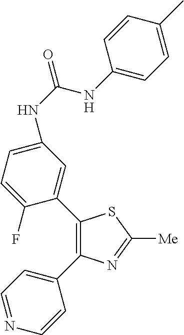 [SMILES: Cc1ccc(NC(=O)Nc2ccc(F)c(-c3sc(C)nc3-c3ccncc3)c2)cc1] | 1-(4-fluoro-3-(2-methyl-4- (pyridin-4-yl)thiazol-5- yl)phenyl)-3-p-tolylurea |
| 9 | 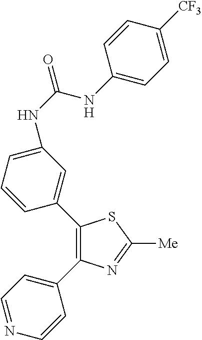 [SMILES: Cc1nc(-c2ccncc2)c(-c2cccc(NC(=O)Nc3ccc(C(F)(F)F)cc3)c2)s1] | 1-(3-(2-methyl-4-(pyridin-4- yl)thiazol-5-yl)phenyl)-3-(4- (trifluoromethyl)phenyl)urea |
| 10 | 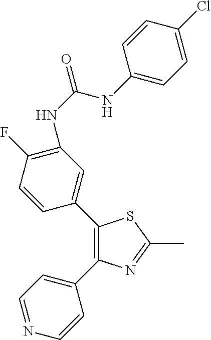 [SMILES: Cc1nc(-c2ccncc2)c(-c2ccc(F)c(NC(=O)Nc3ccc(Cl)cc3)c2)s1] | 1-(3-chlorophenyl)-3-(2-fluoro- 5-(2-methyl-4-(pyridin-4- yl)thiazol-5-yl)phenyl)urea |
| 11 | 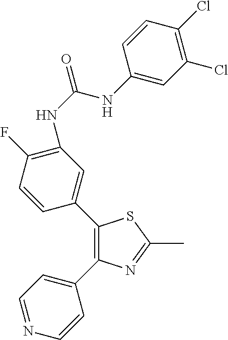 [SMILES: Cc1nc(-c2ccncc2)c(-c2ccc(F)c(NC(=O)Nc3ccc(Cl)c(Cl)c3)c2)s1] | 1-(3,4-dichlorophenyl)-3-(2- fluoro-5-(2-methyl-4-(pyridin-4- yl)thiazol-5-yl)phenyl)urea |
| 12 | 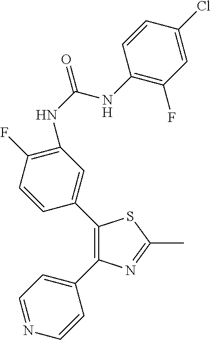 [SMILES: Cc1nc(-c2ccncc2)c(-c2ccc(F)c(NC(=O)Nc3ccc(Cl)cc3F)c2)s1] | 1-(4-chloro-2-fluorophenyl)-3- (2-fluoro-5-(2-methyl-4- (pyridin-4-yl)thiazol-5- yl)phenyl)urea |
| 13 | 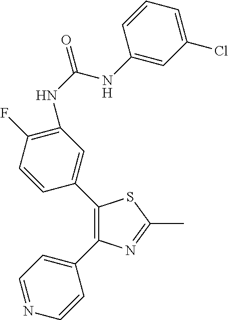 [SMILES: Cc1nc(-c2ccncc2)c(-c2ccc(F)c(NC(=O)Nc3cccc(Cl)c3)c2)s1] | 1-(3-chlorophenyl)-3-(2-fluoro- 5-(2-methyl-4-(pyridin-4- yl)thiazol-5-yl)phenyl)urea |
| 14 | 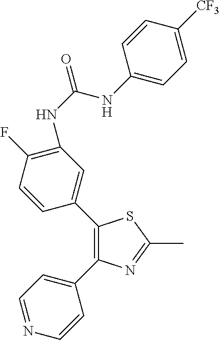 [SMILES: Cc1nc(-c2ccncc2)c(-c2ccc(F)c(NC(=O)Nc3ccc(C(F)(F)F)cc3)c2)s1] | 1-(2-fluoro-5-(2-methyl-4- (pyridin-4-yl)thiazol-5- yl)phenyl)-3-(4- (trifluoromethyl)phenyl)urea |
| 15 | 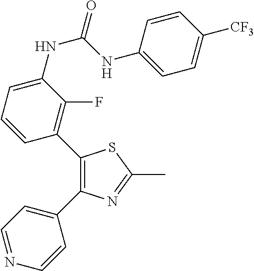 [SMILES: Cc1nc(-c2ccncc2)c(-c2cccc(NC(=O)Nc3ccc(C(F)(F)F)cc3)c2F)s1] | 1-(2-fluoro-3-(2-methyl-4- (pyridin-4-yl)thiazol-5- yl)phenyl)-3-(4- (trifluoromethyl)phenyl)urea |
| 16 | 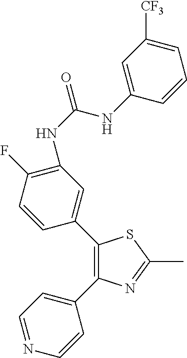 [SMILES: Cc1nc(-c2ccncc2)c(-c2ccc(F)c(NC(=O)Nc3cccc(C(F)(F)F)c3)c2)s1] | 1-(2-fluoro-5-(2-methyl-4- (pyridin-4-yl)thiazol-5- yl)phenyl)-3-(3- (trifluoromethyl)phenyl)urea |
| 17 | 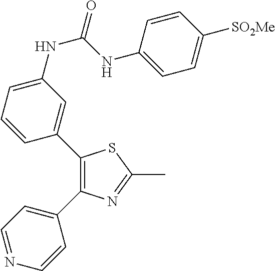 [SMILES: Cc1nc(-c2ccncc2)c(-c2cccc(NC(=O)Nc3ccc(S(C)(=O)=O)cc3)c2)s1] | 1-(3-(2-methyl-4-(pyridin-4- yl)thiazol-5-yl)phenyl)-3-(4- (methylsulfonyl)phenyl)urea |
| 18 | 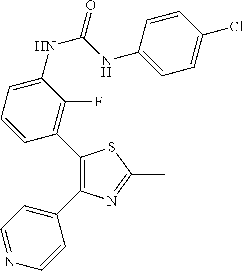 [SMILES: Cc1nc(-c2ccncc2)c(-c2cccc(NC(=O)Nc3ccc(Cl)cc3)c2F)s1] | 1-(4-chlorophenyl)-3-(2-fluoro- 3-(2-methyl-4-(pyridin-4- yl)thiazol-5-yl)phenyl)urea |
| 19 | 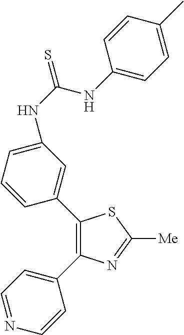 [SMILES: Cc1ccc(NC(=S)Nc2cccc(-c3sc(C)nc3-c3ccncc3)c2)cc1] | 111-(3-(2-methyl-4-(pyridin-4- yl)thiazol-5-yl)phenyl)-3-p- tolylthiourea |
| 20 | 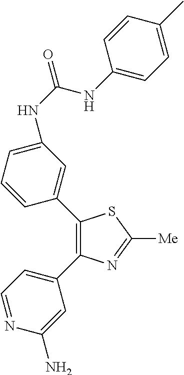 [SMILES: Cc1ccc(NC(=O)Nc2cccc(-c3sc(C)nc3-c3ccnc(N)c3)c2)cc1] | 1-(3-(2-methyl-4-(pyridin-4- yl)thiazol-5-yl)phenyl)-3-p- tolylthiourea |
| 21 | 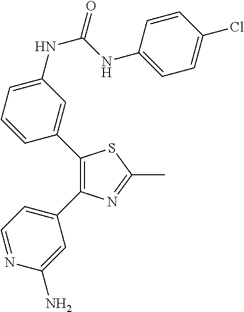 [SMILES: Cc1nc(-c2ccnc(N)c2)c(-c2cccc(NC(=O)Nc3ccc(Cl)cc3)c2)s1] | 1-(3-(4-(2-aminopyridin-4-yl)-2- methylthiazol-5-yl)phenyl)-3-(4- chlorophenyl)urea |
| 22 | 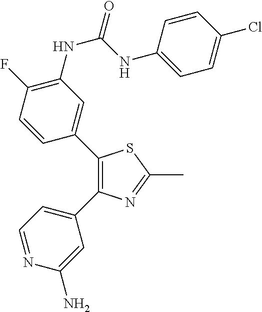 [SMILES: Cc1nc(-c2ccnc(N)c2)c(-c2ccc(F)c(NC(=O)Nc3ccc(Cl)cc3)c2)s1] | 1-(5-(4-(2-aminopyridin-4-yl)-2- methylthiazol-5-yl)-2- fluorophenyl)-3-(4- chlorophenyl)urea |
| 23 | 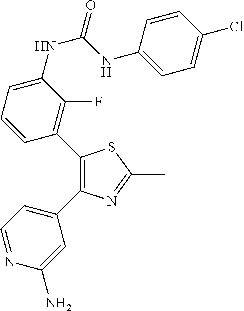 [SMILES: Cc1nc(-c2ccnc(N)c2)c(-c2cccc(NC(=O)Nc3ccc(Cl)cc3)c2F)s1] | 1-(4-chlorophenyl)-3-(2-fluoro- 3-(2-methyl-4-(pyridin-4- yl)thiazol-5-yl)phenyl)urea |
| 24 | 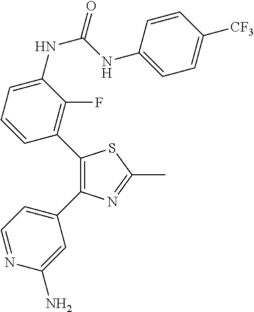 [SMILES: Cc1nc(-c2ccnc(N)c2)c(-c2cccc(NC(=O)Nc3ccc(C(F)(F)F)cc3)c2F)s1] | 1-(3-(4-(2-aminopyridin-4-yl)-2- methylthiazol-5-yl)-2- fluorophenyl)-3-(4- (trifluoromethyl)phenyl)urea |
| 25 | 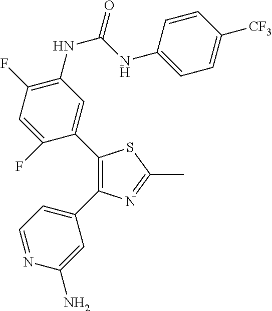 [SMILES: Cc1nc(-c2ccnc(N)c2)c(-c2cc(NC(=O)Nc3ccc(C(F)(F)F)cc3)c(F)cc2F)s1] | 1-(5-(4-(2-aminopyridin-4-yl)-2- methylthiazol-5-yl)-2,4- difluorophenyl)-3-(4- (trifluoromethyl)phenyl)urea |
| 26 | 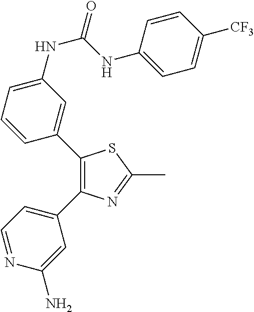 [SMILES: Cc1nc(-c2ccnc(N)c2)c(-c2cccc(NC(=O)Nc3ccc(C(F)(F)F)cc3)c2)s1] | 1-(3-(4-(2-aminopyridin-4-yl)-2- methylthiazol-5-yl)phenyl)-3-(4- (trifluorometbyl)phenyl)urea |
| 27 | 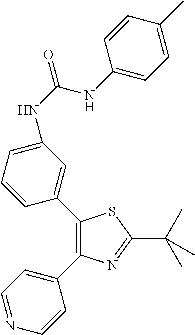 [SMILES: Cc1ccc(NC(=O)Nc2cccc(-c3sc(C(C)(C)C)nc3-c3ccncc3)c2)cc1] | 1-(3-(2-tert-butyl-4-(pyridin-4- yl)thiazol-5-yl)phenyl)-3-p- tolylurea |
| 28 | 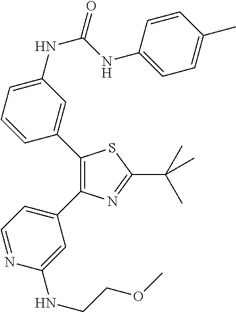 [SMILES: COCCNc1cc(-c2nc(C(C)(C)C)sc2-c2cccc(NC(=O)Nc3ccc(C)cc3)c2)ccn1] | 1-(3-(2-tert-butyl-4-(2-(2- methoxyethylamino)pyridin-4- yl)thiazol-5-yl)phenyl)-3-p- tolylurea |
| 29 | 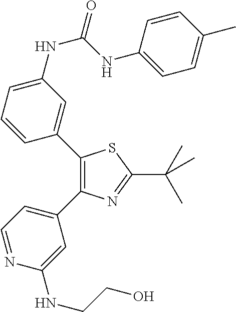 [SMILES: Cc1ccc(NC(=O)Nc2cccc(-c3sc(C(C)(C)C)nc3-c3ccnc(NCCO)c3)c2)cc1] | 1-(3-(2-tert-butyl-4-(2-(2- hydroxyethylamino)pyridin-4- yl)thiazol-5-yl)phenyl)-3-p- tolylurea |
| 30 | 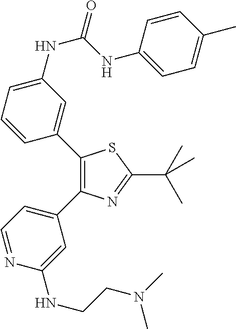 [SMILES: Cc1ccc(NC(=O)Nc2cccc(-c3sc(C(C)(C)C)nc3-c3ccnc(NCCN(C)C)c3)c2)cc1] | 1-(3-(2-tert-butyl-4-(2-(2- (dimethylamino)ethylamino)pyri- din-4-yl)thiazol-5-yl)phenyl)-3- p-tolylurea |
| 31 | 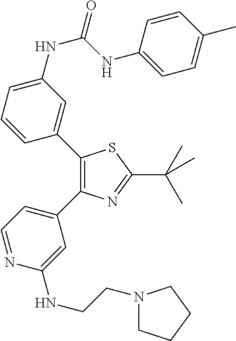 [SMILES: Cc1ccc(NC(=O)Nc2cccc(-c3sc(C(C)(C)C)nc3-c3ccnc(NCCN4CCCC4)c3)c2)cc1] | 1-(3-(2-tert-butyl-4-(2-(2- (pyrrolidin-1- yl)ethylamino)pyridin-4- yl)thiazol-5-yl)phenyl)-3-p- tolylurea |
| 32 | 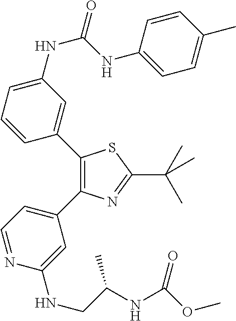 [SMILES: COC(=O)N[C@@H](C)CNc1cc(-c2nc(C(C)(C)C)sc2-c2cccc(NC(=O)Nc3ccc(C)cc3)c2)ccn1] | (S)-methyl 1-(4-(2-tert-butyl-5- (3-(3-p- tolylureido)phenyl)thiazol-4- yl)pyridin-2-ylamino)propan-2- ylcarbamate |
| 33 | 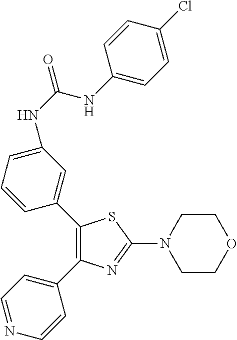 [SMILES: O=C(Nc1ccc(Cl)cc1)Nc1cccc(-c2sc(N3CCOCC3)nc2-c2ccncc2)c1] | 1-(4-chlorophenyl)-3-(3-(2- morpholino-4-(pyridin-4- yl)thiazol-5-yl)phenyl)urea |
| 34 | 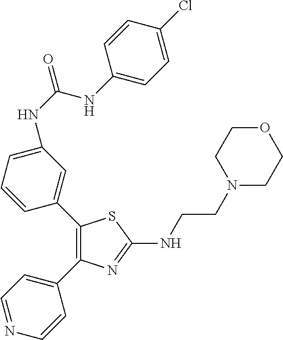 [SMILES: O=C(Nc1ccc(Cl)cc1)Nc1cccc(-c2sc(NCCN3CCOCC3)nc2-c2ccncc2)c1] | 1-(4-chlorophenyl)-3-(3-(2-(2- morpholinoethylamino)-4- (pyridin-4-yl)thiazol-5- yl)phenyl)urea |
| 35 | 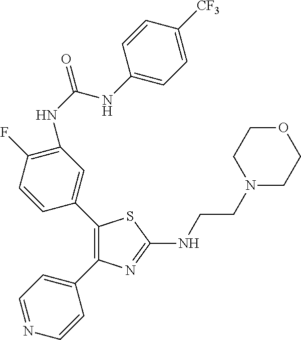 [SMILES: O=C(Nc1ccc(C(F)(F)F)cc1)Nc1cc(-c2sc(NCCN3CCOCC3)nc2-c2ccncc2)ccc1F] | 1-(2-fluoro-5-(2-(2- morpholinoethylamino)-4- (pyridin-4-yl)thiazol-5- yl)phenyl)-3-(4- (trifluoromethyl)phenyl)urea |
| 36 | 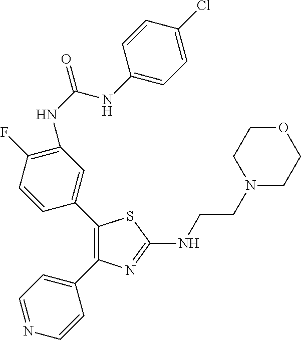 [SMILES: O=C(Nc1ccc(Cl)cc1)Nc1cc(-c2sc(NCCN3CCOCC3)nc2-c2ccncc2)ccc1F] | 1-(4-chlorophenyl)-3-(2-fluoro- 5-(2-(2-morpholinoethylamino)- 4-(pyridin-4-yl)thiazol-5- yl)phenyl)urea |
| 37 | 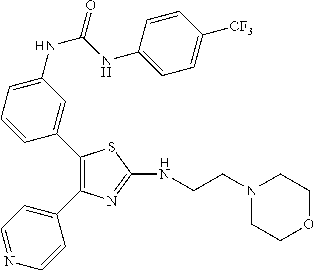 [SMILES: O=C(Nc1ccc(C(F)(F)F)cc1)Nc1cccc(-c2sc(NCCN3CCOCC3)nc2-c2ccncc2)c1] | 1-(3-(2-(2- morpholinoethylamino)-4- (pyridin-4-yl)thiazol-5- yl)phenyl)-3-(4- (trifluoromethyl)phenyl)urea |
| 38 | 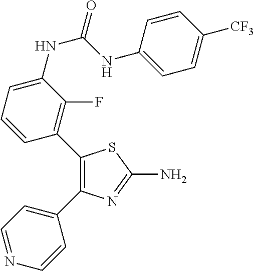 [SMILES: Nc1nc(-c2ccncc2)c(-c2cccc(NC(=O)Nc3ccc(C(F)(F)F)cc3)c2F)s1] | 1-(3-(2-amino-4-(pyridin-4- yl)thiazol-5-yl)-2-fluorophenyl)- 3-(4- (trifluoromethyl)phenyl)urea |
| 39 | 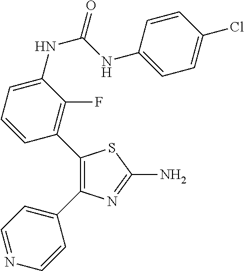 [SMILES: Nc1nc(-c2ccncc2)c(-c2cccc(NC(=O)Nc3ccc(Cl)cc3)c2F)s1] | 1-(3-(2-amino-4-(pyridin-4- yl)thiazol-5-yl)-2-fluorophenyl)- 3-(4-chlorophenyl)urea |
| 40 | 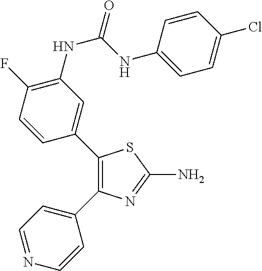 [SMILES: Nc1nc(-c2ccncc2)c(-c2ccc(F)c(NC(=O)Nc3ccc(Cl)cc3)c2)s1] | 1-(5-(2-amino-4-(pyridin-4- yl)thiazol-5-yl)-2-fluorophenyl)- 3-(4-chlorophenyl)urea |
| 41 | 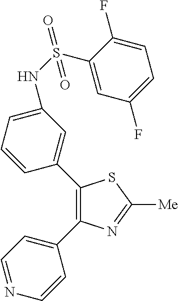 [SMILES: Cc1nc(-c2ccncc2)c(-c2cccc(NS(=O)(=O)c3cc(F)ccc3F)c2)s1] | 2,5-difluoro-N-(3-(2-methyl-4- (pyridin-4-yl)thiazol-5- yl)phenyl)benzenesulfonamide |
| 42 | 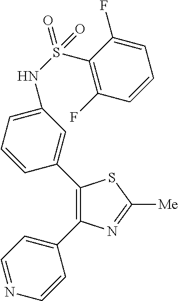 [SMILES: Cc1nc(-c2ccncc2)c(-c2cccc(NS(=O)(=O)c3c(F)cccc3F)c2)s1] | 2,6-difluoro-N-(3-(2-methyl-4- (pyridin-4-yl)thiazol-5- yl)phenyl)benzenesulfonamide |
| 43 | 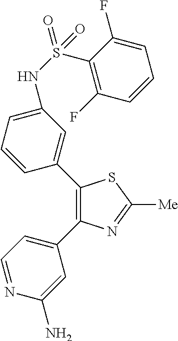 [SMILES: Cc1nc(-c2ccnc(N)c2)c(-c2cccc(NS(=O)(=O)c3c(F)cccc3F)c2)s1] | N-(3-(4-(2-aminopyridin-4-yl)- 2-methylthiazol-5-yl)phenyl)- 2,6-difluorobenzenesulfonamide |
| 44 | 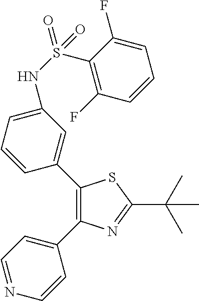 [SMILES: CC(C)(C)c1nc(-c2ccncc2)c(-c2cccc(NS(=O)(=O)c3c(F)cccc3F)c2)s1] | N-(3-(2-tert-butyl-4-(pyridin-4- yl)thiazol-5-yl)phenyl)-2,6- difluorobenzenesulfonamide |
| 45 | 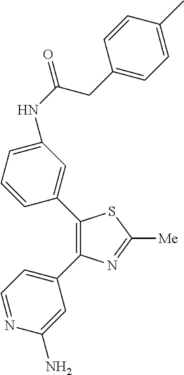 [SMILES: Cc1ccc(CC(=O)Nc2cccc(-c3sc(C)nc3-c3ccnc(N)c3)c2)cc1] | N-(3-(4-(2-aminopyridin-4-yl)- 2-methylthiazol-5-yl)phenyl)-2- p-tolylacetamide |
| 46 | 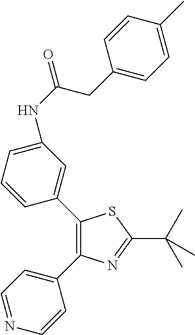 [SMILES: Cc1ccc(CC(=O)Nc2cccc(-c3sc(C(C)(C)C)nc3-c3ccncc3)c2)cc1] | N-(3-(2-tert-butyl-4-(pyridin-4- yl)thiazol-5-yl)phenyl)-2-p- tolylacetamide |
| 47 | 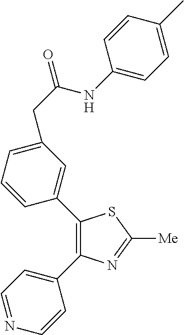 [SMILES: Cc1ccc(NC(=O)Cc2cccc(-c3sc(C)nc3-c3ccncc3)c2)cc1] | 2-(3-(2-methyl-4-(pyridin-4- yl)thiazol-5-yl)phenyl)-N-p- tolylacetamide |
| 48 | 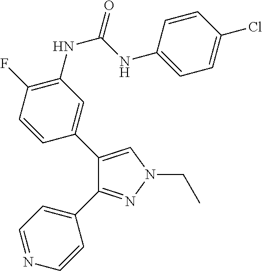 [SMILES: CCn1cc(-c2ccc(F)c(NC(=O)Nc3ccc(Cl)cc3)c2)c(-c2ccncc2)n1] | 1-(4-chlorophenyl)-3-(5-(1- ethyl-3-(pyridin-4-yl)-1H- pyrazol-4-yl)-2- fluorophenyl)urea |
| 49 | 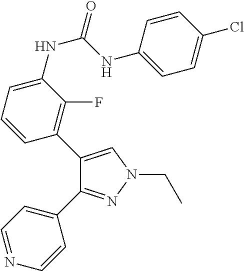 [SMILES: CCn1cc(-c2cccc(NC(=O)Nc3ccc(Cl)cc3)c2F)c(-c2ccncc2)n1] | 1-(4-chlorophenyl)-3-(3-(1- ethyl-3-(pyridin-4-yl)-1H- pyrazol-4-yl)-2- fluorophenyl)urea |
| 50 | 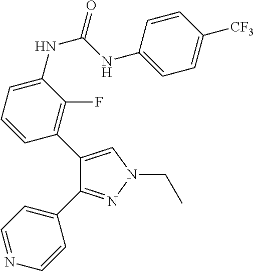 [SMILES: CCn1cc(-c2cccc(NC(=O)Nc3ccc(C(F)(F)F)cc3)c2F)c(-c2ccncc2)n1] | 1-(3-(1-ethyl-3-(pyridin-4-yl)- 1H-pyrazol-4-yl)-2- fluorophenyl)-3-(4- (trifluoromethyl)phenyl)urea |
| 51 | 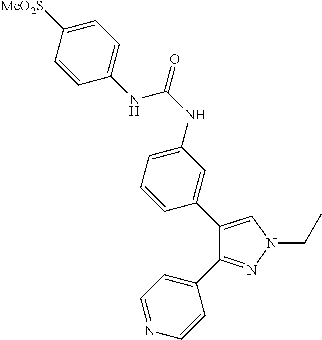 [SMILES: CCn1cc(-c2cccc(NC(=O)Nc3ccc(S(C)(=O)=O)cc3)c2)c(-c2ccncc2)n1] | 1-(3-(1-ethyl-3-(pyridin-4-yl)- 1H-pyrazol-4-yl)phenyl)-3-(4- (methylsulfonyl)phenyl)urea |
| 52 | 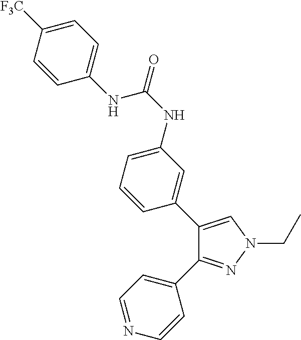 [SMILES: CCn1cc(-c2cccc(NC(=O)Nc3ccc(C(F)(F)F)cc3)c2)c(-c2ccncc2)n1] | 1-(3-(1-ethyl-3-(pyridin-4-yl)- 1H-pyrazol-4-yl)phenyl)-3-(4- (trifluoromethyl)phenyl)urea |
| 53 | 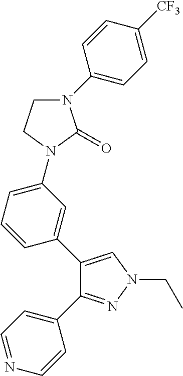 [SMILES: CCn1cc(-c2cccc(N3CCN(c4ccc(C(F)(F)F)cc4)C3=O)c2)c(-c2ccncc2)n1] | 1-(3-(1-ethyl-3-(pyridin-4-yl)- 1H-pyrazol-4-yl)phenyl)-3-(4- (trifluoromethyl)phenyl)imidazol- idin-2-one |
| 54 | 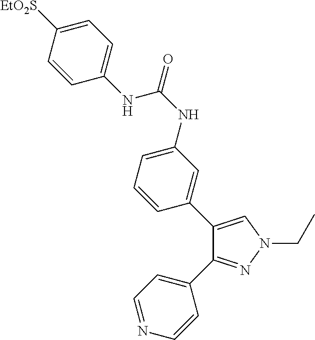 | 1-(3-(1-ethyl-3-(pyridin-4-yl)- 1H-pyrazol-4-yl)phenyl)-3-(4- (ethylsulfonyl)phenyl)urea |
| 55 | 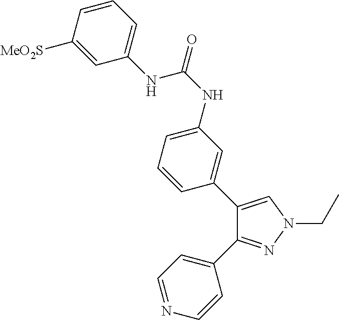 [SMILES: CCn1cc(-c2cccc(NC(=O)Nc3cccc(S(C)(=O)=O)c3)c2)c(-c2ccncc2)n1] | 1-(3-(1-ethyl-3-(pyridin-4-yl)- 1H-pyrazol-4-yl)phenyl)-3-(3- (methylsulfanyl)phenyl)urea |
| 56 | 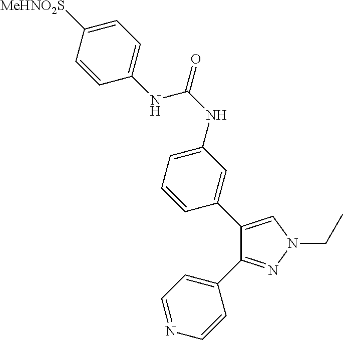 | 4-(3-(3-(1-ethyl-3-(pyridin-4- yl)-1H-pyrazol-4- yl)phenyl)ureido)-N- methylbenzenesulfonamide |
| 57 | 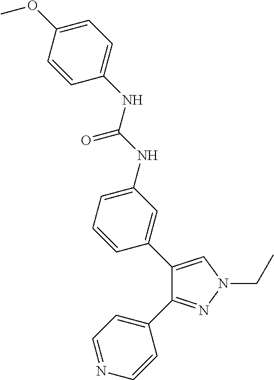 [SMILES: CCn1cc(-c2cccc(NC(=O)Nc3ccc(OC)cc3)c2)c(-c2ccncc2)n1] | 1-(3-(1-ethyl-3-(pyridin-4-yl)- 1H-pyrazol-4-yl)phenyl)-3-(4- methoxyphenyl)urea |
| 58 | 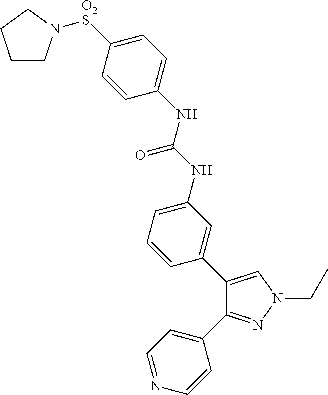 [SMILES: CCn1cc(-c2cccc(NC(=O)Nc3ccc(S(=O)(=O)N4CCCC4)cc3)c2)c(-c2ccncc2)n1] | 1-(3-(1-ethyl-3-(pyridin-4-yl)- 1H-pyrazol-4-yl)phenyl)-3-(4- (pyrrolidin-1- ylsulfonyl)phenyl)urea |
| 59 | 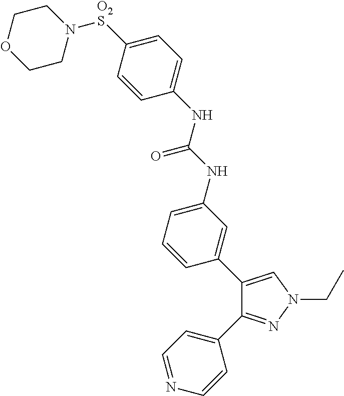 [SMILES: CCn1cc(-c2cccc(NC(=O)Nc3ccc(S(=O)(=O)N4CCOCC4)cc3)c2)c(-c2ccncc2)n1] | 1-(3-(1-ethyl-3-(pyridin-4-yl)- 1H-pyrazol-4-yl)phenyl)-3-(4- (morpholinosulfonyl)phenyl)urea |
| 60 | 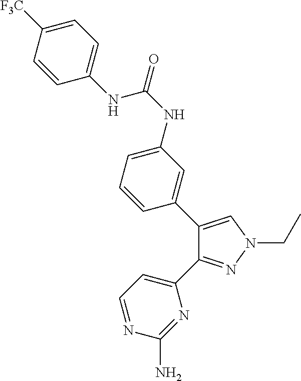 [SMILES: CCn1cc(-c2cccc(NC(=O)Nc3ccc(C(F)(F)F)cc3)c2)c(-c2ccnc(N)n2)n1] | 1-(3-(3-(2-aminopyrimidin-4- yl)-1-ethyl-1H-pyrazol-4- yl)phenyl)-3-(4- (trifluoromethyl)phenyl)urea |
| 61 | 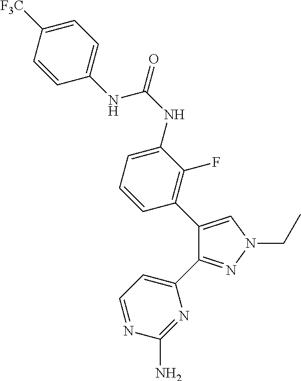 [SMILES: CCn1cc(-c2cccc(NC(=O)Nc3ccc(C(F)(F)F)cc3)c2F)c(-c2ccnc(N)n2)n1] | 1-(3-(3-(2-aminopyrimidin-4- yl)1-ethyl-1H-prazol-4-yl)-2- fluorophenyl)-3-(4- (trifluoromethyl)phenyl)urea |
| 62 | 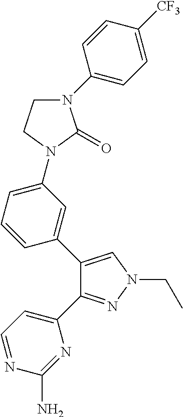 [SMILES: CCn1cc(-c2cccc(N3CCN(c4ccc(C(F)(F)F)cc4)C3=O)c2)c(-c2ccnc(N)n2)n1] | 1-(3-(3-(2-aminopyrimidin-4- yl)-1-ethyl-1H-pyrazol-4- yl)phenyl)-3-(4- (trifluoromethyl)phenyl)imidazol- idin-2-one |
| 63 | 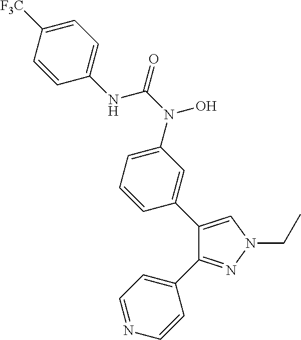 [SMILES: CCn1cc(-c2cccc(N(O)C(=O)Nc3ccc(C(F)(F)F)cc3)c2)c(-c2ccncc2)n1] | 1-(3-(1-ethyl-3-(pyridin-4-yl)- 1H-pyrazol-4-yl)phenyl)-1- hydroxy-3-(4- (trifluoromethyl)phenyl)urea |

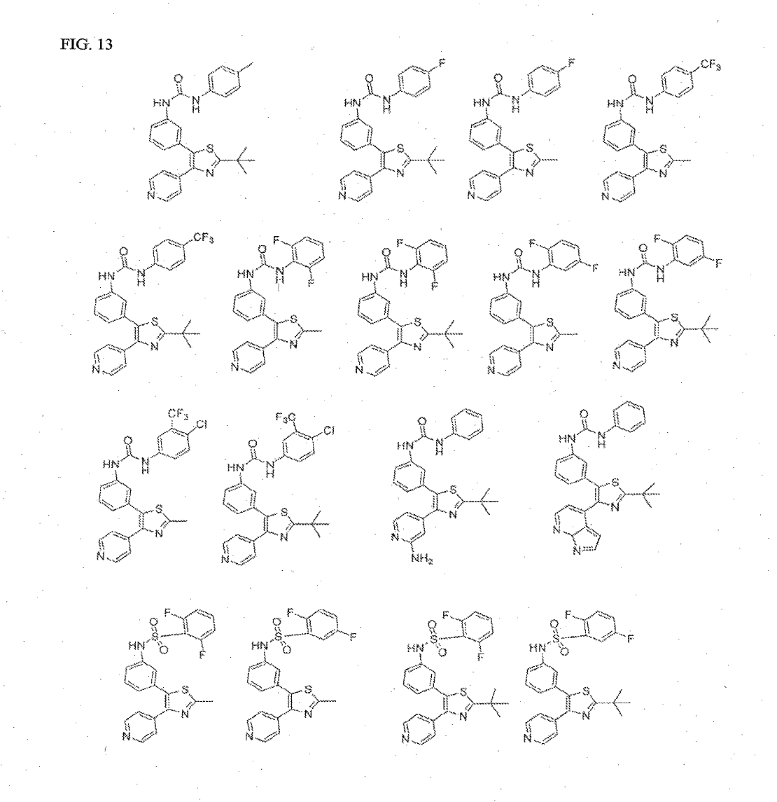

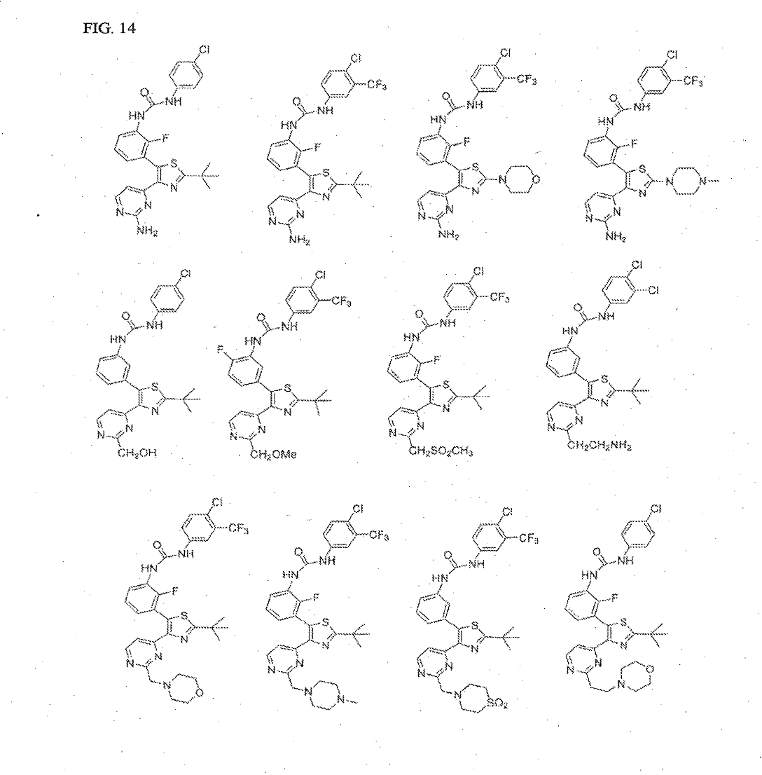

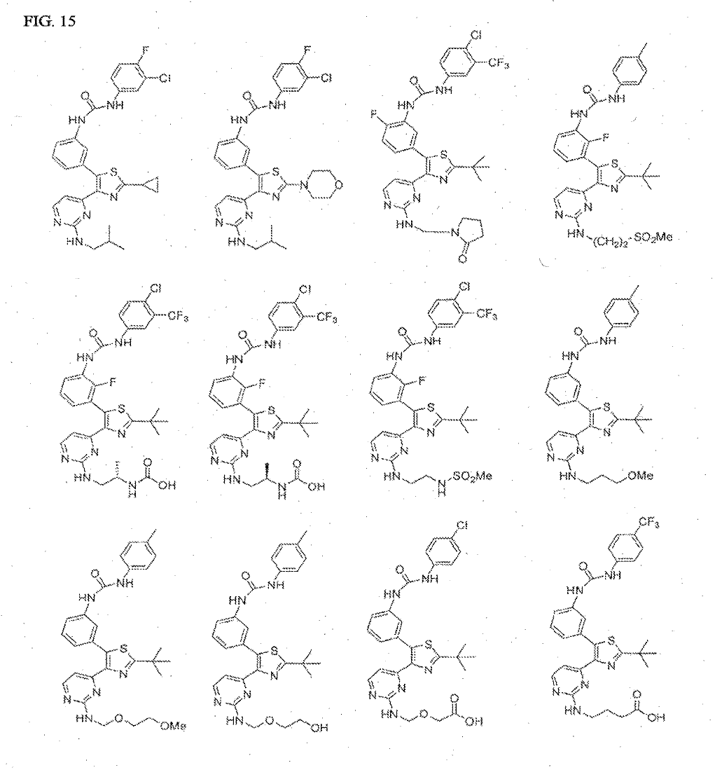

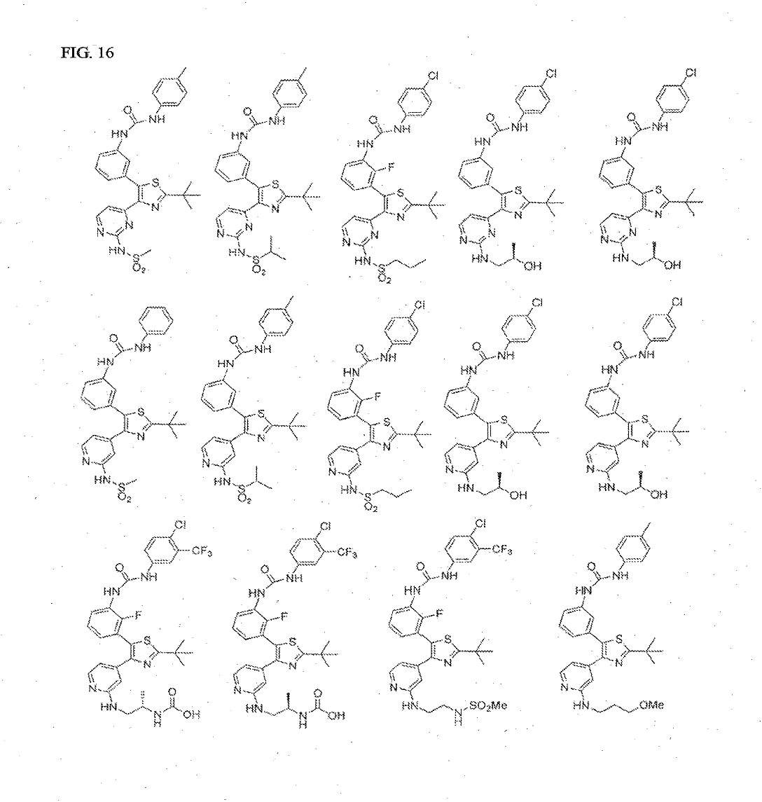

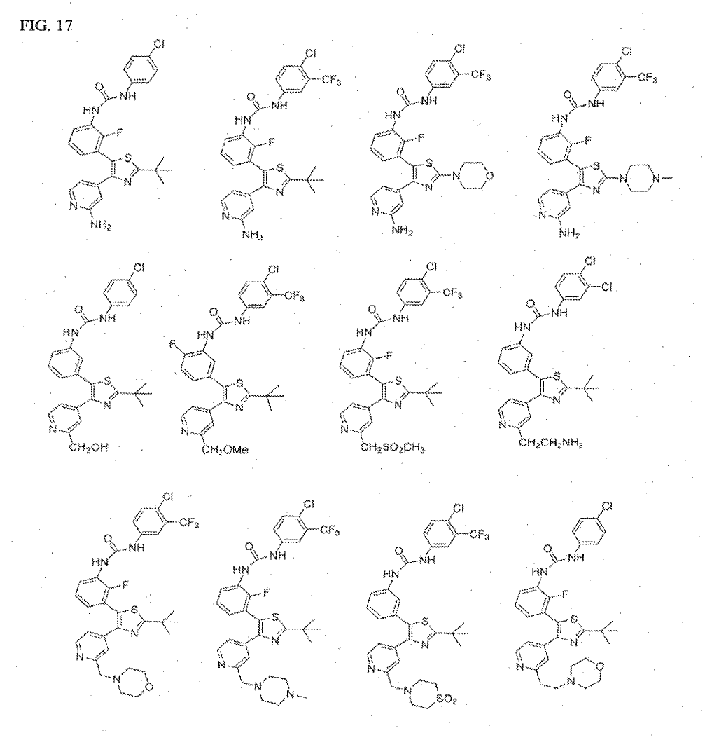

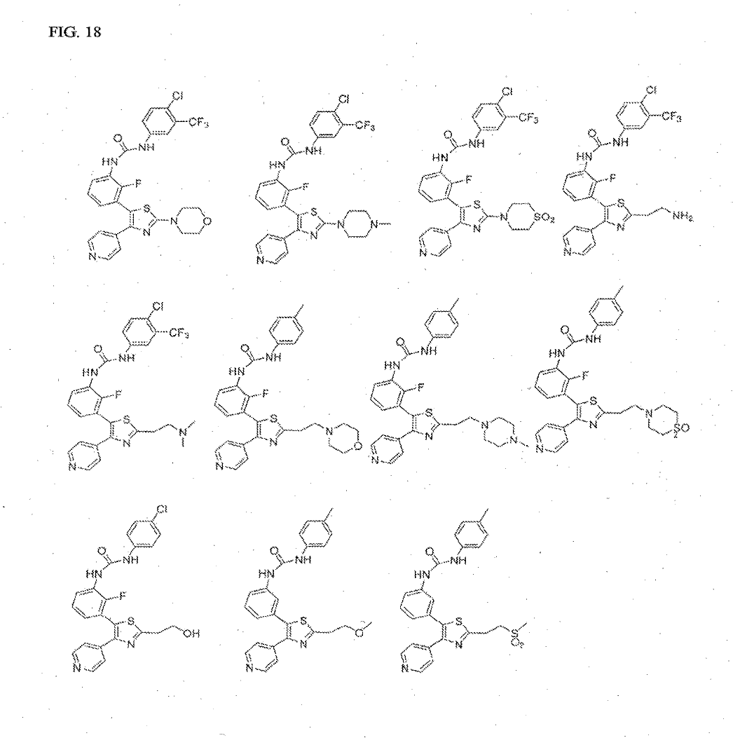

In certain specific embodiments, the compounds of Formula (I)-(IV) have the structures shown in FIGS. 1 to 18.

### Further Forms of Compounds

In one aspect, compounds of Formula (I)-(IV) possess one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof in certain embodiments, compounds of Formula (I)-(IV) are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

The methods and compositions described herein include the use of amorphous forms as well as crystalline forms (also known as polymorphs). In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms With pharmaceutically acceptable solvents such as water, ethanol, and the like. The solvated forms of the compounds presented herein are also considered to be disclosed herein.

In some embodiments, compounds described herein are prepared as prodrugs. A “prodrug” refers to an agent that is converted into the parent drug in vivo. Prodrugs are alien useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration; a prodrug is chemically converted to the biologically; pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal chemistry, Vol. 41, 395-407, 2006; T. Higuchi and V. Stella, Pro-drugs as Navel Delivery Systems, Vol. 14 of the A.C.S. Symposium Series).

Prodrug forms of the herein described, compounds, wherein the prodrug is metabolized in vivo to produce a compound of Formula (I)-(IV) as set forth herein are included within the scope of the claims. In some cases, some of the herein-described compounds may be a prodrug for another derivative or active compound.

In some embodiments, sites on the aromatic ring portion of compounds of Formula (I)-(IV) are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds; which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable;” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (I)-(IV) with acids. Pharmaceutically acceptable salts are also obtained by reacting a compound of Formula (I)-(IV) with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid; gluconic acid, glutamic acid, hydroxynaphthoic acid; salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms in general, the solvated forms are considered equivalent to the unsolvated forms for the purposes of the compounds and methods provided herein.

Compounds described herein; such as compounds of Formula (I)-(IV), may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness; crystal shape, optical properties, stability, and solubility. Various factors such as the recrystallization solvent, rate of crystallization, and storage temperature may cause a single crystal form to dominate.

Throughout the specification, groups and substituents thereof can be chosen by one skilled in the field to provide stable moieties and compounds.

### Certain Terminology

Unless otherwise stated, the following terms used in this application, including the specification and claims, have the definitions given below. It must be noted that, as used in the specification and the appended claims, the singular, forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms; such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group (which means that it does not contain any carbon-carbon double bonds or carbon-carbon triple bonds) or the alkyl group may be an unsaturated alkyl group (which means that it contains at least one carbon-carbon double bonds or carbon-carbon triple bond). The alkyl moiety, whether saturated or unsaturated, may be branched, or straight chain.

The “alkyl” group may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one, two, three, four, five, or six carbon atoms in the alkyl chain. In one aspect the alkyl is selected from the group consisting of methyl, ethyl, propyl, iso-propyl, n-butyl, iso-butyl, sec-butyl, and t-butyl. Typical alkyl groups include; but are in no way limited to, methyl, ethyl, propyl, isopropyl, butyl, isobutyl, sec-butyl, tertiary butyl, pentyl, neopentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₆ alkyl. In one aspect, an alkyl is a C₁-C₄ alkyl. In one aspect, an alkyl is a C₁-C₃ alkyl. In one aspect, an alkyl is a C₁-C₂ alkyl.

The term “alkylene” refers to a divalent alkyl radical. Any of the above mentioned monovalent alkyl groups may be an alkylene by abstraction of a second hydrogen atom from the alkyl. In one aspect, an alkelene is a C₁-C₆alkylene. In another aspect, an alkylene is it C₁-C₄alkylene. Typical-alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

An “alkoxy” group refers to a (alkyl)O— group, where alkyl is as defined herein.

The term “alkylamine” refers to the N(alkyl)ₓH_{y} group, where x and y are selected from the group x=1, y=1 and x=2, y=0. In some embodiments, when x=2 and y=0, the alkyl groups taken together with the nitrogen atom to which they are attached form a cyclic ring system.

The term “aromatic” refers to a planar ring having a delocalized π-electron system containing 4n+2π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or, “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from heterocyclic rings in which the ring backbone contains at least one atom which is different from carbon.

As used herein, the term “aryl” refers to an aromatic ring wherein each of the atoms forming the ring is a carbon atom. Aryl rings are formed by five, six, seven, eight, nine, or more than nine carbon atones. Aryl groups are optionally substituted. In one aspect, an aryl is a phenyl or a naphthalenyl. In one aspect, an aryl is a phenyl. In one aspect, an aryl is a C₆-C₁₀aryl. Depending on the structure, an aryl group can be a monoradical or a diradical (i.e., an arylene group). In one aspect, an arylene is a C₆-C₁₀ arylene. Examplary arylenes include, but are not limited to, phenyl-1,2-ene, phenyl-1,3-ene, and phenyl-1,4-ene.

The term “cycloalkyl” refers to a monocyclic or polycyclic, aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, and cyclooctyl. Cycloalkyl groups may be substituted or unsubstituted. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cyclohexan-1,4-diyl, cycloheptan-1,1-diyl, and the like). In one aspect, a cycloalkyl is a C₃-C₆cycloalkyl.

The term “halo” or, alternatively, “halogen” or “halide” means fluoro, chloro, bromo or dodo.

The term “haloalkyl” refers to an alkyl group in which one or more hydrogen atoms are replaced by one or more halide atoms. In one aspect, a haloalkyl is a C₁-C₄haloalkyl.

The term “haloalkylene” refers to an alkylene group in which one or more hydrogen atoms are replaced by one or more halide atoms. In one aspect, a haloalkylene is a C₁-C₆haloalkylene. In another aspect; a haloalkylene is a C₁-C₄haloalkylene.

The term “fluoroalkyl” refers to an alkyl in which one or more hydrogen atoms are replaced by a fluorine atom. In one aspect, a fluoralkyl is a C₁-C₄-fluoroalkyl.

The term “fluoroalkylene” refers to an alkylene in which one or more hydrogen atoms are replaced by a fluorine atom. In one aspect, a fluoralkylene is a C₁-C₆-fluoroalkylene. In another aspect, a fluoralkylene is a C₁-C₄-fluoroalkylene.

The term “heteroalkyl” refers to an alkyl group in which one or more skeletal-atoms of the alkyl are selected from an atom other than carbon, e.g., oxygen, nitrogen, sulfur, phosphorus or combinations thereof. In one aspect, a heteroalkyl is a C₁-C₆heteroalkyl.

The term “heteroalkylene” refers to an alkylene group in which one or more skeletal atoms of the alkyl are selected from an atom other than carbon, e.g., oxygen, nitrogen, sulfur, phosphorus or combinations thereof. In one aspect, a heteroalkylene is a C₁-C₆heteroalkylene. In another aspect, a heteroalkylene is a C₁-C₄heteroalkylene. Examplary heteroalkylenes include, but are not limited to, —OCH₂—, —OCH(CH₃)—, —OC(CH₃)₂—, —OCH₂CH₂—, —CH₂O—, —CH(CH₃)O—, —C(CH₃)₂O—, —CH₂CH₂O—, —CH₂OCH₂—, —CH₂OCH₂CH₂—, —CH₂CH₂OCH₂—, SCH(CH₃)—, SC(CH₃)₂, —SCH₂CH₂—, —CH₂S—, —CH(CH₃)S—, —C(CH₃)₂S—, —CH₂CH₂S—, —CH₂SCH₂—, —CH₂SCH₂CH₂—, —CH₂CH₂SCH₂—, —SO₂CH₂—, —SO₂CH(CH₃)—, SO₂C(CH₃)₂—, SO₂CH₂CH₂—, —CH₂SO₂—, —CH(CH₃)SO₂—, —C(CH₃)₂SO₂—, —CH₂CH₂SO₂—, —CH₂SO₂CH₂—, —CH₂SO₂CH₂CH₂—, —CH₂CH₂SO₂CH₂—, —NHCH₂—, —NHCH(CH₃)—, —NHC(CH₃)₂—, —NHCH₂CH₂—, —CH₂NH—, —CH(CH₃)NH—, —C(CH₃)₂NH—, —CH₂CH₂NH—, —CH₂NHCH₂—, —CH₂NHCH₂CH₂—, —CH₂CH₂NHCH₂—, and the like.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as, heterocycloalkyls) include groups having only 3 atoms in their ring, system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl; dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl(C-attached). Further, a group derived from imidazole may be imidazol-1-yl or imidazol-3-yl (both N-attached) or imidazol-2-yl, -imidazol-4-yl or imidazol-5-yl (all C-attached). The heterocyclic groups include benzo-fused ring systems. Non-aromatic heterocycles may be substituted with one or two oxo (═O) moieties, such as pyrrolidin-2-one.

The terms “heteroaryl” or, alternatively, “heteroaromatic” refers to an aryl group that includes one or more ring heteroatoms selected from nitrogen, oxygen and sulfur. Illustrative examples of heteroaryl groups include the following moieties:

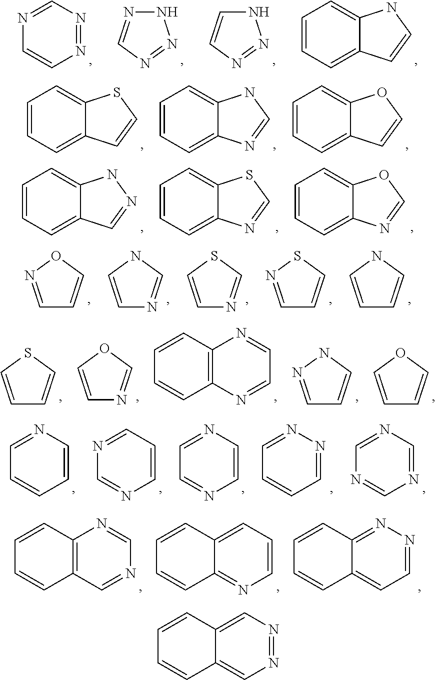

and the like. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In one aspect, a heteroaryl contains 0-3 N atoms. In another aspect, a heteroaryl contains 1-3 N atoms. In another aspect, a heteroaryl contains 0-3N atoms, 0-1 O atoms, and 0-1 S atoms. In another aspect, a heteroaryl is a monocyclic or bicyclic heteroaryl. In one aspect, heteroaryl is a C₁-C₉heteroaryl. In one aspect, monocyclic heteroaryl is a C₁-C₅heteroaryl. In one aspect, monocyclic heteroaryl is a 5-membered or 6-membered heteroaryl. In one aspect, bicyclic heteroaryl is a C₆-C₉heteroaryl. Depending on the structure, a heteroaryl group can be a monoradical or a diradical (i.e., a heteroarylene group).

The term “heteroarylene” refers to a divalent heteroaryl radical. Any of the above mentioned monovalent heteroaryl groups may be a heteroarylene by abstraction of a second hydrogen atom from the heteroaryl group. The divalent heteroaryl radical may be attached through two carbon atoms, or through one carbon atom and one heteroatom, or through two heteroatoms.

The term “heterocycloalkylene” refers to a divalent heterocycloalkyl radical. Any of the above mentioned monovalent heterocycloalkyl groups may be a heterocycloalkylene by abstraction of a second hydrogen atom from the heterocycloalkyl group. The divalent heterocycloalkyl radical may be attached through two carbon atoms, or through one carbon atom and one heteroatom, or through two heteroatoms.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

A “cyano” group refers to a —CN group.

The term “membered ring” includes any cyclic structure. The term.

“membered” is meant to denote the number of skeletal atoms that constitute the ring. Thus, for example, cyclohexyl, pyridinyl, pyranyl and thiopyranyl are 6-membered rings and cyclopentyl, pyrrolyl, furanyl, and thienyl are 5-membered rings.

The term “moiety” refers to a specific segment or functional group of a molecule. Chemical moieties are often recognized chemical entities embedded in or appended to a molecule.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

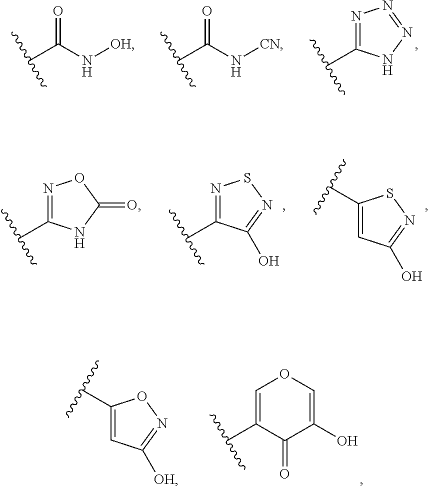

and the like.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and disubstituted amino groups, and the protected derivatives thereof. By way of example, the optional substituents may be halide, —CN, —NO₂, or LₛRₛ, wherein each Lₛ is independently selected from a bond, —O—, —C(═O)—, —C(═O)O—, —S—, —S(═O)—, —S(═O)₂—, —NH—, —NHC(═O)—, —C(═O)NH—, S(═O)₂NH—, —NHS(═O)₂, —OC(═O)NH—, —NHC(═O)O—, or —(C₁-C₆ alkylene)-; and each Rₛ is selected from H, alkyl, fluoroalkyl, heteroalkyl, cycloalkyl, aryl, heteroaryl, or heterocycloallyl. The protecting groups that may form the protective derivatives of the above substituents may be found in sources such as Greene and Wuts, above. In some embodiments, optional substituents are selected from halogen, —CN, —NH₂, —OH, —N(CH₃)₂, alkyl, fluoroalkyl, heteroalkyl, cycloalkyl, heterocycloalkyl, aryl, heteroaryl, alkoxy, aryloxy, alkylthio, arylthio, alkylsulfoxide, arylsulfoxide, alkylsulfone, and arylsulfone. In, some embodiments, an optional substituents is halogen, —CN, —NH₂, —OH, —NH(CH₃), —N(CH₃)₂, alkyl, fluoroalkyl, heteroalkyl, alkoxy, fluoroalkoxy, —S-alkyl, or S(═O)₂alkyl. In some embodiments, an optional substituent is selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic saturated or unsaturated carbon atoms, excluding aromatic carbon atoms) includes oxo (═O).

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, stereoselective synthesis and/or the separation of stereoisomers by chiral chromatographic columns.

The methods and formulations described herein include the use of N-oxides (if appropriate), crystalline forms (also known as polymorphs), or pharmaceutically acceptable salts of compounds having the structure of Formula (I)-(IV), as well as active metabolites of these compounds having the same type of activity. In some situations, compounds may exist as tautomers All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

The compounds, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. When the compounds described herein contain alkene double bonds or other centers of geometric asymmetry, and unless specified otherwise, it is intended that the compounds include both Z and E geocentric isomers (e.g., cis or trans.) Likewise; all possible isomers, as well as their racemic and optically pure forms, and all tautomeric forms are also intended to be included.

A “stereoisomer” refers to the relationship between two or more molecules made up of the same atoms bonded by the same bonds but having different three-dimensional structures, which are not superimposable. The term “enantiomer” refers to two stereoisomers whose molecules are nonsuperimposable mirror images of one another. It is contemplated that the various stereoisomers of the compounds disclosed herein, and mixtures thereof; are within the scope of the present disclosure and specifically includes enantiomers.

A “tautomer” refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

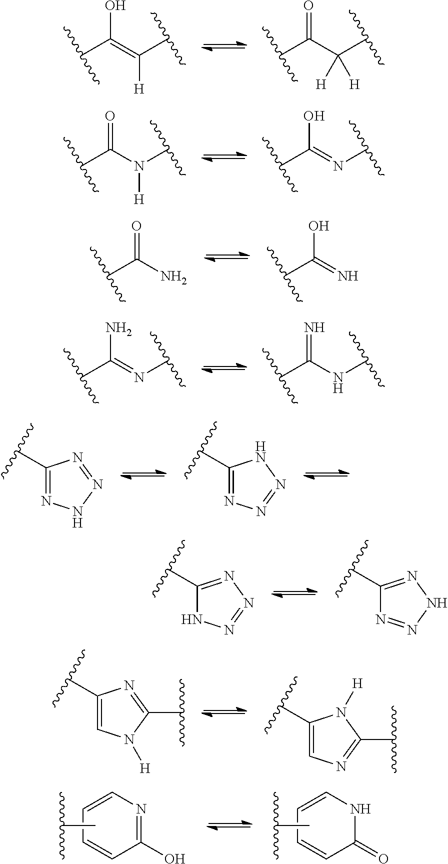

The term “acceptable” with respect to a formulation, composition or ingredient, as used herein, means having no persistent detrimental effect on the general health of the subject being treated.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist, partial agonist, an inverse agonist and antagonist. In one embodiment, a modulator is an antagonist.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals and non-mammals. Examples of mammals include; but are not limited to, any member of the Mammalian class: humans, non-human primates such as chimpanzees, and other apes and monkey species; farm animals such as cattle, horses, sheep, goats, swine, domestic animals such as rabbits, dogs, and cats, laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### General Methods for the Synthesis of Heterocyclic RAF Kinase Inhibitors

The synthetic Schemes 1-39 provided below illustrate general methods for the synthesis of the heterocyclic RAF kinase inhibitors described herein. These schemes are illustrative in nature, and are not intended to be limiting in any manner as to the methods suitable for preparing the compounds described herein.

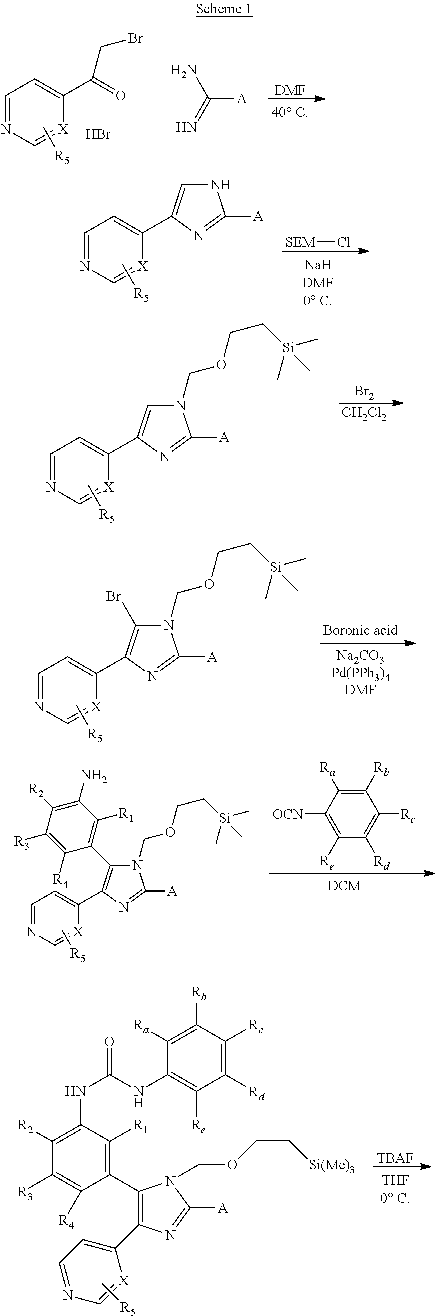

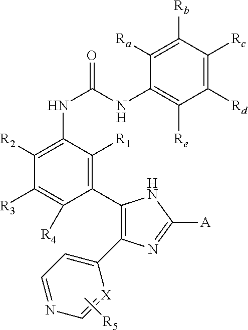

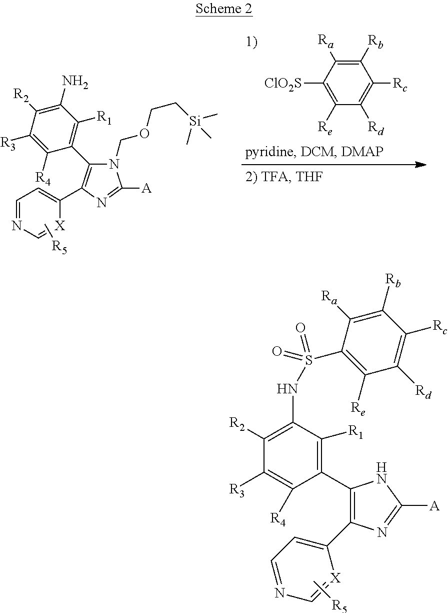

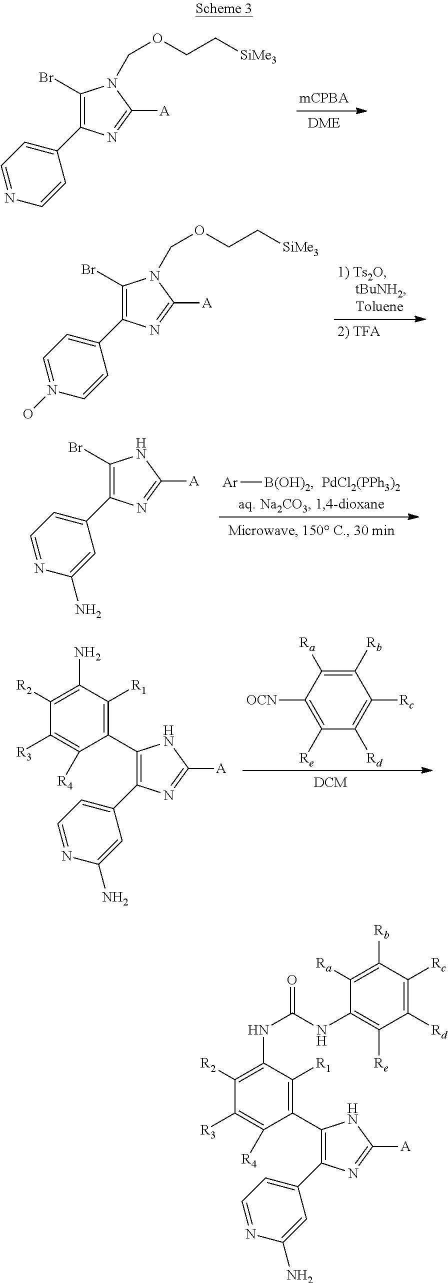

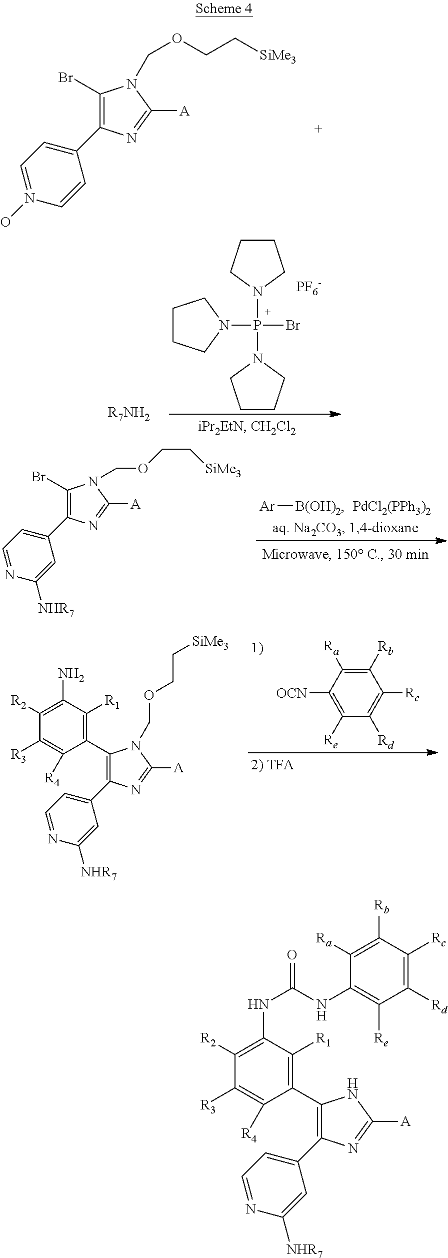

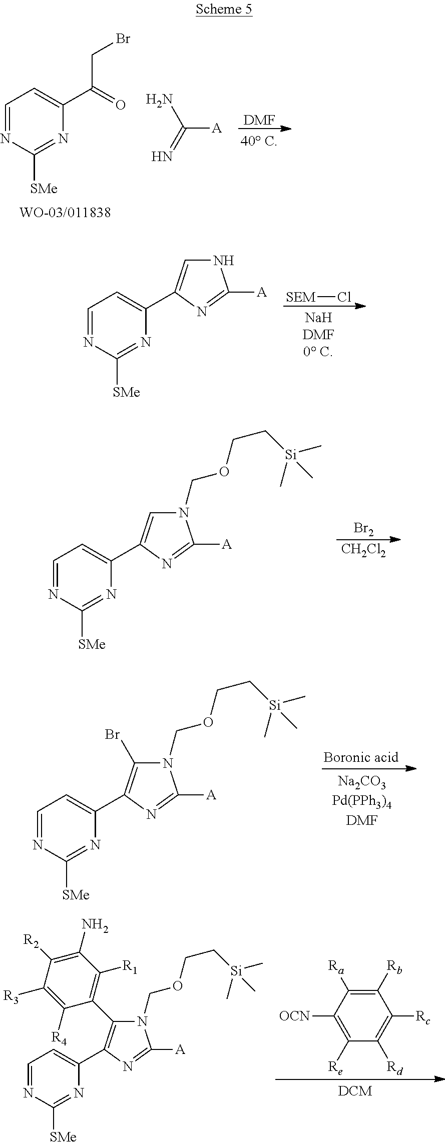

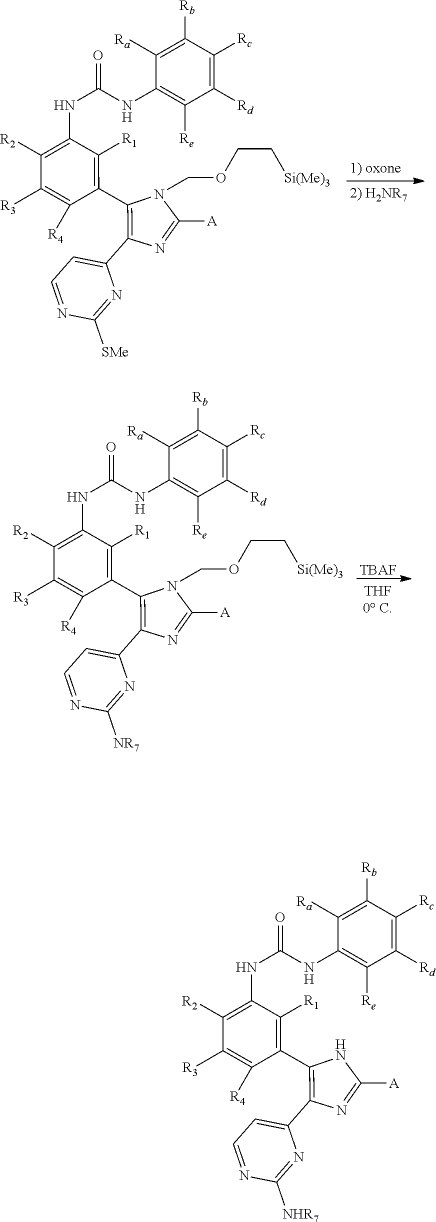

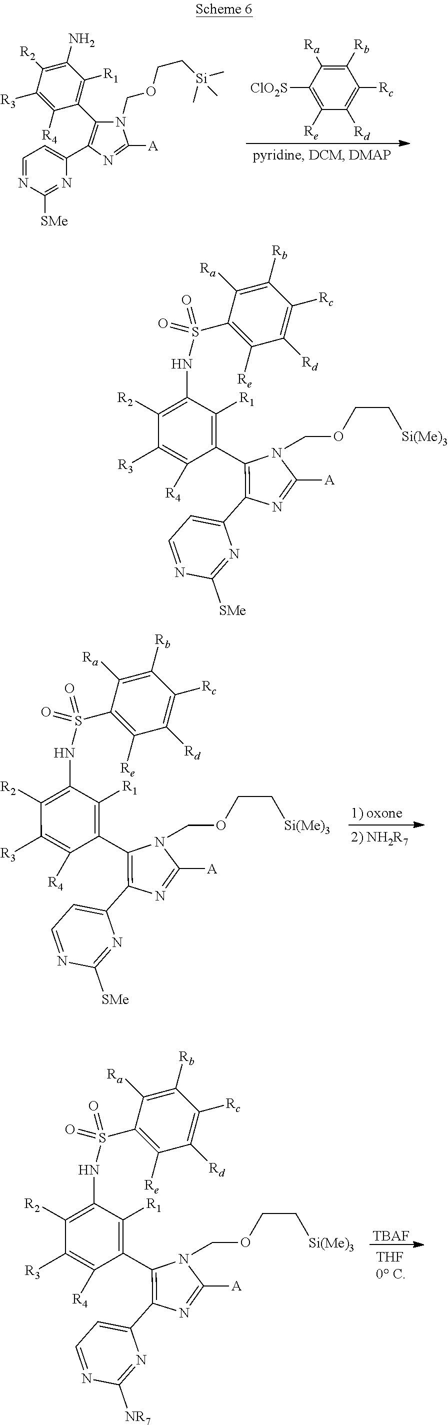

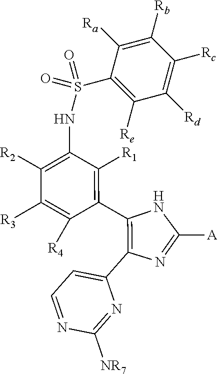

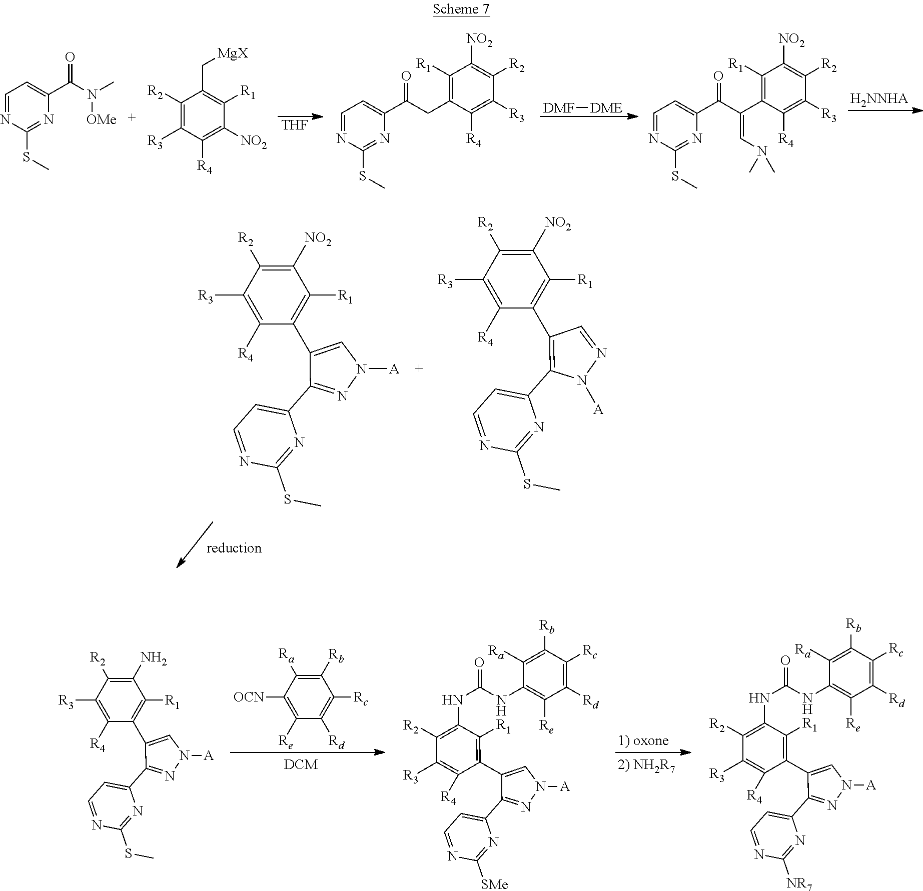

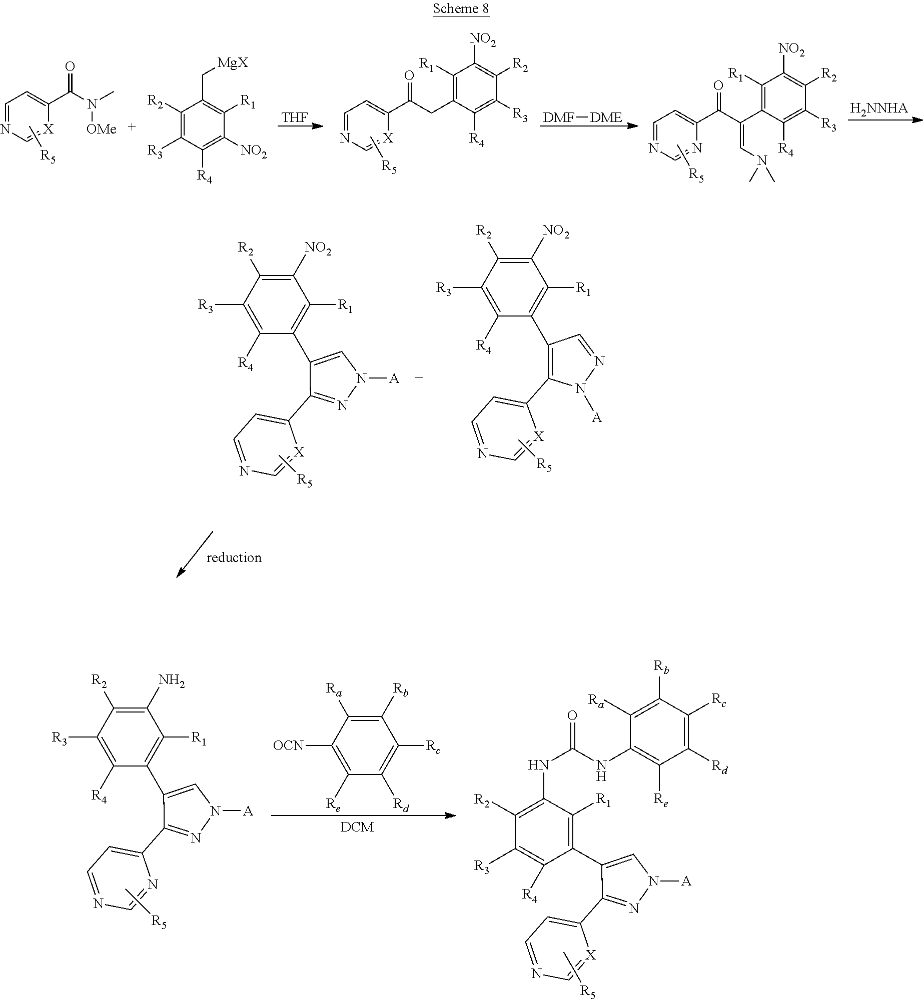

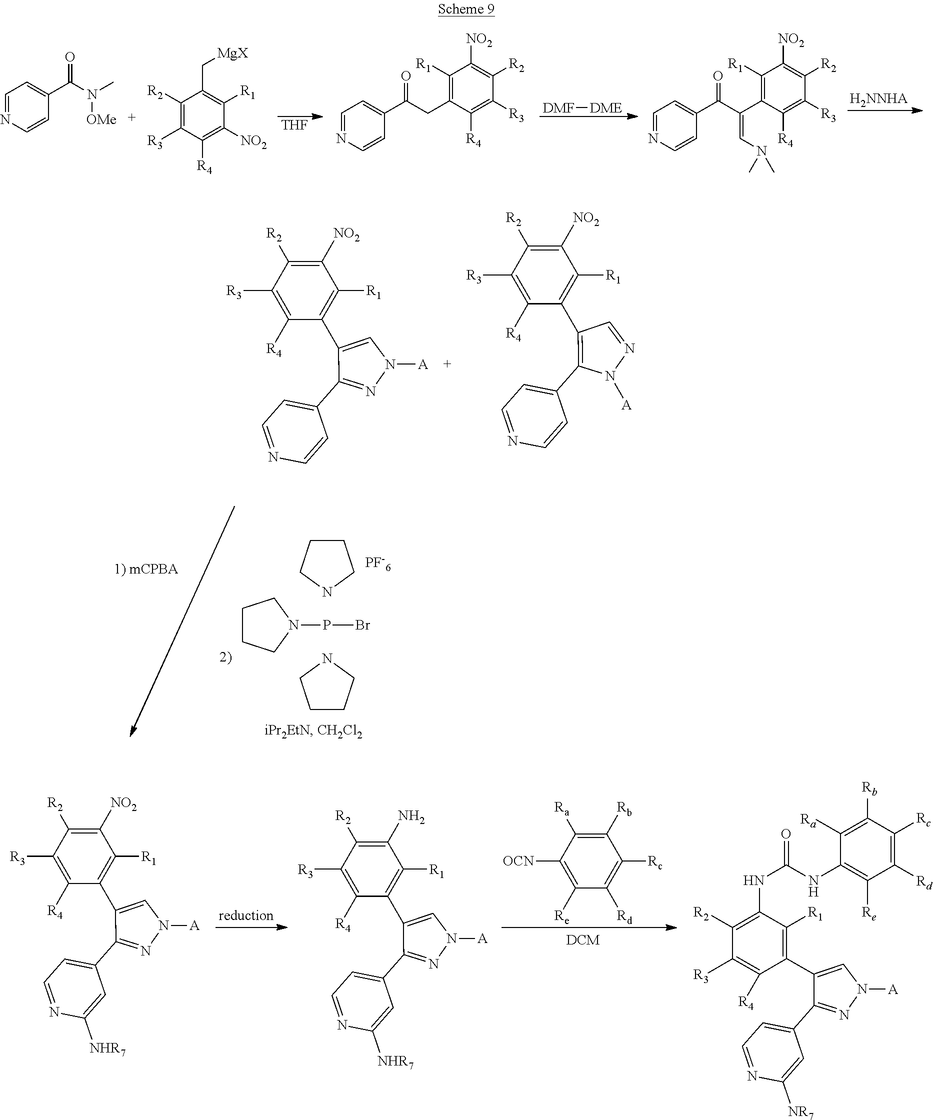

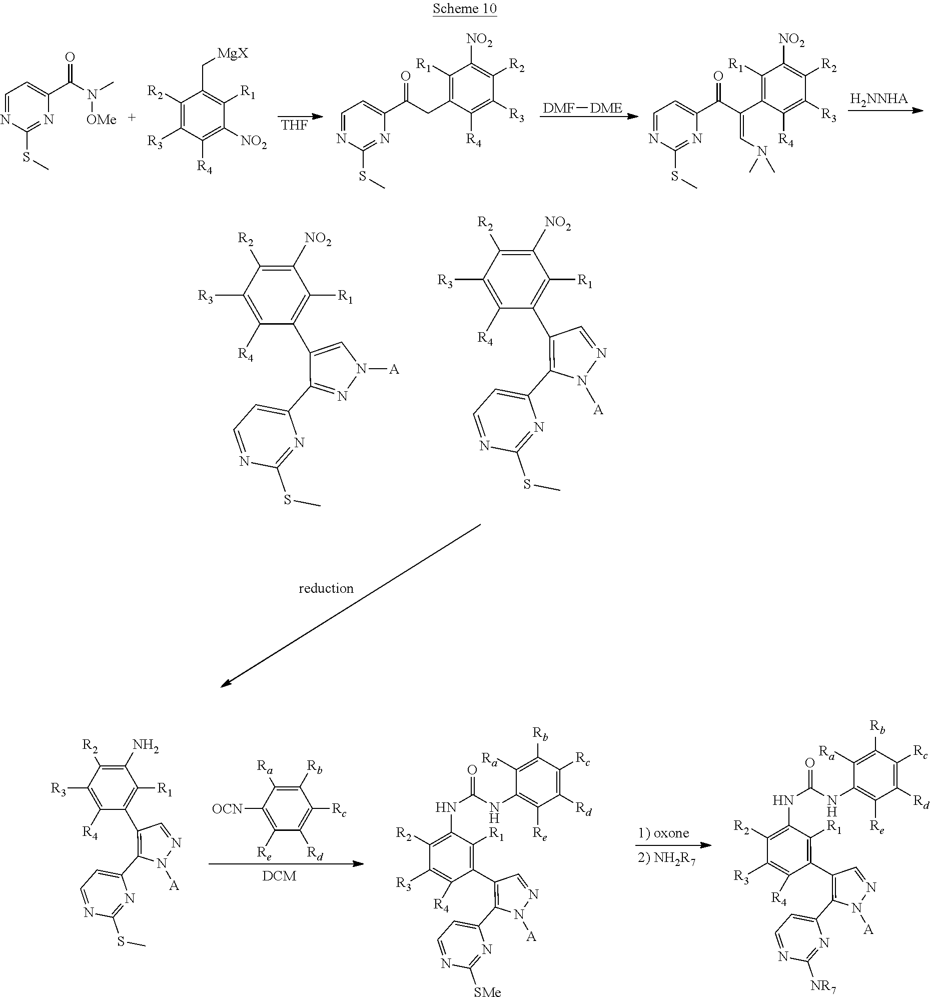

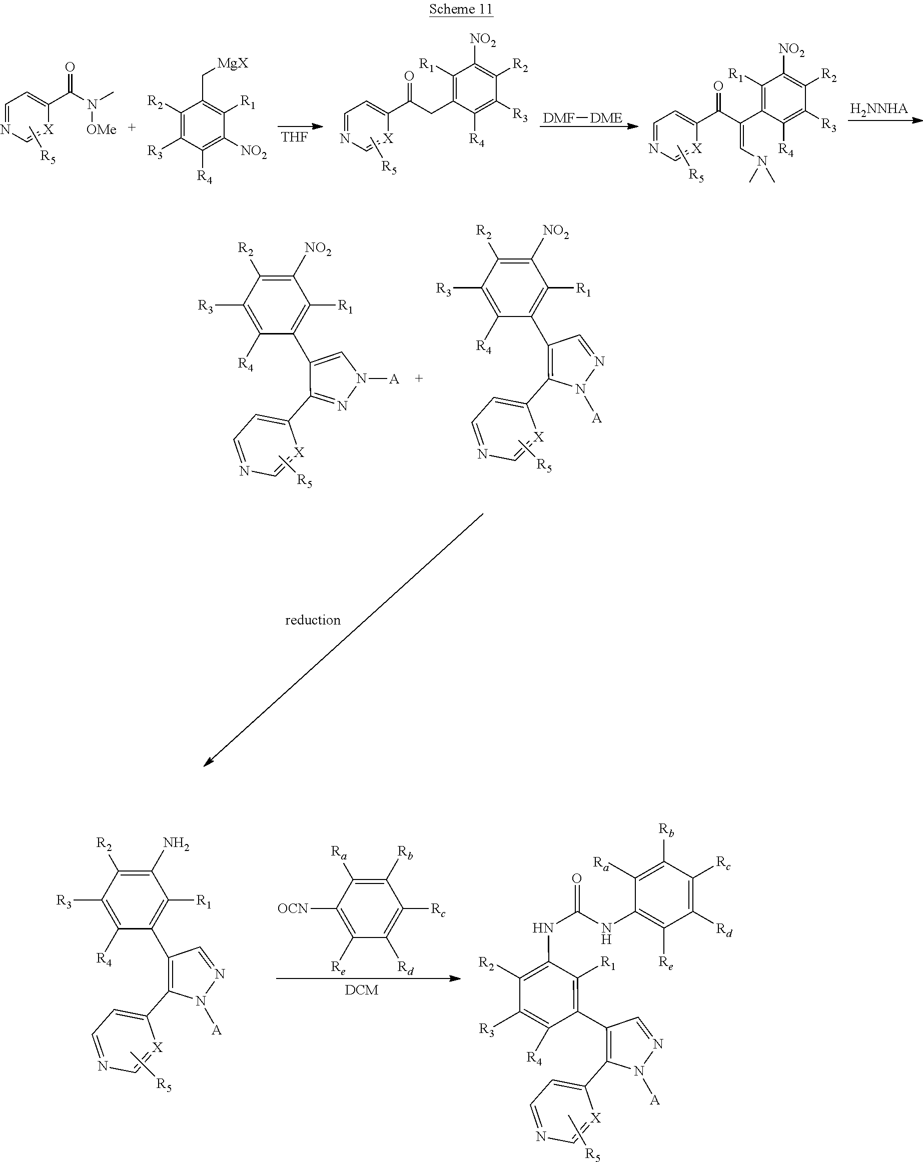

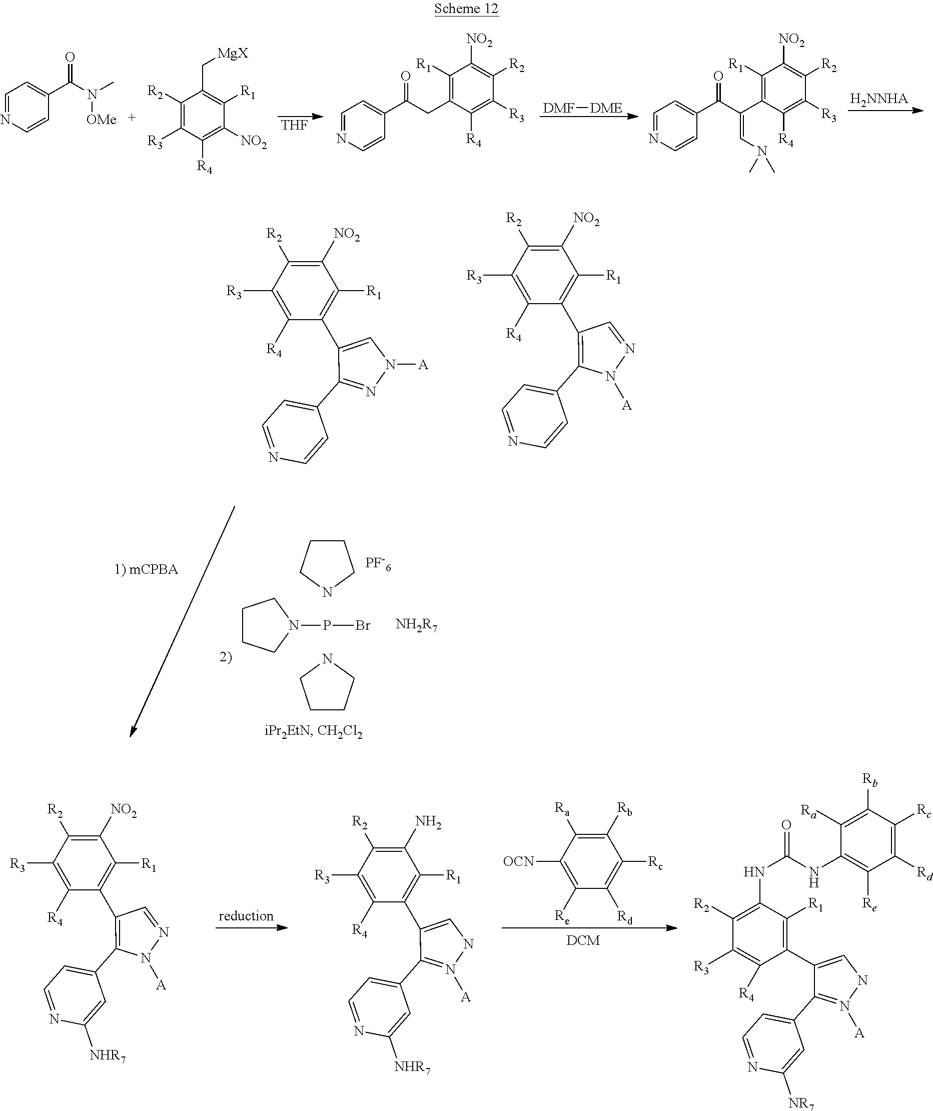

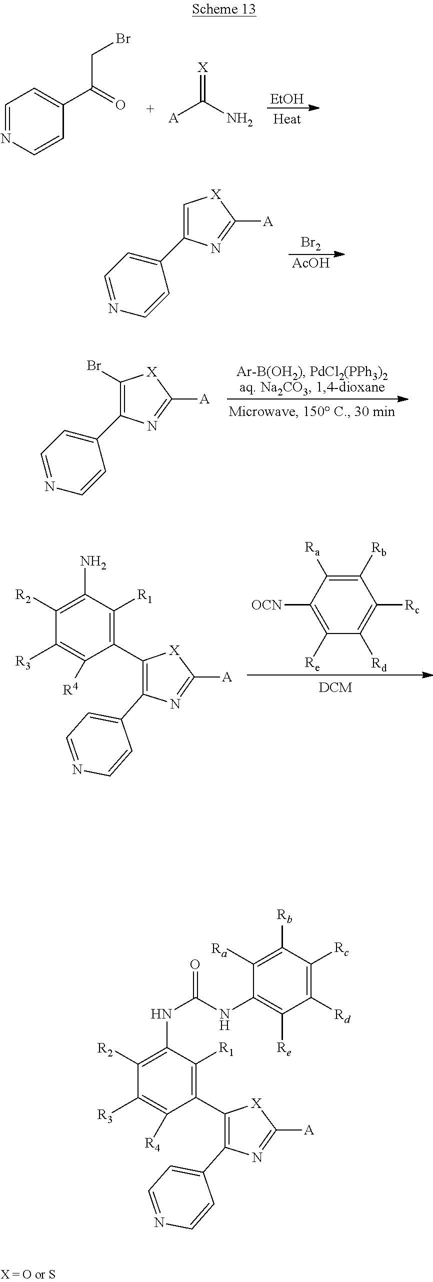

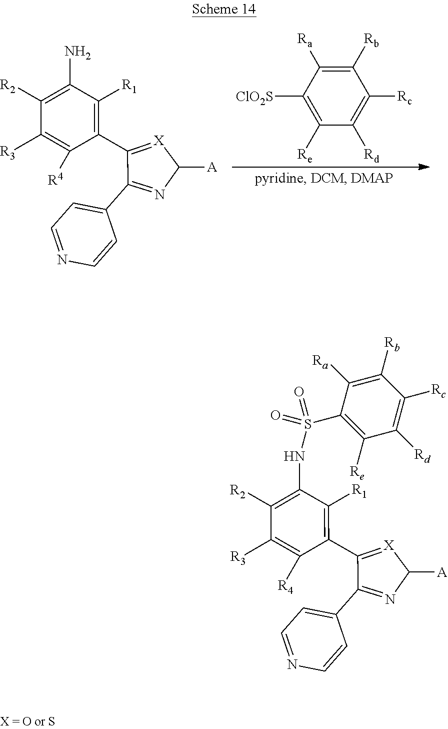

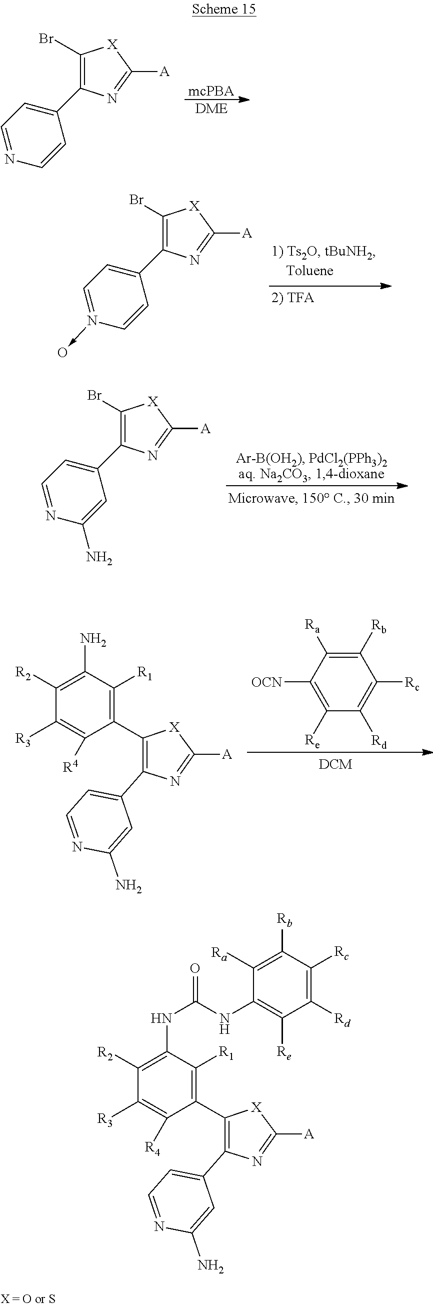

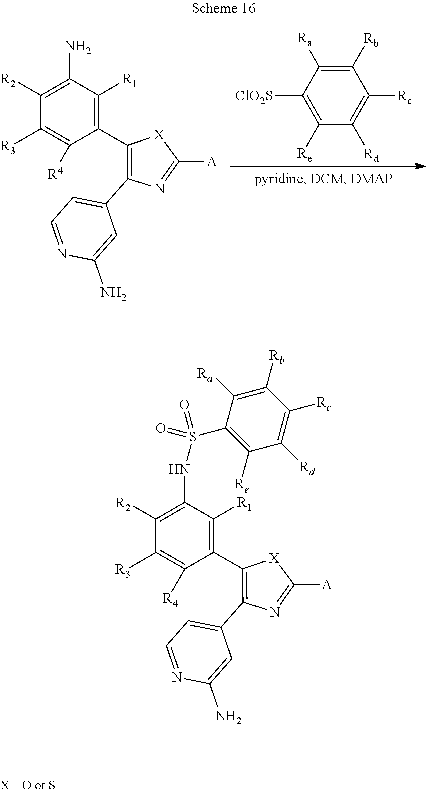

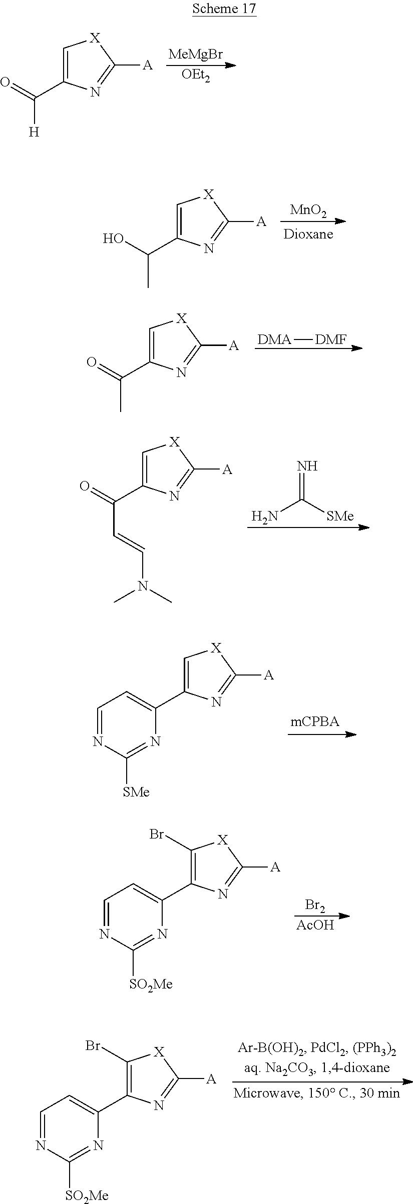

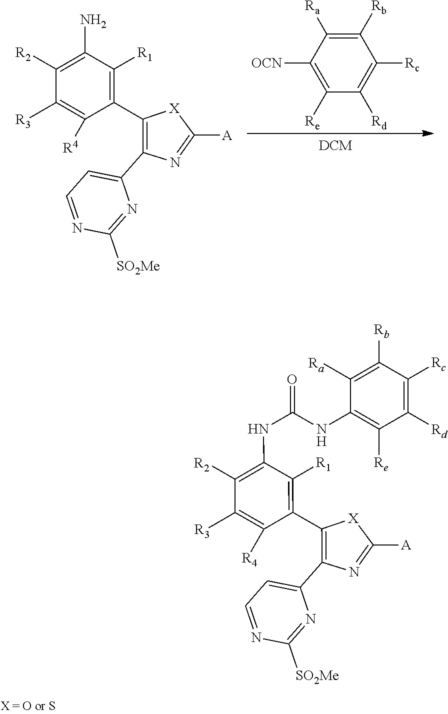

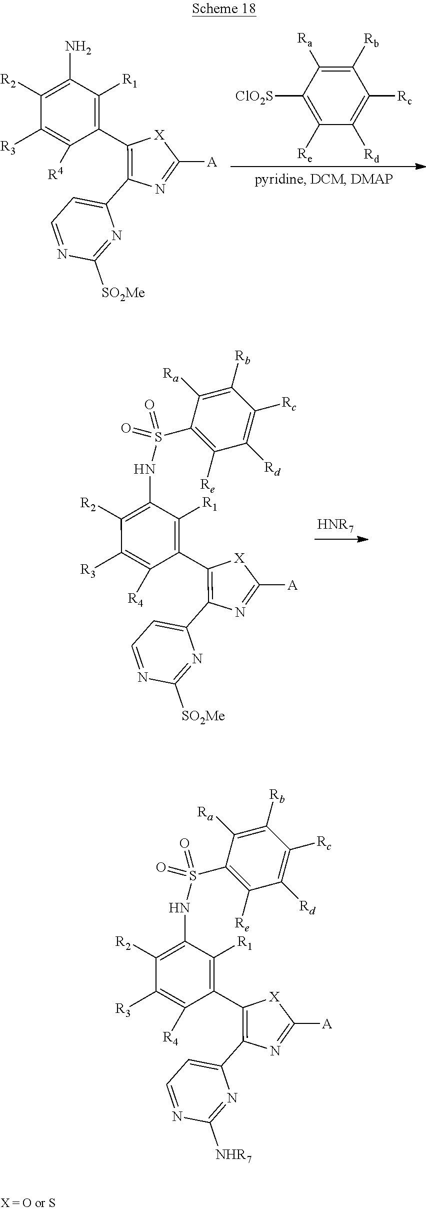

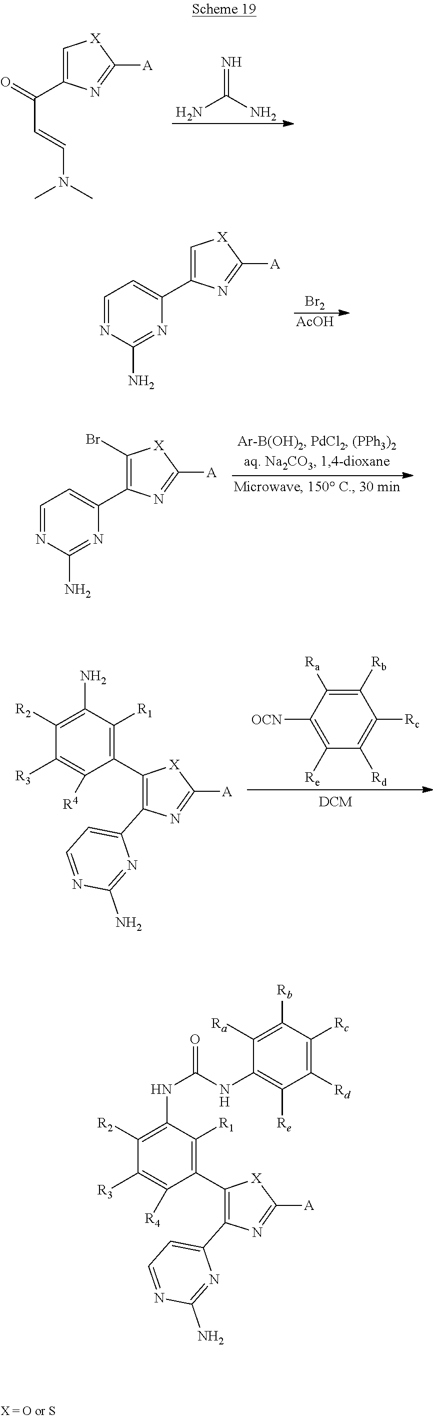

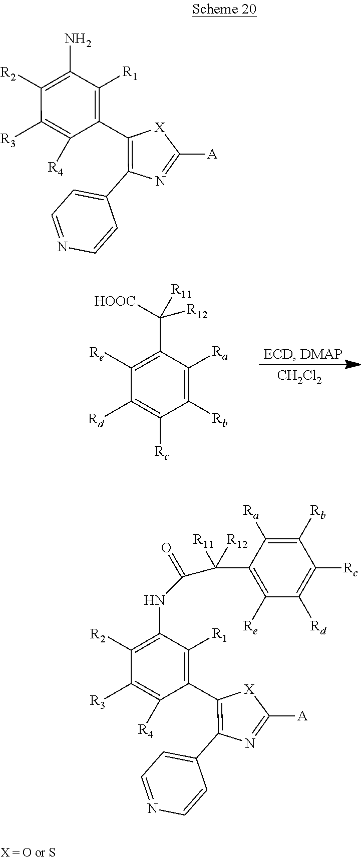

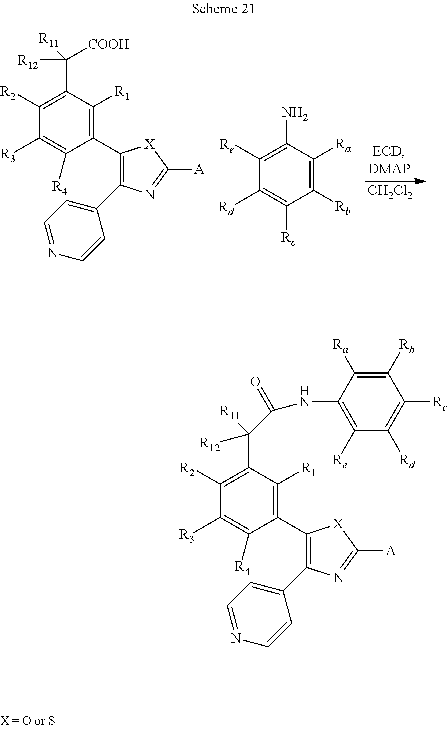

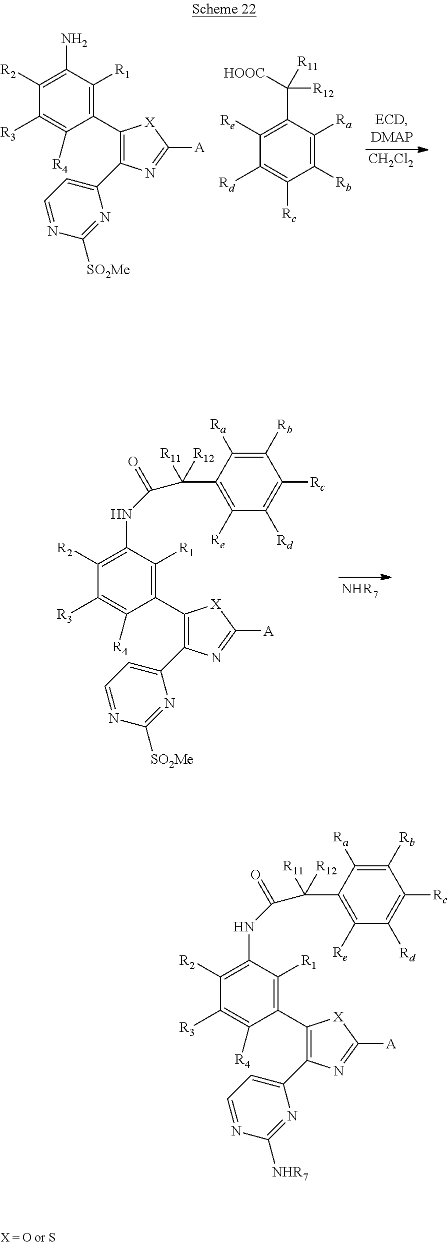

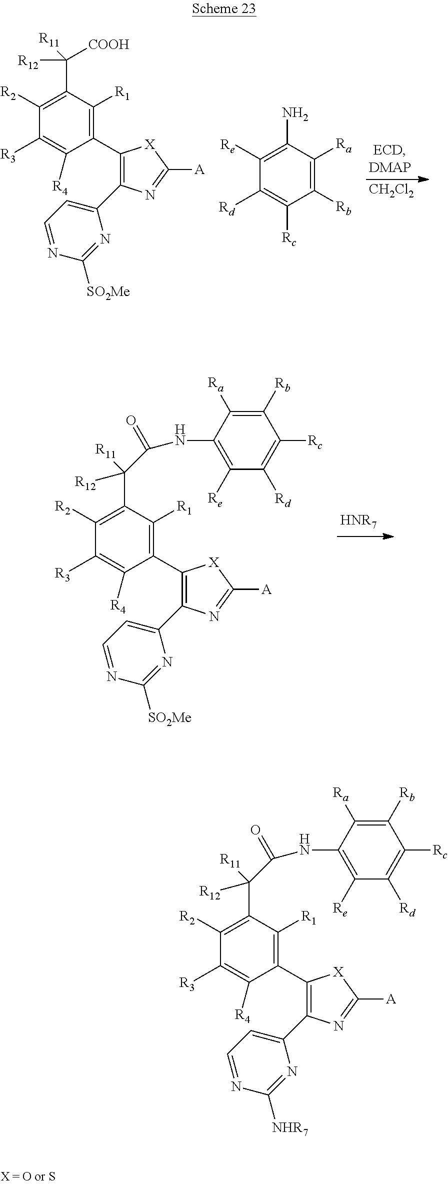

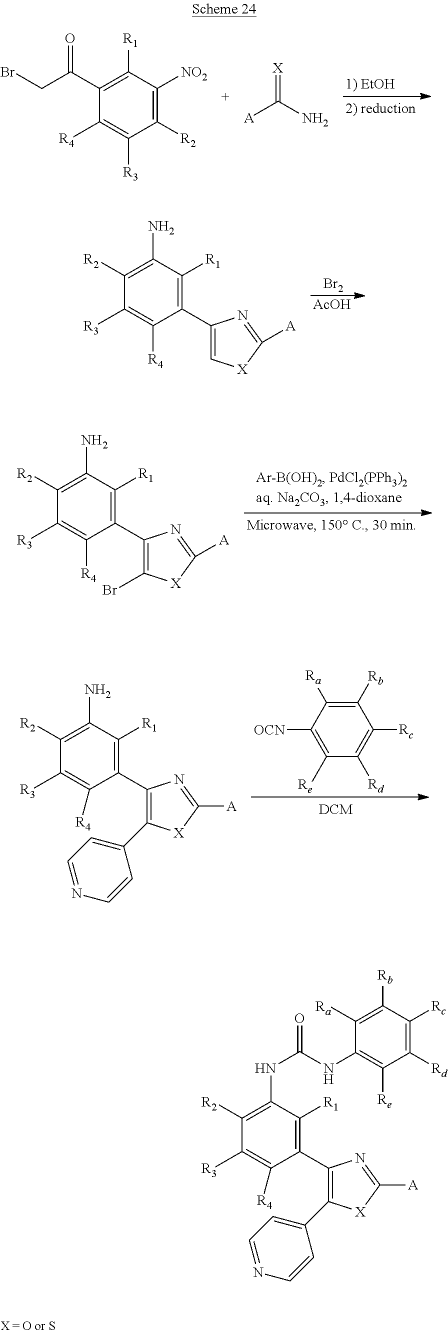

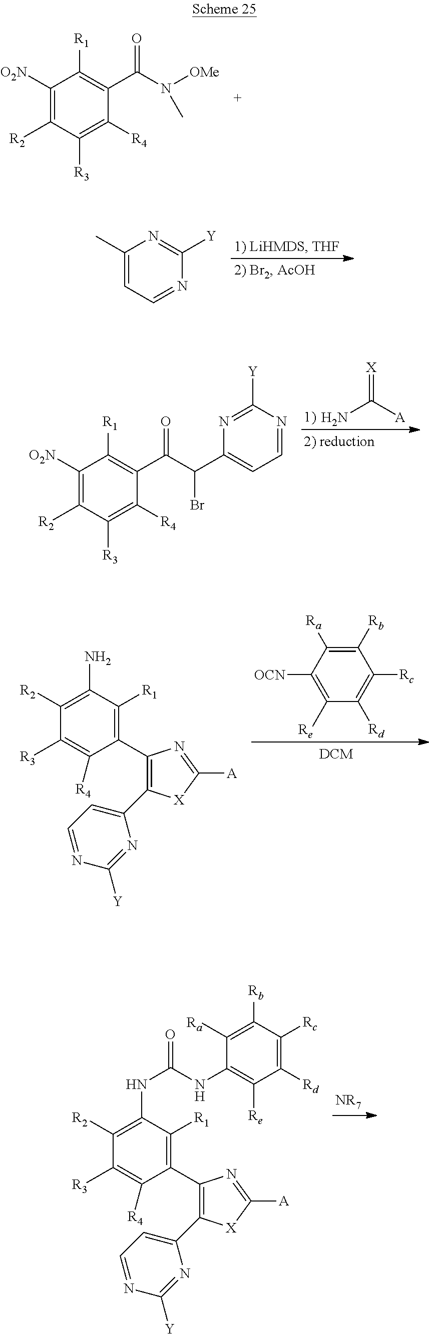

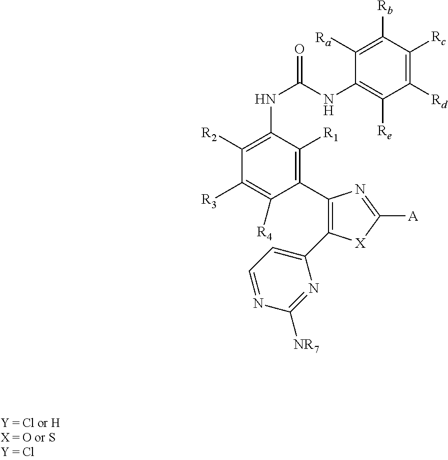

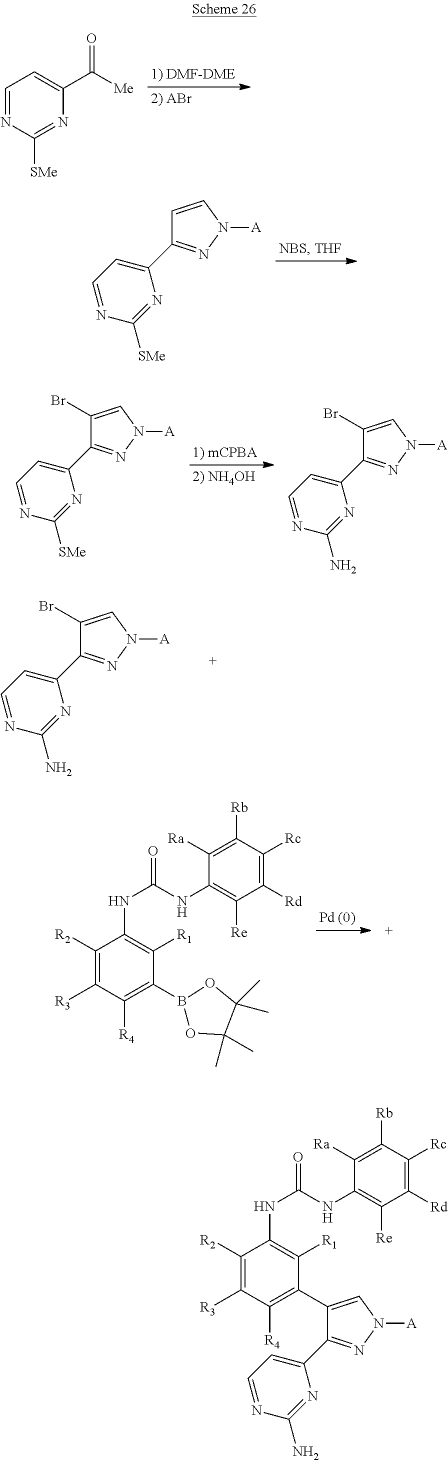

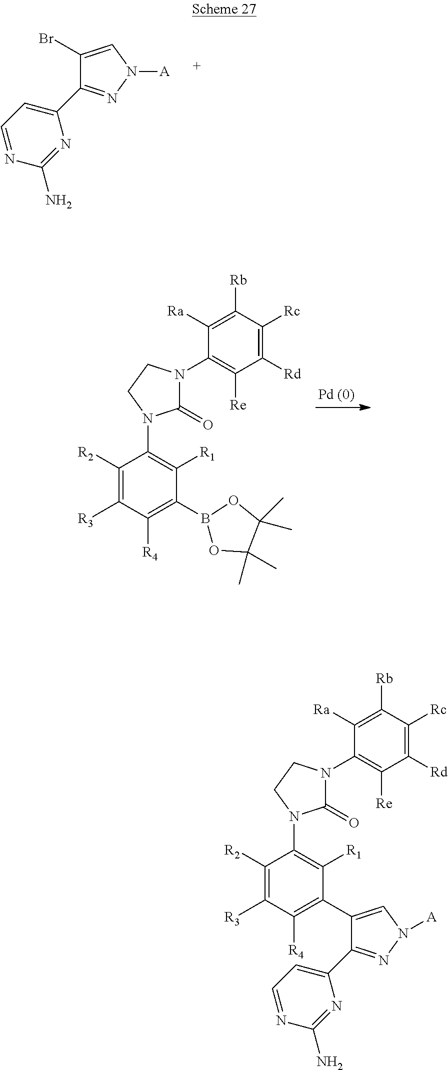

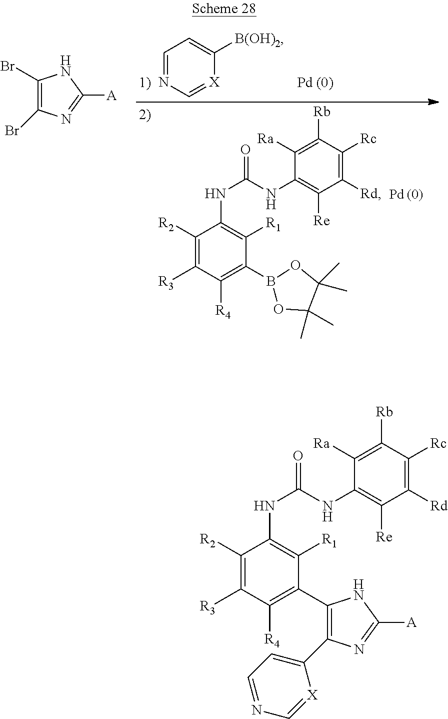

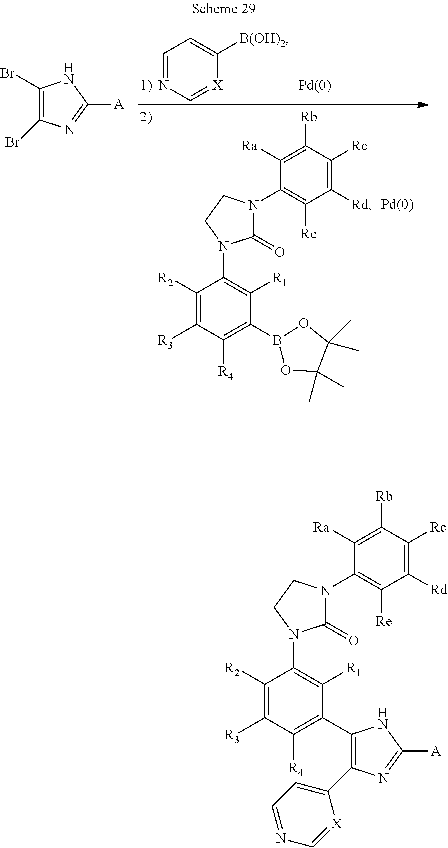

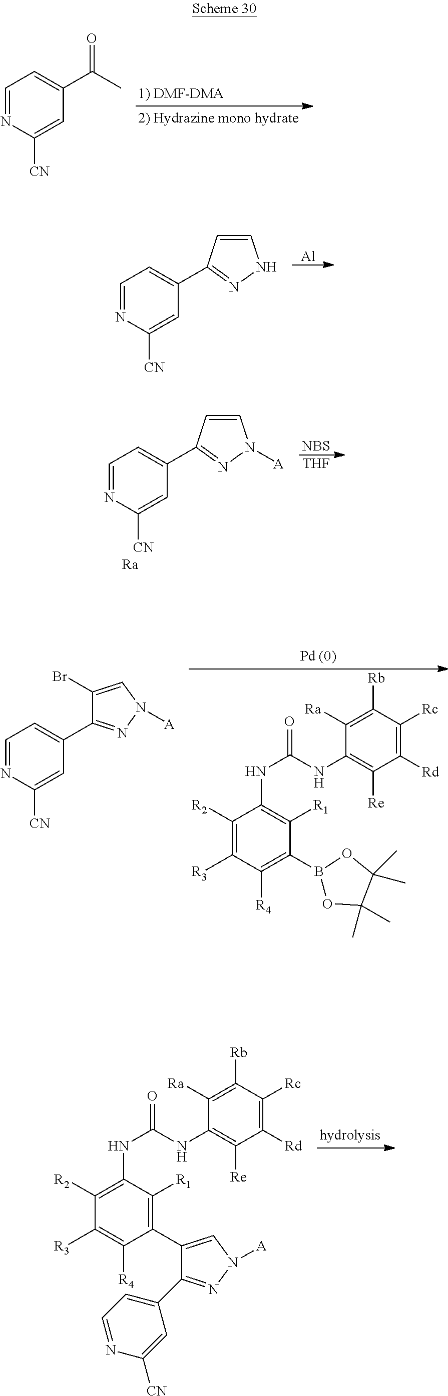

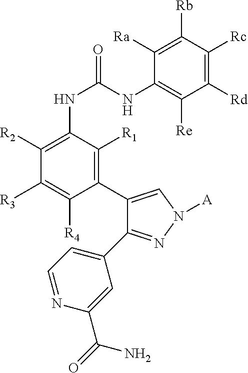

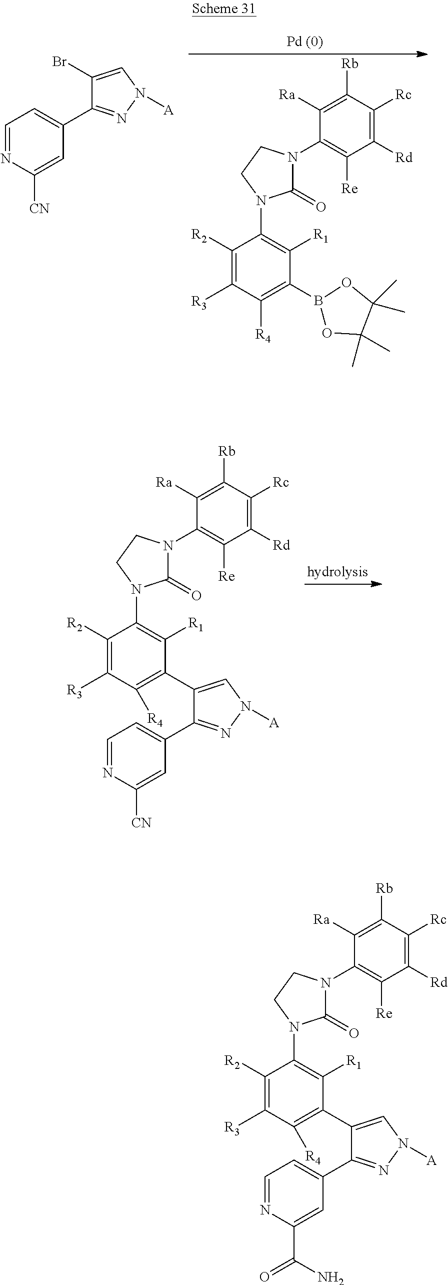

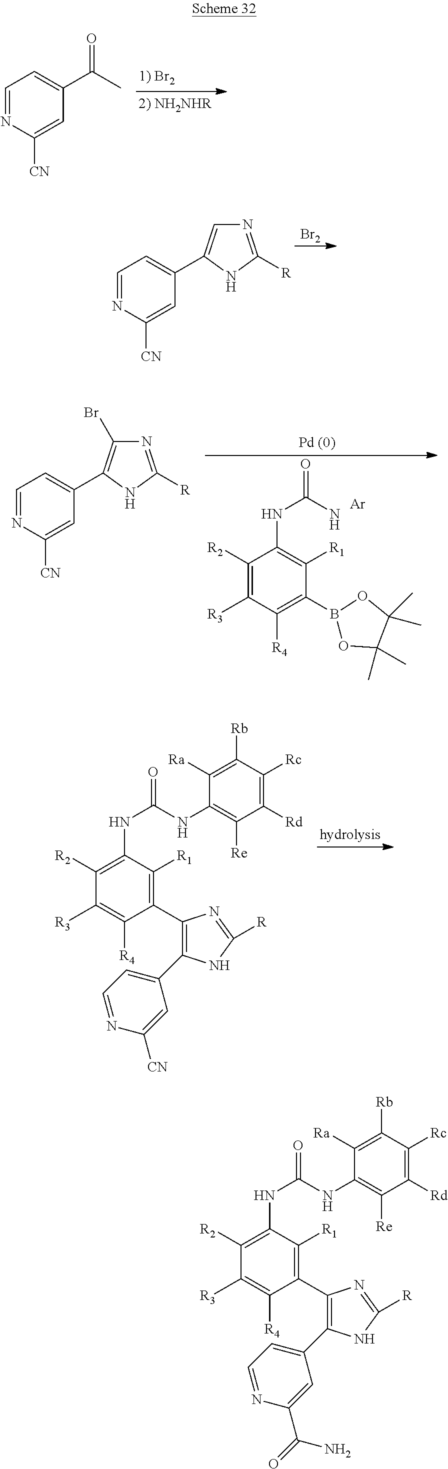

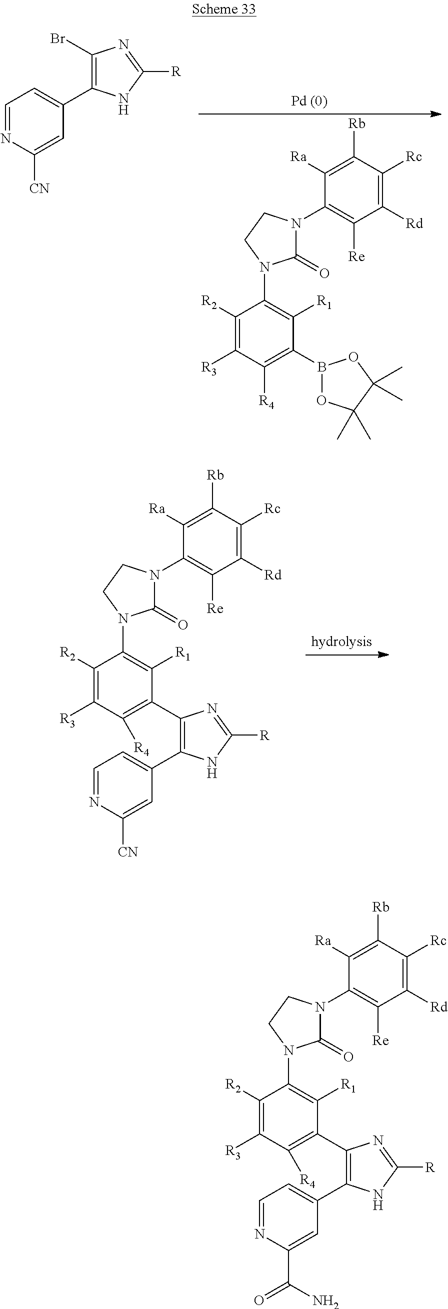

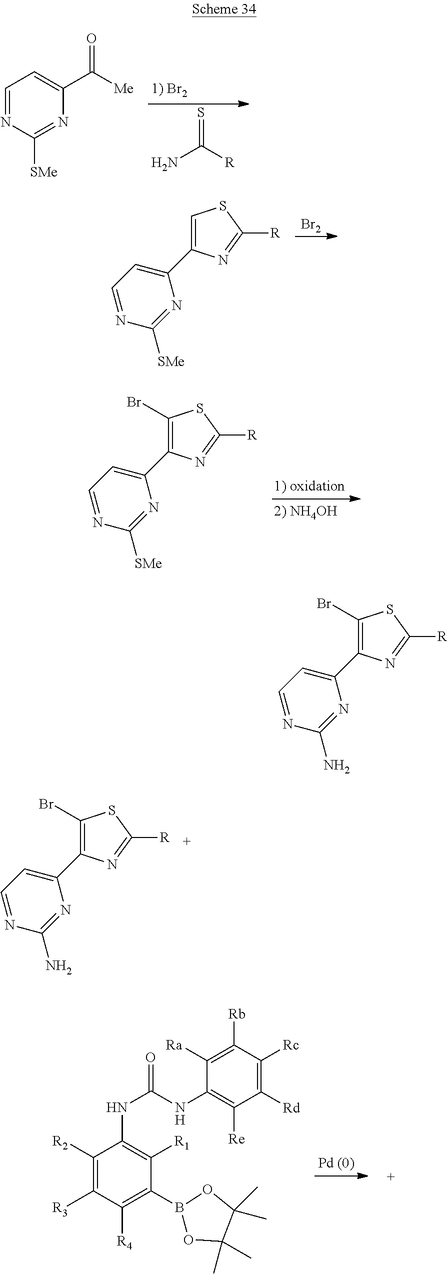

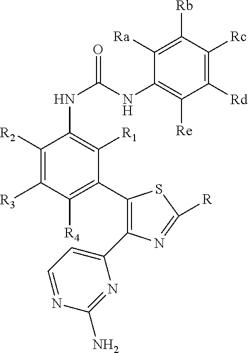

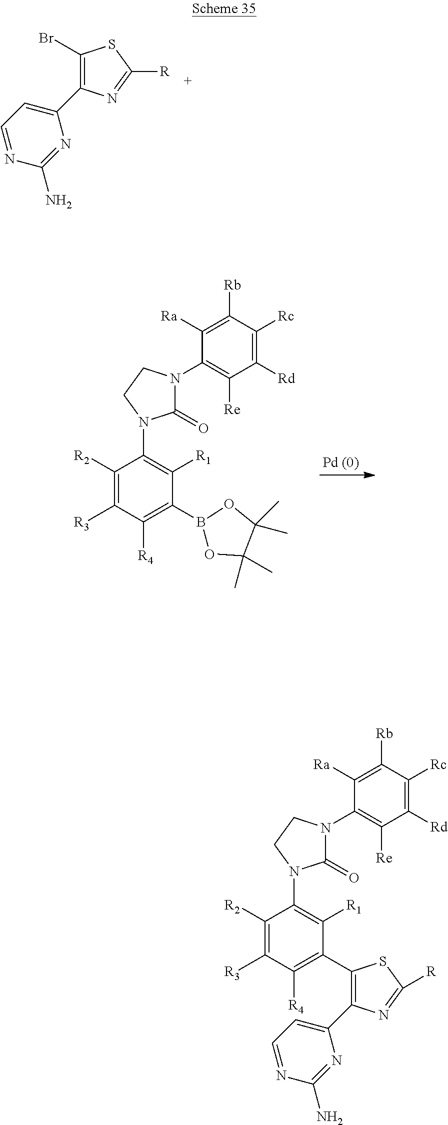

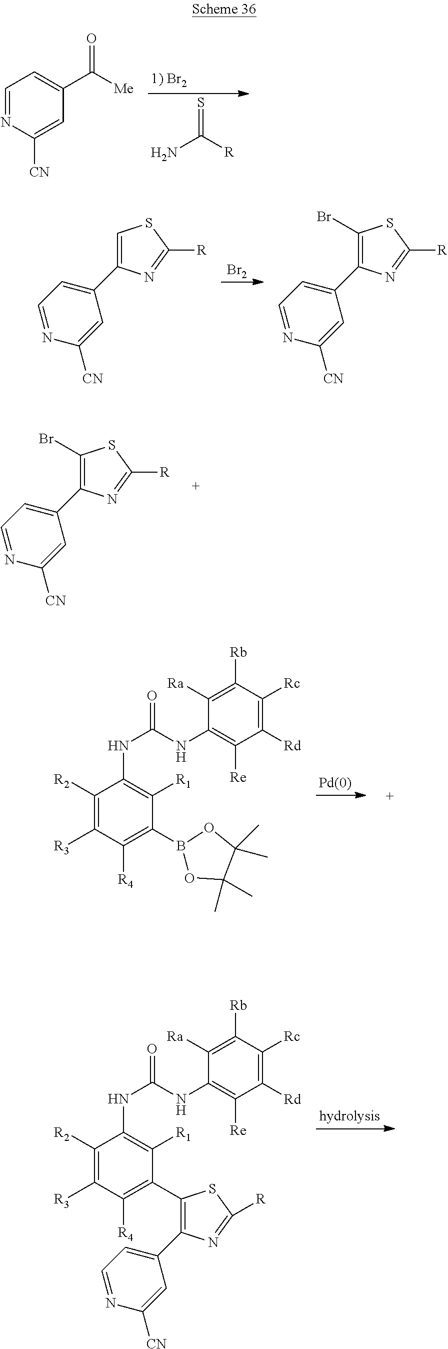

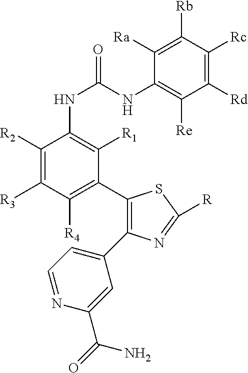

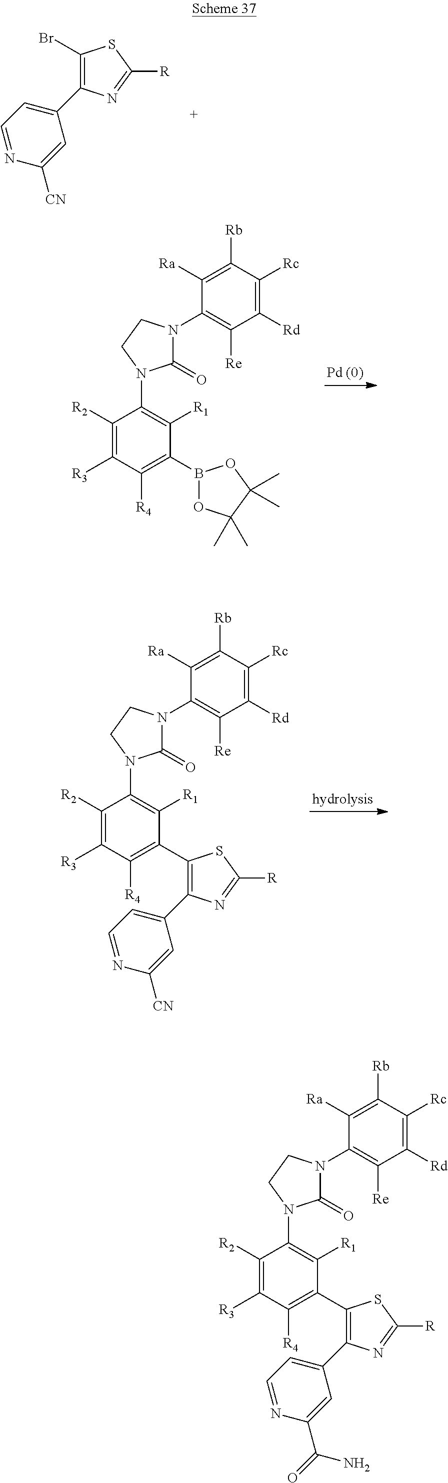

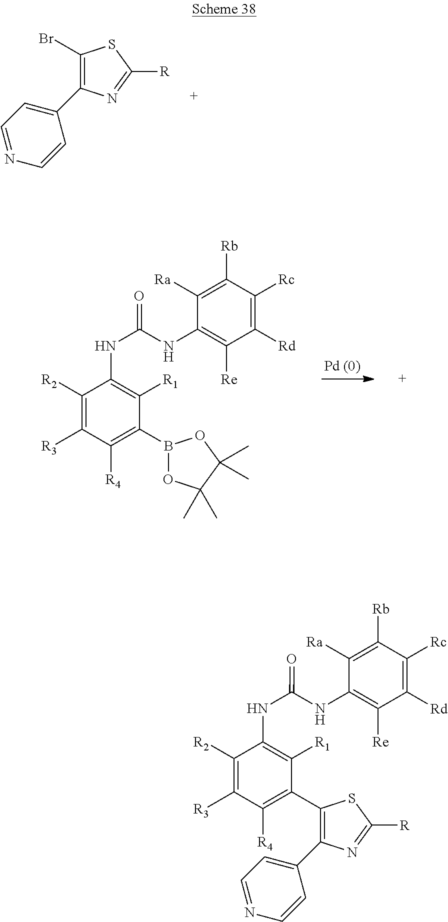

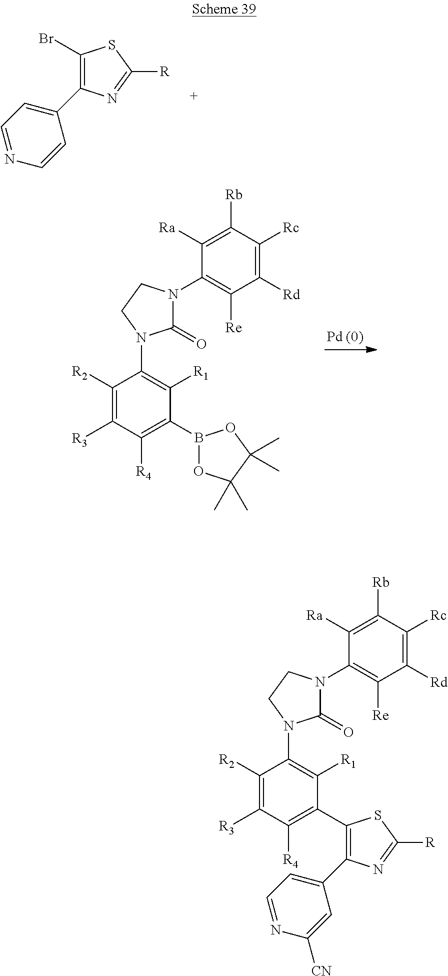

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous; intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody in such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions and Formulations

One embodiment provides a pharmaceutical composition comprising a compound of Formula (I)-(IV), or a stereoisomer, tautomer, hydrate, solvate or pharmaceutically acceptable salt thereof, and at least one pharmaceutically acceptable excipient.

One embodiment provides a pharmaceutical composition comprising a compound of Formula (I), or a stereoisomer, tautomer, hydrate, solvate or pharmaceutically acceptable salt thereof, and at least one pharmaceutically acceptable excipient.

One embodiment provides a pharmaceutical composition comprising a compound of Formula (II), or a stereoisomer, tautomer, hydrate, solvate or pharmaceutically acceptable salt thereof, and at least one pharmaceutically acceptable excipient.

One embodiment provide a pharmaceutical composition comprising a compound of Formula (III), or a stereoisomer, tautomer, hydrate, solvate or pharmaceutically acceptable salt thereof, and at least one pharmaceutically acceptable excipient.

One embodiment provides a pharmaceutical composition comprising a compound of Formula (IV), or a stereoisomer, tautomer, hydrate, solvate or pharmaceutically acceptable salt thereof, and at least one pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, R A, and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include a compound, of Formula (I)-(IV) and at least one pharmaceutically acceptable inactive ingredient. In some embodiments, the compounds described herein are administered as pharmaceutical compositions in which Compounds of Formula (I)-(IV) are mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formula (I)-(IV) with other chemical components (i.e. pharmaceutically acceptable inactive ingredients), such as carriers, excipients, binders, filling agents, suspending agents, flavoring agents, sweetening agents, disintegrating agents; dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound of Formula (I)-(IV) are manufactured in a conventional manner, such as, by way of example only, by means of conventional mixing, dissolving, granulating, dragee-snaking, levigating, emulsifying, encapsulating, entrapping or compression processes.

The pharmaceutical compositions will include at least one compound of Formula (I)-(IV) as an active ingredient in free-acid or free-base form, or in a pharmaceutically acceptable salt form. In addition, the methods and pharmaceutical compositions described herein include the use of N oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

The pharmaceutical compositions described herein, which include a compound of Formula (I)-(IV) are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical preparations that are administered orally include push-fit capsules made of gelatin, as well as soft, sealed capsules made of gelatin and a plasticizer, such as glycerol or sorbitol. The push-fit capsules contain the active ingredients in admixture with filler such as lactose; binders such as starches, and/or lubricants such as talc or magnesium stearate and, optionally, stabilizers. In soft capsules; the active compounds are dissolved or suspended in suitable liquids, such as fatty oils, liquid paraffin, or liquid polyethylene glycols. In some embodiments, stabilizers are added.

All formulations for oral administration are in dosages suitable for such administration.

In one aspect, solid oral dosage forms are prepared by mixing a compound of Formula (I)-(IV) with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a in capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. In still other embodiments, the pharmaceutical formulation is in the form of a tablet. In other embodiments, pharmaceutical formulations of the compounds of Formula (I)-(IV) are in the form of a capsule.

In some embodiments, solid dosage forms, e.g., tablets, effervescent tablets, and capsules, are prepared by mixing particles of a compound of Formula (I)-(IV) with one or more pharmaceutical excipients to form a bulk blend composition. The bulk blend is readily subdivided into equally effective unit dosage forms, such as tablets, pills, and capsules. In some embodiments, the individual-unit dosages include filth coatings. These formulations are manufactured by conventional formulation techniques.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium, lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate; microcrystalline cellulose, cellulose powder, dextrose, dextrates, dextran, starches; pregelatinized starch, hydroxypropylmethycellulose (HPMC), hydroxypropylmethycellulose phthalate, hydroxypropylmethylcellulose acetate stearate-(HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose; microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol, husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, cornstarch, sodium stearyl fumerate, alkali-metal and alkaline earth metal salts, such as aluminum, calcium, magnesium, zinc, stearic acid, Sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethyleric sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, poly vinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/Vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

Compressed tablets are solid dosage forms prepared by compacting the bulk blend of the formulations described above.

In various embodiments, tablets will include one or more flavoring agents.

In other embodiments, the tablets will include a film surrounding the final compressed tablet. In some embodiments, the film coating can provide a delayed release of the compound of Formula (I)-(IV) from the formulation. In other embodiments, the film coating aids in patient compliance (e.g., Opadry® coatings or sugar coating). Film coatings including Opadry® typically range from about 1% to about 3% of the tablet weight.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

In various embodiments, the particles of the compound of Formula (I)-(IV) and one or more excipients are dry blended and compressed into a mass, such as a tablet, having a hardness sufficient to provide a pharmaceutical composition that substantially disintegrates within less than about 30 minutes less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a compound of Formula (I)-(IV) is formulated to include one or more pharmaceutical excipients and flavors. Such a powder is prepared; for example, by mixing the compound of Formula (I)-(IV) and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical solid oral dosage forms are formulated to provide a controlled release of the compound of Formula (I)-(IV). Controlled release refers to the release of the compound of Formula (I)-(IV) froth dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, as an oral dosage form of a pharmaceutical composition as described herein which utilizes an enteric coating to affect release in the small intestine or large intestine. In one aspect, the enteric coated-dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a compound of Formula (I)-(IV), that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following:

Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit-series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, multiparticulate) each containing the formulation described herein. The first group of particles provides a substantially immediate dose of the compound of Formula (I)-(IV) upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions, are described herein or known in the art.

In some embodiments, pharmaceutical formulations are provided that include particles of a compound of Formula (I)-(IV) and at least one dispersing agent or suspending agent for oral administration to a subject. The formulations may be a powder and/or granules for suspension, and upon admixture with water, a substantially uniform suspension is obtained.

In one aspect, liquid formulation dosage forms for oral administration are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of the compound of Formula (I)-(IV), the liquid dosage forms include additives; such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

Furthermore, pharmaceutical compositions optionally include one or more pH adjusting agents or buffering agents, including acids such as acetic, boric, citric, lactic; phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride; sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl, methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl, cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such-as benzalkonium chloride. Preservatives, as used heroin, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), mannitol, menthol, neohesperidin DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphatidylcholine, oils, such as cottonseed oil, groundnut oil, corn germ oil, olive oil, castor oil, and sesame-oil, glycerol, tetrahydrofurfuryl alcohol, polyethylene glycols, fatty acid esters of sorbitan, or mixtures of these substances, and the like.

Representative intranasal formulations are described in, for example, U.S. Pat. Nos. 4,476,116, 5,116,817 and 6,391,452. Formulations that include a compound of Formula (I)-(IV) are prepared as solutions in saline, employing benzyl alcohol or other suitable preservatives, fluorocarbons; and/or other solubilizing of dispersing agents known in the art. See, for example, Ansel, H. C. et al., Pharmaceutical Dosage Forms and Drug Delivery Systems, Sixth Ed. (1995); Preferably these compositions and formulations are prepared with suitable nontoxic pharmaceutically acceptable ingredients. These ingredients are known to those skilled in the preparation of nasal dosage forms and some of these can be found in REMINGTON THE SCIENCE AND PRACTICE OF PHARMACY, 21st edition, 2005. The choice of suitable carriers is dependent upon the exact nature of the nasal dosage form desired, e.g., solutions, suspensions, ointments; or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

For administration by inhalation, a compound of Formula (I)-(IV) is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of such as, by way of example only, gelatin for use in an inhaler or insufflator may be formulated containing a powder mix of the compound described herein and a suitable powder base such as lactose or starch.

Buccal formulations that include a compound of Formula (I)-(IV) are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

In some embodiments, compounds of Formula (I)-(IV) are prepared as transdermal dosage forms. In one embodiments, the transdermal formulations described herein include at least three components: (1) a formulation of a compound of Formula (I)-(IV); (2) a penetration enhancer; and (3) an aqueous adjuvant. In some embodiments the transdermal formulations include additional components such as, but not limited to, gelling agents, creams and ointment bases, and the like. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of the compound of Formula (I)-(IV). In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In one aspect, a compound of Formula (I)-(IV) is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium, chloride, and the like. Prolonged absorption of the injectable pharmaceutical form can be brought about by the use of agents delaying absorption, such as aluminum monostearate and gelatin.

For intravenous injections, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with, an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in, powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein, can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers; stabilizers, tonicity enhancing agents, buffers and preservatives.

In some embodiments, the compounds of Formula (I)-(IV) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polyethers such as polyvinylpyrrolidone, PEG, and the like. In suppository forms of the compositions, a low-melting wax such as, but not limited to, a mixture of fatty acid glycerides, optionally in combination with cocoa butter is first melted.

### Methods of Inhibiting RAF Kinase Signaling

One embodiment provides a method of inhibiting a protein kinase comprising contacting the protein kinase with an inhibitory concentration of a compound of Formula (I)-(IV), or a tautomer, steroisomer, geometric isomer; a pharmaceutically acceptable salt, solvate, or hydrate thereof.

One embodiment provides a method of inhibiting a protein kinase comprising contacting the protein kinase with an inhibitory concentration of a compound of Formula (I).

One embodiment provides a method of inhibiting a protein kinase comprising contacting the protein kinase with an inhibitory concentration of a compound of Formula (II).

One embodiment provides a method of inhibiting a protein kinase comprising contacting the protein kinase with an inhibitory concentration of a compound of Formula (III).

One embodiment provides a method of inhibiting a protein kinase comprising contacting the protein kinase with an inhibitory concentration of a compound of Formula (IV).

Another embodiment provides a Method of inhibiting a protein kinase, wherein the protein kinase is selected from A-RAF, B-RAF and C-RAF. Another embodiment provides a method of inhibiting a protein kinase, wherein the protein kinase is B-RAF. Another embodiment provides a method of inhibiting a protein kinase, wherein the protein kinase is C-RAF. Another embodiment provides a method of inhibiting a protein kinase, wherein the protein kinase is a B-RAF mutant. Another embodiment provides a method of inhibiting a protein kinase, wherein the protein kinase is the B-RAF V600E mutant.

One embodiment provides a method of inhibiting RAF kinase mediated signalling in a cell comprising contacting the cell with an inhibitory concentration of a compound of Formula (II). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (II). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated, signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (III). Another embodiment provides a method of inhibiting RAF kinase mediated signalling in a cell, wherein the cell is characterized by increased activity of the RAS-RAF-MEK-ERK pathway compared to anon-transformed cell. Another embodiment provides a method of inhibiting RAF kinase mediated signalling in a cell, wherein the cell is characterized, by a B-RAF gain-of-function mutation. Another embodiment provides a method of inhibiting RAF kinase mediated signalling in a cell, wherein the cell is characterized by the presence of the B-RAF V600E mutant.

One embodiment provides a method of inhibiting RAF kinase mediated signalling in a cell comprising contacting the cell with an inhibitory concentration of a compound of Formula (IV). Another embodiment provides a method of inhibiting RAF kinase mediated signalling in a cell, wherein the cell is characterized by increased activity of the RAS-RAF-MEK-ERK pathway compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

Another embodiment provides the method of inhibiting a protein kinase wherein the protein kinase is selected from A-RAF, B-RAF and C-RAF. Another embodiment provides a method of inhibiting a protein kinase, wherein the protein kinase is selected from human A-RAF, B-RAF and C-RAF, or a homolog or an ortholog thereof. Another embodiment provides the method of inhibiting a protein kinase wherein the protein kinase is B-RAF. Another embodiment provides the method of inhibiting a protein kinase wherein the protein kinase is the B-RAF V600E mutant. Another embodiment provides the method of inhibiting a protein kinase wherein the protein kinase is the B-RAF G464V mutant. Another embodiment provides the method of inhibiting a protein kinase wherein the protein kinase is C-RAF.

One embodiment provides a method of inhibiting RAF kinase mediated signalling in a cell comprising contacting the cell with an inhibitory concentration of a compound of Formula (I)-(IV). Another embodiment provides a method of inhibiting RAF kinase mediated signalling in a cell, wherein the cell is characterized by increased activity of the RAS-RAF-MEK-ERK pathway-compared to a non-transformed cell. Another embodiment provides a method of inhibiting RAF kinase mediated signalling in a cell, wherein the cell is characterized by a B-RAF gain-of-function mutation. Another embodiment provides a method of inhibiting RAF kinase mediated signalling in a cell, wherein the cell is characterized by the presence of the B-RAF V600E mutant.

### Methods of Treatment

One embodiment provides a method of treating a human disease or disorder mediated by RAF kinase signalling comprising administering to a patient a therapeutically effective amount of a composition comprising a compound of Formula (I). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a composition comprising a compound of Formula (II). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a Composition comprising a compound of Formula (III). Another embodiment provides the method wherein the RAF kinase is B-RAF kinase. Another embodiment provides the method wherein the RAF kinase, is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by RAF kinase signalling comprising administering to a patient a therapeutically effective amount of a composition comprising a compound of Formula (IV). Another embodiment provides the method wherein the RAF kinase is B-RAF kinase. Another embodiment provides the method wherein the RAF kinase is selected from human A-RAF, B-RAF and C-RAF, or a homolog or an ortholog thereof. Another embodiment provides the method of treating human disease or disorder wherein the disease or disorder is a proliferative disease. Another embodiment provides the method of treating human disease or disorder wherein the proliferative disease is selected from melanoma, ovarian cancer, colorectal cancer, thyroid cancer, cholangiocarcinoma, or lung adenocarcinoma.

One embodiment provides a method of treating a human disease or disorder mediated by the RAF kinase signalling pathway comprising administering to a patient a therapeutically effective amount of a composition comprising a compound of Formula (I)-(IV), or a tautomer, Steroisomer, geometric isomer, a pharmaceutically acceptable salt, solvate, or hydrate thereof.

One embodiment provides a method of treating a human-disease or disorder mediated by RAF kinase signalling comprising administering to a patient a therapeutically effective amount of a composition comprising a compound of Formula (II)-(IV). Another embodiment provides a method of treating a human disease or disorder mediated by RAF kinase signalling, wherein the RAF kinase is B-RAF kinase.

Another embodiment provides a method of treating a human disease or disorder mediated by RAF kinase signalling, wherein the disease or disorder is a proliferative disease. Another embodiment provides a method of treating a human proliferative disease, wherein the proliferative disease is selected from melanoma, ovarian cancer, colorectal cancer, thyroid cancer, cholangiocarcinoma, or lung adenocarcinoma.

Another embodiment provides a method of treating a human disease or disorder mediated by RAF kinase signalling wherein the disease or disorder is a proliferative disease. A further embodiment provides a method of treating proliferative disease wherein the proliferative disease is melanoma, ovarian cancer, colorectal cancer, thyroid cancer, cholangiocarcinoma, or lung adenocarcinoma.

One embodiment provides a method of treating a human proliferative disease or disorder selected from the group consisting of oral cancer, prostate cancer, rectal cancer, non-small cell lung cancer, lip and oral cavity cancer, liver cancer, lung cancer, anal cancer, kidney cancer, vulvar cancer; breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

One embodiment provides a method of treating cancer wherein the cancer is a carcinoma, a tumor, a neoplasm, a lymphoma, a melanoma, a glioma, a sarcoma, and a blastoma.

In another embodiment the carcinoma is selected from the group consisting of carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma; hepatocellular carcinoma, soft tissue carcinomas, bronchial gland carcinomas, capillary carcinoma, Bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment the tumor is selected from the group consisting of: astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In another embodiment the neoplasm is selected from the group consisting of: intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma and malignant thymoma.

In another embodiment the lymphoma is selected from the group consisting of nervous system lymphoma, AIDS-related lymphoma, cutaneous T-cell lymphoma, non-Hodgkin's lymphoma, lymphoma, and Waldenstrom'S macroglobulinemia.

In another embodiment the melanoma is selected from the group consisting of: acral lentiginous melanoma, superficial spreading melanoma, uveal melanoma, lentigo maligna melanomas, melanoma, intraocular melanoma, adenocarcinoma nodular Melanoma, and hemangioma.

In another embodiment the sarcoma is selected from the group consisting of adenomas, adenosarcoma; chondosarcoma endometrial stromal-sarcoma, Ewing's sarcoma, Kaposi's sarcoma, leiomyosarcoma, rhabdomyosarcoma, sarcoma, uterine sarcoma, osteosarcoma, and pseudosarcoma.

In another embodiment the glioma is selected from the group consisting of: glioma, brain stern glioma, and hypothalamic and visual pathway glioma.

In another embodiment the blastoma is selected from the group consisting of: pulmonary blastoma, pleuropulmonary blastoma, retinoblastoma neuroblastoma, medulloblastoma, glioblastoma, and hemangiblastomas.

One embodiment provides a method of treating a veterinary disease or disorder mediated by the RAF kinase signalling pathway comprising administering to a patient a therapeutically effective amount of a composition comprising a compound of Formula (I)-(IV), or a tautomer, steroisomer, geometric isomer, a pharmaceutically acceptable salt, solvate, or hydrate thereof.

One embodiment provides a method of treating a parasitic disease or fungal infection in humans or animals comprising administering to a subject a therapeutically effective amount of a composition comprising a compound of Formula (I)-(IV), or a tautomer, stereoisomer, geometric isomer, a pharmaceutically acceptable salt, solvate, or hydrate thereof.

### EXAMPLES

### I. Chemical Synthesis

### Synthesis of 4-(5-bromo-2-methyl-thiazol-4-yl)-pyridine (Intermediate 1)

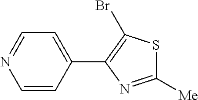 [SMILES: Cc1nc(-c2ccncc2)c(Br)s1]

Step 1: 2-bromo-1-pyridin-4-yl-ethanoate hydrobromide (2.0 g, 7.1.1 mmol) and thioacetamide (0.535 g, 7.11 mmol) were dissolved in EtOH (35 mL) and the mixture was heated to reflux for 1 h. After cooling, the precipitated solids were collected by filtration. [M+H]+ m/z 177.

Step 2: 4-(2-methyl-thiazol-4-yl)-pyridine (1.0 g; 5.67 mmol) was dissolved in 30 mL acetic acid and bromine was added (0.32 mL, 6.24 mmol). The mixture was heated at 80° C. for 3 days. The mixture was diluted with aqueous KOH to pH 10-12. The aqueous solution was extracted with ethyl acetate (3×), the organic layer was dried over MgSO₄ and the material was purified by silica gel column chromatography using 1:1 hexanes/ethyl acetate. LCMS indicated desired product and 10% dibrominated material. The material was dried at 50° C. under vacuum for 16 h to afford a black solid, 505 mg. ¹H NMR (DMSO-d6, ppm) δ 8.76 (d, 2H), 7.93 (d, 2H), 2.73 (s, 3H). [M+H]+ m/z 255.

### Synthesis of 3-(2-methyl-4-pyridin-4-yl-thiazol-5-yl)-phenylamine (Intermediate 2)

A Microwave vessel was charged with 4-(5-bromo-2-methyl-thiazol-4-yl)-pyridine (intermediate 1) (40 mg, 0.126 mmol), 3-aminophenylboronic acid (25 mg, 0.138 mmol) and PdCl₂(PPh₃)₂ (4.4 mg, 0.006 mmol) under nitrogen atmosphere. Saturated aqueous sodium carbonate (0.5 mL) and 1,4-dioxane (0.5 mL) were added. The vessel was capped and heated in a Biotage Initiator microwave instrument at 150° C. for 30 min. The reaction mixture was diluted with saturated aqueous sodium carbonate and was filtered through celite. The filtrate was extracted with EtOAc (3×). The aqueous layer was isolated and acidified to pH 4.5 with concentrated HCl. The resulting precipitate was filtered, washed With water, and dried in vacuo to give 38 mg of the title compound as a white solid (84% yield).

### Synthesis of 4-(5-bromo-2-(1,1-dimethylethyl)-thiazol-4-yl)-pyridine (Intermediate 3)

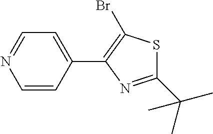 [SMILES: CC(C)(C)c1nc(-c2ccncc2)c(Br)s1]

Step 1: 4-(2-(1,1-dimethylethyl)-thiazol-4-yl)-pyridine. A stirring suspension of 2-bromo-1-pyridin-4-yl-ethanone hydrobromide (5 g, 17.81 nmol) and 2,2,2-trimethylthioacetamide (2.086 g, 17.8 mmol) in absolute EtOH (100 mL) was heated to reflux overnight. After cooling, the reaction mixture was concentrated in vacuo and the resulting residue was partitioned between DCM and saturated aqueous sodium carbonate solution. The aqueous layer was extracted with DCM (3×). The combined organics were dried over magnesium sulfate, filtered, and concentrated in vacuo to provide 3.8 g of the titled product as an orange solid (97% yield): ¹H NMR (DMSO-d6, ppm) δ 1.47 (s, 9H), 7.92 (d, 2H), 8.36 (s, 1H), 8.64 (d, 2H); [M+H]⁺ m/z 219.

Step 2: 4-(5-bromo-2-(1,1-dimethylethyl)-thiazol-4-yl)-pyridine. 4-(2-(1,1-Dimethylethyl)-thiazol-4-yl)-pyridine (3.8 g, 17.4 mmol) was dissolved in 100 mL acetic acid and bromine was added (1.8 mL, 34.81 mmol). The mixture was heated at 80° C. for 5 days, with more bromine added throughout, up to 2.7 mL. After 5 days, 6 mL of pyridine and 1 mL of bromine were added, and the reaction mixture was heated to 100° C. overnight. After cooling, the reaction mixture was poured onto iced 1M aqueous sodium bisulfate solution (200 mL). The solution was then basified to pH 12 with 8 N aqueous NaOH, and then extracted with DCM (3×). The combined organics were dried over magnesium sulfate, filtered, and adsorbed on silica gel. Purification by flash silica gel chromatography using a to gradient of 0-60% EtOAc/hexane afforded 3.8 g of the titled product as a dark oil (73% yield): ¹H NMR (DMSO-d6, ppm) δ 1.43 (s, 9H), 7.92 (d, 2H), 8.73 (d, 2H); [MαH]⁺ m/z 297, 299.

### Synthesis of 3-(2-(1,1-dimethylethyl)-4-pyridin-4-yl-thiazol-5-yl)-phenylamine (Intermediate 4)

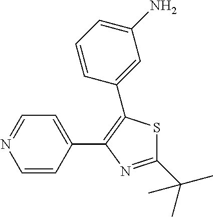 [SMILES: CC(C)(C)c1nc(-c2ccncc2)c(-c2cccc(N)c2)s1]

Intermediate 4 was prepared following the same procedure used for the preparation of intermediate 2 with the use of 4-(5-bromo-2-(1,1-dimethylethyl)-thiazol-4-yl)-pyridine as the starting material. ¹H NMR (DMSO-d6, ppm) δ 1.47 (s, 9H), 5.31 (s, 2H), 6.48 (d, 1H), 6.59 (s, 1H), 6.62 (d, 1H), 7.08 (t, 1H), 7.47 (d, 2H), 8.52 (d, 2H); [M+H]⁺ m/z 310.

### Synthesis of 3-(2-methyl-4-pyridin-4-yl-thiazol-5-yl)-phenylacetic acid (Intermediate 5)

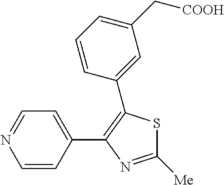 [SMILES: Cc1nc(-c2ccncc2)c(-c2cccc(CC(=O)O)c2)s1]

To a microwave vessel under nitrogen atmosphere was added 4-(5-bromo-2-methyl-thiazol-4-yl)-pyridine (100 mg, 0.392 mmol), 3-(carboxymethyl)phenylboronic acid pinacol ester (113 mg, 0.431 mmol), and PdCl₂(PPh₃)₂ (14 mg, 0.0196 mmol), 1,4-dioxane (1 mL) and saturated aqueous sodium carbonate solution (1 mL). The reaction mixture was heated in a Biotage Initiator microwave instrument at 150° C. for 30 min, diluted with 15% aqueous NaOH (3 mL) and filtered thru a plug of celite. The filtrate was extracted with EtOAc (3×), and the aqueous layer was acidified to pH 4.5 with conc. HCl. The resulting precipitate was filtered, washed with water, and dried in vacuo to yield 71 mg of the titled product as a brown solid (58% yield): ¹H NMR (DMSO-d6, ppm) δ 2.57 (s, 3H), 3.62 (s, 2H), 7.25 (d, 1H), 7.34 (m, 2H), 7.41 (m, 3H), 8.50 (d, 2H); [M+H]⁺ m/z 311.

### Synthesis of 2-amino-4-(5-bromo-2-methyl-thiazol-4-yl)-pyridine (Intermediate 6)

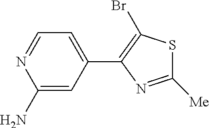 [SMILES: Cc1nc(-c2ccnc(N)c2)c(Br)s1]

Step 1: 4-(5-bromo-2-methyl-thiazol-4-yl)-pyridine N-oxide. To a stirring solution of 4-(5-bromo-2-methyl-thiazol-4-yl)-pyridine (765 mg, 3 mmol) in DME (20 mL) was added 3-chloroperbenzoic acid (518 mg, 6 mmol). Additional 3-chloroperbenzoic acid (577 mg) was added after 21 h. After 24 h, the reaction mixture was concentrated in vacuo and the resulting residue was partitioned between 15% aqueous NaOH and EtOAc. The organic layer was washed with 15% aqueous NaOH and brine, dried over magnesium sulfate, filtered, and concentrated in vacuo to give 749 mg of the titled product as a white solid (92% yield): ¹H NMR (DMSO-d6, ppm) δ 2.71 (s, 3H), 7.98 (d, 2H), 8.32 (d, 2H); [M+H]⁺ m/z 271, 273.

Step 2: 2-amino-4-(5-bromo-2-methyl-thiazol-4-yl)-pyridine. To a suspension of 4-(5-bromo-2-methyl-thiazol-4-yl)-pyridine N-oxide (745 mg, 2.75 mmol) and tert-butylamine (1.45 mL, 13.74 mmol) in trifluoromethylbenzene (15 mL) at 0° C. was added p-toluenesulfonic anhydride (1.8 g, 5.5 mmol) in one portion. After 2 h at 0° C., the reaction mixture was concentrated in vacuo and the residue was partitioned between EtOAc and water. The organic layer was washed with water and brine, then dried over magnesium sulfate, filtered and concentrated in vacuo. The residue was dissolved in TEA (15 mL) and the reaction mixture was stirred at 65° C. for 3 h. The reaction mixture was concentrated in vacuo, treated with ice and 15% aqueous NaOH to basic pH. The aqueous layer was extracted with EtOAc, and the organic layer was washed with water and brine, and dried over magnesium sulfate, filtered, and adsorbed on silica gel. Purification by flash silica gel chromatography using a gradient of 20-100% EtOAc/hexane afforded 449 mg of the titled product as a tan solid (60% yield): ¹H NMR (DMSO-d6, ppm) δ 2.70 (s, 3H), 6.12 (s, 2H), 7.00 (m, 2H), 8.00 (d, 1H); [M+H]⁺ m/z 270.

### Synthesis of 3-(2-methyl-4-(2-aminopyridin-4-yl)-thiazol-5-yl)-phenylamine (Intermediate 7)

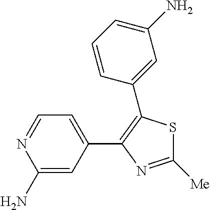 [SMILES: Cc1nc(-c2ccnc(N)c2)c(-c2cccc(N)c2)s1]

Intermediate 7 was prepared following the same procedure used for the preparation of intermediate 2 with the use of 2-amino-4-(5-bromo-2-methyl-thiazol-4-yl)-pyridine as starting material: ¹H NMR (DMSO-d6, ppm) δ 2.70 (s, 3H), 5.27 (s, 2H), 5.92 (s, 2H), 6.39 (dd, 1H), 6.45 (d, 1H), 6.57 (m, 2H), 6.77 (s, 1H), 7.05 (t, 1H), 7.77 (d, 1H); [M+H]⁺ m/z 283.

### Synthesis of 2-fluoro-5-(2-methyl-4-pyridin-4-yl-thiazol-5-yl)-phenylamine (Intermediate 8)

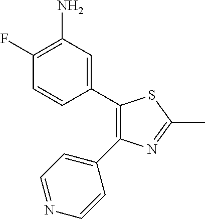 [SMILES: Cc1nc(-c2ccncc2)c(-c2ccc(F)c(N)c2)s1]

Intermediate 8 was prepared following the same procedure used for the preparation of intermediate 2 with the use of 3-amino-4-fluorophenylboronic acid as starting material: [M+H]⁺ m/z 286.

### Synthesis of 4-(5-bromo-4-(pyridin-4-yl)thiazol-2-yl)morpholine (Intermediate 9)

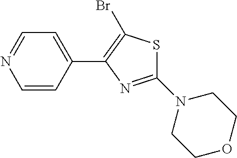 [SMILES: Brc1sc(N2CCOCC2)nc1-c1ccncc1]

Step 1: 2-Bromo-1-pyridin-4-yl-ethanone hydrobromide (1.0 g, 3.55 mmol) and morpholine-4-carbothioamide (0.520 g, 3.55 mmol) were dissolved in. EtOH (20 mL) and the mixture was refluxed for 1 hr. After cooling, the yellow solids were collected by filtration, 74.8% yield. [M+H]⁺ m/z 248.

Step 2: 4-(4-(pyridin-4-yl)thiazol-2-yl)morpholine hydrobromide (0.454 g, 1.38 mmol) was dissolved in 15 mL THF and NBS was added (0.32 mg, 1.79 mmol). The mixture was heated at 70° C. for 2 h and the solids were collected to afford 4-(5-promo-4-(pyridin-4-yl)thiazol-2-yl)morpholine as an orange solid: 533 mg. [M+H]⁺ m/z 326.

### Synthesis of 4 3-(2-morpholin-4-yl-4-pyridin-4-yl-thiazol-5-yl)-phenylamine (Intermediate 10)

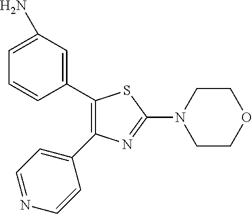 [SMILES: Nc1cccc(-c2sc(N3CCOCC3)nc2-c2ccncc2)c1]

Intermediate 10 was prepared following the same procedure used for the preparation of intermediate 2 starting with intermediate 9 as starting material: [M+H]⁺ m/z 339.

### Synthesis of 4-(2-(5-bromo-4-(pyridin-4-yl)thiazol-2-yl)ethyl)morpholine (Intermediate 11)

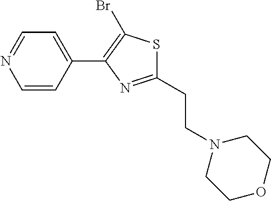 [SMILES: Brc1sc(CCN2CCOCC2)nc1-c1ccncc1]

Step 1: 2-Bromo-1-pyridin-4-yl-ethanone hydrobromide (0.742 g, 2.64 mmol) and 3-morpholinopropanethioamide (0.5 g, 2.64 mmol) were dissolved in EtOH (15 mL) and the mixture was refluxed for 1 h. After cooling, the solids were collected by filtration, 100% yield. [M+H]+ m/z 291.

Step 2: 4-(2-(4-(pyridin-4-yl)thiazol-2-yl)ethyl)morpholine (0.482 g, 1.30 mmol) was dissolved in 5 mL dichloromethane and 5 mL saturated sodium bicarbonate, then bromine was added (0.74 mL, 1.43 mmol). The mixture was stirred rapidly and judged complete by LCMS after about 90 minutes. The layers were separated and the organic layer was washed with NaHSO₃ (1 M) and dried over MgSO₄. Concentration in vacuo afforded desired product. ¹H NMR (DMSO-d6, ppm) δ 2.44 (br s, 4H), 3.42 (q, 2H), 3.59 (m, 4H), 7.85 (d, 2H), 7.86 (t, 1H), 8.66 (d, 2H); [M+H]⁺ m/z 370.

### Synthesis of [5-(3-amino-phenyl)-4-pyridin-4-yl-thiazol-2-yl]-(2-morpholin-4-yl-ethyl)-amine (Intermediate 12)

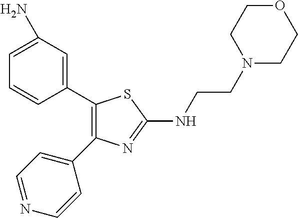 [SMILES: Nc1cccc(-c2sc(NCCN3CCOCC3)nc2-c2ccncc2)c1]

Intermediate 12 was prepared following the same procedure used for the preparation of intermediate 2 starting with intermediate 1.1 as starting material: [M+H]⁺ m/z 382.

### Synthesis of 1-[2-Fluoro-5-(4,4,5,5-tetramethyl-[1,3,2]dioxaborolan-2-yl)-phenyl]-3-(4-chlorophenyl)-urea (Intermediate 13)

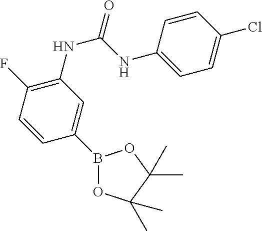 [SMILES: CC1(C)OB(c2ccc(F)c(NC(=O)Nc3ccc(Cl)cc3)c2)OC1(C)C]

A vial was charged with 5-bromo-2-fluoroaniline (1 g, 5.26 mmol), bis(pinacolato)diboron (1.6 g, 6.31 mmol), potassium acetate (1.03 g, 10.52 mmol), Pd(dppf)Cl₂.CH₂Cl₂ (129 mg, 0.158 mmol), and DMF (10 mL) under nitrogen atmosphere. After stirring for 2 h at 100° C. the reaction mixture was concentrated in vacuo, the residue was triturated with EtOAc and filtered through a pad of celite. The filtrate was adsorbed on silica gel. Purification by flash silica gel chromatography using a gradient of 0-30% EtOAc/hexane afforded 1.25 g of pinacol 3-amino-4-fluoroboronate as a light yellow oil (quant.): ¹H NMR (CDCl₃, ppm) δ 1.36 (s, 12H), 3.71 (broad s, 2H), 7.00 (dd, 1H), 7.19 (m, 1H), 7.26 (dd, 1H); [M+H]⁺ m/z 238.

To a solution of pinacol 3-amino-4-fluoroboronate (300 mg, 1.013 mmol) in THF (3 mL) under nitrogen atmosphere was added 4-chlorophenylisocyanate (171 mg, 1.11 mmol). The reaction mixture was stirred at room temperature for 22 h then it was adsorbed on silica gel. Purification by flash silica gel chromatography using a gradient of 0-25% EtOAc/hexane afforded 307 mg of 1-[2-fluoro-5-(4,4,5,5-tetramethyl-[1,3,2]dioxaborolan-2-yl)-phenyl]-3-(4-chlorophenyl)-urea as a white solid (77% yield): ¹H NMR (DMSO-d6, ppm) δ 1.32 (s, 12H), 7.27 (dd, 1H), 7.35 (m, 3H), 7.51 (d, 2H), 8.52 (d, 1H), 8.62 (s, 1H), 9.24 (s, 1H); [M+H]⁺ m/z 391.

### Synthesis of 1-[2-Fluoro-3-(4,4,5,5-tetramethyl-[1,3,2]dioxaborolan-2-yl)-phenyl]-3-(4-chlorophenyl)-urea (Intermediate 14)

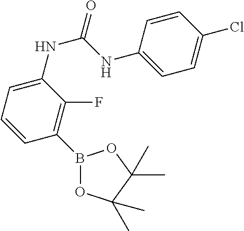 [SMILES: CC1(C)OB(c2cccc(NC(=O)Nc3ccc(Cl)cc3)c2F)OC1(C)C]

Step 1: A solution of 3-bromo-2-fluoro-nitrobenzene (8.4 g, 38.2 mmol) in (2:2:1) EtOH/AcOH/H₂O (175 mL) was treated with iron powder (10.6 g, 191 mmol). The reaction mixture was stirred at 85° C. for 1.5 h, then cooled to room temperature and filtered through celite. The filtrate was concentrated in vacuo and the residue was partitioned between EtOAc and 1 N aqueous KOH. The aqueous layer was extracted with EtOAc (2×), and the combined organics were dried over MgSO₄, filtered, and concentrated in vacuo to give 3.84 g of 3-bromo-2-fluoroaniline as an orange-pink liquid (53% yield).

Step 2: A flask was charged with 3-bromo-2-fluoroaniline (3.84 g, 20.2 mmol), bis(pinacolato)diboron (6.16 g, 24.25 mmol), potassium acetate (3.96 g, 40.4 mmol), Pd(dppf)Cl₂.CH₂Cl₂ (495 mg, 0.606 mmol), and DMF (40 mL) under nitrogen atmosphere. After stirring for 23 h at 100° C. the reaction mixture was concentrated in vacuo, the residue was triturated with hexanes and filtered through a pad of celite. The filtrate was adsorbed on silica gel. Purification by flash silica gel chromatography using a gradient of 0-30% EtOAc/hexane afforded 3.65 g of pinacol 3-amino-2-fluoroboronate as an off white solid (76% yield): ¹H NMR (CDCl₃, ppm) δ 1.39 (s, 12H), 3.73 (broad s, 2H), 6.91 (dt, 1H), 6.97 (t, 1H), 7.11 (m, 1H).

Step 3: To a solution of pinacol 3-amino-2-fluoroboronate (286 mg, 1.206 mmol) in THF (5 mL) under nitrogen atmosphere was added 4-chlorophenylisocyanate (204 mg, 1.327 mmol). The reaction mixture was stirred at room temperature for 23 h, quenched with a few drops of MeOH and concentrated in vacuo to give 471 mg of 1-[2-fluoro-3-(4,4,5,5-tetramethyl-[1,3,2]dioxaborolan-2-yl)-phenyl]-3-(4-chlorophenyl)-urea as a tan solid (quant.): ¹H NMR (DMSO-d6, ppm) δ 1.33 (s, 12H), 7.17 (t, 1H), 7.26 (m, 1H), 7.36 (d, 2H), 7.51 (d, 2H), 8.24 (t, 1H), 8.52 (s, 1H), 9.26 (s, 1H); [M+H]⁺ m/z 391.

### Synthesis of 1-[2-Fluoro-3-(4,4,5,5-tetramethyl-[1,3,2]dioxaborolan-2-yl)-phenyl]-3-(4-trifluoromethylphenyl)-urea (Intermediate 15)

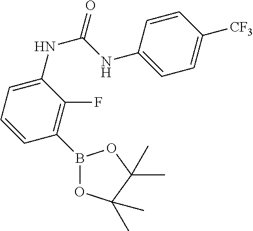 [SMILES: CC1(C)OB(c2cccc(NC(=O)Nc3ccc(C(F)(F)F)cc3)c2F)OC1(C)C]

Intermediate 15 was synthesized in a similar fashion to 1-[2-fluoro-3-(4,4,5,5-tetramethyl-[1,3,2]dioxaborolan-2-yl)-phenyl]-3-(4-chlorophenyl)-urea (Intermediate 13) using 4-trifluorophenylisocyanate as reagent. ¹H NMR (DMSO-d6, ppm) δ 1.33 (s, 12H), 7.18 (t, 1H), 7.29 (m, 1H), 7.68 (m, 4H), 8.25 (dt, 1H), 8.61 (s, 1H), 9.52 (s, 1H).

### Synthesis of 2,4-Difluoro-5-(3-(4-(trifluoromethyl)phenyl)ureido)phenylboronic acid (Intermediate 16)

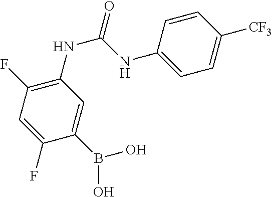 [SMILES: O=C(Nc1ccc(C(F)(F)F)cc1)Nc1cc(B(O)O)c(F)cc1F]

To a solution of 5-amino-2,4-difluorophenylboronic acid (104 mg, 0.497 mmol) in THF (2 mL) under nitrogen atmosphere was added 4-trifluoromethylphenyl isocyanate (0.07 mL, 0.497 mmol) dropwise. The reaction mixture was stirred for 1 h, then concentrated and dried in vacuo to provide 173 mg of the titled compound as dark solid (quant.).

### Synthesis of 1-[3-(4,4,5,5-Tetramethyl-[1,3,2]dioxaborolan-2-yl)-phenyl]-3-(4-trifluoromethylphenyl)-urea (Intermediate 17)

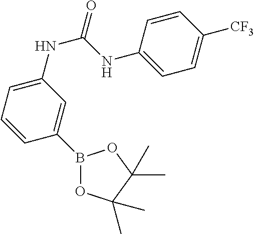 [SMILES: CC1(C)OB(c2cccc(NC(=O)Nc3ccc(C(F)(F)F)cc3)c2)OC1(C)C]

To a solution of 3-(4,4,5,5-tetramethyl-[1,3,2]dioxaborolan-2-yl)-aniline (500 mg, 2.28 mmol) in THF (5 mL) under nitrogen atmosphere was added 4-trifluoromethylphenyl isocyanate (0.32 mL, 2.28 mmol) dropwise. The reaction mixture was stirred for 1 h, then concentrated and dried in vacuo to provide 1.02 g of the titled compound as an off white solid (quant.): ¹H NMR (DMSO-d6, ppm) δ 1.33 (s, 12H), 7.34 (m, 2H), 7.52 (m, 1H), 7.65 (d, 2H), 7.69 (d, 2H), 7.92 (s, 1H), 8.88 (s, 1H), 9.08 (s, 1H); [M+H]⁺ m/z 407.

### Synthesis of 1-(3-(2-tert-butyl-4-(pyridin-4-yl)thiazol-5-yl)phenyl)-3-p-tolylurea, pyridine N-oxide (Intermediate 18)

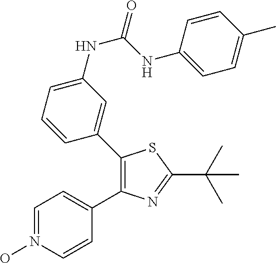

To a solution of 1-(3-(2-tert-butyl-4-(pyridin-4-yl)thiazol-5-yl)phenyl)-3-p-tolylurea (715 mg, 11.615 mmol) in DME (15 mL) was added mCPBA (837 mg, 4.85 mmol). The reaction mixture was stirred for 2 h, then it was diluted With EtOAc (100 mL) and washed with 1N aqueous NaOH (2×) and brine. The organic layer was dried over magnesium sulfate, filtered, and concentrated in vacuo to give 530 mg of the titled product.

### Synthesis of 4-(5-Bromo-2-amino-thiazol-4-yl)-pyridine (Intermediate 19)

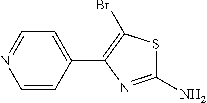 [SMILES: Nc1nc(-c2ccncc2)c(Br)s1]

A mixture of 2-bromo-1-pyridin-4-yl-ethanone hydrobromide (10 g, 35.59 mmol) and thiourea (2.71 g, 35.59 mmol) in absolute EtOH (100 mL) was refluxed overnight. After cooling, the reaction mixture was diluted with water (400 mL), the pH was adjusted to 11 with ammonium hydroxide solution, and it was further stirred for 2 h. The resulting precipitate was filtered, washed with water, and dried in vacuo to provide 5.85 g of 4-(2-amino-thiazol-4-yl)-pyridine as a pinkish solid (93% yield): ¹H NMR (DMSO-d6, ppm) δ 7.23 (broad s, 2H), 7.42 (s, 1H), 7.74 (d, 2H), 8.56 (d, 2H); [M+H]⁺ m/z 178.

A suspension of 4-(2-amino-thiazol-4-yl)-pyridine (5.8 g, 32.72 mmol) in (1:1) THF/DMF (100 mL) was treated with NBS (6.4 g, 36 mmol) at 0° C. Ice bath was removed, and the reaction mixture was stirred at room temperature for 1 h, before pouring onto iced water (600 mL). After stirring for 30 min., the resulting precipitate was filtered, washed with water, 1M aqueous NaHSO₃, then water again, and dried in vacuo to provide 6.68 g of the titled product as a light brown solid (80% yield): ¹H NMR (DMSO-d6, ppm) δ 7.48 (broad s, 2H), 7.83 (d, 2H), 8.66 (d, 2H); [M+H]⁺ m/z 256, 258.

### Synthesis of 1-(4-methanesulfonylphenyl)-3-[3-(4,4,5,5-tetramethyl-[1,3,2]dioxaborolan-2-yl)-phenyl]-urea (Intermediate 20)

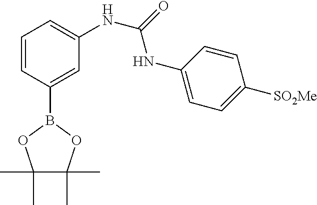 [SMILES: CC1(C)OB(c2cccc(NC(=O)Nc3ccc(S(C)(=O)=O)cc3)c2)OC1(C)C]

4-Methanesulfonyl-phenylamine hydrochloride (0.5 g, 2.4 mmol) was dissolved in 24 mL dichloromethane and diisopropylethylamine (0.419 mL, 2.4 mmol), then. Solid triphosgene (249 mg, 0.84 mmol) was added. Additional diisopropylethylamine (0.419 mL, 2.4 mmol) was added. By LCMS (MeOH quench), <10% starting material remained so 3-(4,4,5,5-tetramethyl-[1,3,2]dioxaborolan-2-yl)-phenylamine (526 mg, 2.4 mmol) and diisopropylethylamine (0.419 mL, 2.4 mmol) were added. The reaction was judged complete in 1 h by LCMS. MeOH (1 mL) was added and the reaction was stirred 18 h. The mixture was washed with 1 N HCl (1×) and saturated NaHCO₃, wherein a precipitate formed. The solids were collected by filtration and determined to be product by LCMS. [M+H]⁺ m/z 417.

### Synthesis of 4-(4-bromo-1-ethyl-1H-pyrazol-3-yl)pyridine (Intermediate 21)

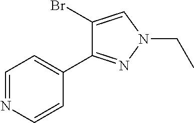

Step 1: A flask was charged with 4-acetylpyridine (10 mL, 90 mmol) and DMF-DMA (20 mL, 150 mmol). The reaction mixture was stirred at 100° C. for 1 h, then it was cooled and concentrated in vacuo to a dark orange solid. The solid was dissolved in absolute EtOH (100 mL) and hydrazine monohydrate (4.8 mL, 99 mmol) was added. The reaction mixture was stirred at 80° C. for 21 h, then it was cooled and concentrated in vacuo. The residue was dissolved in EtOAc, washed with brine (3×), dried over magnesium sulfate, filtered, and concentrated in vacuo to give 5.96 g of 4-(1H-pyrazol-3-yl)pyridine as a yellow solid (46% yield): ¹H NMR (DMSO-d6, ppm) δ 6.94 (s, 1H), 7.80 (d, 2H), 7.89 (s, 1H), 8.59 (d, 2H), 1.3.2 (broad s, 1H).

Step 2: To a mixture of 4-(1H-pyrazol-3-yl)pyridine (4.9 g, 33.75 mmol), tetrabutylammonium bromide (1.09 g, 3.375 mmol), and 8M aqueous NaOH (170 mL) in DCM (170 mL) was added ethyl iodide (4.07 mL, 50.63 mmol) dropwise. The reaction mixture was stirred for 22 h, then it was diluted with DCM and washed with water (2×) then brine. The organics were adsorbed on silica gel. Purification by flash silica gel chromatography using a gradient of 0-100% EtOAc/hexane afforded 4.54 g of 4-(1-ethyl-1H-pyrazol-3-yl)pyridine as a yellow oil.

Step 3: The oil was dissolved in THF (100 mL) and NBS (5.6 g, 31.35 mmol) was added. The reaction mixture was stirred for 5 h, then it was partitioned between EtOAc and 1 N aqueous NaOH. The organic layer was washed with 1N aqueous NaOH, brine, then it was adsorbed on silica gel. Purification by flash silica gel chromatography using a gradient of 0-100% EtOAc/hexane afforded 4.5 g of a pink oil that was further purified by flash silica gel chromatography using a gradient of 0-20% acetonitrile/DCM to provide 3.75 g of 4-(4-bromo-1-ethyl-1H-pyrazol-3-yl)pyridine as a light yellow solid (57% yield): ¹H NMR (DMSO-d6, ppm) δ 1.44 (t, 3H), 4.23 (q, 2H), 7.86 (d, 2H), 8.22 (s, 1H), 8.67 (d, 2H); [M+H]⁺ m/z 252, 254.

### Synthesis of 1-[3-(4,4,5,5-Tetramethyl-[1,3,2]dioxaborolan-2-yl)-phenyl]-3-(4-trifluoromethyl-phenyl)-imidazolidin-2-one (Intermediate 22)

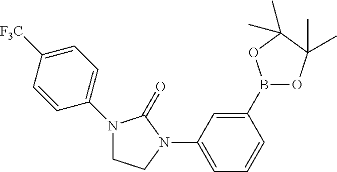 [SMILES: CC1(C)OB(c2cccc(N3CCN(c4ccc(C(F)(F)F)cc4)C3=O)c2)OC1(C)C]

Step 1: To a solution of 4-trifluoromethylaniline (0.5 mL, 4.02 mmol) in THF (5 mL) under nitrogen atmosphere was added 2-chloroethyl isocyanate (0.343 mL, 4.02 mmol) dropwise. The reaction mixture was stirred at 50° C. for 22 h then it was concentrated in vacuo to a yellow solid. The solid was dissolved in DMF (5 mL) and potassium carbonate (278 mg, 2.01 mmol) was added. The reaction mixture was stirred at room temperature for 22 h, then KOtBu (902 mg) was added and the reaction mixture was stirred for another 2 h before pouring onto 1N aqueous HCl. The resulting precipitate was filtered, washed with water, and dried in vacuo to give 730 mg of 1-(4-trifluoromethyl-phenyl)-imidazolidin-2-one as a white solid (79% yield): ¹H NMR (DMSO-d6, ppm) δ 3.46 (t, 2H), 3.92 (t, 2H), 7.26 (s, 1H), 7.67 (d, 2H), 7.78 (d, 2H); [M+H]⁺ m/z 231.

Step 2: To a suspension of 1-(4-trifluoromethyl-phenyl)-imidazolidin-2-one (300 mg, 1.303 mmol), CuI (50 mg, 0.261 mmol), and K₃PO₄ (553 mg, 2.606 mmol) in 1,4-dioxane (5 mL) under nitrogen atmosphere was added 3-bromo iodobenzene (0.167 mL, 1.303 mmol) followed by N,N′-dimethylethylenediamine (0.028 mL, 0.261 mmol). The reaction mixture was stirred at 110° C. for 24 h, then cooled, filtered through celite, and the cake was washed with EtOAc. The filtrate was adsorbed-on silica gel. Purification by flash silica get chromatography using a gradient of 0-50% EtOAc/hexane afforded 314 mg of a mixture of bromo and iodo coupling product as an off white solid. The material was charged into a vial along with bis(pinacolato)diboron (229 mg, 0.9 mmol), potassium acetate (161 mg, 1.64 mmol), Pd(dppf)Cl₂.CH₂Cl₂ (20 mg, 0.0246 mmol), and DMF (3 mL) under nitrogen atmosphere. After stirring for 26 h at 100° C. the reaction mixture was concentrated in vacuo, the residue was triturated with EtOAc and filtered through a pad of celite. The filtrate was adsorbed on silica gel. Purification by flash silica gel chromatography using a gradient of 0-40% EtOAc/hexane afforded 223 mg of 1-[3-(4,4,5,5-tetramethyl-[1,3,2]dioxaborolan-2-yl)-phenyl]-3-(4-trifluoromethyl-phenyl)-imidazolidin-2-one as a foam: ¹H NMR (DMSO-d6; ppm) δ 1.33 (s, 12H), 4.05 (s, 4H), 7.43 (d, 2H), 7.74 (m, 3H), 7.88 (d, 2H), 8.00 (s, 1H); [M+H]⁺ m/z 433.

### Synthesis of 1-(4-ethanesulfonyl-phenyl)-3-[3-(4,4,5,5-tetramethyl-[1,3,2]dioxaborolan-2-yl)-phenyl]-urea (Intermediate 23)

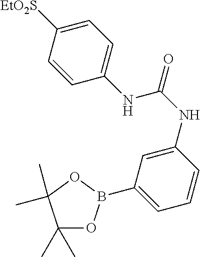

4-Ethanesulfonyl-phenylamine (0.1 g, 0.54 mmol) was dissolved in 5 mL dichloromethane and diisopropylethylamine (0.188 mL, 1.08 mmol), then solid triphosgene (64 mg, 0.22 mmol) was added. By LCMS (MeOH quench), <10% starting material remained so 3-(4,4,5,5-tetramethyl-[1,3,2]dioxaborolan-2-yl)-phenylamine (118 mg, 0.54 mmol) and diisopropylethylamine (0.188 mL, 1.08 mmol) were added. The reaction was judged complete in 1 h by LCMS. MeOH (1 mL) was added and the reaction was stirred 18 h. The mixture was washed with DI water (1×), saturated NaHCO₃ (1×), and 1 N HCl (1×) and was dried over MgSO₄. After concentration in vacuo, an off-white solid was obtained. [M+H]⁺ m/z 431.

### Synthesis of 1-(3-methanesulfonyl-phenyl)-3-[3-(4,4,5,5-tetramethyl-[1,3,2]dioxaborolan-2-yl)-phenyl]urea (Intermediate 24)

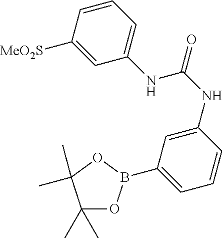 [SMILES: CC1(C)OB(c2cccc(NC(=O)Nc3cccc(S(C)(=O)=O)c3)c2)OC1(C)C]

3-Methanesulfonyl-phenylamine (0.3 g, 1.44 mmol) was dissolved in 10 mL dichloromethane and diisopropylethylamine (0.376 mL, 2.15 mmol), then solid triphosgene (171 mg, 0.58 mmol) was added. By LCMS (MeOH quench), <10% starting material remained so 3-(4,4,5,5-tetramethyl-[1,3,2]dioxaborolan-2-yl)-phenylamine (315.5 mg, 1.44 mmol) and diisopropylethylamine (0.276 mL, 1.58 mmol) were added. The reaction was judged complete in 1 h by LCMS. MeOH (1 mL) was added and the reaction was stirred 18 h. The mixture was washed with DI water, saturated NaHCO₃, and 1 N HCl and was dried over MgSO₄. After concentration in vacuo, an off-white solid was obtained. [M+H]⁺ m/z 417.

### Synthesis of N-methyl-4-{3-[3-(4,4,5,5-tetramethyl-[1,3,2]dioxaborolan-2-yl)-phenyl]-ureido}-benzenesulfonamide (Intermediate 25)

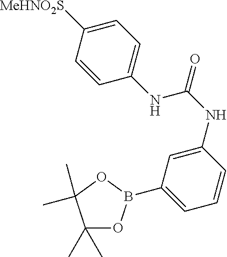

4-Amino-N-methyl-benzenesulfonamide (0.4 g, 2.15 mmol) was dissolved in 20 mL dichloromethane and diisopropylethylamine (0.512 mL, 2.48 mmol), then solid triphosgene (237 mg, 0.86 mmol) was added. By LCMS (MeOH quench), <10% starting material remained so 3-(4,4,5,5-tetramethyl-[1,3,2]dioxaborolan-2-yl)-phenylamine (471 mg, 2.15 mmol) and diisopropylethylamine (0.412 mL, 2.36 mmol) were added. The reaction was judged complete in 1 h by LCMS-MeOH (1 mL) was added and the reaction was stirred 18 h. The mixture was washed with DI water, saturated NaHCO₃, and 1 N HCl and was dried over MgSO₄. After concentration in vacuo, an off-white solid was obtained. [M+H]⁺ m/z 433.

### Synthesis of 3-(1-ethyl-3-(pyridin-4-yl)-1H-pyrazol-4-yl)aniline (Intermediate 26)

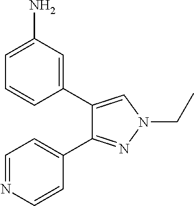 [SMILES: CCn1cc(-c2cccc(N)c2)c(-c2ccncc2)n1]

Synthesized according to the procedure described for the intermediate 2 using intermediate 21 as the starting material. [M+H]⁺ m/z 265.

### Synthesis of [4-(pyrrolidine-1-sulfonyl)-phenyl]-carbamoyl chloride (Intermediate 27)

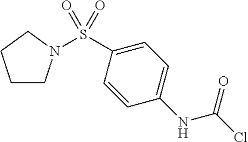 [SMILES: O=C(Cl)Nc1ccc(S(=O)(=O)N2CCCC2)cc1]

4-(Pyrrolidine-1-sulfonyl)-phenylamine (50.8 mg, 0.224 mmol) and DIEA (100 uL) were dissolved in DCM (1.5 mL). Triphosgene (26.6 mg, 0.089 mmol) was added and the mixture was stirred 1 h. LCMS analysis in MeOH indicated complete formation of methyl carbamate indicating the reaction was complete. This crude solution was used in method A in place of commercial isocyanate.

### Synthesis of [4-(morpholine-4-sulfonyl)-phenyl]-carbamoyl chloride (Intermediate 28)

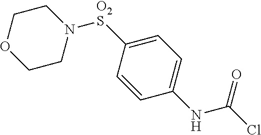 [SMILES: O=C(Cl)Nc1ccc(S(=O)(=O)N2CCOCC2)cc1]

4-(Morpholine-4-sulfonyl)-phenylamine (55.0 mg, 0.227 mmol) and DIEA (100 uL) were dissolved in DCM (1.5 mL). Triphosgene (27.0 mg, 0.091 mmol) was added and the mixture was stirred 1 h. LCMS analysis in MeOH indicated complete formation of methyl carbamate indicating the reaction was complete. This crude solution was used in method A in place of commercial isocyanate.

### Synthesis of 4-(4-bromo-4-ethyl-1H-pyrazol-3-yl)-2-(methylthio)pyrimidine (Intermediate 29)

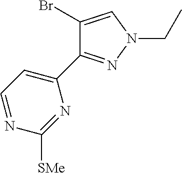 [SMILES: CCn1cc(Br)c(-c2ccnc(SC)n2)n1]

Step 1: A flask was charged with 1-(2-(methylthio)pyrimidin-4-yl)ethanone (23.7 mmol, 4 g) and DMF-DMA (40 mmol, 4.7 g. 5.33 mL). The reaction mixture was stirred at 80° C. for 2 hours, then it was cooled and concentrated in vacuo to a dark orange solid. The solid was dissolved in absolute EtOH (20 mL) and hydrazine monohydrate (1.26 mL, 26 mmol) was added. The reaction mixture was stirred at 80° C. overnight, then it was cooled and concentrated in vacuo. The residue was dissolved in EtOAc, washed with brine (3×), dried over magnesium sulfate, filtered, and concentrated in vacuo to give 4.42 g of 4-(4-bromo-1-ethyl-1H-pyrazol-3-yl)-2-(methylthio)pyrimidine as a yellow solid (97% yield): ¹H NMR (CDCl₃, ppm) δ 8.67 (d, 1H), 8.46 (d, 1H), 7.62 (s, 1H), 6.89 (s, 1H), 2.51 (s, 3H).

Step 2: To a mixture of 4-(4-bromo-1-ethyl-1H-pyrazol-3-yl)-2-(methylthio)pyrimidine (4.42 g, 23 mmol), tetrabutyl ammonium bromide (1.26 g, 3.91 mmol), and 8M aqueous NaOH (115 mL) in DCM (115 mL) was added ethyl iodide (2.77 mL, 34.5 mmol) dropwise. The reaction, mixture was stirred for 22 h, then it was diluted with DCM and washed with water (2×) then brine. The organics were adsorbed on silica gel. Purification by flash silica gel chromatography using a gradient of 10-50% EtOAc/hexane afforded 3.6 g (16.3 mmole, 71%) of 4-(1-ethyl-1H-pyrazol-3-yl)-2-(methylthio)pyrimidine as a yellow oil contamined by about 10% of the undesired isomer (4-(1-ethyl-1H-pyrazol-5-yl)-2-(methylthio)pyrimidine).

Step 3: The oil was dissolved in THF (100 mL) and NBS (3.49 g, 19 mmol) was added. The reaction mixture was stirred overnight at room temperature, then it was partitioned between EtOAc and 1N aqueous NaOH. The organic layer was washed with brine, then it was adsorbed on silica gel. Purification by flash silica gel chromatography using a gradient of 10-60% EtOAc/hexane afforded 3.83 g of 4-(4-bromo-1-ethyl-1H-pyrazol-3-yl)-2-(methylthio)pyrimidine the desired isomer: ¹H NMR (CDCl₃ ppm) δ 1.46 (t, 3H), 3.35 (s, 3H) 4.18 (q, 2H), 7.48 (s, 1H), 7.51 (d, 1H), 8.42 (d, 2H); [M+H]⁺ m/z 300.

### Synthesis of 4-(4-bromo-1-ethyl-1H-pyrazol-3-yl)pyrimidin-2-amine (Intermediate 30)

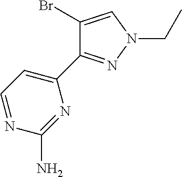

Step 1: 4-(4-bromo-1-ethyl-1H-pyrazol-3-yl)-2-(methylthio)pyrimidine (1.02 g, 3.4 mmole) previously dissolved in DCM (50 mL) and mCPBA (1.17 g, 6.8 mmol) was combined and stirred at room temperature overnight. The resulting reaction mixture was washed with aqueous saturated NaHCO₃, extracted with DCM and the organic layers were washed with brine, dried (MgSO₄), concentrated under reduced pressure to obtain 4-(4-bromo-1-ethyl-1H-pyrazol-3-yl)-2-(methylsulfonyl)pyrimidine (1 g, 3.0 mmol, 88%).

Step 2: 4-(4-bromo-1-ethyl-1H-pyrazol-3-yl)-2-(methylsulfonyl)pyrimidine (630 mg, 1.90 mmol) was dissolved in 1,4 dioxane (3 mL) and NH₄OH (2 mL). The mixture was irradiated in a microwave apparatus for 1 hour at 80° C. The mixture was cooled, partitioned between water and EtOAc. The organic layer was washed with brine, dried (MgSO₄), concentrated under reduced pressure to obtain the title compound in a quantitative yield (500 mg). ¹H NMR (CDCl₃ ppm) δ 1.47 (t, 3H), 4.03 (q, 2H) 5.18 (brs, 2H), 7.19 (d, 1H), 7.26 (s, 1H), 8.29 (d, 2H); [M+H]⁺ m/z 269.

### Synthesis of N-[3-(1-ethyl-3-pyridin-4-yl-1H-pyrazol-4-yl)-phenyl]-hydroxylamine (Intermediate 31)

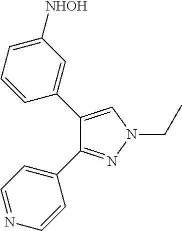

Step 1: Under nitrogen a microwave vial was charged with 4-(4-bromo-1-ethyl-1H-pyrazol-3-yl)-pyridine (300 mg, 1.19 mmol), 4,4,5,5-tetramethyl-2-(3-nitro-phenyl)-[1,3,2]dioxaborolane (238 mg, 1.42 mmol), PdCl₂(PPh₃)₂ (41.7 mg, 0.059 mmol), 1,2-dimethoxyethane (3 mL) and 2 M sodium carbonate (1 mL). The vial was capped and, heated in the microwave for 30 min. at 150° C. and the crude material was adsorbed onto silica gel and purified by chromatography using 0-100% hexanes/ethyl acetate to elute the product as an oil: (305 mg, 87.4%). [M+H]⁺ m/z 295.

Step 2: 4-(1-ethyl-4-(3-nitrophenyl)-1H-pyrazol-3-yl)pyridine (108 mg, 0.137 mmol) was dissolved in a mixture of THF (0.2 mL) and EtOH (0.2 ml). Pd/C (10 mg) and hydrazine (1.4 eq) were added at 0° C. to the mixture. The reaction mixture was stirred for 1 hour at this temperature, filtrated through celite and concentrated under reduced pressure to afford the desired material [M+H]⁺ m/z 281.

### Synthesis of 4-(5-bromo-2-tert-butylthiazol-4-yl)pyrimidin-2-amine (Intermediate 32)

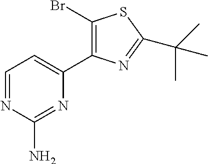 [SMILES: CC(C)(C)c1nc(-c2ccnc(N)n2)c(Br)s1]

Step 1: 1-(2-chloropyrimidin-4-yl)ethanone (1 eq) is dissolved in HBr/HOAc (1 mL/mmol) and Br₂ (1.1 eq) is added dropwise. The reaction mixture is stirred for 1 hour at room temperature, ether (10 mL/mmol) was added and the mixture was cooled at 0° C. The solid is collected by filtration to afford 2-bromo-1-(2-chloropyrimidin-4-yl)ethanone.

Step 2: 2-bromo-1-(2-chloropyrimidin-4-yl)ethanone (1 eq) is dissolved in EtOH (5 mL/mmol) and 2,2-diethylpropanethioamide (1.1 eq) is added and the mixture is stirred at 60° C. for 2 hours. The solvent is removed under reduced pressure; the crude material is dissolved in DCM. The organic phase is washed with aq NaOH (1 N), brine, dried (MgSO₄) and the solvent is removed. Purification by flash silica gel chromatography using a gradient of 10-40%-EtOAc/hexane affords 2-tert-butyl-4-(2-chloropyrimidin-4-yl)thiazole.

Step 3: 2-tert-butyl-4-(2-chloropyrimidin-4-yl)thiazole is dissolved in THF and NBS (1.2 eq) is added. The reaction mixture is stirred at room temperature overnight and the mixture is diluted with EtOAc. The organic layer is washed with brine, dried (MgSO₄) and concentrated under reduced pressure. Purification by flash silica gel chromatography using a gradient of 10-60% EtOAc/hexane affords 5-bromo-2-tert-butyl-4-(2-chloropyrimidin-4-yl)thiazole.

Step 4: 5-bromo-2-tert-butyl-4-(2-chloropyrimidin-4-yl)thiazole is dissolved in 1,4 dioxane (1.5 mmol) and ammonium hydroxide (1 mL/mmol). The reaction is irradiated in a microwave apparatus at 120° C. (7, for 1 hour to afford the title compound

### Synthesis of 4-(5-bromo-2-morpholinothiazol-4-yl)pyrimidin-2-amine (Intermediate 33)

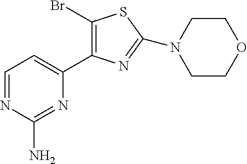 [SMILES: Nc1nccc(-c2nc(N3CCOCC3)sc2Br)n1]

The intermediate 33 is synthesized using the same procedure as intermediate 32 starting with morpholine-4-carbothioamide.

Synthetic Method A (Isocyanate Condensation)

The corresponding phenylamine (1 eq) and benzoisocyanate (1.1 eq) were dissolved in DCM and stirred overnight at room temperature. The mixture was concentrated under reduced pressure and loaded onto SiO₂ gel. The desired product was eluted using a gradient of EtOAc/hexanes to afford the desire carbamate.

Synthetic Method B (Palladium-Mediated Coupling)

A vial was charged with desired heterocyclic bromine (1 eq), the corresponding boronate ester (1.2 equiv.), Pd(PPh₃)₂Cl₂ (0.05 equiv.), saturated aqueous sodium bicarbonate (1.5 mL/mmol) and 1,4-dioxane (3.5 mL/mmol) under nitrogen atmosphere. After stirring for 2 h to 18 h at 80 (at which time the reaction was judged complete by LCMS). The aqueous layer was removed, the organic phase was diluted with MeOH and filtered. The filtrate was adsorbed on silica gel. Purification by flash silica gel chromatography using a gradient of 10-80% EtOAc/hexane afforded the final product.

Synthetic Method C (Amination)

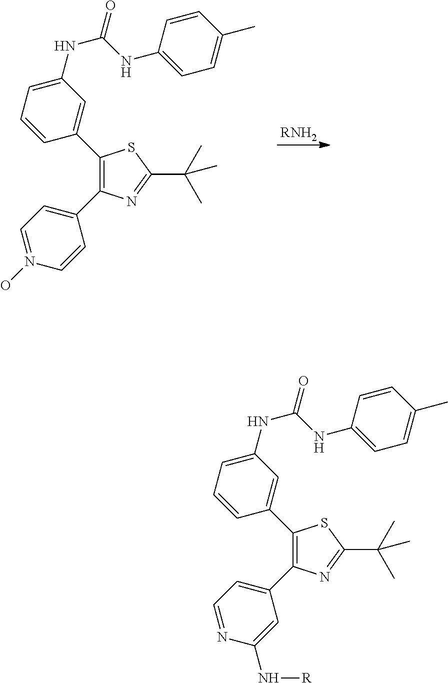

To a solution of 1-(3-(2-tert-butyl-4-(pyridin-4-yl)thiazol-5-yl)phenyl)-3-p-tolylurea, pyridine N-oxide (50 mg, 0.109 mmol), corresponding amine (1.25 equiv.), and DIEA (3.75 equiv.) in DCM (0.5 mL) was added PyBrop (1.3 equiv.). The reaction mixture was stirred overnight then it was adsorbed on silica gel. Purification by flash chromatography and preparative HPLC afforded the final product.

Synthetic Method D (Palladium-Mediated Coupling)

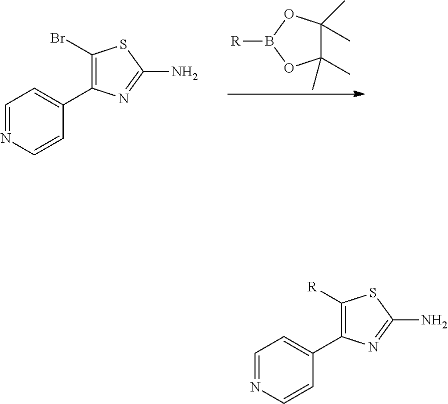

A vial was charged with 4-(5-bromo-2-amino-thiazol-4-yl)-pyridine, the corresponding boronate ester (1.2 equiv.), Pd(dppf)Cl₂.CH₂Cl₂ (0.05 equiv.), saturated aqueous sodium bicarbonate (0.3 mL) and 1,4-dioxane (0.7 mL) under nitrogen atmosphere. After stirring for 2 h at 80° C. the reaction mixture was diluted with MeOH and filtered. The filtrate was adsorbed on silica gel. Purification by flash silica gel chromatography afforded the final product.

Synthetic Method E (Palladium-Mediated Coupling)

A vial was charged with desired heterocyclic bromine (1 eq), the corresponding boronate ester (1.2 equiv.), Pd(OAc)₂ (0.05 equiv.), (Cy)₃P (0.1 eq), K₂CO₃ (2 eq), toluene (3.5 mL/mmol) or isopropyl acetate (3.5 mL/mmol) (depending of the solubility of the boronic acid) and H₂O (3.5 mL/mmol) under nitrogen atmosphere The mixture was irradiated in a microwave apparatus for 12 h at 120° C. The aqueous layer was removed and the organic layer was filtered and the filtrate was adsorbed on silica gel. Purification by flash silica gel chromatography using a gradient of 10-100% EtOAc/hexane afforded the final product.

### Example 1

### 1-[3-(2-Methyl-4-pyridin-4-yl-thiazol-5-yl)-phenyl]-3-p-tolyl-urea

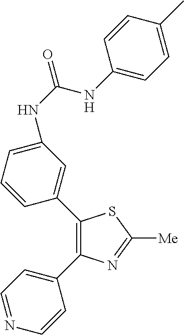 [SMILES: Cc1ccc(NC(=O)Nc2cccc(-c3sc(C)nc3-c3ccncc3)c2)cc1]

3-(2-Methyl-4-pyridin-4-yl-thiazol-5-yl)-phenylamine (14.7 mg, 0.055 mmol) and 3-aminophenylboronic acid were dissolved in DCM (0.5 mL). 4-Methylbenzoisocyanate (8.06 mg, 0.06 mmol) was added and the mixture was stirred for 15 h. The mixture was loaded onto SiO₂ gel and the product eluted using a gradient of EtOAc/hexanes to afford 12.8 mg (58.1%) 1-[3-(2-Methyl-4-pyridin-4-yl-thiazol-5-yl)-phenyl]-3-p-tolyl-urea: ¹H: NMR (CDCl₃, ppm) δ 2.34 (s, 3H), 2.77 (s, 3H), 6.73 (br s, 1H), 7.03 (m, 2H), 7.14 (d, 2H), 7.20 (d, 24), 7.31 (m, 2H), 7.45 (d, 2H), 7.46 (br d, 1H), 8.49 (d, 2H); [M+H]+ m/z 401.

### Example 2

### 1-(4-chloro-3-(trifluoromethyl)phenyl)-3-(3-(2-methyl-4-(pyridin-4-yl)thiazol-5-yl)phenyl)urea

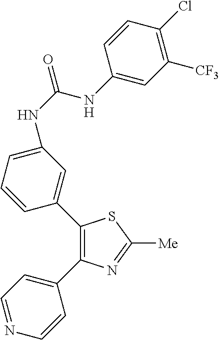 [SMILES: Cc1nc(-c2ccncc2)c(-c2cccc(NC(=O)Nc3ccc(Cl)c(C(F)(F)F)c3)c2)s1]

Synthesized from intermediate 2 using general synthetic method A.

### Example 3

### 1-(4-chlorophenyl)-3-(3-(2-methyl-4-(pyridin-4-yl)thiazol-5-yl)phenyl)urea

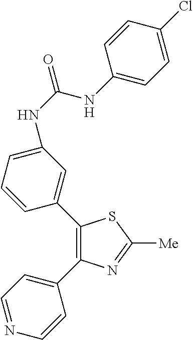 [SMILES: Cc1nc(-c2ccncc2)c(-c2cccc(NC(=O)Nc3ccc(Cl)cc3)c2)s1]

Synthesized from intermediate 2 using general synthetic method A.

### Example 4

### 1-(4-fluorophenyl)-3-(3-(2-methyl-4-(pyridin-4-yl)thiazol-5-yl)phenyl)urea

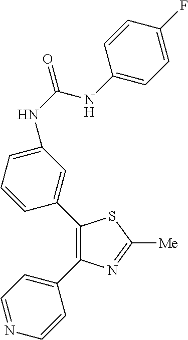 [SMILES: Cc1nc(-c2ccncc2)c(-c2cccc(NC(=O)Nc3ccc(F)cc3)c2)s1]

Synthesized from intermediate 2 using general synthetic method A.

### Example 5

### 1-(2,5-difluorophenyl)-3-(3-(2-methyl-4-(pyridin-4-yl)thiazol-5-yl)phenyl)urea

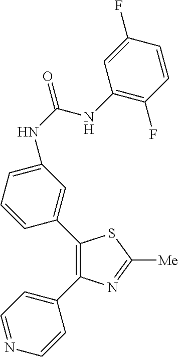 [SMILES: Cc1nc(-c2ccncc2)c(-c2cccc(NC(=O)Nc3cc(F)ccc3F)c2)s1]

Synthesized from intermediate 2 using general synthetic method A.

### Example 6

### 1-(2,6-difluorophenyl)-3-(3-(2-methyl-4-(pyridin-4-yl)thiazol-5-yl)phenyl)urea

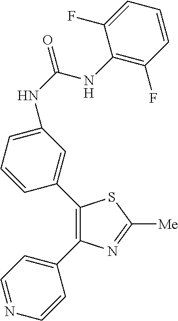 [SMILES: Cc1nc(-c2ccncc2)c(-c2cccc(NC(=O)Nc3c(F)cccc3F)c2)s1]

Synthesized from intermediate 2 using general synthetic method A.

### Example 7

### 1-(2-fluoro-5-(2-methyl-4-(pyridin-4-yl)thiazol-5-yl)phenyl)-3-p-tolylurea

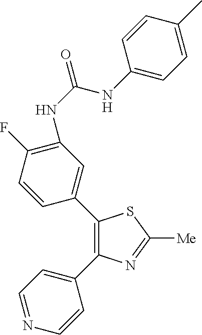 [SMILES: Cc1ccc(NC(=O)Nc2cc(-c3sc(C)nc3-c3ccncc3)ccc2F)cc1]

Synthesized from intermediate 8 using general synthetic method A.

### Example 8

### 1-(4-fluoro-3-(2-methyl-4-(pyridin-4-yl)thiazol-5-yl)phenyl)-3-p-tolylurea

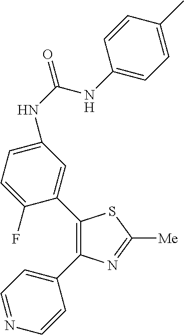 [SMILES: Cc1ccc(NC(=O)Nc2ccc(F)c(-c3sc(C)nc3-c3ccncc3)c2)cc1]

Synthesized using method A from 4-fluoro-3-(2-methyl-4-(pyridin-4-yl)thiazol-5-yl)aniline obtained by coupling the intermediate 1 with 3-amino-4-fluorophenylboronic acid (method B)

### Example 9

### 1-(3-(2-methyl-4-(pyridin-4-yl)thiazol-5-yl)phenyl)-3-(4-(trifluoromethyl)phenyl)urea

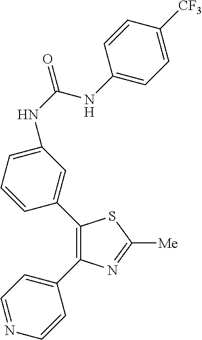 [SMILES: Cc1nc(-c2ccncc2)c(-c2cccc(NC(=O)Nc3ccc(C(F)(F)F)cc3)c2)s1]

Synthesized from intermediate 2 using general synthetic method A

### Example 10

### 1-(3-chlorophenyl)-3-(2-fluoro-5-(2-methyl-4-(pyridin-4-yl)thiazol-5-yl)phenyl)urea

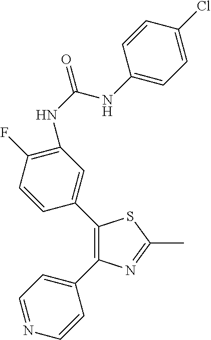 [SMILES: Cc1nc(-c2ccncc2)c(-c2ccc(F)c(NC(=O)Nc3ccc(Cl)cc3)c2)s1]

Synthesized from intermediate 8 using general synthetic method A

### Example 11

### 1-(3,4-dichlorophenyl)-3-(2-fluoro-5-(2-methyl-4-(pyridin-4-yl)thiazol-5-yl)phenyl)urea

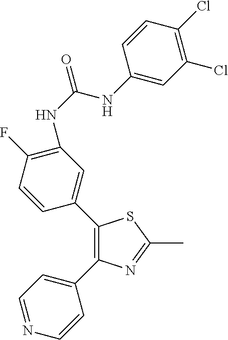 [SMILES: Cc1nc(-c2ccncc2)c(-c2ccc(F)c(NC(=O)Nc3ccc(Cl)c(Cl)c3)c2)s1]

Synthesized from intermediate 8 using general synthetic method A

### Example 12

### 1-(4-chloro-2-fluorophenyl)-3-(2-fluoro-5-(2-methyl-4-(pyridin-4-yl)thiazol-5-yl)phenyl)urea

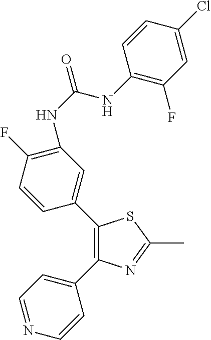 [SMILES: Cc1nc(-c2ccncc2)c(-c2ccc(F)c(NC(=O)Nc3ccc(Cl)cc3F)c2)s1]

Synthesized from intermediate 8 using general synthetic method A

### Example 13

### 1-(3-chlorophenyl)-3-(2-fluoro-5-(2-methyl-4-(pyridin-4-yl)thiazol-5-yl)phenyl)urea

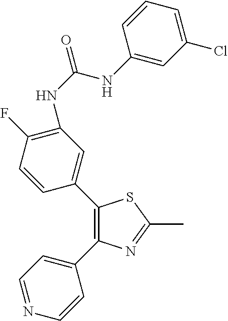 [SMILES: Cc1nc(-c2ccncc2)c(-c2ccc(F)c(NC(=O)Nc3cccc(Cl)c3)c2)s1]

Synthesized from intermediate 8 using general synthetic method A

### Example 14

### 1-(2-fluoro-5-(2-methyl-4-(pyridin-4-yl)thiazol-5-yl)phenyl)-3-(4-(trifluoromethyl)phenyl)urea

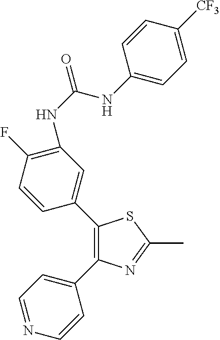 [SMILES: Cc1nc(-c2ccncc2)c(-c2ccc(F)c(NC(=O)Nc3ccc(C(F)(F)F)cc3)c2)s1]

Synthesized from intermediate 8 using general synthetic method A

### Example 15

### 1-(2-fluoro-3-(2-methyl-4-(pyridin-4-yl)thiazol-5-yl)phenyl)-3-(4-(trifluoromethyl)phenyl)urea

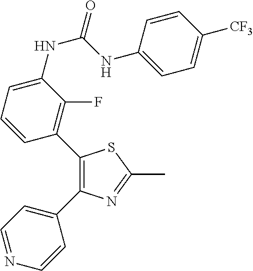 [SMILES: Cc1nc(-c2ccncc2)c(-c2cccc(NC(=O)Nc3ccc(C(F)(F)F)cc3)c2F)s1]

Synthesized from intermediate 1 and 15 using method B

### Example 16

### 1-(2-fluoro-5-(2-methyl-4-(pyridin-4-yl)thiazol-5-yl)phenyl)-3-(3-(trifluoromethyl)phenyl)urea

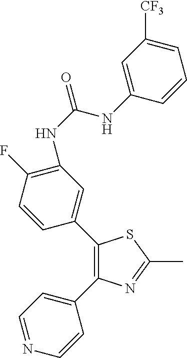 [SMILES: Cc1nc(-c2ccncc2)c(-c2ccc(F)c(NC(=O)Nc3cccc(C(F)(F)F)c3)c2)s1]

Synthesized from intermediate 8 using method A

### Example 17

### 1-(3-(2-methyl-4-(pyridin-4-yl)thiazol-5-yl)phenyl)-3-(4-(methylsulfonyl)phenyl)urea

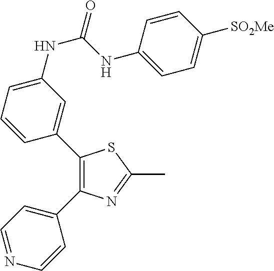 [SMILES: Cc1nc(-c2ccncc2)c(-c2cccc(NC(=O)Nc3ccc(S(C)(=O)=O)cc3)c2)s1]

Synthesized from intermediates 1 and 20 using method B

### Example 18

### 1-(4-chlorophenyl)-3-(2-fluoro-3-(2-methyl-4-(pyridin-4-yl)thiazol-5-yl)phenyl)urea

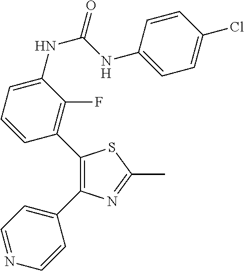 [SMILES: Cc1nc(-c2ccncc2)c(-c2cccc(NC(=O)Nc3ccc(Cl)cc3)c2F)s1]

Synthesized from intermediates 1 and 14 using method B

### Example 19

### 1-(3-(2-methyl-4-(pyridin-4-yl)thiazol-5-yl)phenyl)-3-p-tolylthiourea

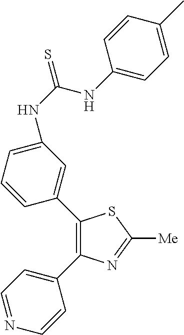 [SMILES: Cc1ccc(NC(=S)Nc2cccc(-c3sc(C)nc3-c3ccncc3)c2)cc1]

Synthesized from intermediate 2 and 1-isothiocyanato-4-methylbenzene using general synthetic method A

### Example 20

### 1-(3-(4-(2-aminopyridin-4-yl)-2-methylthiazol-5-yl)phenyl)-3-p-tolylurea

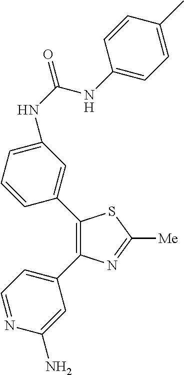 [SMILES: Cc1ccc(NC(=O)Nc2cccc(-c3sc(C)nc3-c3ccnc(N)c3)c2)cc1]

Synthesized from intermediate 7 using general synthetic method A.

### Example 21

### 1-(3-(4-(2-aminopyridin-4-yl)-2-methylthiazol-5-yl)phenyl)-3-(4-chlorophenyl)urea

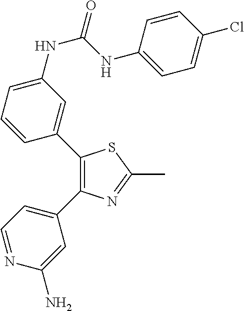 [SMILES: Cc1nc(-c2ccnc(N)c2)c(-c2cccc(NC(=O)Nc3ccc(Cl)cc3)c2)s1]

Synthesized from intermediate 7 using general synthetic method A.

### Example 22

### 1-(5-(4-(2-aminopyridin-4-yl)-2-methylthiazol-5-yl)-2-fluorophenyl)-3-(4-chlorophenyl)urea

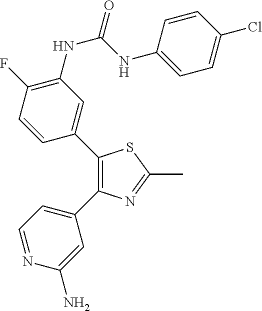 [SMILES: Cc1nc(-c2ccnc(N)c2)c(-c2ccc(F)c(NC(=O)Nc3ccc(Cl)cc3)c2)s1]

Synthesized from intermediate 13 and intermediate 6 using general synthetic method B

### Example 23

### 1-(3-(4-(2-aminopyridin-4-yl)-2-methylthiazol-5-yl)-2-fluorophenyl)-3-(4-chlorophenyl)urea

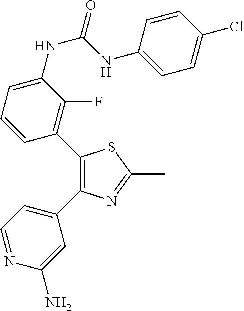 [SMILES: Cc1nc(-c2ccnc(N)c2)c(-c2cccc(NC(=O)Nc3ccc(Cl)cc3)c2F)s1]

Synthesized from intermediate 14 and intermediate 6 using general synthetic method B

### Example 24

### 1-(3-(4-(2-aminopyridin-4-yl)-2-methylthiazol-5-yl)-2-fluorophenyl)-3-(4-(trifluoromethyl)phenyl)urea

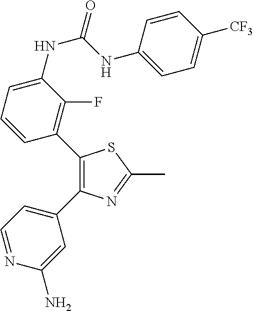 [SMILES: Cc1nc(-c2ccnc(N)c2)c(-c2cccc(NC(=O)Nc3ccc(C(F)(F)F)cc3)c2F)s1]

Synthesized from intermediate 15 and intermediate 6 using general synthetic method B

### Example 25

### 1-(5-(4-(2-aminopyridin-4-yl)-2-methylthiazol-5-yl)-2,4-difluorophenyl)-3-(4-(trifluoromethyl)phenyl)urea

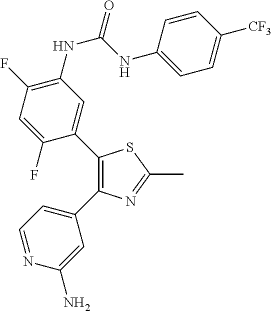 [SMILES: Cc1nc(-c2ccnc(N)c2)c(-c2cc(NC(=O)Nc3ccc(C(F)(F)F)cc3)c(F)cc2F)s1]

Synthesized from intermediate 16 and intermediate 6 using general synthetic method B.

### Example 26

### 1-(3-(4-(2-aminopyridin-4-yl)-2-methylthiazol-5-yl)phenyl)-3-(4-(trifluoromethyl)phenyl)urea

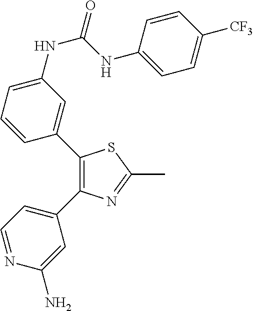 [SMILES: Cc1nc(-c2ccnc(N)c2)c(-c2cccc(NC(=O)Nc3ccc(C(F)(F)F)cc3)c2)s1]

Synthesized from intermediate 17 and intermediate 6 using general synthetic method B

### Example 27

### 1-(3-(2-tert-butyl-4-(pyridin-4-yl)thiazol-5-yl)phenyl)-3-p-tolylurea

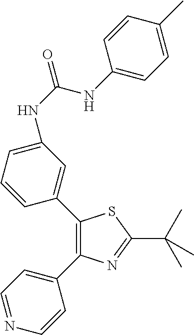 [SMILES: Cc1ccc(NC(=O)Nc2cccc(-c3sc(C(C)(C)C)nc3-c3ccncc3)c2)cc1]

Synthesized from intermediate 4 using general synthetic method A

### Example 28

### 1-(3-(2-tert-butyl-4-(2-(2-methoxyethylamino)pyridin-4-yl)thiazol-5-yl)phenyl)-3-p-tolylurea

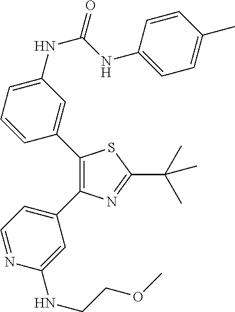 [SMILES: COCCNc1cc(-c2nc(C(C)(C)C)sc2-c2cccc(NC(=O)Nc3ccc(C)cc3)c2)ccn1]

Synthesized from intermediate 18 using general synthetic method C

### Example 29

### 1-(3-(2-tert-butyl-4-(2-(2-hydroxyethylamino)pyridin-4-yl)thiazol-5-yl)phenyl)-3-p-tolylurea

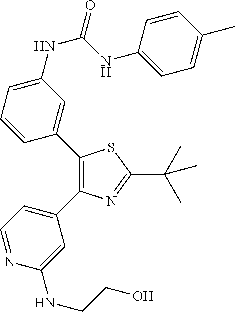 [SMILES: Cc1ccc(NC(=O)Nc2cccc(-c3sc(C(C)(C)C)nc3-c3ccnc(NCCO)c3)c2)cc1]

Synthesized from intermediate 18 using general synthetic method C.

### Example 30

### 1-(3-(2-tert-butyl-4-(2-(2-(dimethylamino)ethylamino)pyridin-4-yl)thiazol-5-yl)phenyl)-3-p-tolylurea

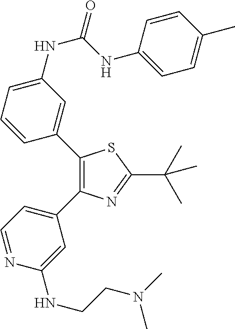 [SMILES: Cc1ccc(NC(=O)Nc2cccc(-c3sc(C(C)(C)C)nc3-c3ccnc(NCCN(C)C)c3)c2)cc1]

Synthesized from intermediate 18 using general synthetic method C.

### Example 31

### 1-(3-(2-tert-butyl-4-(2-(2-(pyrrolidin-1-yl)ethylamino)pyridin-4-yl)thiazol-5-yl)phenyl)-3-p-tolylurea

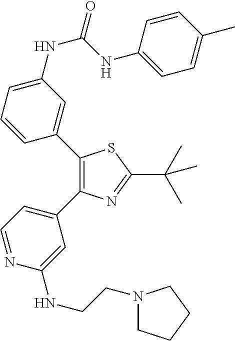 [SMILES: Cc1ccc(NC(=O)Nc2cccc(-c3sc(C(C)(C)C)nc3-c3ccnc(NCCN4CCCC4)c3)c2)cc1]

Synthesized from intermediate 18 using general synthetic method C.

### Example 32

### (S)-methyl 1-(4-(2-tert-butyl-5-(3-(3-p-tolylureido)phenyl)thiazol-4-yl)pyridin-2-ylamino)propan-2-ylcarbamate

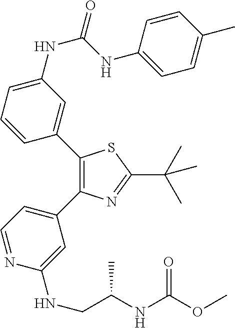 [SMILES: COC(=O)N[C@@H](C)CNc1cc(-c2nc(C(C)(C)C)sc2-c2cccc(NC(=O)Nc3ccc(C)cc3)c2)ccn1]

Synthesized from intermediate 18 using general synthetic method C

### Example 33

### 1-(4-chlorophenyl)-3-(3-(2-morpholino-4-(pyridin-4-yl)thiazol-5-yl)phenyl)urea

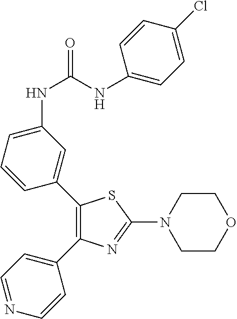 [SMILES: O=C(Nc1ccc(Cl)cc1)Nc1cccc(-c2sc(N3CCOCC3)nc2-c2ccncc2)c1]

Synthesized from intermediate 10 using general synthetic method A

### Example 34

### 1-(4-chlorophenyl)-3-(3-(2-(2-morpholinoethylamino)-4-(pyridin-4-yl)thiazol-5-yl)phenyl)urea

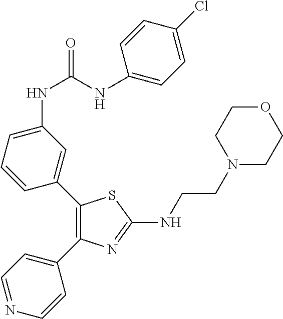 [SMILES: O=C(Nc1ccc(Cl)cc1)Nc1cccc(-c2sc(NCCN3CCOCC3)nc2-c2ccncc2)c1]

Synthesized from intermediate 12 using general synthetic method A.

### Example 35

### 1-(2-fluoro-5-(2-(2-morpholinoethylamino)-4-(pyridin-4-yl)thiazol-5-yl)phenyl)-3-(4-(trifluoromethyl)phenyl)urea

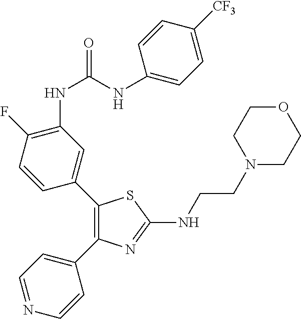 [SMILES: O=C(Nc1ccc(C(F)(F)F)cc1)Nc1cc(-c2sc(NCCN3CCOCC3)nc2-c2ccncc2)ccc1F]

Synthesized from intermediate 12 using general synthetic method A

### Example 36

### 1-(4-chlorophenyl)-3-(2-fluoro-5-(2-(2-morpholinoethylamino)-4-(pyridin-4-yl)thiazol-5-yl)phenyl)urea

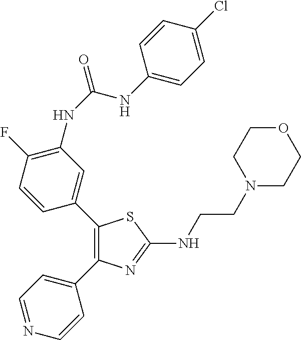 [SMILES: O=C(Nc1ccc(Cl)cc1)Nc1cc(-c2sc(NCCN3CCOCC3)nc2-c2ccncc2)ccc1F]

Synthesized from intermediate 12 using general synthetic method A.

### Example 37

### 1-(3-(2-(2-morpholinoethylamino)-4-(pyridin-4-yl)thiazol-5-yl)phenyl)-3-(4-(trifluoromethyl)phenyl)urea

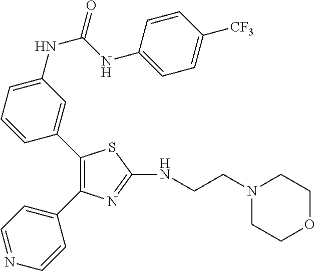 [SMILES: O=C(Nc1ccc(C(F)(F)F)cc1)Nc1cccc(-c2sc(NCCN3CCOCC3)nc2-c2ccncc2)c1]

Synthesized from intermediate 12 using general synthetic method A

### Example 38

### 1-(3-(2-amino-4-(pyridin-4-yl)thiazol-5-yl)-2-fluorophenyl)-3-(4-(trifluoromethyl)phenyl)urea

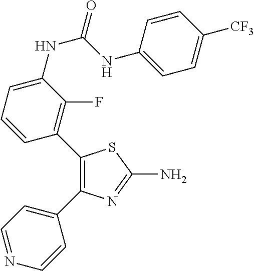 [SMILES: Nc1nc(-c2ccncc2)c(-c2cccc(NC(=O)Nc3ccc(C(F)(F)F)cc3)c2F)s1]

Synthesized from intermediate 15 and intermediate 19 using general synthetic method D

### Example 39

### 1-(3-(2-amino-4-(pyridin-4-yl)thiazol-5-yl)-2-fluorophenyl)-3-(4-chlorophenyl)urea

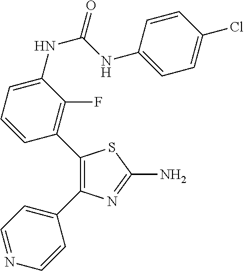 [SMILES: Nc1nc(-c2ccncc2)c(-c2cccc(NC(=O)Nc3ccc(Cl)cc3)c2F)s1]

Synthesized from intermediate 14 and intermediate 19 using general synthetic method D

### Example 40

### 1-(5-(2-amino-4-(pyridin-4-yl)thiazol-5-yl)-2-fluorophenyl)-3-(4-chlorophenyl)urea

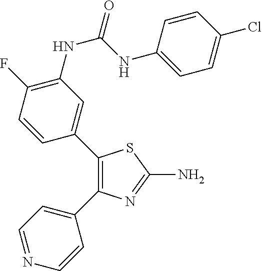 [SMILES: Nc1nc(-c2ccncc2)c(-c2ccc(F)c(NC(=O)Nc3ccc(Cl)cc3)c2)s1]

Synthesized from intermediate 13 and intermediate 19 using general synthetic method D

### Example 41

### 2,5-Difluoro-N-[3-(2-methyl-4-pyridin-4-yl-thiazol-5-yl)-phenyl]-benzenesulfonamide

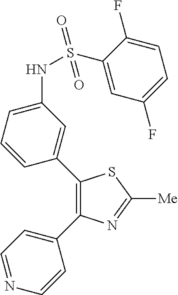 [SMILES: Cc1nc(-c2ccncc2)c(-c2cccc(NS(=O)(=O)c3cc(F)ccc3F)c2)s1]

3-(2-Methyl-4-pyridin-4-yl-thiazol-5-yl)-phenylamine (Intermediate 2) (27.9 mg, 0.105 mmol), pyridine (0.093 mL, 0.116 mmol), and DMAP (1.2 mg, 0.10 mmol) were dissolved to in DCM (0.5 mL). Sulfonyl chloride was added and the mixture was stirred 15-72 hrs. The compounds was purified by silica gel chromatography using a gradient od hexanes and ethyl acetate to afford 2,5-difluoro-N-[3-(2-methyl-4-pyridin-4-yl-thiazol-5-yl)-phenyl]-benzenesulfonamide as a solid (14.3 mg, 63.9%). ¹H NMR (DMSO-d6, ppm) δ □2.74 (s, 1H), 7.05 (d, 1H), 7.09 (s, 1H), 7.18 (dd, 1H), 7.25 (d, 2H), 7.26 (t, 1H), 7.37 (m, 3H), 8.46 (d, 2H), 10.97 (s, 1H); [M+H]+m/z 444.

### Example 42

### 2,6-difluoro-N-(3-(2-methyl-4-(pyridin-4-yl)thiazol-5-yl)phenyl)benzenesulfonamide

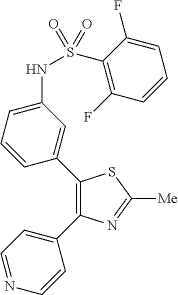 [SMILES: Cc1nc(-c2ccncc2)c(-c2cccc(NS(=O)(=O)c3c(F)cccc3F)c2)s1]

Synthesized using the same method as example 41

### Example 43

### N-(3-(4-(2-aminopyridin-4-yl)-2-methylthiazol-5-yl)phenyl)-2,6-difluorobenzenesulfonamide

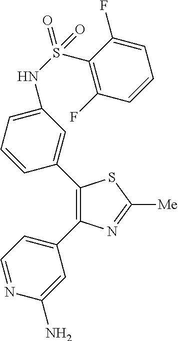 [SMILES: Cc1nc(-c2ccnc(N)c2)c(-c2cccc(NS(=O)(=O)c3c(F)cccc3F)c2)s1]

Synthesized using the same method as example 41 using intermediate 7

### Example 44

### N-(3-(2-tert-butyl-4-(pyridin-4-yl)thiazol-5-yl)phenyl)-2,6-difluorobenzenesulfonamide

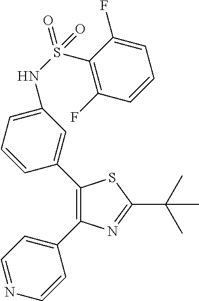 [SMILES: CC(C)(C)c1nc(-c2ccncc2)c(-c2cccc(NS(=O)(=O)c3c(F)cccc3F)c2)s1]

Synthesized using the same method as example 41 using intermediate 4

### Example 45

### N-[3-(2-Methyl-4-(2-aminopyridin-4-yl)-thiazol-5-yl)-phenyl]-p-tolylacetamide

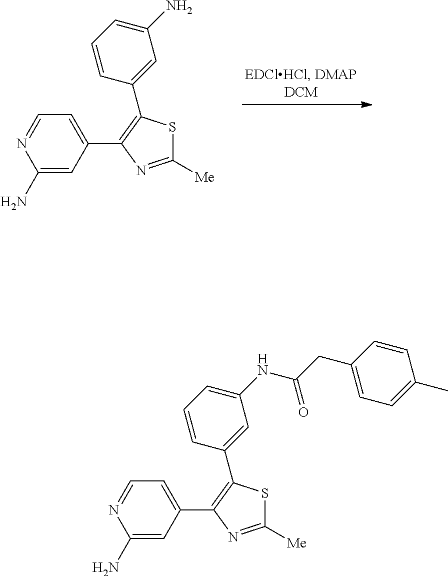 [SMILES: Cc1nc(-c2ccnc(N)c2)c(-c2cccc(N)c2)s1 ; Cc1ccc(CC(=O)Nc2cccc(-c3sc(C)nc3-c3ccnc(N)c3)c2)cc1]

To a stirring suspension of 3-(2-methyl-4-(2-aminopyridin-4-yl)-thiazol-5-yl)-phenylamine (Intermediate 7) (30 mg, 0.106 mmol), p-tolylacetic acid (18 mg, 0.117 mmol), and DMAP (1.2 mg, 0.0106 mml) in DCM (1 mL) was added EDCI.HCl (31 mg, 0.159 mmol). After stirring for 2 h, the reaction mixture was directly adsorbed on silica gel. Purification by flash silica gel chromatography using a gradient of 0-10% MeOH/DCM afforded 35 mg of the titled product as a white solid (80% yield).

### Example 46

### N-(3-(2-tert-butyl-4-(pyridin-4-yl)thiazol-5-yl)phenyl)-2-p-tolylacetamide

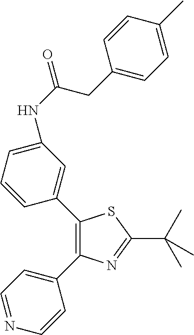 [SMILES: Cc1ccc(CC(=O)Nc2cccc(-c3sc(C(C)(C)C)nc3-c3ccncc3)c2)cc1]

Synthesized using the same method as example 45 using intermediate 4

### Example 47

### 2-(3-(2-methyl-4-(pyridin-4-yl)thiazol-5-yl)phenyl)-N-p-tolylacetamide

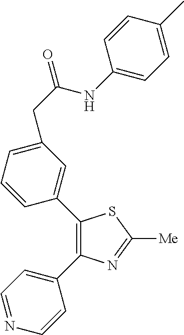 [SMILES: Cc1ccc(NC(=O)Cc2cccc(-c3sc(C)nc3-c3ccncc3)c2)cc1]

Synthesized using the same method as example 45 using intermediate 5

### Example 48

### 1-(4-chlorophenyl)-3-(5-(1-ethyl-3-(pyridin-4-yl)-1H-pyrazol-4-yl)-2-fluorophenyl)urea

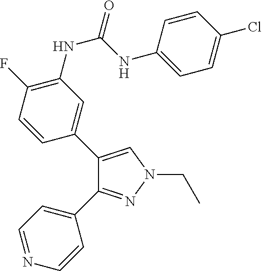 [SMILES: CCn1cc(-c2ccc(F)c(NC(=O)Nc3ccc(Cl)cc3)c2)c(-c2ccncc2)n1]

Synthesized from intermediate 13 and intermediate 21 using general synthetic method B

### Example 49

### 1-(4-chlorophenyl)-3-(3-(1-ethyl-3-(pyridin-4-yl)-1H-pyrazol-4-yl)-2-fluorophenyl)urea

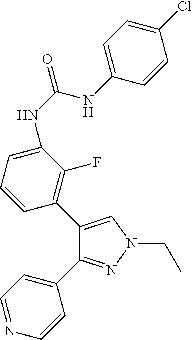 [SMILES: CCn1cc(-c2cccc(NC(=O)Nc3ccc(Cl)cc3)c2F)c(-c2ccncc2)n1]

Synthesized from intermediate 14 and intermediate 21 using general synthetic method B

### Example 50

### 1-(3-(1-ethyl-3-(pyridin-4-yl)-1H-pyrazol-4-yl)-2-fluorophenyl)-3-(4-(trifluoromethyl)phenyl)urea

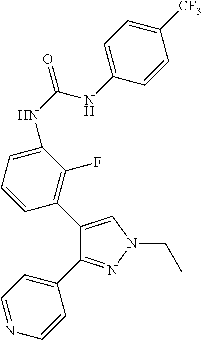 [SMILES: CCn1cc(-c2cccc(NC(=O)Nc3ccc(C(F)(F)F)cc3)c2F)c(-c2ccncc2)n1]

Synthesized from intermediate 15 and intermediate 21 using general synthetic method B

### Example 51

### 1-(3-(1-ethyl-3-(pyridin-4-yl)-1H-pyrazol-4-yl)phenyl)-3-(4-(methylsulfonyl)phenyl)urea

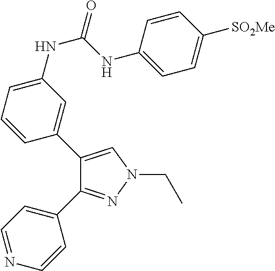 [SMILES: CCn1cc(-c2cccc(NC(=O)Nc3ccc(S(C)(=O)=O)cc3)c2)c(-c2ccncc2)n1]

Synthesized from intermediate 20 and intermediate 21 using general synthetic method B

### Example 52

### 1-(3-(1-ethyl-3-(pyridin-4-yl)-1H-pyrazol-4-yl)phenyl)-3-(4-(trifluoromethyl)phenyl)urea

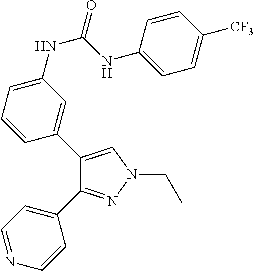 [SMILES: CCn1cc(-c2cccc(NC(=O)Nc3ccc(C(F)(F)F)cc3)c2)c(-c2ccncc2)n1]

Synthesized from intermediate 17 and intermediate 21 using general synthetic method B

### Example 53

### 1-(3-(1-ethyl-3-(pyridin-4-yl)-1H-pyrazol-4-yl)phenyl)-3-(4-(trifluoromethyl)phenyl)imidazolidin-2-one

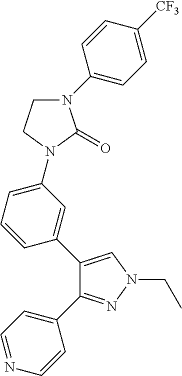 [SMILES: CCn1cc(-c2cccc(N3CCN(c4ccc(C(F)(F)F)cc4)C3=O)c2)c(-c2ccncc2)n1]

Synthesized from intermediate 22 and intermediate 21 using general synthetic method B

### Example 54

### 1-(3-(1-ethyl-3-(pyridin-4-yl)-1H-pyrazol-4-yl)phenyl)-3-(4-(ethylsulfonyl)phenyl)urea

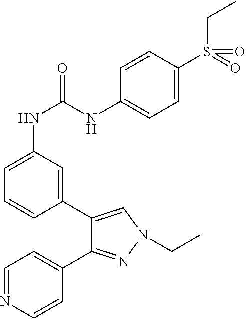 [SMILES: CCn1cc(-c2cccc(NC(=O)Nc3ccc(S(=O)(=O)CC)cc3)c2)c(-c2ccncc2)n1]

Synthesized from intermediate 23 and intermediate 21 using general synthetic method A

### Example 55

### 1-(3-(1-ethyl-3-(pyridin-4-yl)-1H-pyrazol-4-yl)phenyl)-3-(3-(methylsulfonyl)phenyl)urea

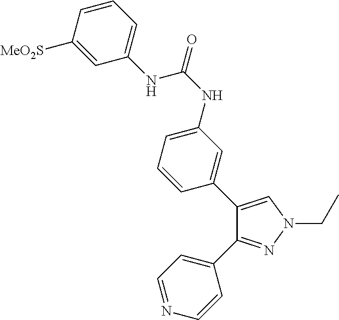 [SMILES: CCn1cc(-c2cccc(NC(=O)Nc3cccc(S(C)(=O)=O)c3)c2)c(-c2ccncc2)n1]

Synthesized from intermediate 24 and intermediate 21 using general synthetic method B

### Example 56

### 4-(3-(3-(1-ethyl-3-(pyridin-4-yl)-1H-pyrazol-4-yl)phenyl)ureido)-N-methylbenzenesulfonamide

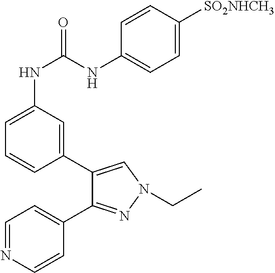

Synthesized from intermediate 25 and intermediate 21 using general synthetic method B

### Example 57

### 1-(3-(1-ethyl-3-(pyridin-4-yl)-1H-pyrazol-4-yl)phenyl)-3-(4-methoxyphenyl)urea

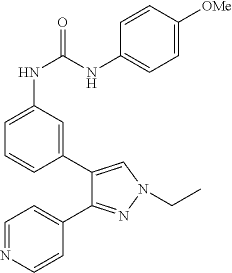 [SMILES: CCn1cc(-c2cccc(NC(=O)Nc3ccc(OC)cc3)c2)c(-c2ccncc2)n1]

Synthesized from intermediate 26 and 1-isocyanato-4-methoxybenzene using general synthetic method A

### Example 58

### 1-(3-(1-ethyl-3-(pyridin-4-yl)-1H-pyrazol-4-yl)phenyl)-3-(4-(pyrrolidin-1-ylsulfonyl)phenyl)urea

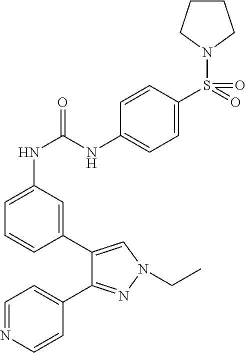 [SMILES: CCn1cc(-c2cccc(NC(=O)Nc3ccc(S(=O)(=O)N4CCCC4)cc3)c2)c(-c2ccncc2)n1]

Synthesized from intermediate 26 and intermediate 27 using general synthetic method A

### Example 59

### 1-(3-(1-ethyl-3-(pyridin-4-yl)-1H-pyrazol-4-yl)phenyl)-3-(4-(morpholinosulfonyl)phenyl)urea

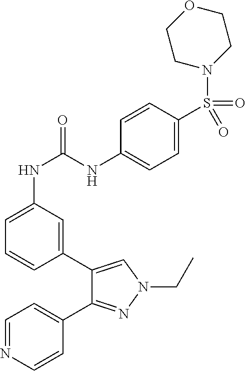 [SMILES: CCn1cc(-c2cccc(NC(=O)Nc3ccc(S(=O)(=O)N4CCOCC4)cc3)c2)c(-c2ccncc2)n1]

Synthesized from intermediate 26 and intermediate 28 using general synthetic method A

### Example 60

### 1-(3-(3-(2-aminopyrimidin-4-yl)-1-ethyl-1H-pyrazol-4-yl)phenyl)-3-(4-(trifluoromethyl)phenyl)urea

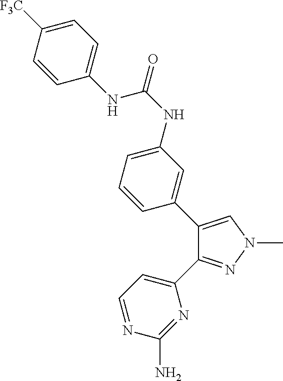 [SMILES: Cn1cc(-c2cccc(NC(=O)Nc3ccc(C(F)(F)F)cc3)c2)c(-c2ccnc(N)n2)n1]

Synthesized from intermediate 31 and intermediate 17 using general synthetic method E

### Example 61

### 1-(3-(3-(2-aminopyrimidin-4-yl)-1-ethyl-1H-pyrazol-4-yl)-2-fluorophenyl)-3-(4-(trifluoromethyl)phenyl)urea

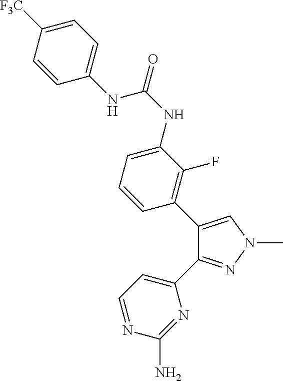 [SMILES: Cn1cc(-c2cccc(NC(=O)Nc3ccc(C(F)(F)F)cc3)c2F)c(-c2ccnc(N)n2)n1]

Synthesized from intermediate 30 and intermediate 15 using general synthetic method E

### Example 62

### 1-(3-(3-(2-aminopyrimidin-4-yl)-1-ethyl-1H-pyrazol-4-yl)phenyl)-3-(4-(trifluoromethyl)phenyl) imidazolidin-2-one

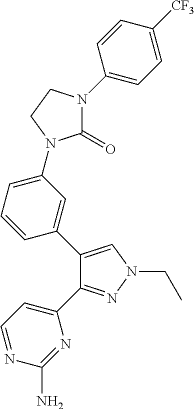 [SMILES: CCn1cc(-c2cccc(N3CCN(c4ccc(C(F)(F)F)cc4)C3=O)c2)c(-c2ccnc(N)n2)n1]

Synthesized from intermediate 30 and intermediate 22 using general synthetic method E

### Example 63

### 1-(3-(1-ethyl-3-(pyridin-4-yl)-1H-pyrazol-4-yl)phenyl)-1-hydroxy-3-(4-(trifluoromethyl)phenyl)urea

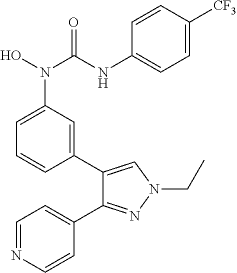 [SMILES: CCn1cc(-c2cccc(N(O)C(=O)Nc3ccc(C(F)(F)F)cc3)c2)c(-c2ccncc2)n1] [0720] Synthesized from intermediate 31 and 1-isocyanato-4-(trifluoromethyl)benzene using general synthetic method A.

Examples 64 to 427 are prepared according to the methods described below.

The following examples are synthesized according the following synthetic scheme using the procedure described to synthesize Example 60 using intermediate 30 and the appropriate boronic ester.

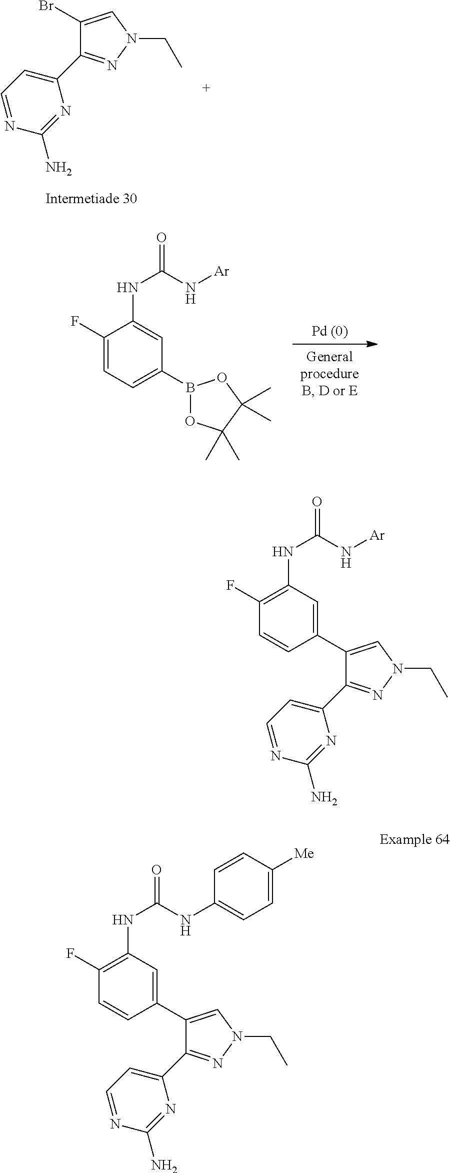

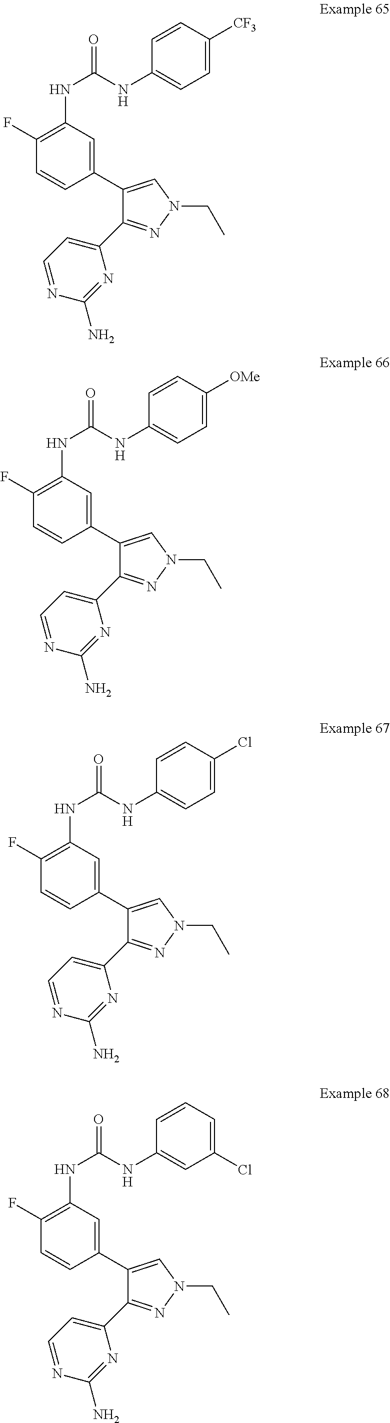 [SMILES: CCn1cc(-c2ccc(F)c(NC(=O)Nc3ccc(C(F)(F)F)cc3)c2)c(-c2ccnc(N)n2)n1 ; CCn1cc(-c2ccc(F)c(NC(=O)Nc3ccc(OC)cc3)c2)c(-c2ccnc(N)n2)n1 ; CCn1cc(-c2ccc(F)c(NC(=O)Nc3ccc(Cl)cc3)c2)c(-c2ccnc(N)n2)n1 ; CCn1cc(-c2ccc(F)c(NC(=O)Nc3cccc(Cl)c3)c2)c(-c2ccnc(N)n2)n1]

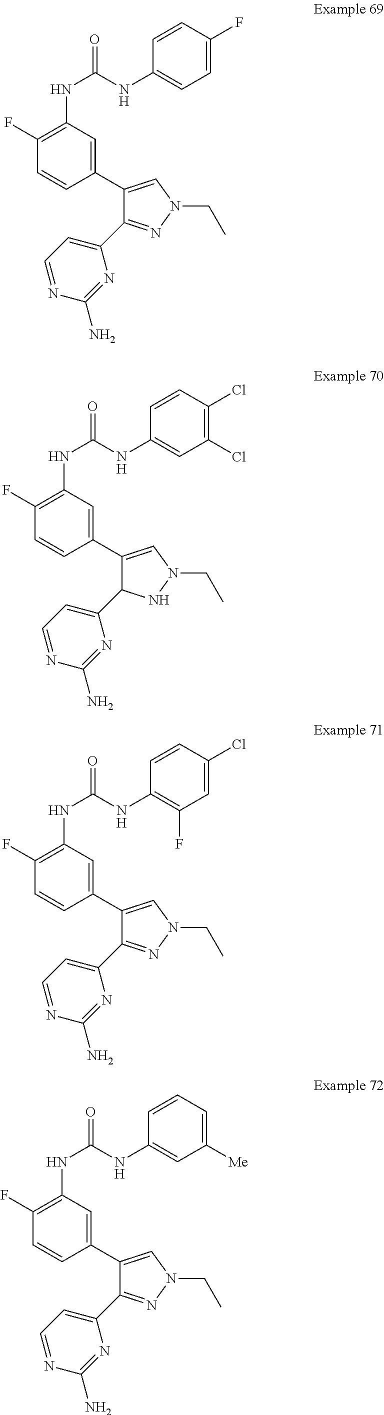 [SMILES: CCn1cc(-c2ccc(F)c(NC(=O)Nc3ccc(F)cc3)c2)c(-c2ccnc(N)n2)n1 ; CCN1C=C(c2ccc(F)c(NC(=O)Nc3ccc(Cl)c(Cl)c3)c2)C(c2ccnc(N)n2)N1 ; CCn1cc(-c2ccc(F)c(NC(=O)Nc3ccc(Cl)cc3F)c2)c(-c2ccnc(N)n2)n1 ; CCn1cc(-c2ccc(F)c(NC(=O)Nc3cccc(C)c3)c2)c(-c2ccnc(N)n2)n1]

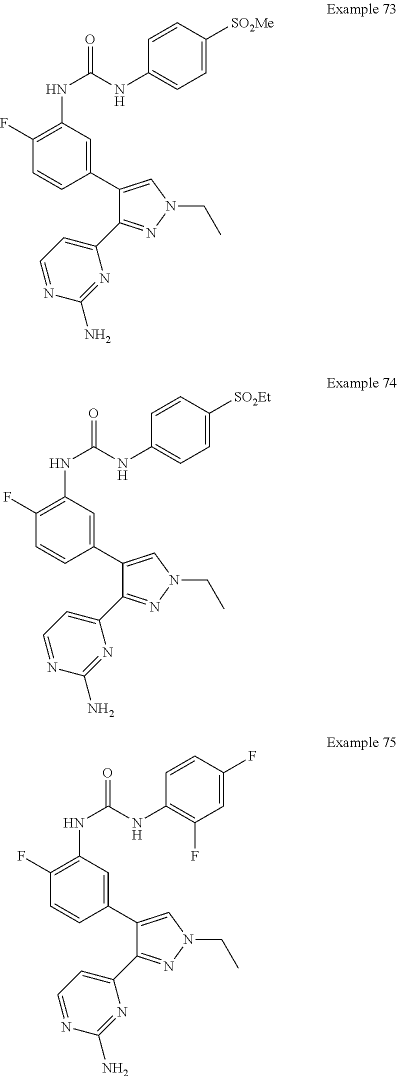

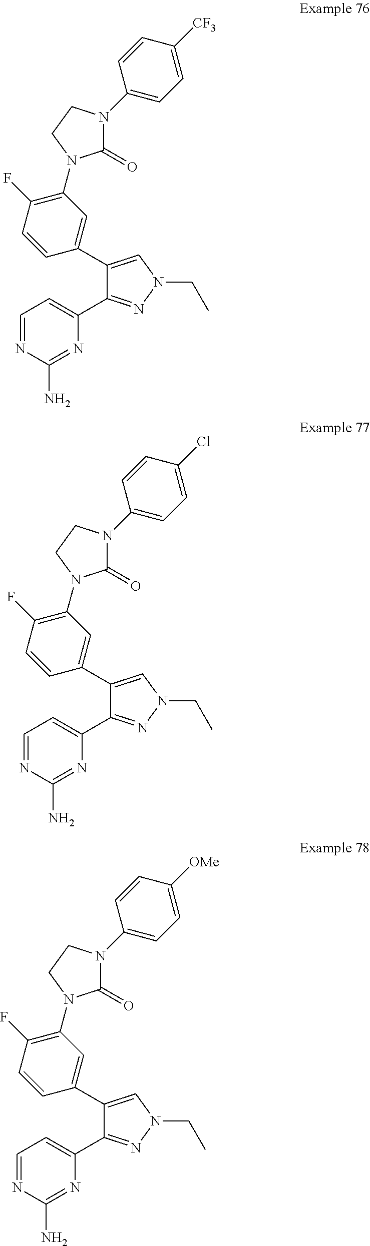 [SMILES: CCn1cc(-c2ccc(F)c(N3CCN(c4ccc(C(F)(F)F)cc4)C3=O)c2)c(-c2ccnc(N)n2)n1 ; CCn1cc(-c2ccc(F)c(N3CCN(c4ccc(Cl)cc4)C3=O)c2)c(-c2ccnc(N)n2)n1 ; CCn1cc(-c2ccc(F)c(N3CCN(c4ccc(OC)cc4)C3=O)c2)c(-c2ccnc(N)n2)n1]

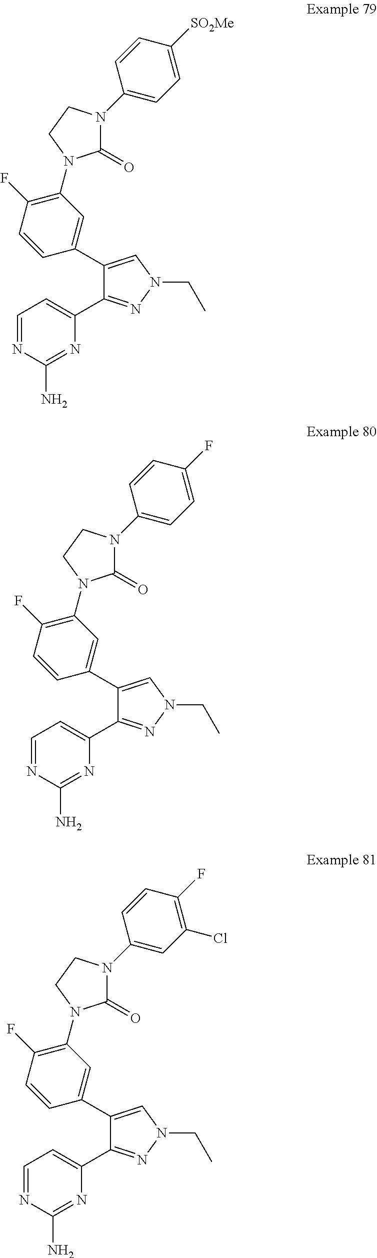 [SMILES: CCn1cc(-c2ccc(F)c(N3CCN(c4ccc(S(C)(=O)=O)cc4)C3=O)c2)c(-c2ccnc(N)n2)n1 ; CCn1cc(-c2ccc(F)c(N3CCN(c4ccc(F)cc4)C3=O)c2)c(-c2ccnc(N)n2)n1 ; CCn1cc(-c2ccc(F)c(N3CCN(c4ccc(F)c(Cl)c4)C3=O)c2)c(-c2ccnc(N)n2)n1]

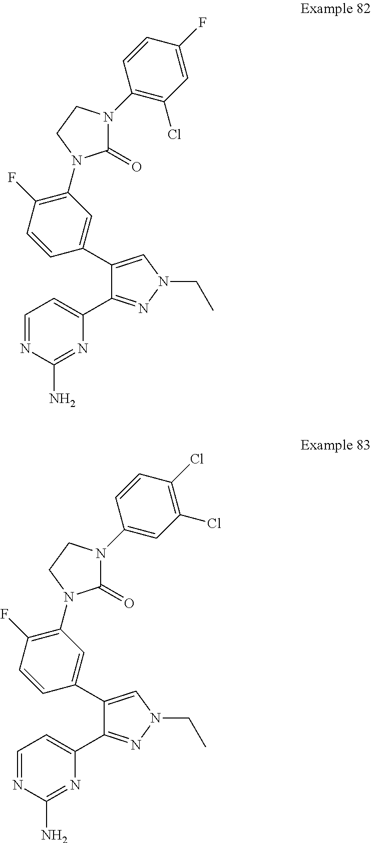 [SMILES: CCn1cc(-c2ccc(F)c(N3CCN(c4ccc(F)cc4Cl)C3=O)c2)c(-c2ccnc(N)n2)n1 ; CCn1cc(-c2ccc(F)c(N3CCN(c4ccc(Cl)c(Cl)c4)C3=O)c2)c(-c2ccnc(N)n2)n1]

The following examples are synthesized according to the following synthetic scheme using the procedure described to synthesize Example 60 using intermediate 30 and the appropriate boronic ester.

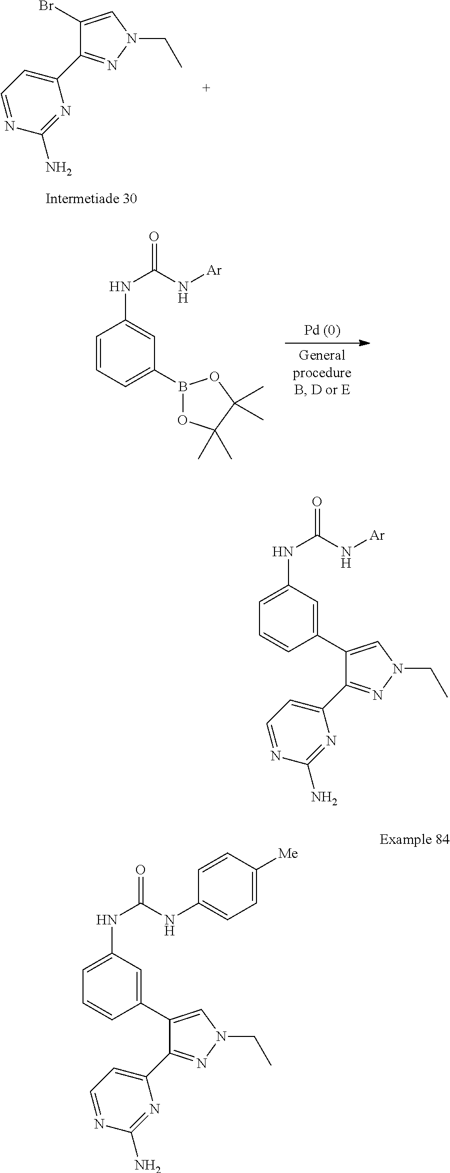

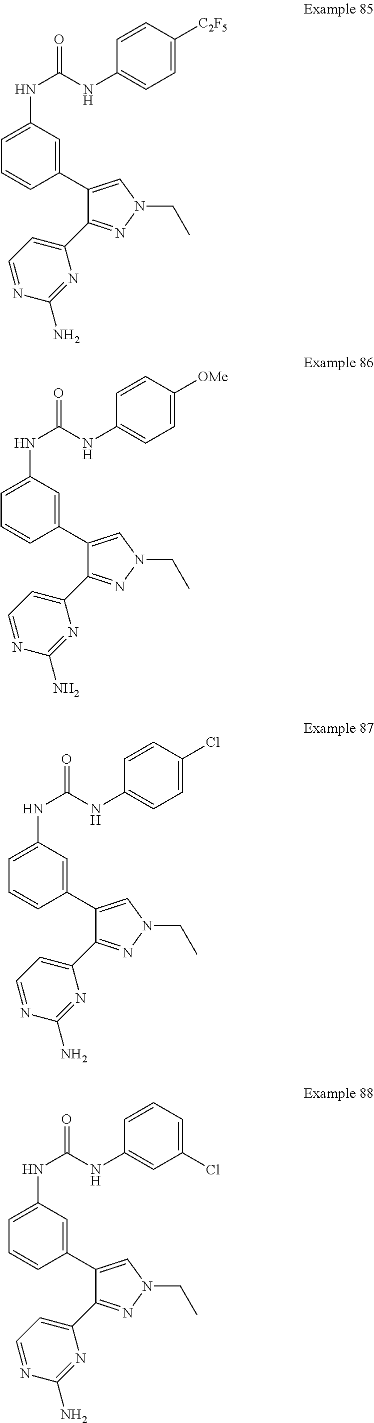

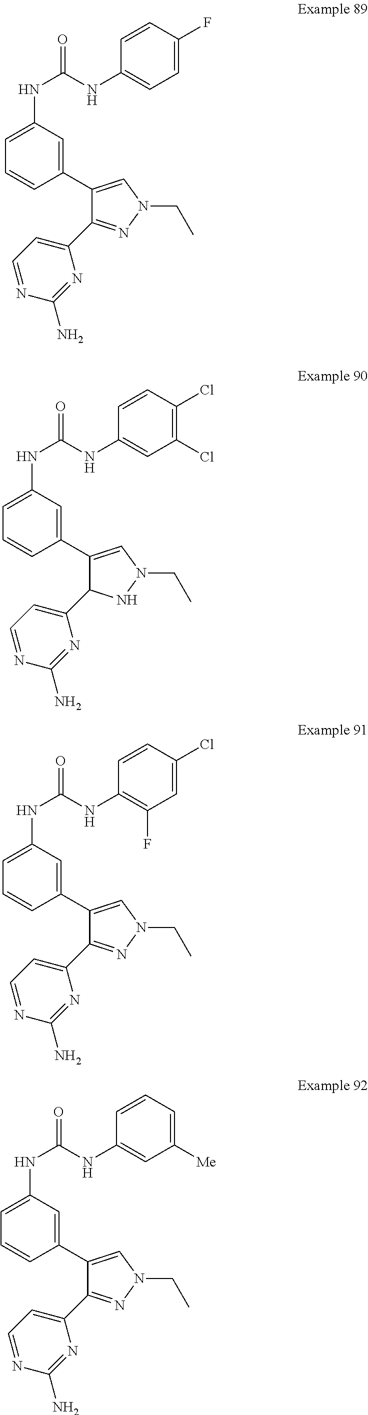 [SMILES: CCn1cc(-c2cccc(NC(=O)Nc3ccc(F)cc3)c2)c(-c2ccnc(N)n2)n1 ; CCN1C=C(c2cccc(NC(=O)Nc3ccc(Cl)c(Cl)c3)c2)C(c2ccnc(N)n2)N1 ; CCn1cc(-c2cccc(NC(=O)Nc3ccc(Cl)cc3F)c2)c(-c2ccnc(N)n2)n1 ; CCn1cc(-c2cccc(NC(=O)Nc3cccc(C)c3)c2)c(-c2ccnc(N)n2)n1]

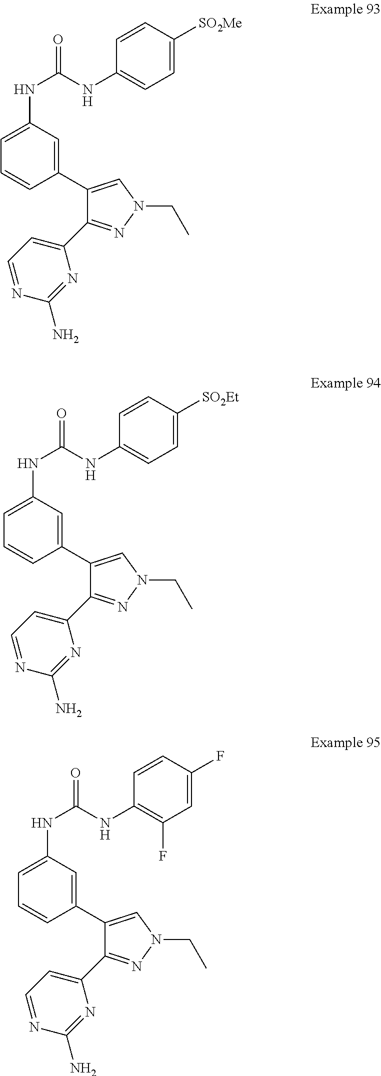

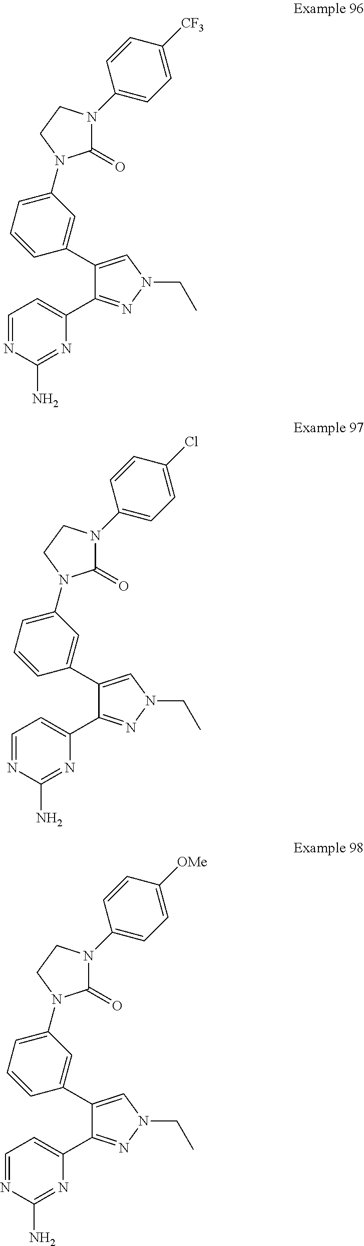 [SMILES: CCn1cc(-c2cccc(N3CCN(c4ccc(C(F)(F)F)cc4)C3=O)c2)c(-c2ccnc(N)n2)n1 ; CCn1cc(-c2cccc(N3CCN(c4ccc(Cl)cc4)C3=O)c2)c(-c2ccnc(N)n2)n1 ; CCn1cc(-c2cccc(N3CCN(c4ccc(OC)cc4)C3=O)c2)c(-c2ccnc(N)n2)n1]

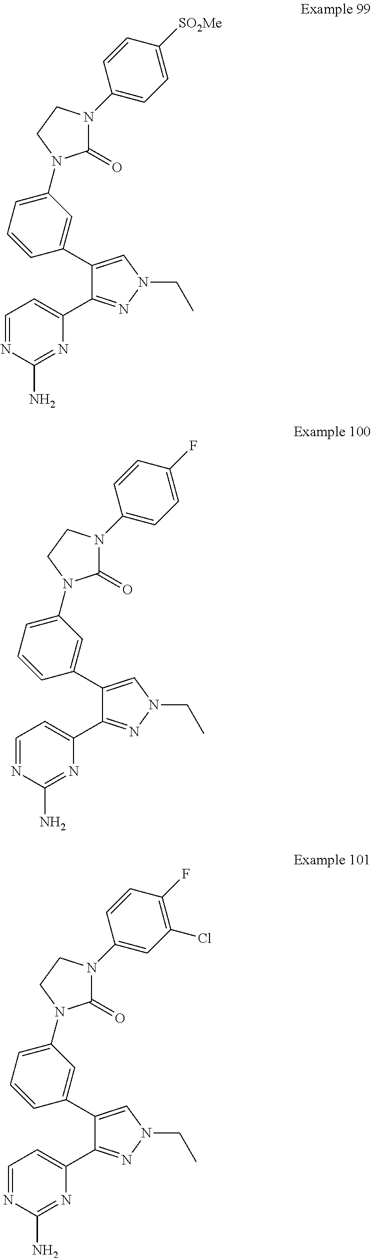 [SMILES: CCn1cc(-c2cccc(N3CCN(c4ccc(S(C)(=O)=O)cc4)C3=O)c2)c(-c2ccnc(N)n2)n1 ; CCn1cc(-c2cccc(N3CCN(c4ccc(F)cc4)C3=O)c2)c(-c2ccnc(N)n2)n1 ; CCn1cc(-c2cccc(N3CCN(c4ccc(F)c(Cl)c4)C3=O)c2)c(-c2ccnc(N)n2)n1]

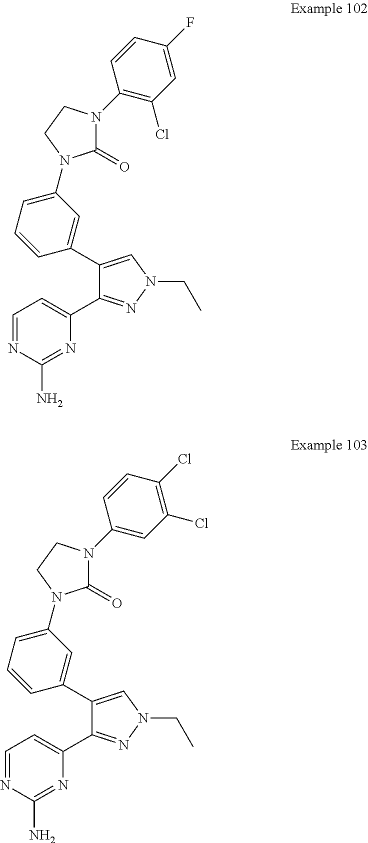 [SMILES: CCn1cc(-c2cccc(N3CCN(c4ccc(F)cc4Cl)C3=O)c2)c(-c2ccnc(N)n2)n1 ; CCn1cc(-c2cccc(N3CCN(c4ccc(Cl)c(Cl)c4)C3=O)c2)c(-c2ccnc(N)n2)n1]

The following examples are synthesized according to the following synthetic scheme using the procedure described to synthesize Example 60 using intermediate 30 and the appropriate boronic ester.

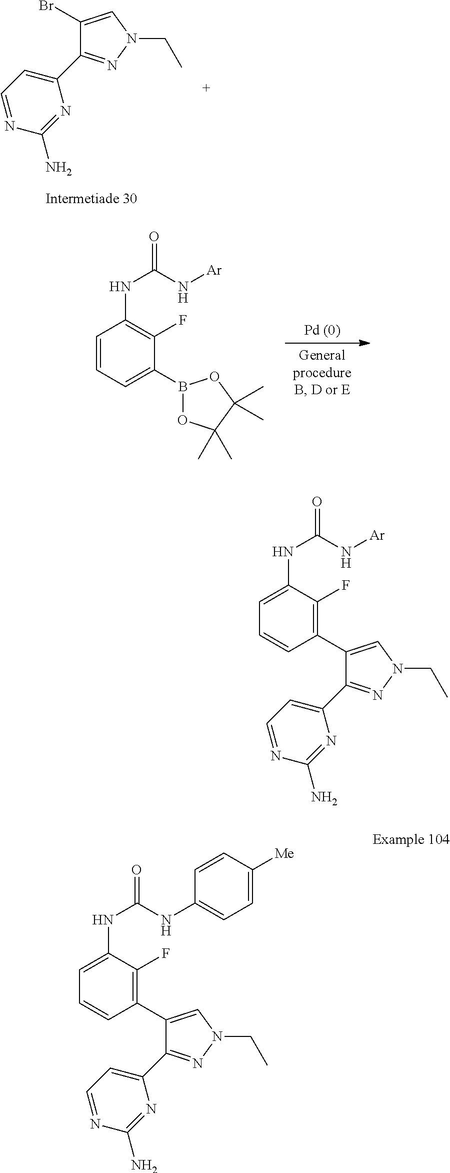

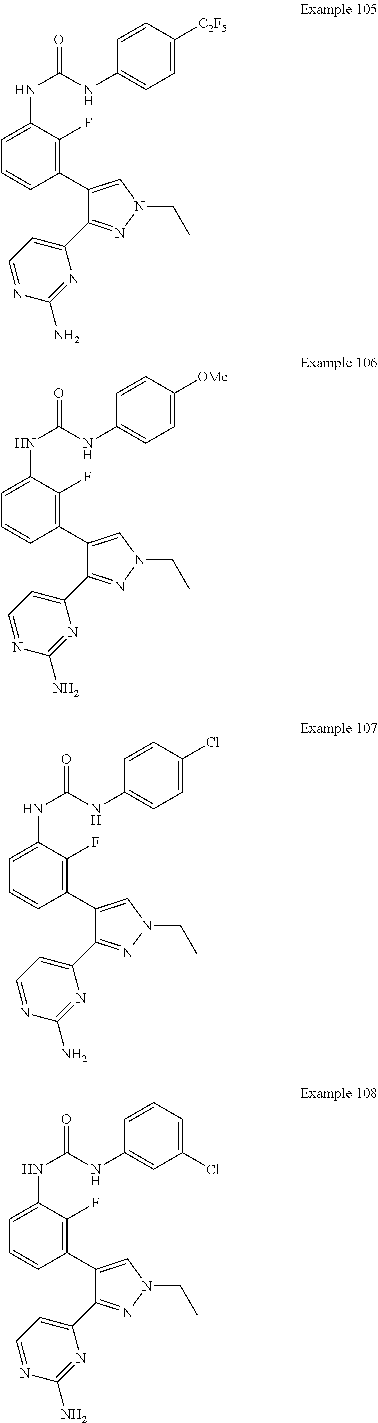

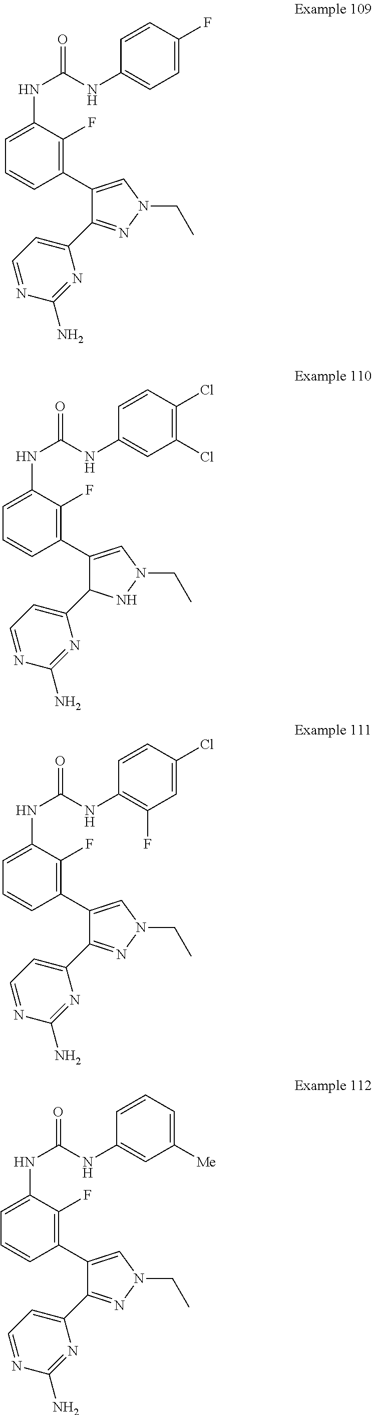 [SMILES: CCn1cc(-c2cccc(NC(=O)Nc3ccc(F)cc3)c2F)c(-c2ccnc(N)n2)n1 ; CCN1C=C(c2cccc(NC(=O)Nc3ccc(Cl)c(Cl)c3)c2F)C(c2ccnc(N)n2)N1 ; CCn1cc(-c2cccc(NC(=O)Nc3ccc(Cl)cc3F)c2F)c(-c2ccnc(N)n2)n1 ; CCn1cc(-c2cccc(NC(=O)Nc3cccc(C)c3)c2F)c(-c2ccnc(N)n2)n1]

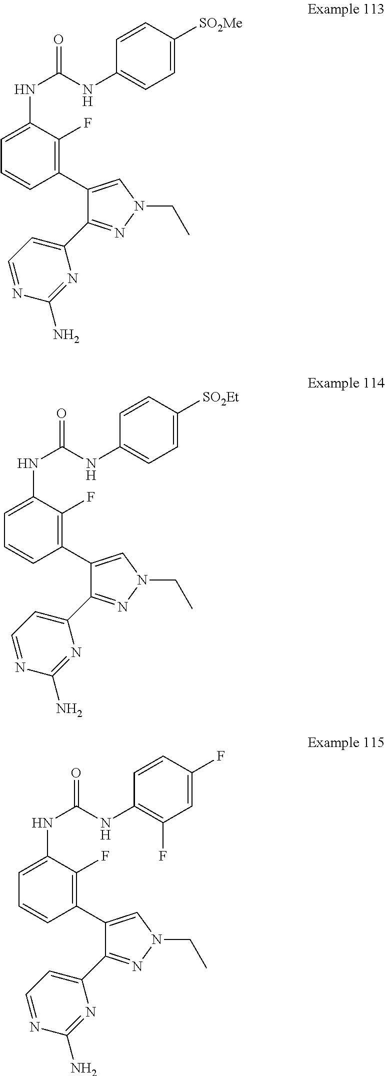

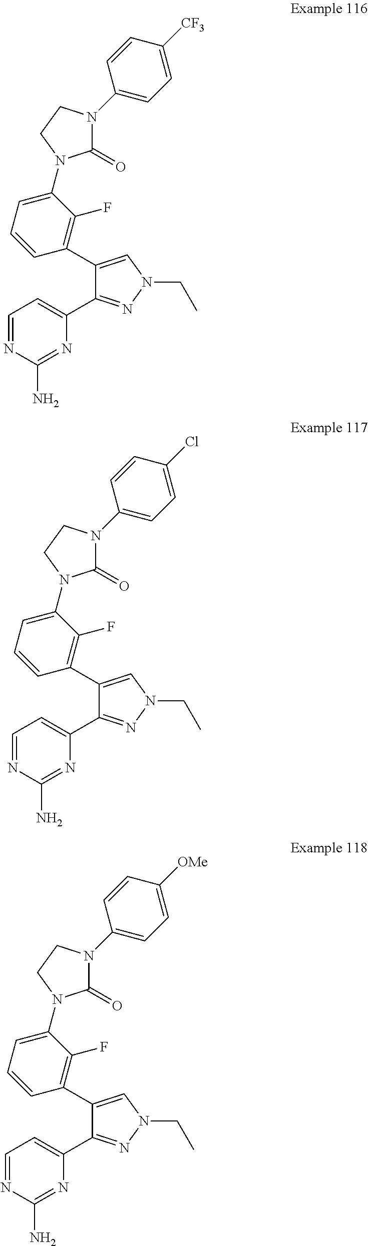 [SMILES: CCn1cc(-c2cccc(N3CCN(c4ccc(C(F)(F)F)cc4)C3=O)c2F)c(-c2ccnc(N)n2)n1 ; CCn1cc(-c2cccc(N3CCN(c4ccc(Cl)cc4)C3=O)c2F)c(-c2ccnc(N)n2)n1 ; CCn1cc(-c2cccc(N3CCN(c4ccc(OC)cc4)C3=O)c2F)c(-c2ccnc(N)n2)n1]

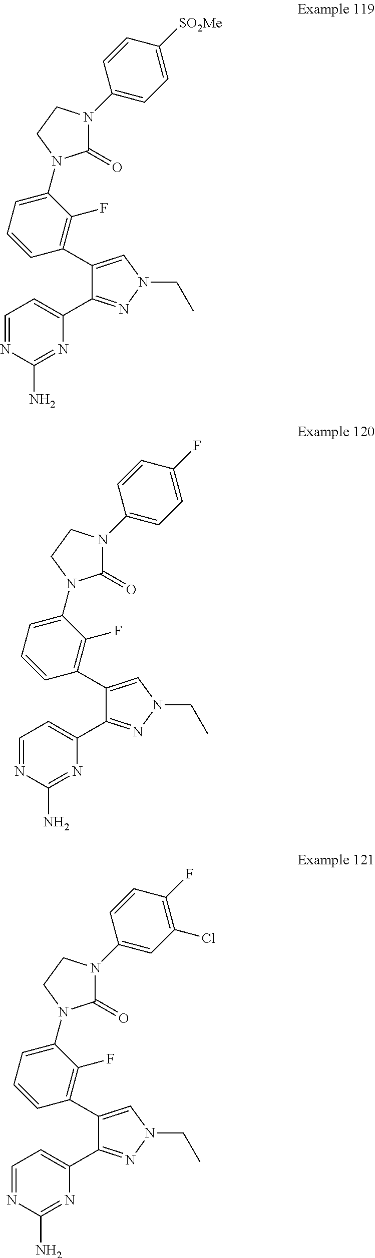 [SMILES: CCn1cc(-c2cccc(N3CCN(c4ccc(S(C)(=O)=O)cc4)C3=O)c2F)c(-c2ccnc(N)n2)n1 ; CCn1cc(-c2cccc(N3CCN(c4ccc(F)cc4)C3=O)c2F)c(-c2ccnc(N)n2)n1 ; CCn1cc(-c2cccc(N3CCN(c4ccc(F)c(Cl)c4)C3=O)c2F)c(-c2ccnc(N)n2)n1]

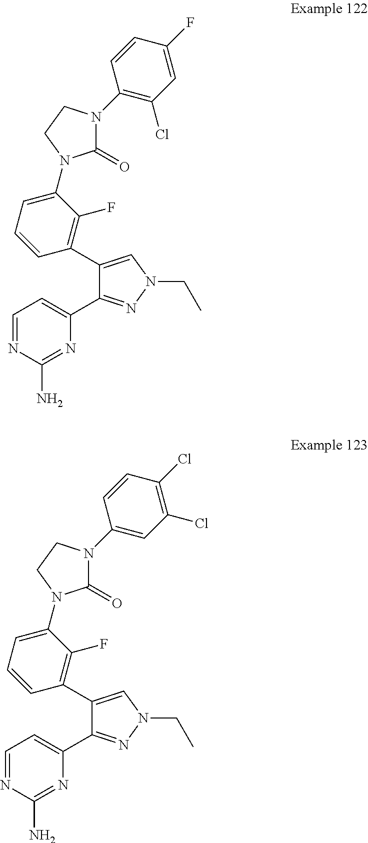 [SMILES: CCn1cc(-c2cccc(N3CCN(c4ccc(F)cc4Cl)C3=O)c2F)c(-c2ccnc(N)n2)n1 ; CCn1cc(-c2cccc(N3CCN(c4ccc(Cl)c(Cl)c4)C3=O)c2F)c(-c2ccnc(N)n2)n1]

The following examples are synthesized according to the following synthetic scheme using the procedure described to synthesize Example 60 using intermediate 32 and the appropriate boronic ester.

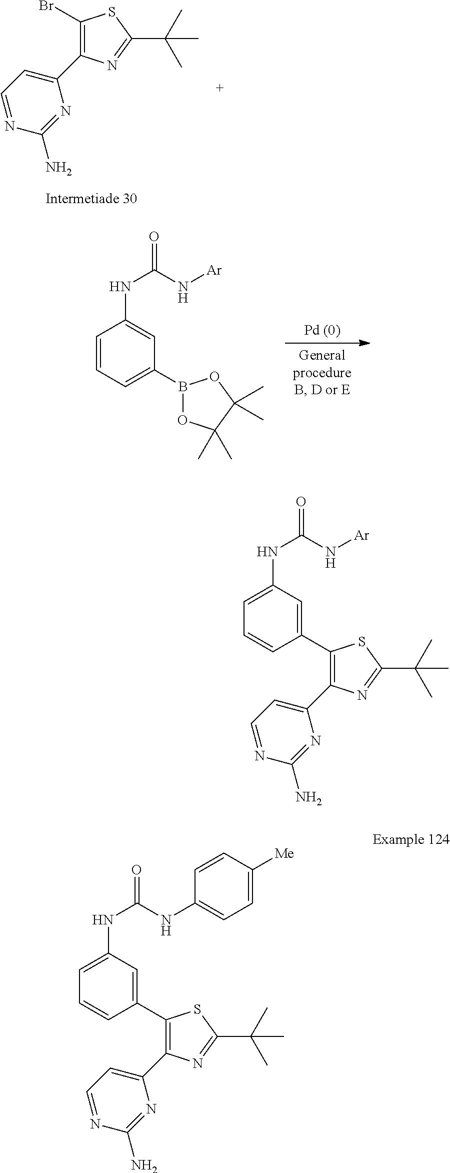

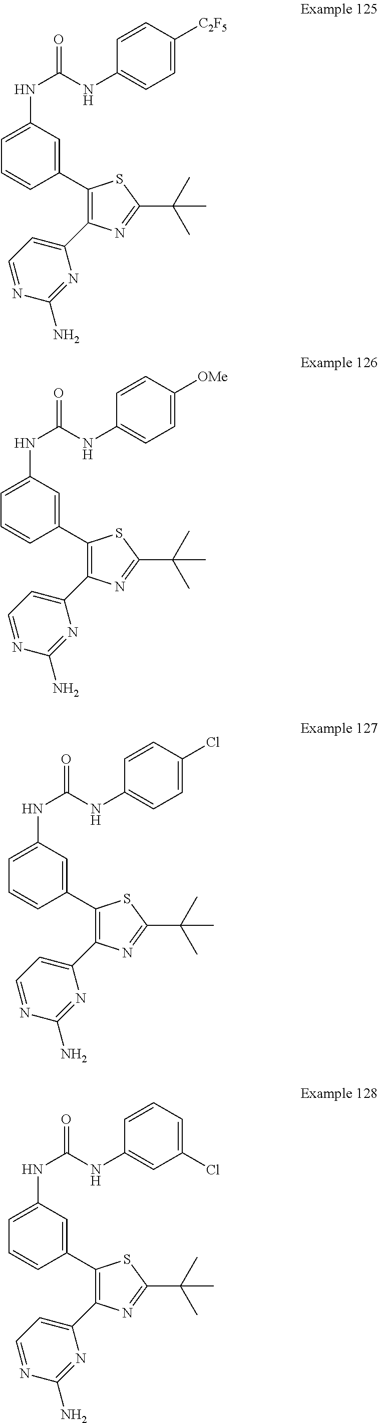

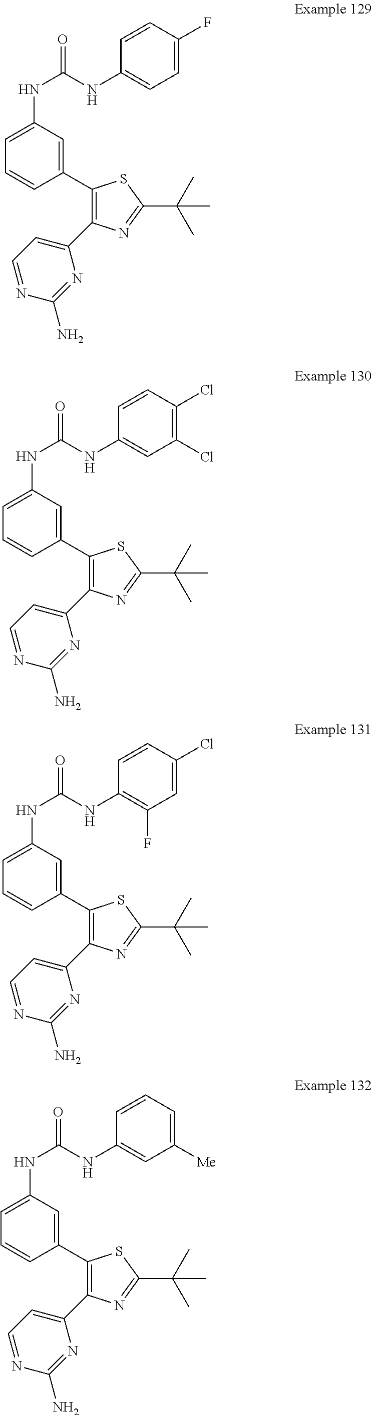 [SMILES: CC(C)(C)c1nc(-c2ccnc(N)n2)c(-c2cccc(NC(=O)Nc3ccc(F)cc3)c2)s1 ; CC(C)(C)c1nc(-c2ccnc(N)n2)c(-c2cccc(NC(=O)Nc3ccc(Cl)c(Cl)c3)c2)s1 ; CC(C)(C)c1nc(-c2ccnc(N)n2)c(-c2cccc(NC(=O)Nc3ccc(Cl)cc3F)c2)s1 ; Cc1cccc(NC(=O)Nc2cccc(-c3sc(C(C)(C)C)nc3-c3ccnc(N)n3)c2)c1]

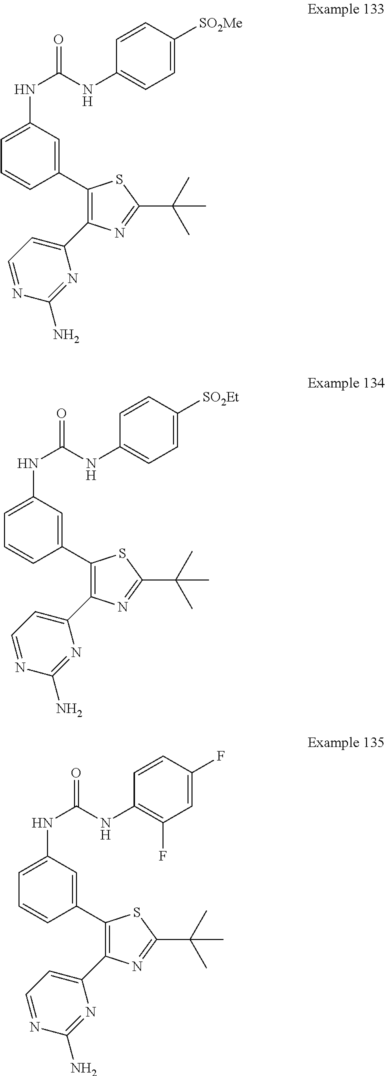

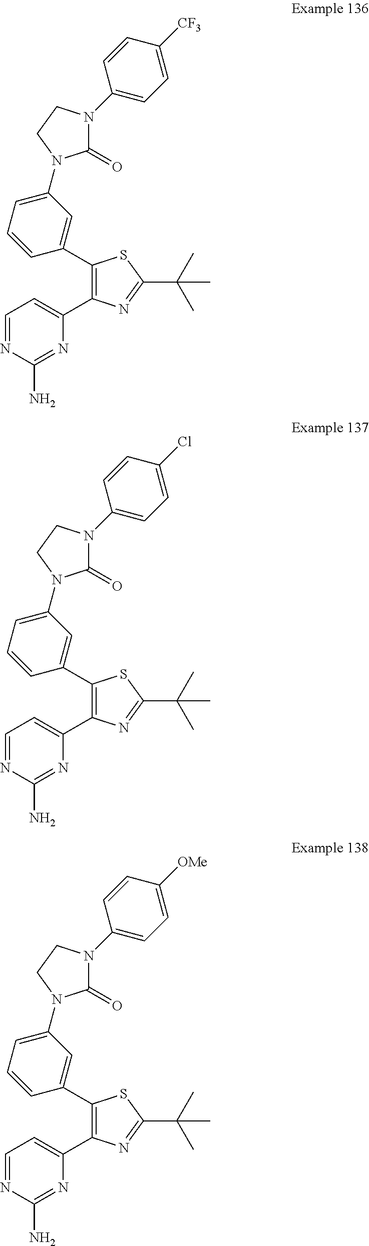 [SMILES: CC(C)(C)c1nc(-c2ccnc(N)n2)c(-c2cccc(N3CCN(c4ccc(C(F)(F)F)cc4)C3=O)c2)s1 ; CC(C)(C)c1nc(-c2ccnc(N)n2)c(-c2cccc(N3CCN(c4ccc(Cl)cc4)C3=O)c2)s1 ; COc1ccc(N2CCN(c3cccc(-c4sc(C(C)(C)C)nc4-c4ccnc(N)n4)c3)C2=O)cc1]

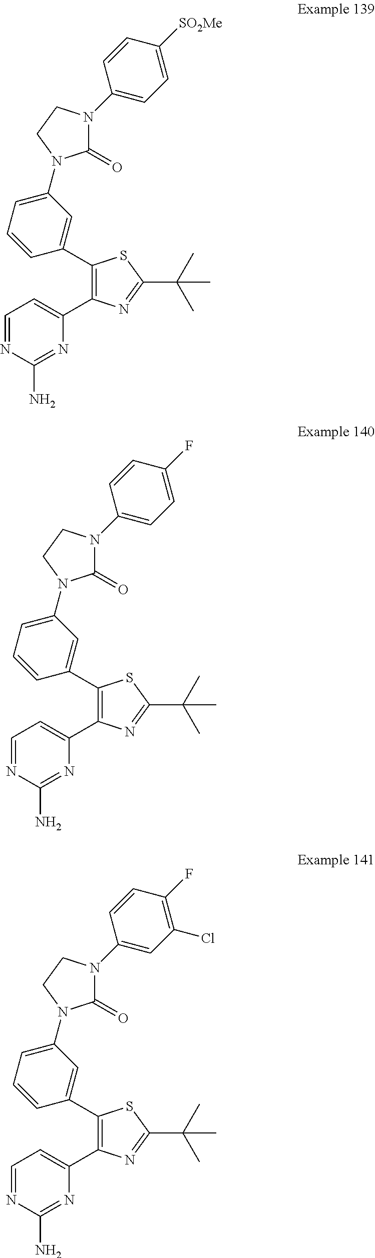 [SMILES: CC(C)(C)c1nc(-c2ccnc(N)n2)c(-c2cccc(N3CCN(c4ccc(S(C)(=O)=O)cc4)C3=O)c2)s1 ; CC(C)(C)c1nc(-c2ccnc(N)n2)c(-c2cccc(N3CCN(c4ccc(F)cc4)C3=O)c2)s1 ; CC(C)(C)c1nc(-c2ccnc(N)n2)c(-c2cccc(N3CCN(c4ccc(F)c(Cl)c4)C3=O)c2)s1]

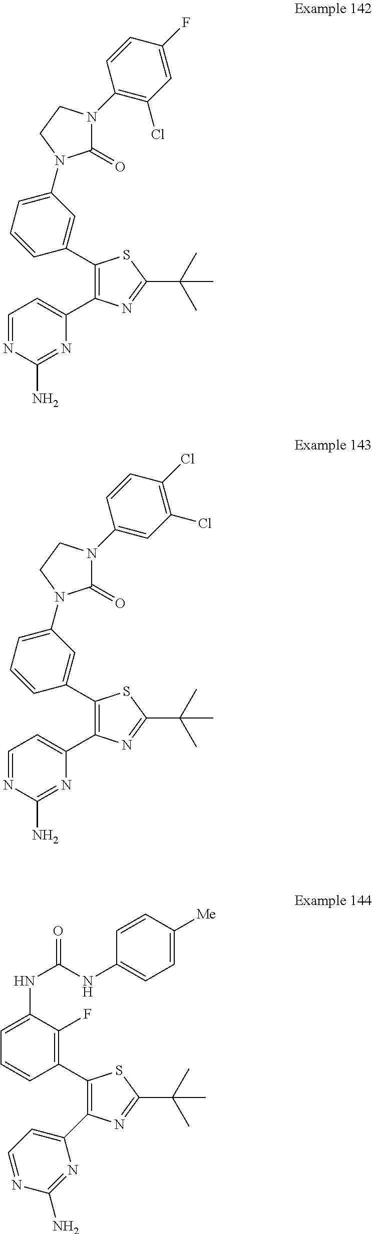 [SMILES: CC(C)(C)c1nc(-c2ccnc(N)n2)c(-c2cccc(N3CCN(c4ccc(F)cc4Cl)C3=O)c2)s1 ; CC(C)(C)c1nc(-c2ccnc(N)n2)c(-c2cccc(N3CCN(c4ccc(Cl)c(Cl)c4)C3=O)c2)s1 ; Cc1ccc(NC(=O)Nc2cccc(-c3sc(C(C)(C)C)nc3-c3ccnc(N)n3)c2F)cc1]

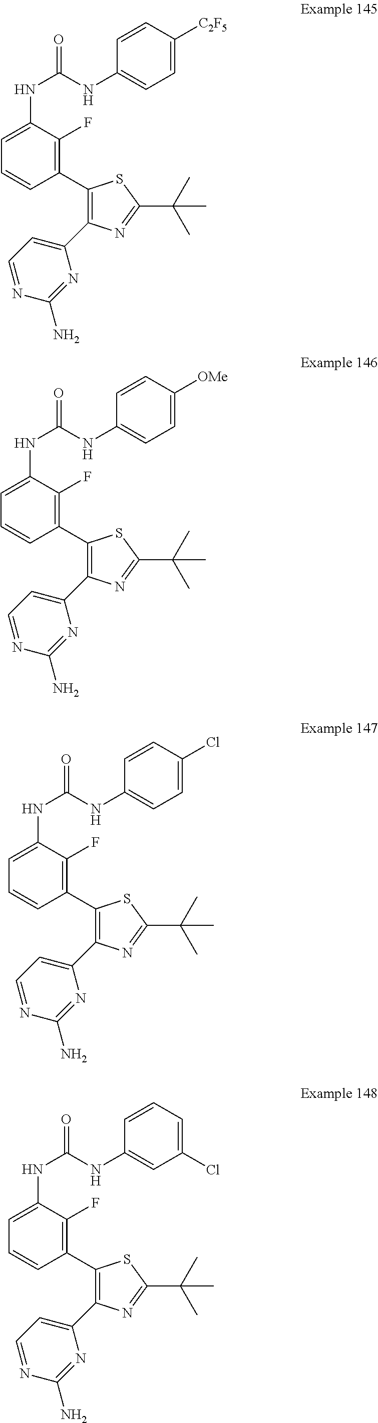

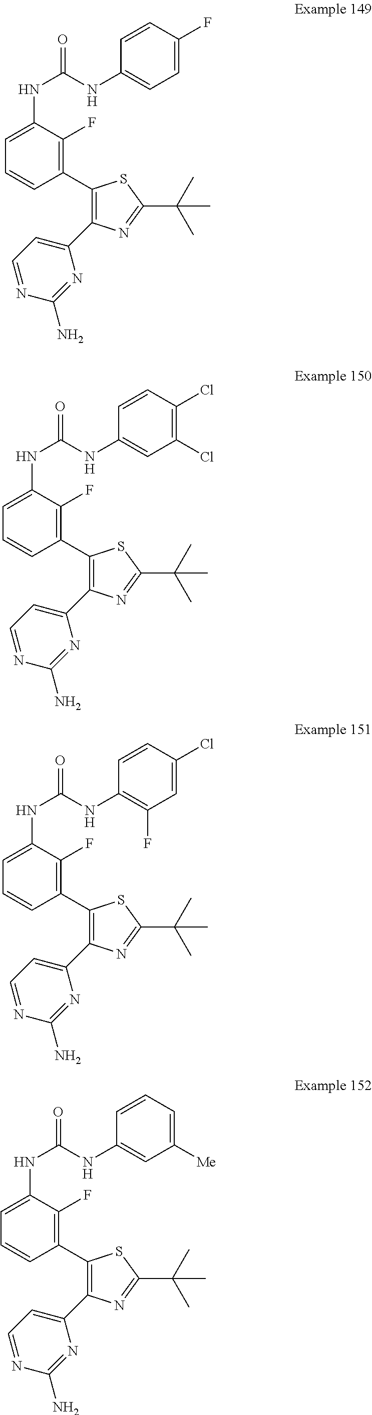 [SMILES: CC(C)(C)c1nc(-c2ccnc(N)n2)c(-c2cccc(NC(=O)Nc3ccc(F)cc3)c2F)s1 ; CC(C)(C)c1nc(-c2ccnc(N)n2)c(-c2cccc(NC(=O)Nc3ccc(Cl)c(Cl)c3)c2F)s1 ; CC(C)(C)c1nc(-c2ccnc(N)n2)c(-c2cccc(NC(=O)Nc3ccc(Cl)cc3F)c2F)s1 ; Cc1cccc(NC(=O)Nc2cccc(-c3sc(C(C)(C)C)nc3-c3ccnc(N)n3)c2F)c1]

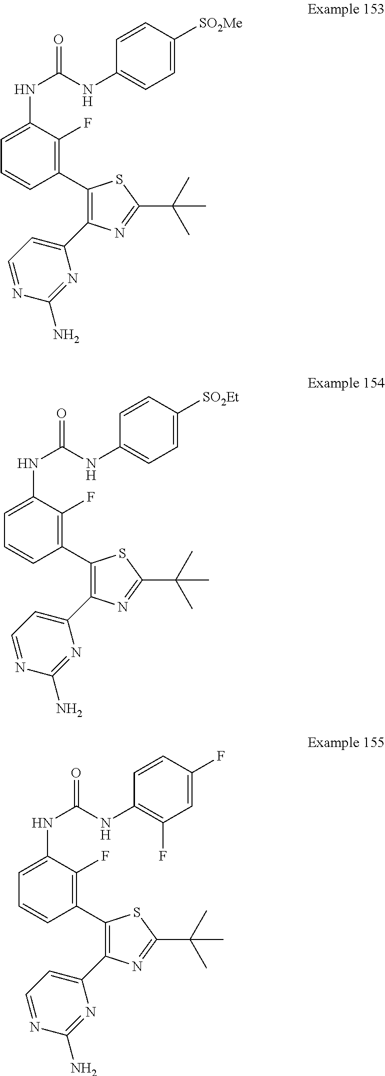

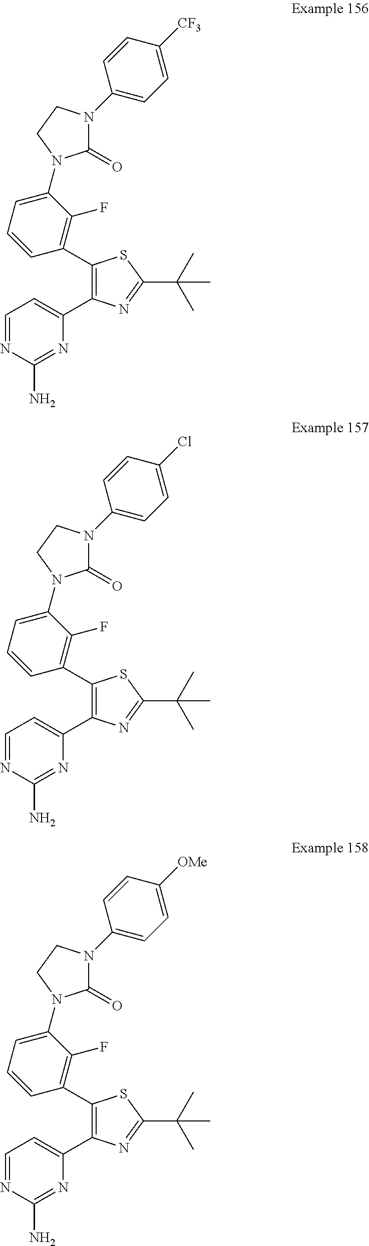 [SMILES: CC(C)(C)c1nc(-c2ccnc(N)n2)c(-c2cccc(N3CCN(c4ccc(C(F)(F)F)cc4)C3=O)c2F)s1 ; CC(C)(C)c1nc(-c2ccnc(N)n2)c(-c2cccc(N3CCN(c4ccc(Cl)cc4)C3=O)c2F)s1 ; COc1ccc(N2CCN(c3cccc(-c4sc(C(C)(C)C)nc4-c4ccnc(N)n4)c3F)C2=O)cc1]

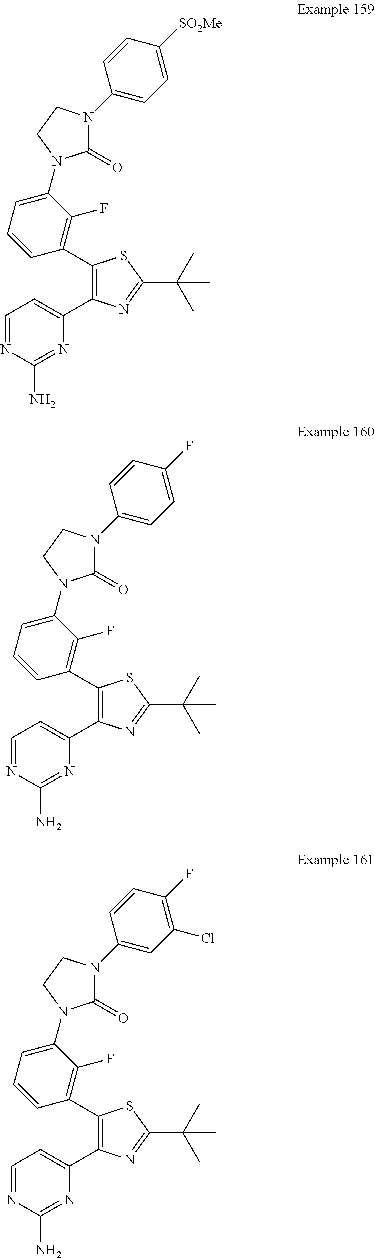 [SMILES: CC(C)(C)c1nc(-c2ccnc(N)n2)c(-c2cccc(N3CCN(c4ccc(S(C)(=O)=O)cc4)C3=O)c2F)s1 ; CC(C)(C)c1nc(-c2ccnc(N)n2)c(-c2cccc(N3CCN(c4ccc(F)cc4)C3=O)c2F)s1 ; CC(C)(C)c1nc(-c2ccnc(N)n2)c(-c2cccc(N3CCN(c4ccc(F)c(Cl)c4)C3=O)c2F)s1]

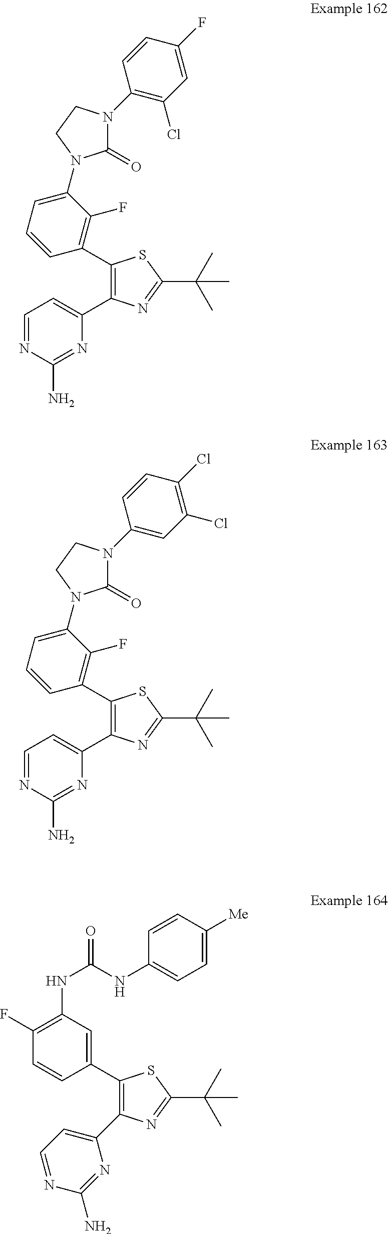 [SMILES: CC(C)(C)c1nc(-c2ccnc(N)n2)c(-c2cccc(N3CCN(c4ccc(F)cc4Cl)C3=O)c2F)s1 ; CC(C)(C)c1nc(-c2ccnc(N)n2)c(-c2cccc(N3CCN(c4ccc(Cl)c(Cl)c4)C3=O)c2F)s1 ; Cc1ccc(NC(=O)Nc2cc(-c3sc(C(C)(C)C)nc3-c3ccnc(N)n3)ccc2F)cc1]

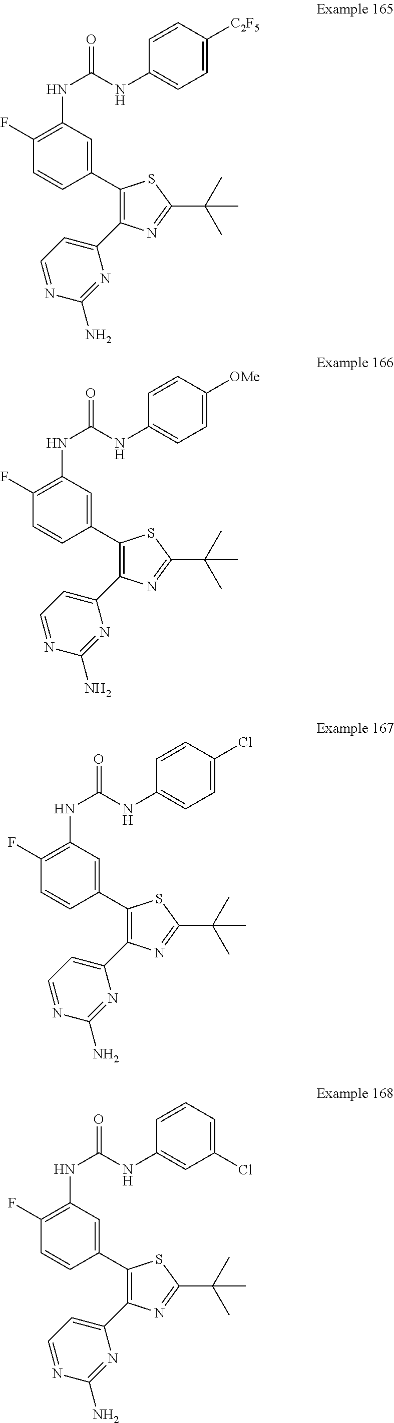

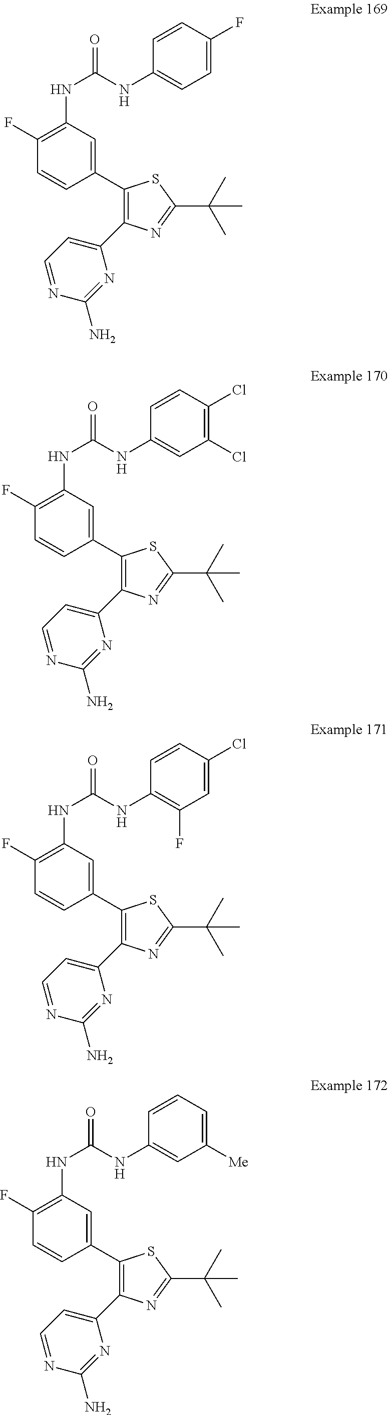 [SMILES: CC(C)(C)c1nc(-c2ccnc(N)n2)c(-c2ccc(F)c(NC(=O)Nc3ccc(F)cc3)c2)s1 ; CC(C)(C)c1nc(-c2ccnc(N)n2)c(-c2ccc(F)c(NC(=O)Nc3ccc(Cl)c(Cl)c3)c2)s1 ; CC(C)(C)c1nc(-c2ccnc(N)n2)c(-c2ccc(F)c(NC(=O)Nc3ccc(Cl)cc3F)c2)s1 ; Cc1cccc(NC(=O)Nc2cc(-c3sc(C(C)(C)C)nc3-c3ccnc(N)n3)ccc2F)c1]

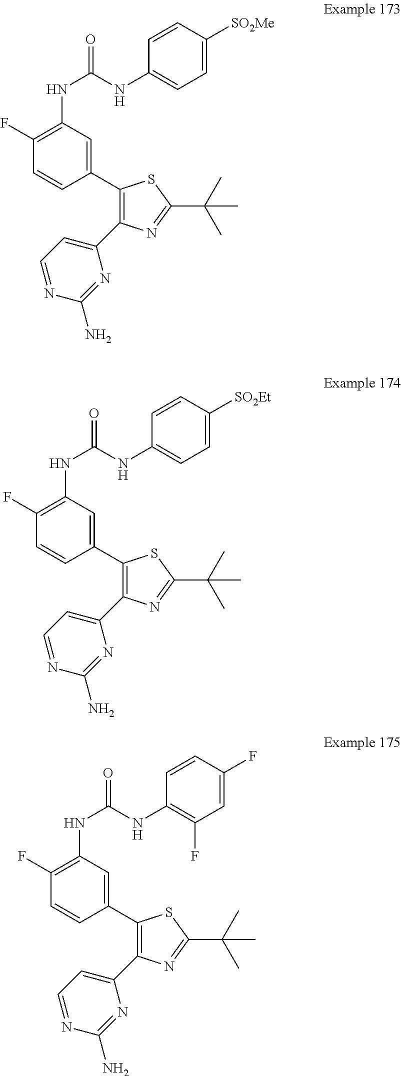

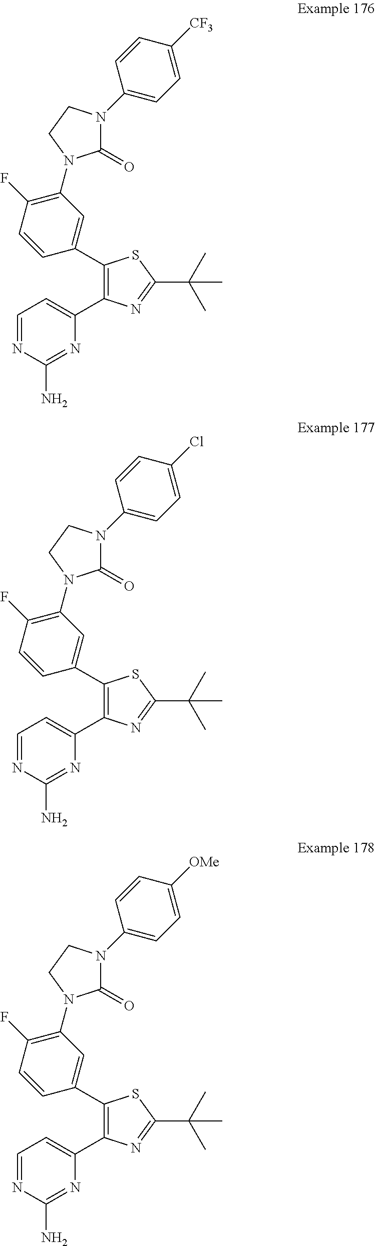 [SMILES: CC(C)(C)c1nc(-c2ccnc(N)n2)c(-c2ccc(F)c(N3CCN(c4ccc(C(F)(F)F)cc4)C3=O)c2)s1 ; CC(C)(C)c1nc(-c2ccnc(N)n2)c(-c2ccc(F)c(N3CCN(c4ccc(Cl)cc4)C3=O)c2)s1 ; COc1ccc(N2CCN(c3cc(-c4sc(C(C)(C)C)nc4-c4ccnc(N)n4)ccc3F)C2=O)cc1]

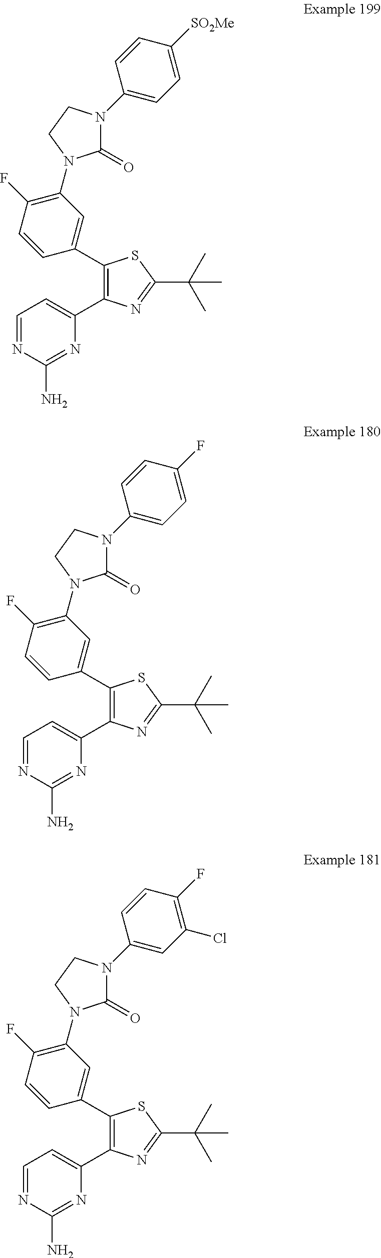 [SMILES: CC(C)(C)c1nc(-c2ccnc(N)n2)c(-c2ccc(F)c(N3CCN(c4ccc(S(C)(=O)=O)cc4)C3=O)c2)s1 ; CC(C)(C)c1nc(-c2ccnc(N)n2)c(-c2ccc(F)c(N3CCN(c4ccc(F)cc4)C3=O)c2)s1 ; CC(C)(C)c1nc(-c2ccnc(N)n2)c(-c2ccc(F)c(N3CCN(c4ccc(F)c(Cl)c4)C3=O)c2)s1]

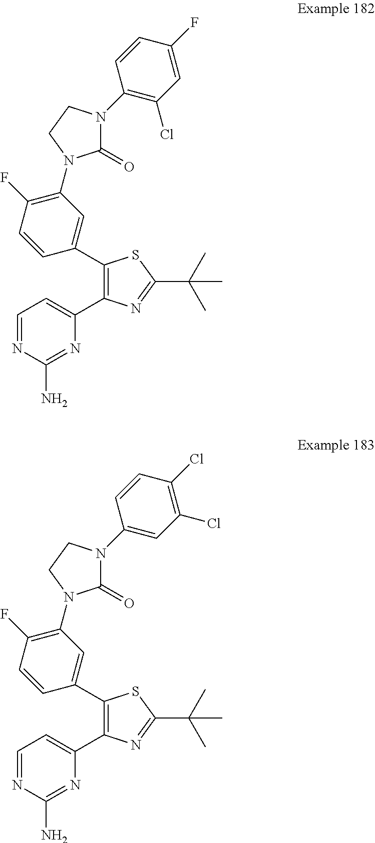 [SMILES: CC(C)(C)c1nc(-c2ccnc(N)n2)c(-c2ccc(F)c(N3CCN(c4ccc(F)cc4Cl)C3=O)c2)s1 ; CC(C)(C)c1nc(-c2ccnc(N)n2)c(-c2ccc(F)c(N3CCN(c4ccc(Cl)c(Cl)c4)C3=O)c2)s1]

The following examples are synthesized according to the following synthetic scheme using the procedure described to synthesize the example 60 using the intermediate 33 (synthesized using the same methodology as 32) and the appropriate boronic ester.

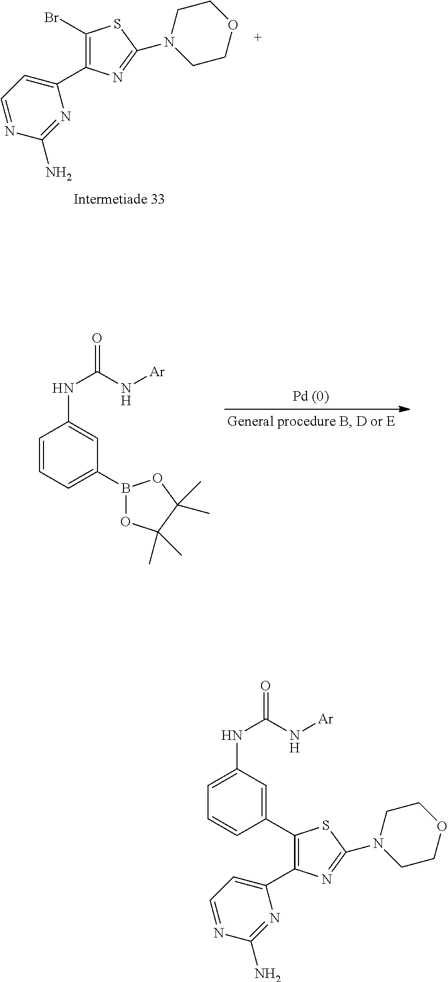

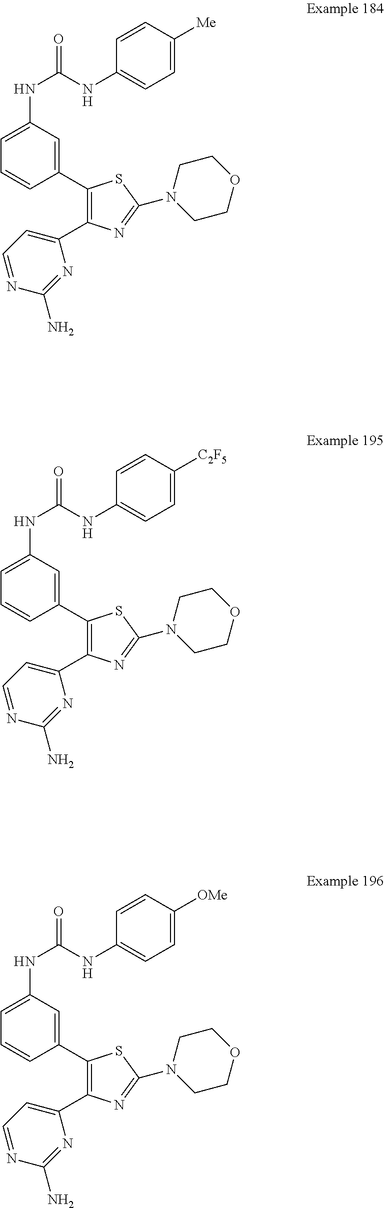

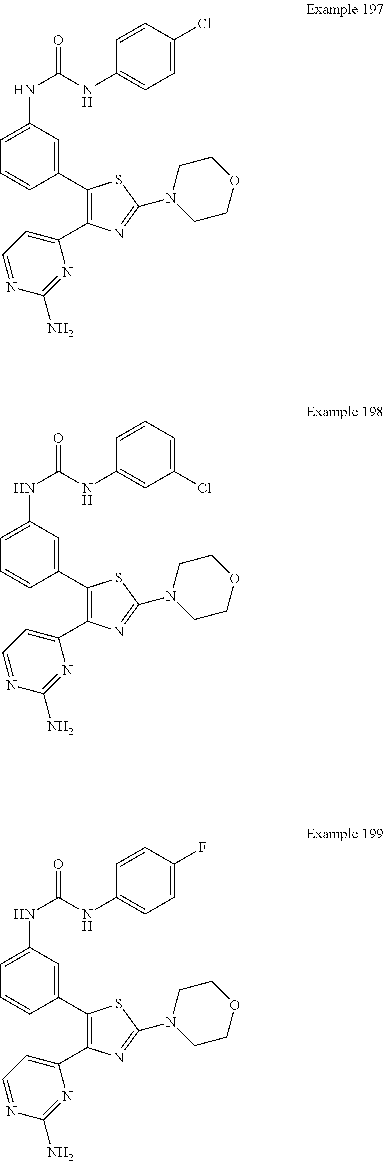 [SMILES: Nc1nccc(-c2nc(N3CCOCC3)sc2-c2cccc(NC(=O)Nc3ccc(Cl)cc3)c2)n1 ; Nc1nccc(-c2nc(N3CCOCC3)sc2-c2cccc(NC(=O)Nc3cccc(Cl)c3)c2)n1 ; Nc1nccc(-c2nc(N3CCOCC3)sc2-c2cccc(NC(=O)Nc3ccc(F)cc3)c2)n1]

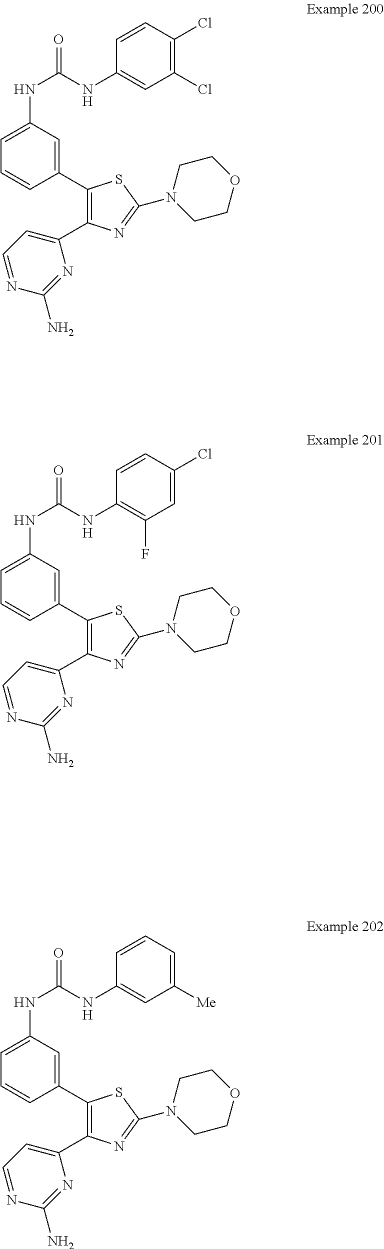 [SMILES: Nc1nccc(-c2nc(N3CCOCC3)sc2-c2cccc(NC(=O)Nc3ccc(Cl)c(Cl)c3)c2)n1 ; Nc1nccc(-c2nc(N3CCOCC3)sc2-c2cccc(NC(=O)Nc3ccc(Cl)cc3F)c2)n1 ; Cc1cccc(NC(=O)Nc2cccc(-c3sc(N4CCOCC4)nc3-c3ccnc(N)n3)c2)c1]

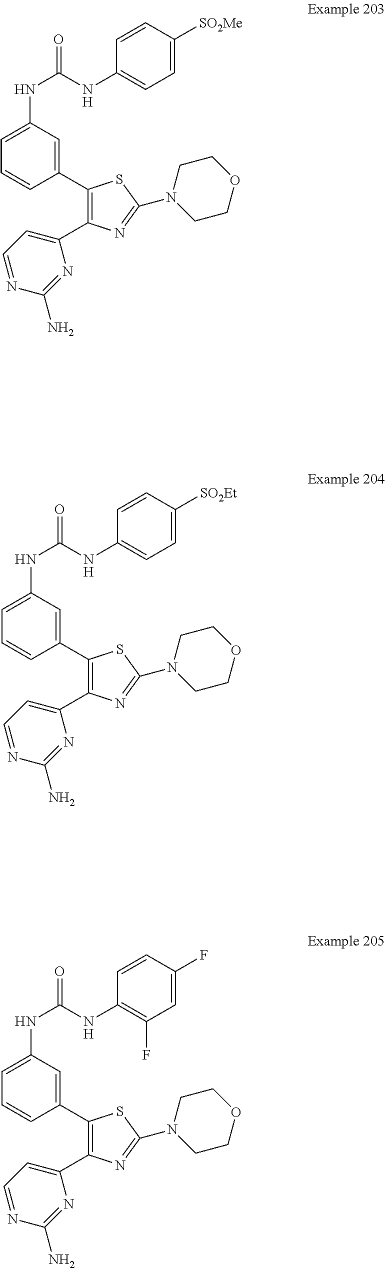

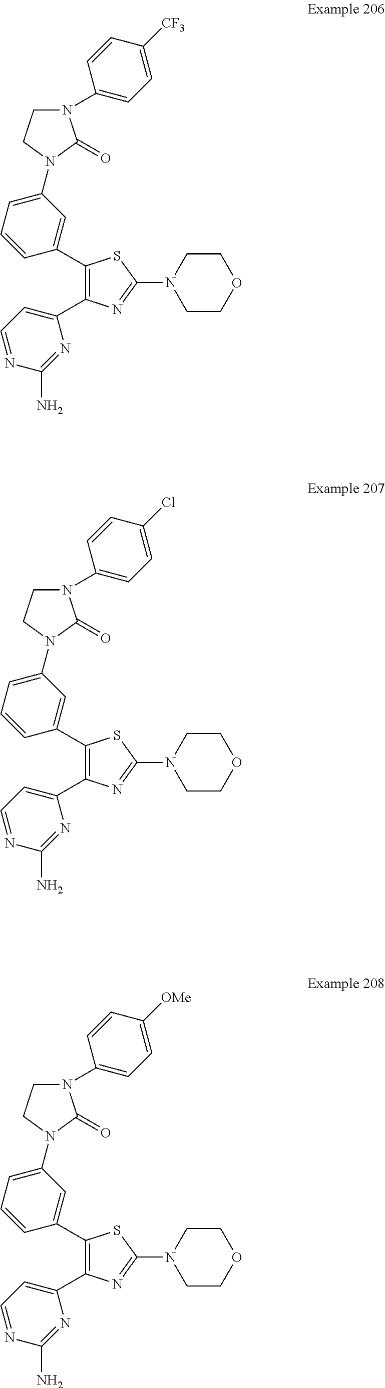 [SMILES: Nc1nccc(-c2nc(N3CCOCC3)sc2-c2cccc(N3CCN(c4ccc(C(F)(F)F)cc4)C3=O)c2)n1 ; Nc1nccc(-c2nc(N3CCOCC3)sc2-c2cccc(N3CCN(c4ccc(Cl)cc4)C3=O)c2)n1 ; COc1ccc(N2CCN(c3cccc(-c4sc(N5CCOCC5)nc4-c4ccnc(N)n4)c3)C2=O)cc1]

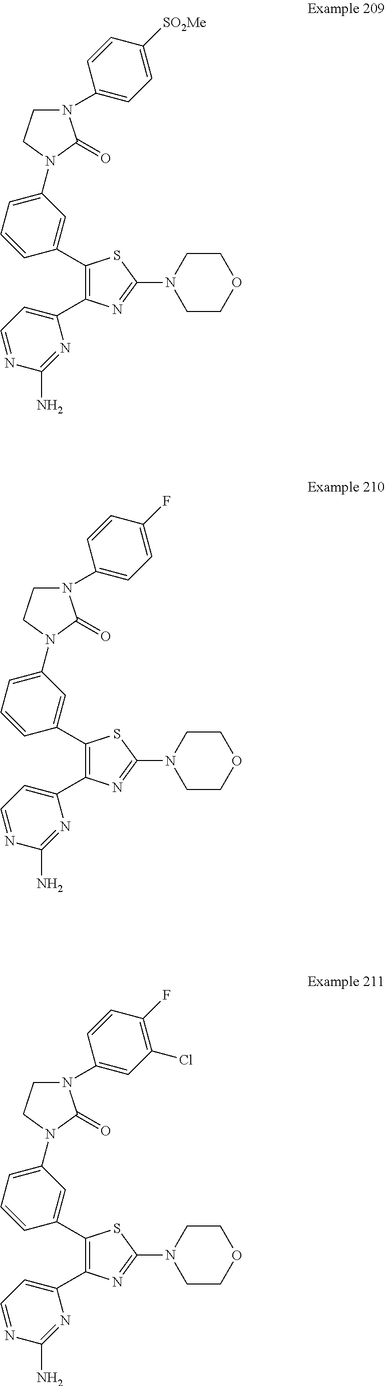 [SMILES: CS(=O)(=O)c1ccc(N2CCN(c3cccc(-c4sc(N5CCOCC5)nc4-c4ccnc(N)n4)c3)C2=O)cc1 ; Nc1nccc(-c2nc(N3CCOCC3)sc2-c2cccc(N3CCN(c4ccc(F)cc4)C3=O)c2)n1 ; Nc1nccc(-c2nc(N3CCOCC3)sc2-c2cccc(N3CCN(c4ccc(F)c(Cl)c4)C3=O)c2)n1]

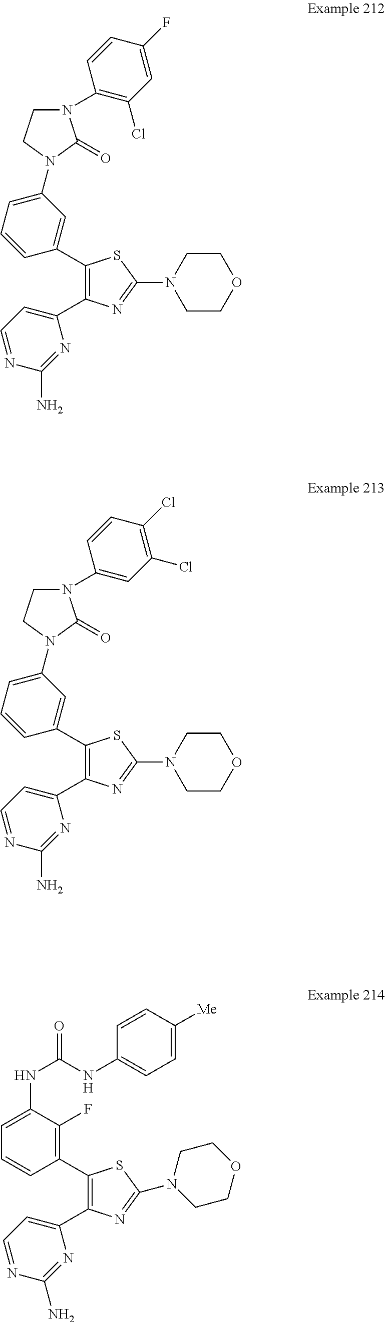 [SMILES: Nc1nccc(-c2nc(N3CCOCC3)sc2-c2cccc(N3CCN(c4ccc(F)cc4Cl)C3=O)c2)n1 ; Nc1nccc(-c2nc(N3CCOCC3)sc2-c2cccc(N3CCN(c4ccc(Cl)c(Cl)c4)C3=O)c2)n1 ; Cc1ccc(NC(=O)Nc2cccc(-c3sc(N4CCOCC4)nc3-c3ccnc(N)n3)c2F)cc1]

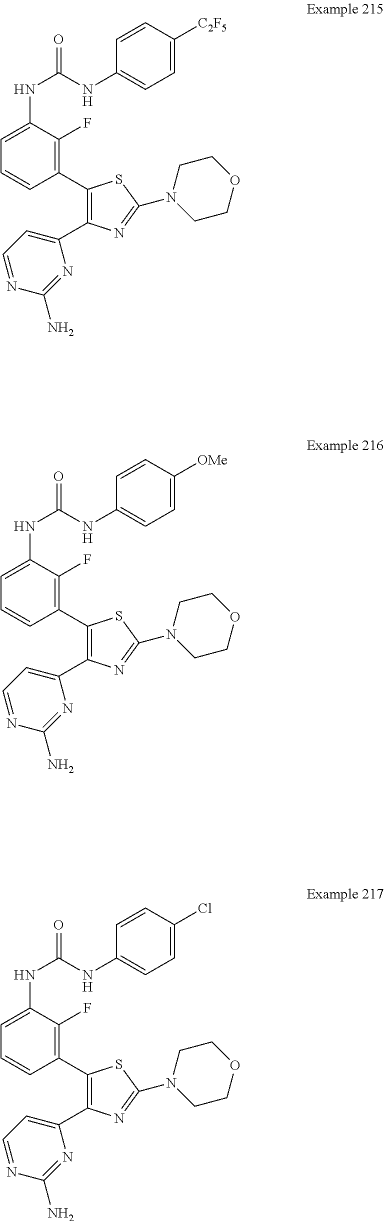

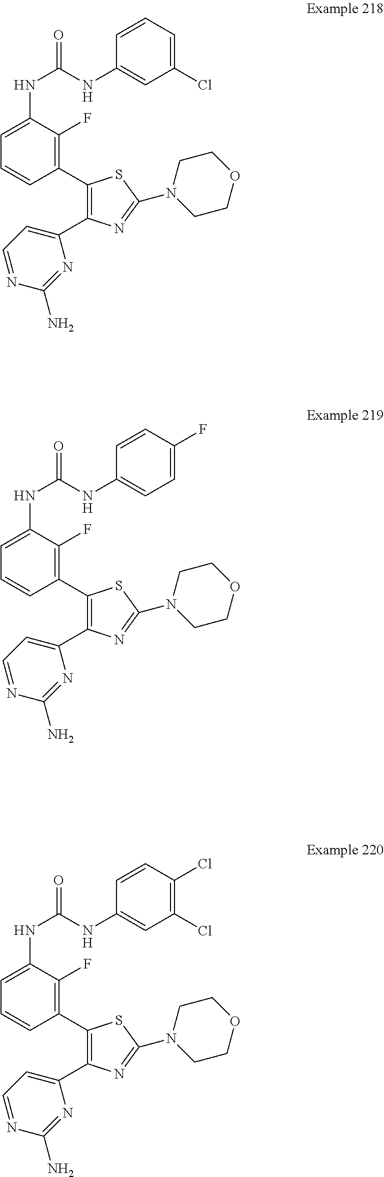 [SMILES: Nc1nccc(-c2nc(N3CCOCC3)sc2-c2cccc(NC(=O)Nc3cccc(Cl)c3)c2F)n1 ; Nc1nccc(-c2nc(N3CCOCC3)sc2-c2cccc(NC(=O)Nc3ccc(F)cc3)c2F)n1 ; Nc1nccc(-c2nc(N3CCOCC3)sc2-c2cccc(NC(=O)Nc3ccc(Cl)c(Cl)c3)c2F)n1]

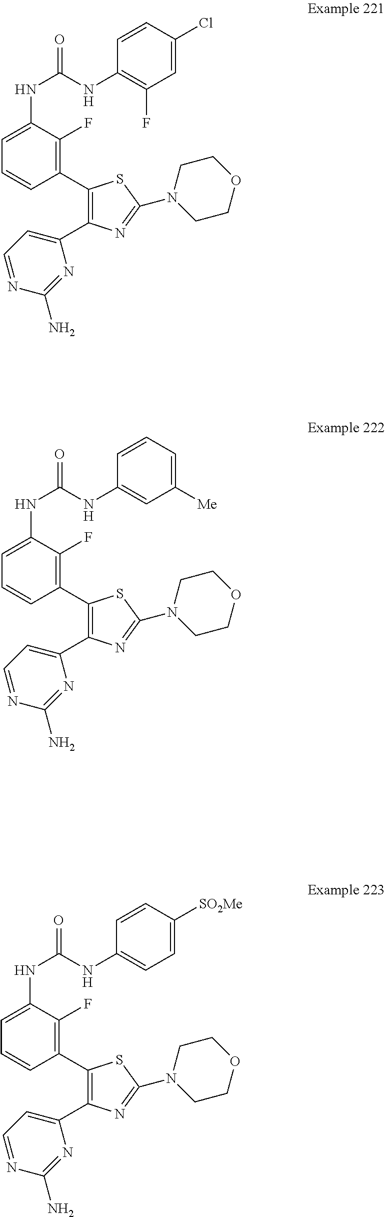 [SMILES: Nc1nccc(-c2nc(N3CCOCC3)sc2-c2cccc(NC(=O)Nc3ccc(Cl)cc3F)c2F)n1 ; Cc1cccc(NC(=O)Nc2cccc(-c3sc(N4CCOCC4)nc3-c3ccnc(N)n3)c2F)c1 ; CS(=O)(=O)c1ccc(NC(=O)Nc2cccc(-c3sc(N4CCOCC4)nc3-c3ccnc(N)n3)c2F)cc1]

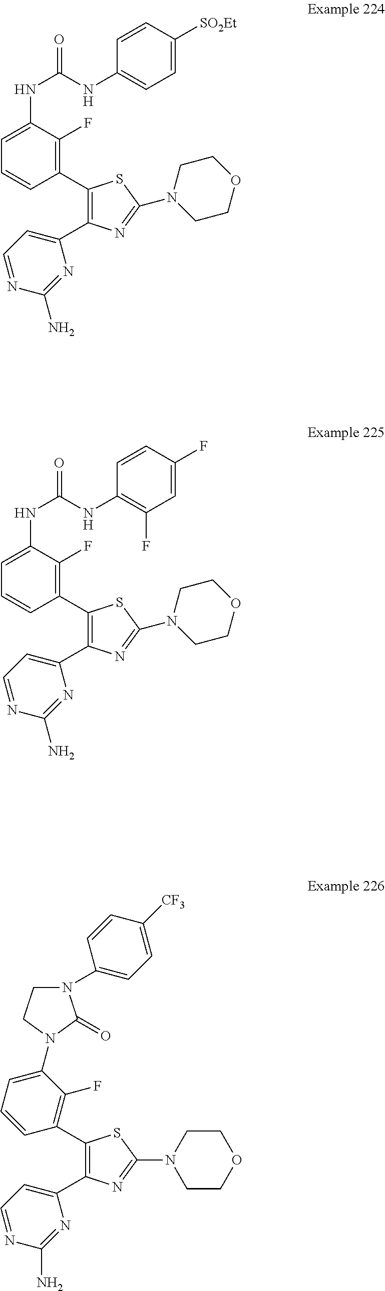

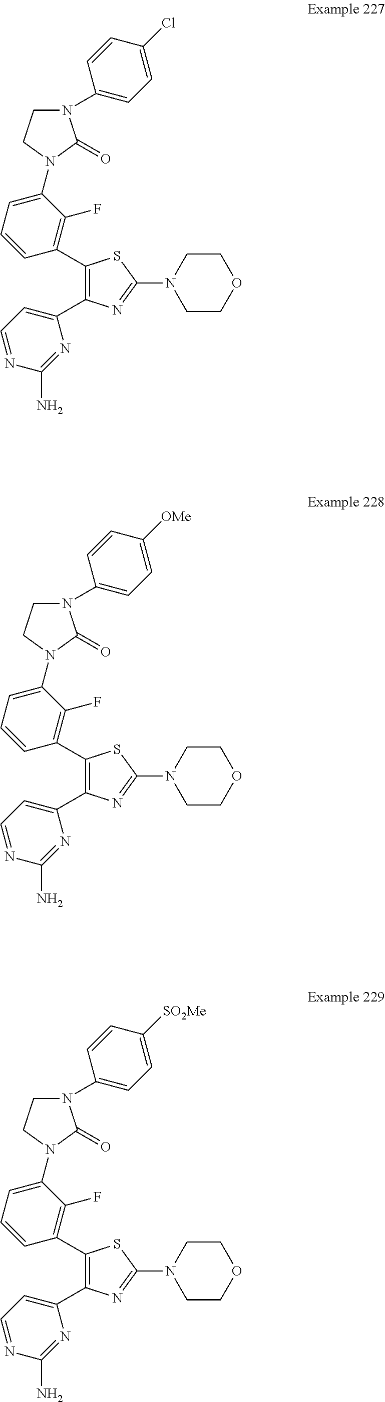 [SMILES: Nc1nccc(-c2nc(N3CCOCC3)sc2-c2cccc(N3CCN(c4ccc(Cl)cc4)C3=O)c2F)n1 ; COc1ccc(N2CCN(c3cccc(-c4sc(N5CCOCC5)nc4-c4ccnc(N)n4)c3F)C2=O)cc1 ; CS(=O)(=O)c1ccc(N2CCN(c3cccc(-c4sc(N5CCOCC5)nc4-c4ccnc(N)n4)c3F)C2=O)cc1]

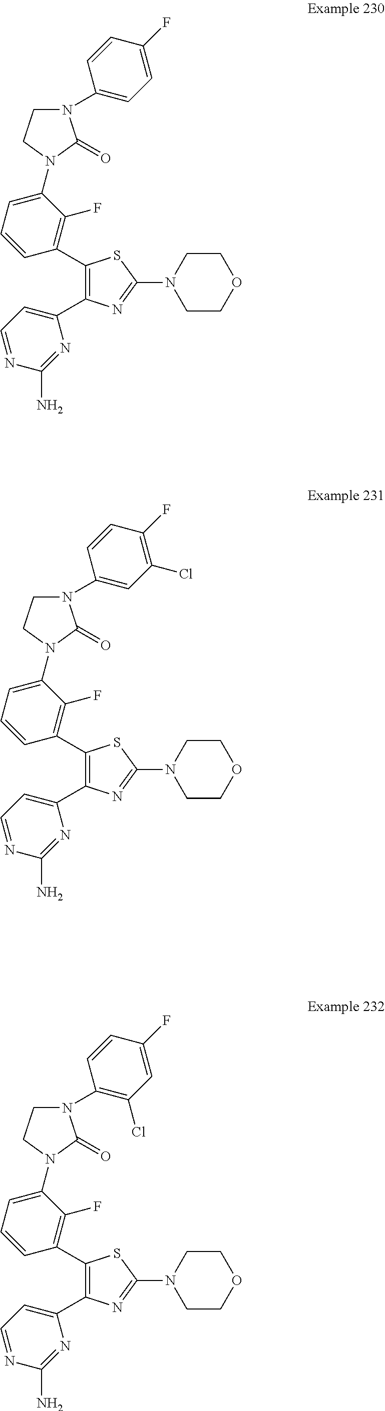 [SMILES: Nc1nccc(-c2nc(N3CCOCC3)sc2-c2cccc(N3CCN(c4ccc(F)cc4)C3=O)c2F)n1 ; Nc1nccc(-c2nc(N3CCOCC3)sc2-c2cccc(N3CCN(c4ccc(F)c(Cl)c4)C3=O)c2F)n1 ; Nc1nccc(-c2nc(N3CCOCC3)sc2-c2cccc(N3CCN(c4ccc(F)cc4Cl)C3=O)c2F)n1]

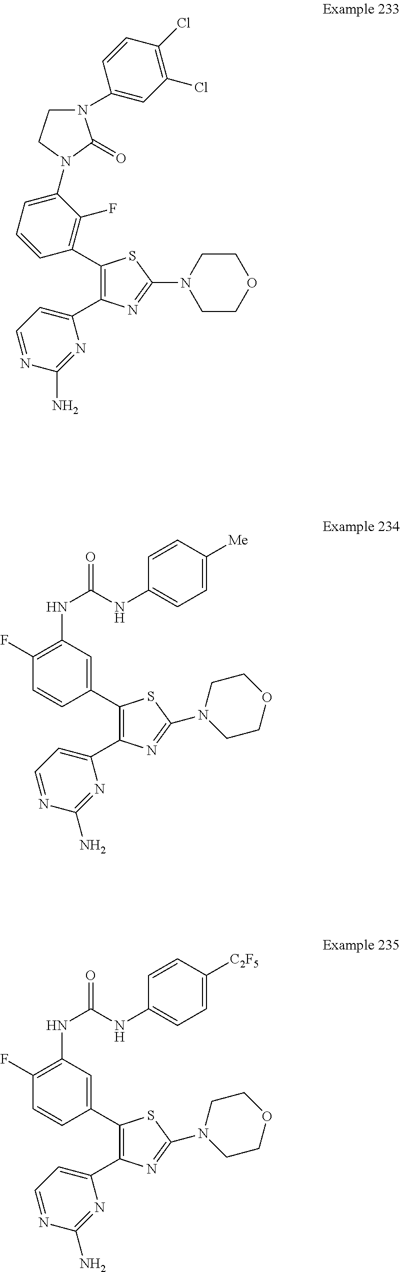

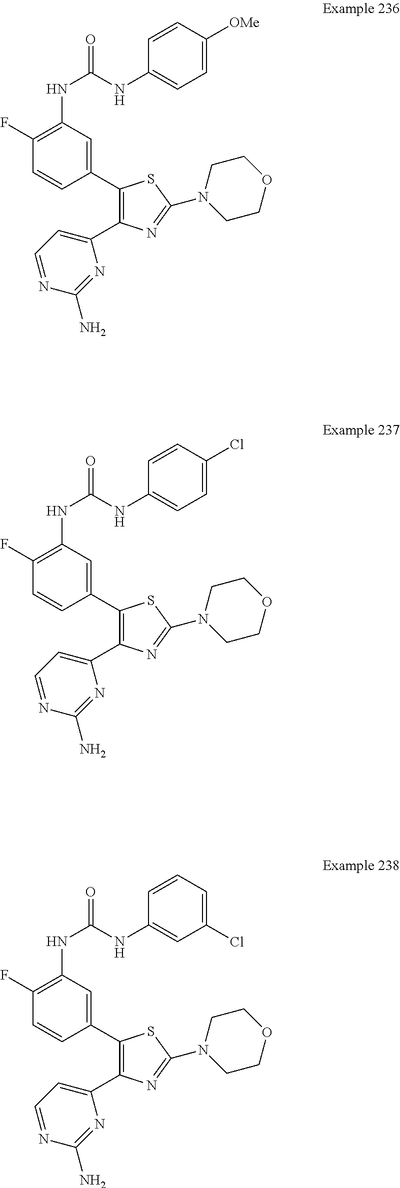 [SMILES: COc1ccc(NC(=O)Nc2cc(-c3sc(N4CCOCC4)nc3-c3ccnc(N)n3)ccc2F)cc1 ; Nc1nccc(-c2nc(N3CCOCC3)sc2-c2ccc(F)c(NC(=O)Nc3ccc(Cl)cc3)c2)n1 ; Nc1nccc(-c2nc(N3CCOCC3)sc2-c2ccc(F)c(NC(=O)Nc3cccc(Cl)c3)c2)n1]

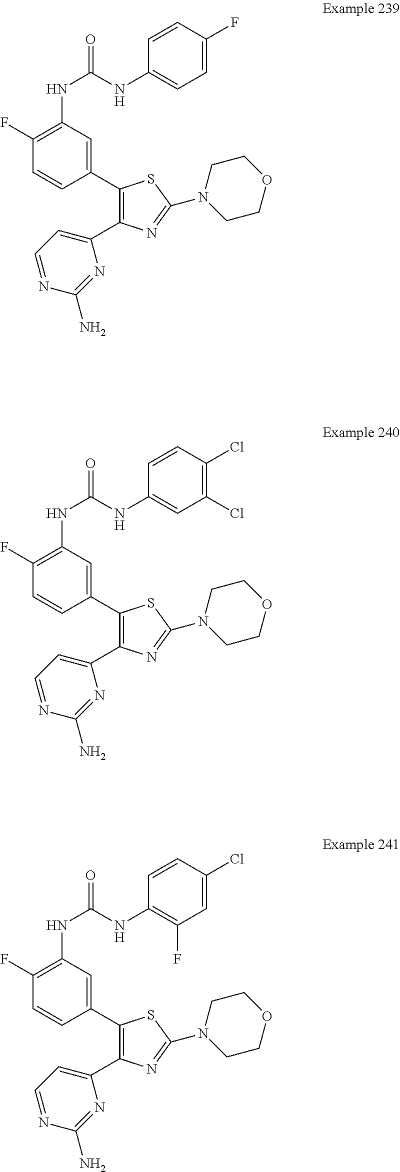 [SMILES: Nc1nccc(-c2nc(N3CCOCC3)sc2-c2ccc(F)c(NC(=O)Nc3ccc(F)cc3)c2)n1 ; Nc1nccc(-c2nc(N3CCOCC3)sc2-c2ccc(F)c(NC(=O)Nc3ccc(Cl)c(Cl)c3)c2)n1 ; Nc1nccc(-c2nc(N3CCOCC3)sc2-c2ccc(F)c(NC(=O)Nc3ccc(Cl)cc3F)c2)n1]

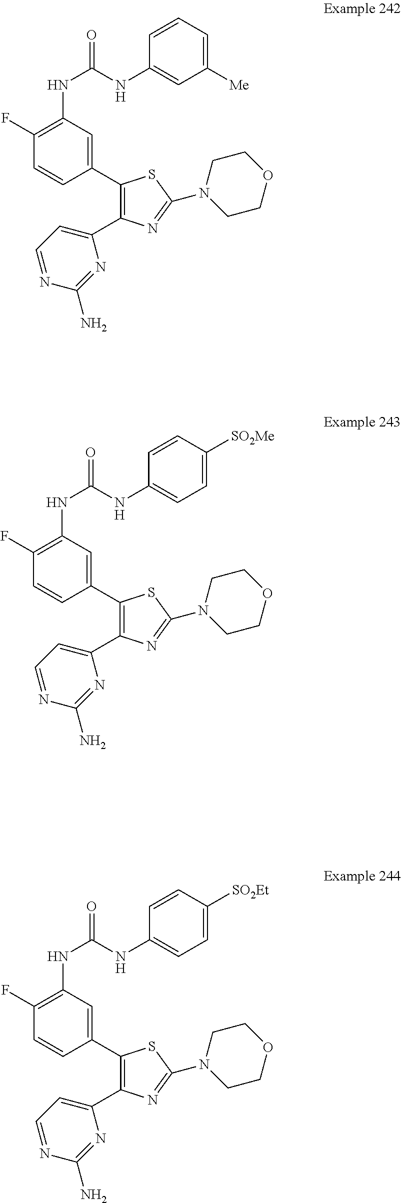

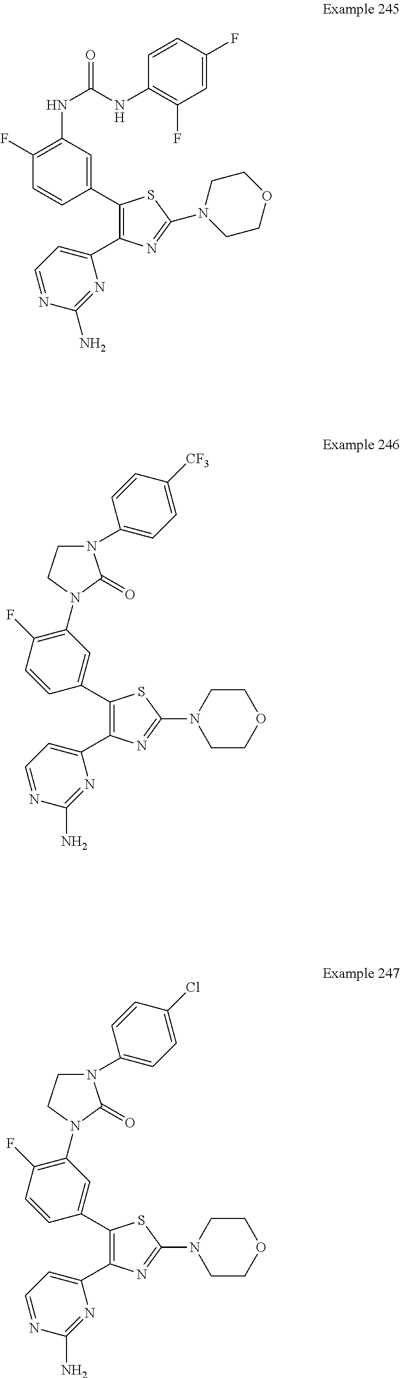 [SMILES: Nc1nccc(-c2nc(N3CCOCC3)sc2-c2ccc(F)c(NC(=O)Nc3ccc(F)cc3F)c2)n1 ; Nc1nccc(-c2nc(N3CCOCC3)sc2-c2ccc(F)c(N3CCN(c4ccc(C(F)(F)F)cc4)C3=O)c2)n1 ; Nc1nccc(-c2nc(N3CCOCC3)sc2-c2ccc(F)c(N3CCN(c4ccc(Cl)cc4)C3=O)c2)n1]

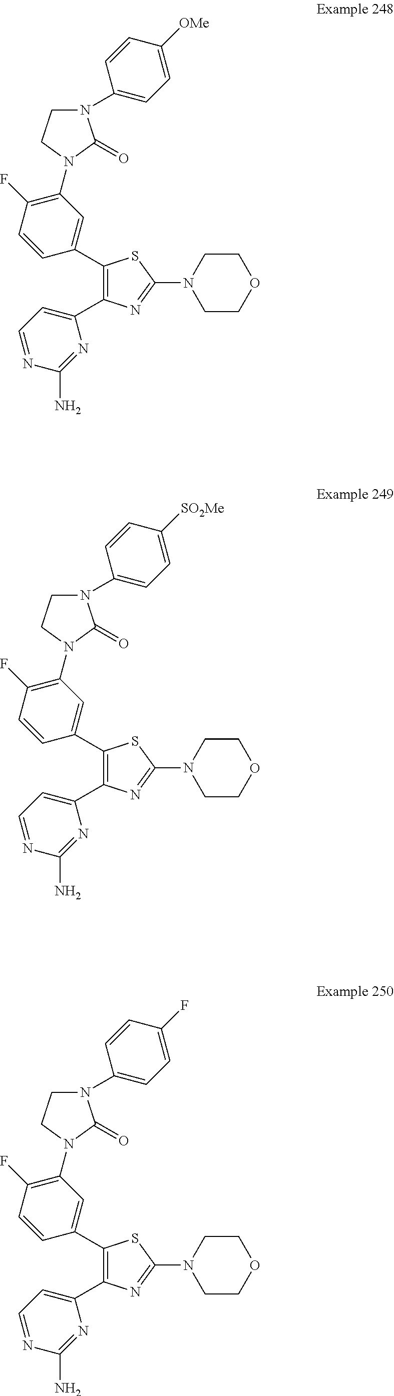 [SMILES: COc1ccc(N2CCN(c3cc(-c4sc(N5CCOCC5)nc4-c4ccnc(N)n4)ccc3F)C2=O)cc1 ; CS(=O)(=O)c1ccc(N2CCN(c3cc(-c4sc(N5CCOCC5)nc4-c4ccnc(N)n4)ccc3F)C2=O)cc1 ; Nc1nccc(-c2nc(N3CCOCC3)sc2-c2ccc(F)c(N3CCN(c4ccc(F)cc4)C3=O)c2)n1]

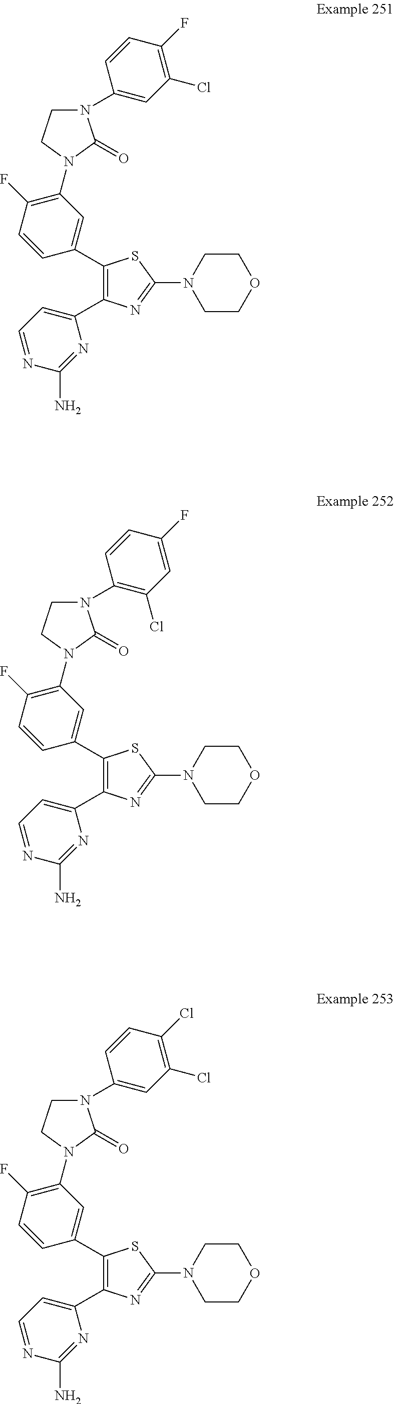 [SMILES: Nc1nccc(-c2nc(N3CCOCC3)sc2-c2ccc(F)c(N3CCN(c4ccc(F)c(Cl)c4)C3=O)c2)n1 ; Nc1nccc(-c2nc(N3CCOCC3)sc2-c2ccc(F)c(N3CCN(c4ccc(F)cc4Cl)C3=O)c2)n1 ; Nc1nccc(-c2nc(N3CCOCC3)sc2-c2ccc(F)c(N3CCN(c4ccc(Cl)c(Cl)c4)C3=O)c2)n1]

The following examples are synthesized according to the following synthetic scheme starting from 4,5-dibromo-2-tert-butyl-1H-imidazole (WO 2011023773) and the corresponding boronic acid using method B or E.

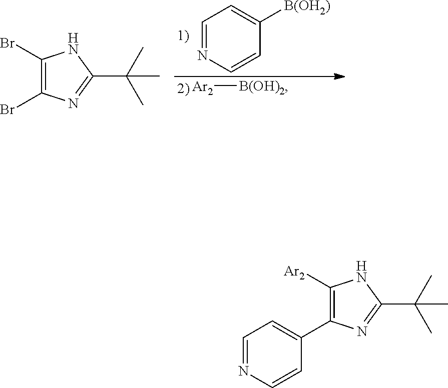

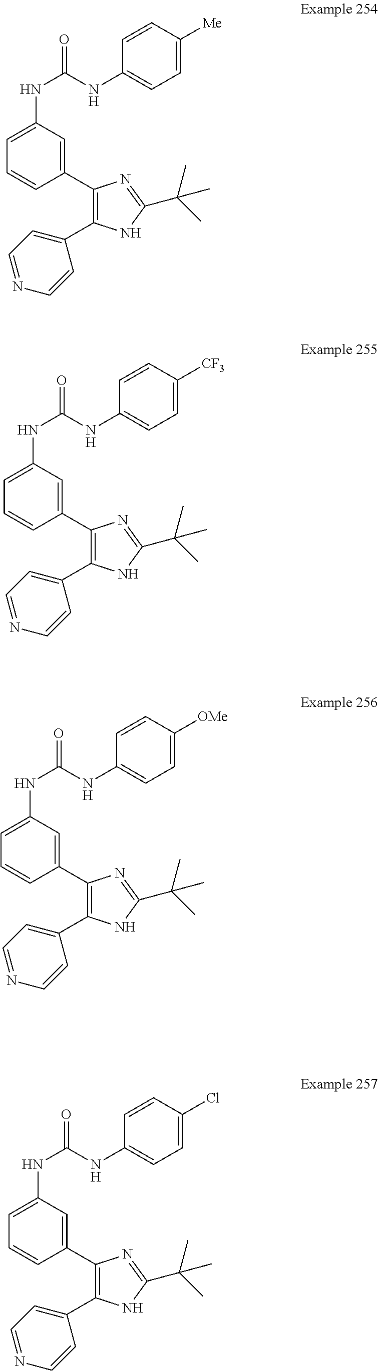 [SMILES: Cc1ccc(NC(=O)Nc2cccc(-c3nc(C(C)(C)C)[nH]c3-c3ccncc3)c2)cc1 ; CC(C)(C)c1nc(-c2cccc(NC(=O)Nc3ccc(C(F)(F)F)cc3)c2)c(-c2ccncc2)[nH]1 ; COc1ccc(NC(=O)Nc2cccc(-c3nc(C(C)(C)C)[nH]c3-c3ccncc3)c2)cc1 ; CC(C)(C)c1nc(-c2cccc(NC(=O)Nc3ccc(Cl)cc3)c2)c(-c2ccncc2)[nH]1]

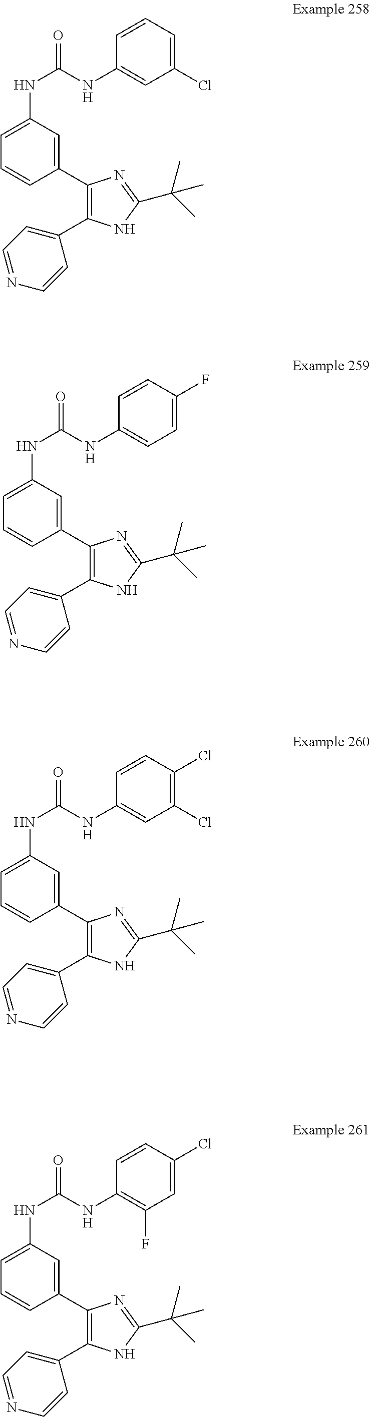 [SMILES: CC(C)(C)c1nc(-c2cccc(NC(=O)Nc3cccc(Cl)c3)c2)c(-c2ccncc2)[nH]1 ; CC(C)(C)c1nc(-c2cccc(NC(=O)Nc3ccc(F)cc3)c2)c(-c2ccncc2)[nH]1 ; CC(C)(C)c1nc(-c2cccc(NC(=O)Nc3ccc(Cl)c(Cl)c3)c2)c(-c2ccncc2)[nH]1 ; CC(C)(C)c1nc(-c2cccc(NC(=O)Nc3ccc(Cl)cc3F)c2)c(-c2ccncc2)[nH]1]

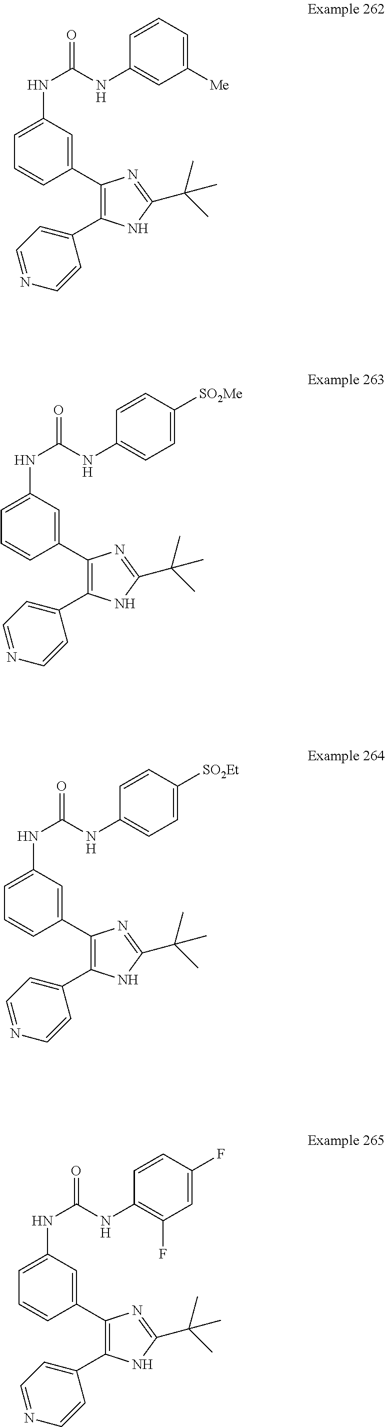

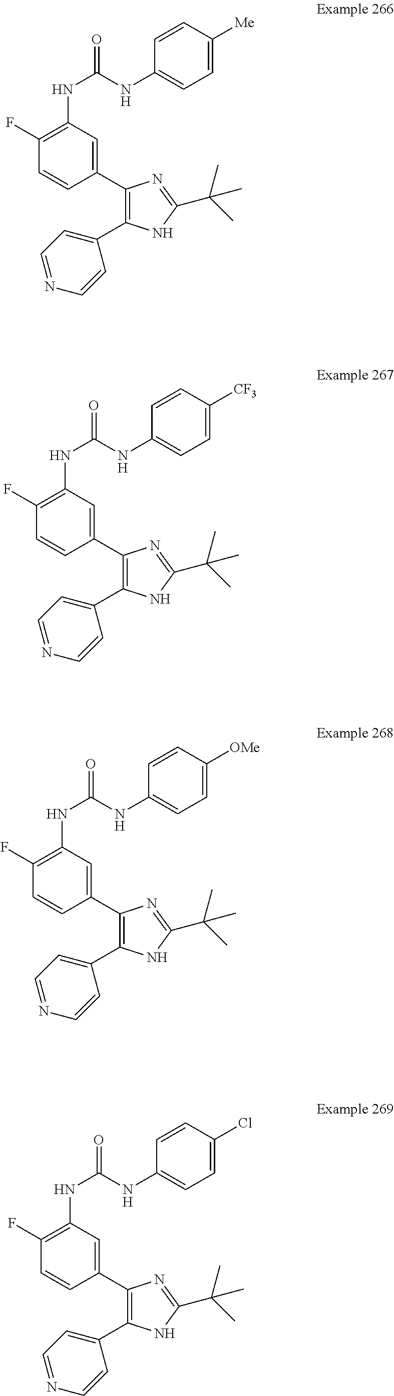 [SMILES: Cc1ccc(NC(=O)Nc2cc(-c3nc(C(C)(C)C)[nH]c3-c3ccncc3)ccc2F)cc1 ; CC(C)(C)c1nc(-c2ccc(F)c(NC(=O)Nc3ccc(C(F)(F)F)cc3)c2)c(-c2ccncc2)[nH]1 ; COc1ccc(NC(=O)Nc2cc(-c3nc(C(C)(C)C)[nH]c3-c3ccncc3)ccc2F)cc1 ; CC(C)(C)c1nc(-c2ccc(F)c(NC(=O)Nc3ccc(Cl)cc3)c2)c(-c2ccncc2)[nH]1]

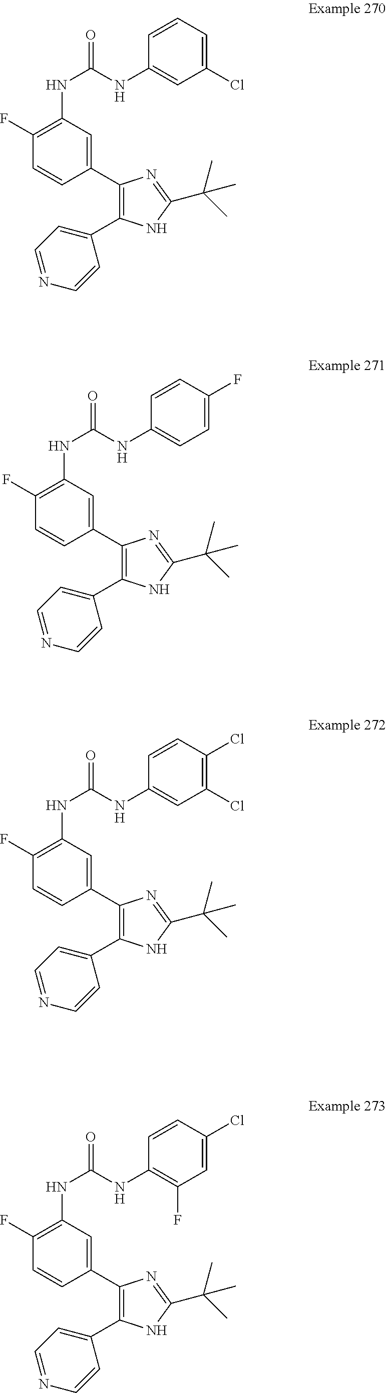 [SMILES: CC(C)(C)c1nc(-c2ccc(F)c(NC(=O)Nc3cccc(Cl)c3)c2)c(-c2ccncc2)[nH]1 ; CC(C)(C)c1nc(-c2ccc(F)c(NC(=O)Nc3ccc(F)cc3)c2)c(-c2ccncc2)[nH]1 ; CC(C)(C)c1nc(-c2ccc(F)c(NC(=O)Nc3ccc(Cl)c(Cl)c3)c2)c(-c2ccncc2)[nH]1 ; CC(C)(C)c1nc(-c2ccc(F)c(NC(=O)Nc3ccc(Cl)cc3F)c2)c(-c2ccncc2)[nH]1]

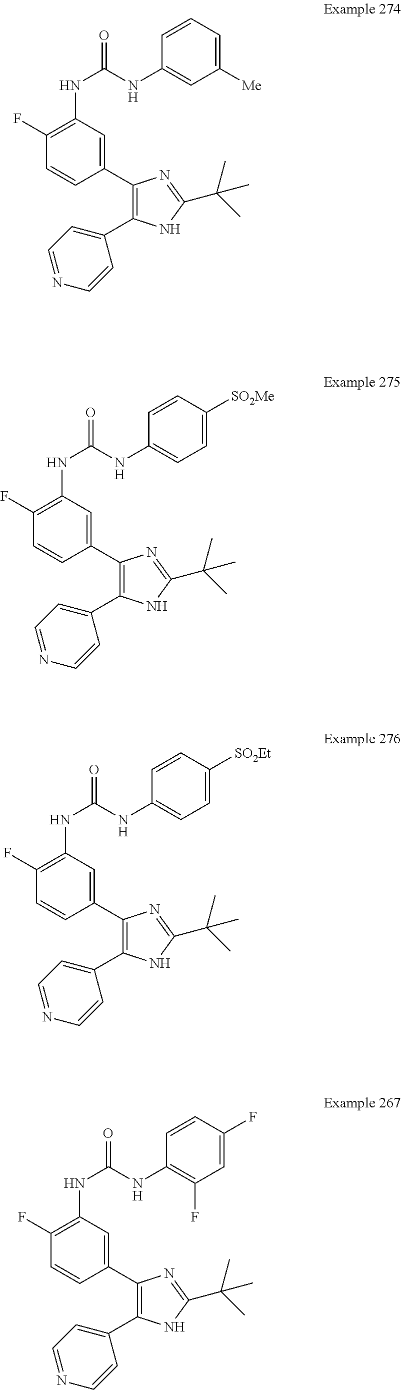

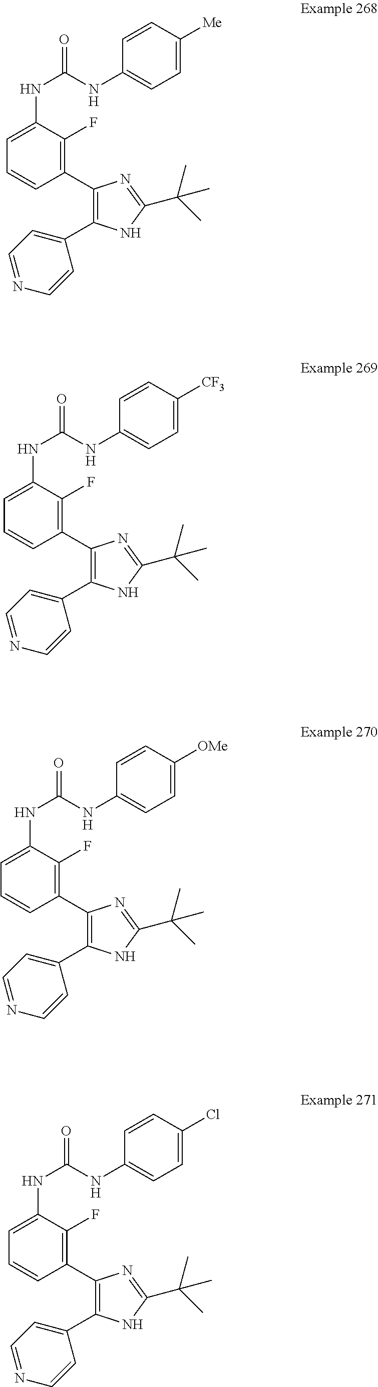 [SMILES: Cc1ccc(NC(=O)Nc2cccc(-c3nc(C(C)(C)C)[nH]c3-c3ccncc3)c2F)cc1 ; CC(C)(C)c1nc(-c2cccc(NC(=O)Nc3ccc(C(F)(F)F)cc3)c2F)c(-c2ccncc2)[nH]1 ; COc1ccc(NC(=O)Nc2cccc(-c3nc(C(C)(C)C)[nH]c3-c3ccncc3)c2F)cc1 ; CC(C)(C)c1nc(-c2cccc(NC(=O)Nc3ccc(Cl)cc3)c2F)c(-c2ccncc2)[nH]1]

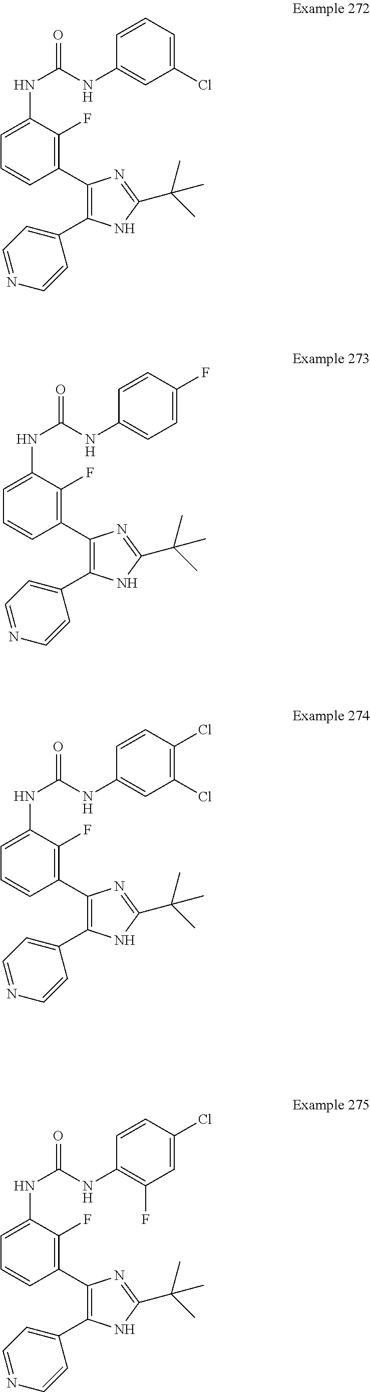 [SMILES: CC(C)(C)c1nc(-c2cccc(NC(=O)Nc3cccc(Cl)c3)c2F)c(-c2ccncc2)[nH]1 ; CC(C)(C)c1nc(-c2cccc(NC(=O)Nc3ccc(F)cc3)c2F)c(-c2ccncc2)[nH]1 ; CC(C)(C)c1nc(-c2cccc(NC(=O)Nc3ccc(Cl)c(Cl)c3)c2F)c(-c2ccncc2)[nH]1 ; CC(C)(C)c1nc(-c2cccc(NC(=O)Nc3ccc(Cl)cc3F)c2F)c(-c2ccncc2)[nH]1]

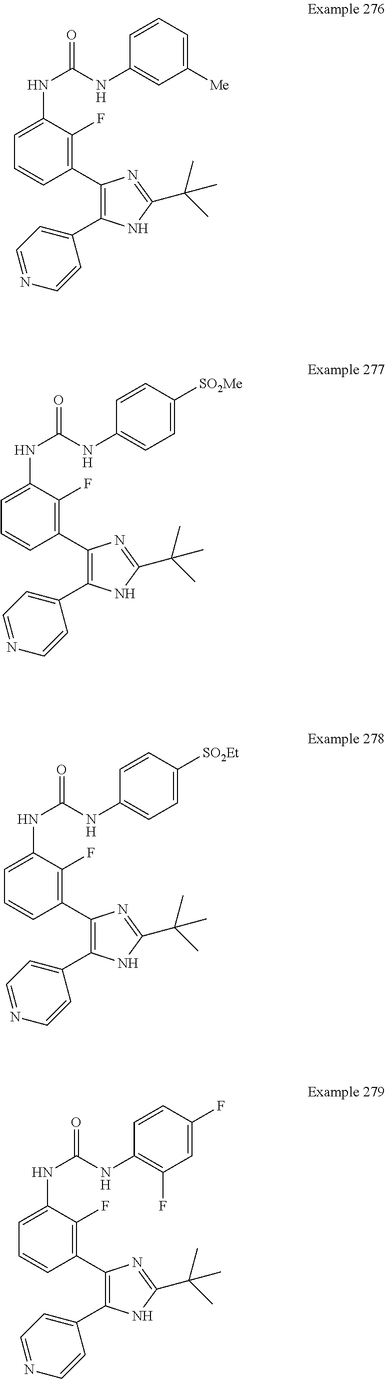

The following examples are synthesized according to the following synthetic scheme.

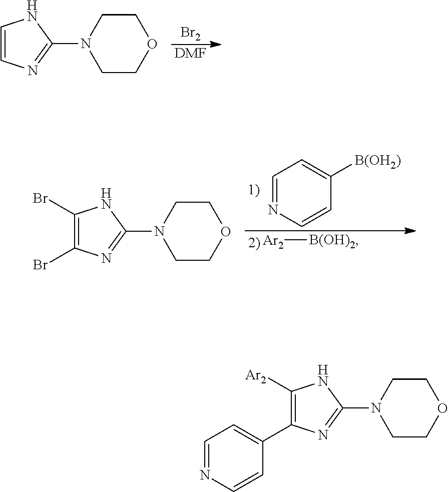

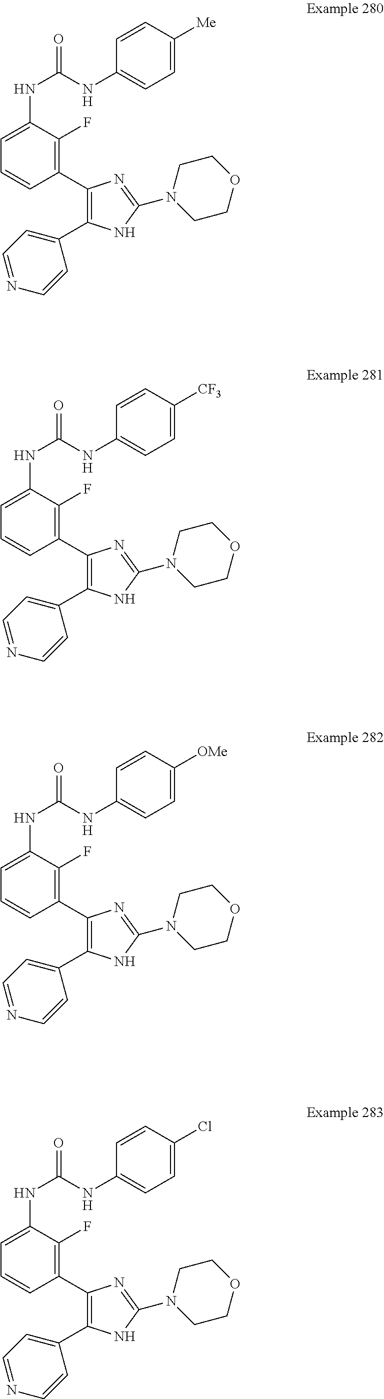 [SMILES: Cc1ccc(NC(=O)Nc2cccc(-c3nc(N4CCOCC4)[nH]c3-c3ccncc3)c2F)cc1 ; O=C(Nc1ccc(C(F)(F)F)cc1)Nc1cccc(-c2nc(N3CCOCC3)[nH]c2-c2ccncc2)c1F ; COc1ccc(NC(=O)Nc2cccc(-c3nc(N4CCOCC4)[nH]c3-c3ccncc3)c2F)cc1 ; O=C(Nc1ccc(Cl)cc1)Nc1cccc(-c2nc(N3CCOCC3)[nH]c2-c2ccncc2)c1F]

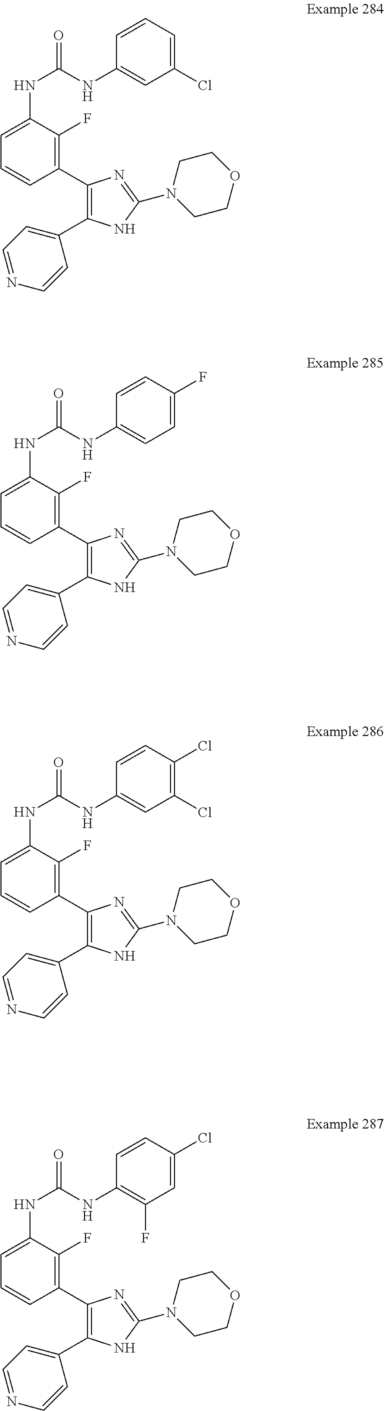 [SMILES: O=C(Nc1cccc(Cl)c1)Nc1cccc(-c2nc(N3CCOCC3)[nH]c2-c2ccncc2)c1F ; O=C(Nc1ccc(F)cc1)Nc1cccc(-c2nc(N3CCOCC3)[nH]c2-c2ccncc2)c1F ; O=C(Nc1ccc(Cl)c(Cl)c1)Nc1cccc(-c2nc(N3CCOCC3)[nH]c2-c2ccncc2)c1F ; O=C(Nc1ccc(Cl)cc1F)Nc1cccc(-c2nc(N3CCOCC3)[nH]c2-c2ccncc2)c1F]

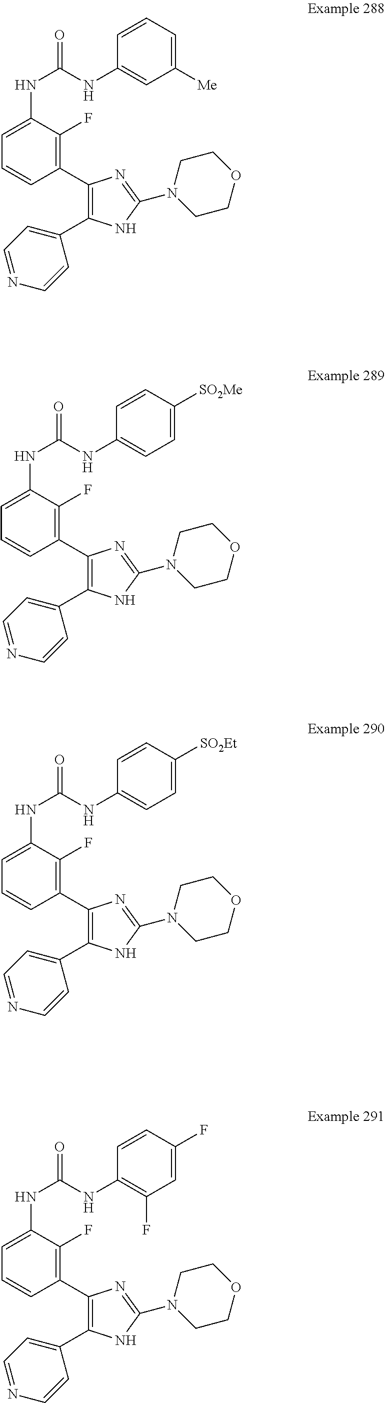

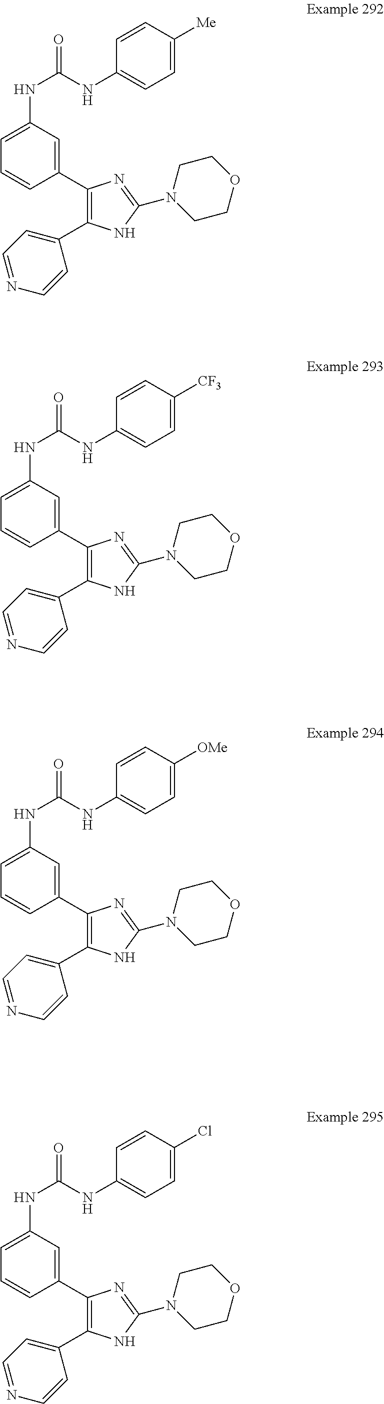 [SMILES: Cc1ccc(NC(=O)Nc2cccc(-c3nc(N4CCOCC4)[nH]c3-c3ccncc3)c2)cc1 ; O=C(Nc1ccc(C(F)(F)F)cc1)Nc1cccc(-c2nc(N3CCOCC3)[nH]c2-c2ccncc2)c1 ; COc1ccc(NC(=O)Nc2cccc(-c3nc(N4CCOCC4)[nH]c3-c3ccncc3)c2)cc1 ; O=C(Nc1ccc(Cl)cc1)Nc1cccc(-c2nc(N3CCOCC3)[nH]c2-c2ccncc2)c1]

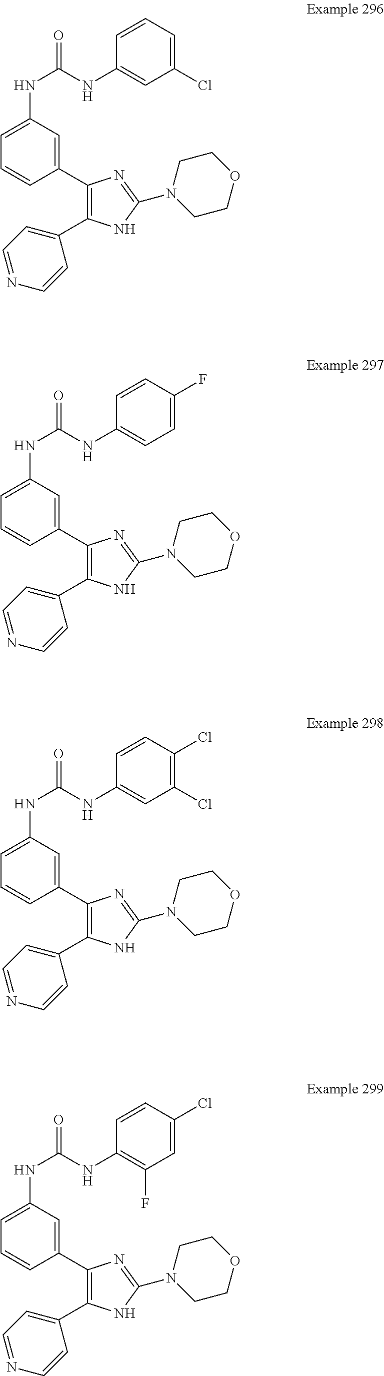 [SMILES: O=C(Nc1cccc(Cl)c1)Nc1cccc(-c2nc(N3CCOCC3)[nH]c2-c2ccncc2)c1 ; O=C(Nc1ccc(F)cc1)Nc1cccc(-c2nc(N3CCOCC3)[nH]c2-c2ccncc2)c1 ; O=C(Nc1cccc(-c2nc(N3CCOCC3)[nH]c2-c2ccncc2)c1)Nc1ccc(Cl)c(Cl)c1 ; O=C(Nc1cccc(-c2nc(N3CCOCC3)[nH]c2-c2ccncc2)c1)Nc1ccc(Cl)cc1F]

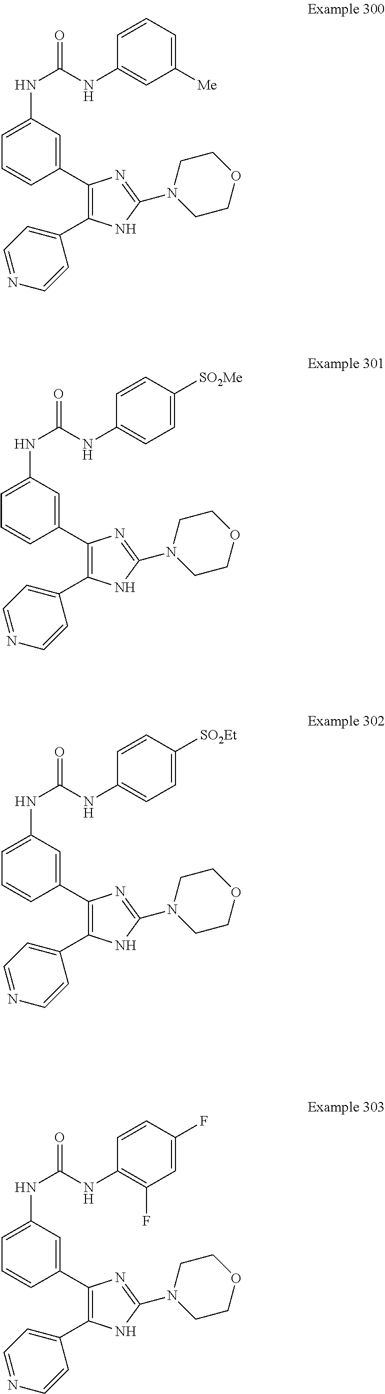

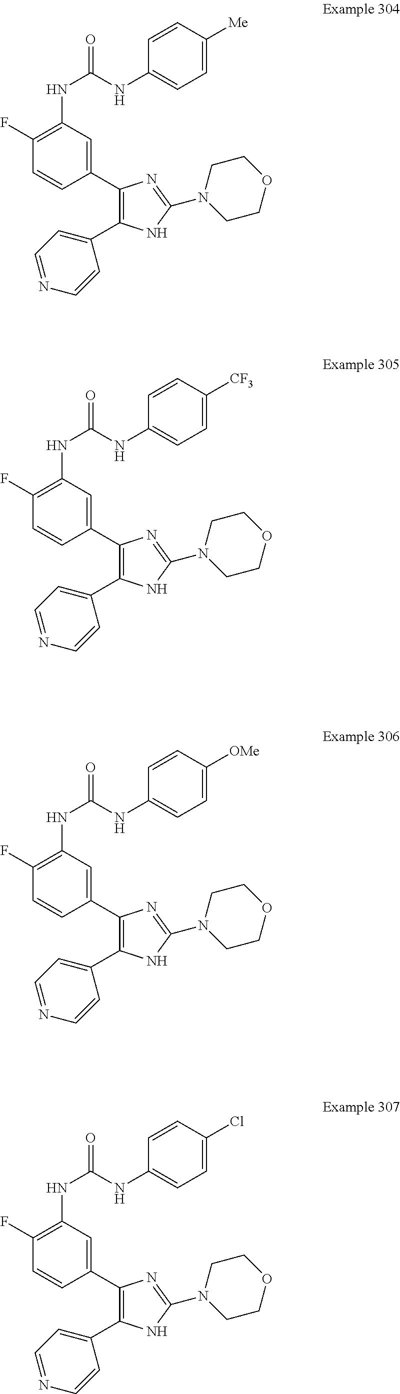 [SMILES: Cc1ccc(NC(=O)Nc2cc(-c3nc(N4CCOCC4)[nH]c3-c3ccncc3)ccc2F)cc1 ; O=C(Nc1ccc(C(F)(F)F)cc1)Nc1cc(-c2nc(N3CCOCC3)[nH]c2-c2ccncc2)ccc1F ; COc1ccc(NC(=O)Nc2cc(-c3nc(N4CCOCC4)[nH]c3-c3ccncc3)ccc2F)cc1 ; O=C(Nc1ccc(Cl)cc1)Nc1cc(-c2nc(N3CCOCC3)[nH]c2-c2ccncc2)ccc1F]

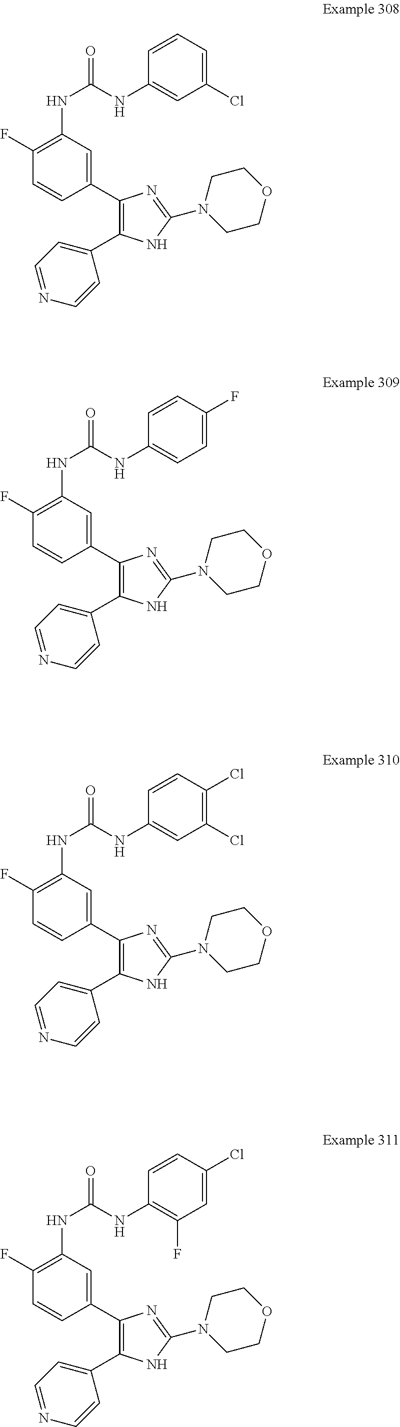 [SMILES: O=C(Nc1cccc(Cl)c1)Nc1cc(-c2nc(N3CCOCC3)[nH]c2-c2ccncc2)ccc1F ; O=C(Nc1ccc(F)cc1)Nc1cc(-c2nc(N3CCOCC3)[nH]c2-c2ccncc2)ccc1F ; O=C(Nc1ccc(Cl)c(Cl)c1)Nc1cc(-c2nc(N3CCOCC3)[nH]c2-c2ccncc2)ccc1F ; O=C(Nc1ccc(Cl)cc1F)Nc1cc(-c2nc(N3CCOCC3)[nH]c2-c2ccncc2)ccc1F]

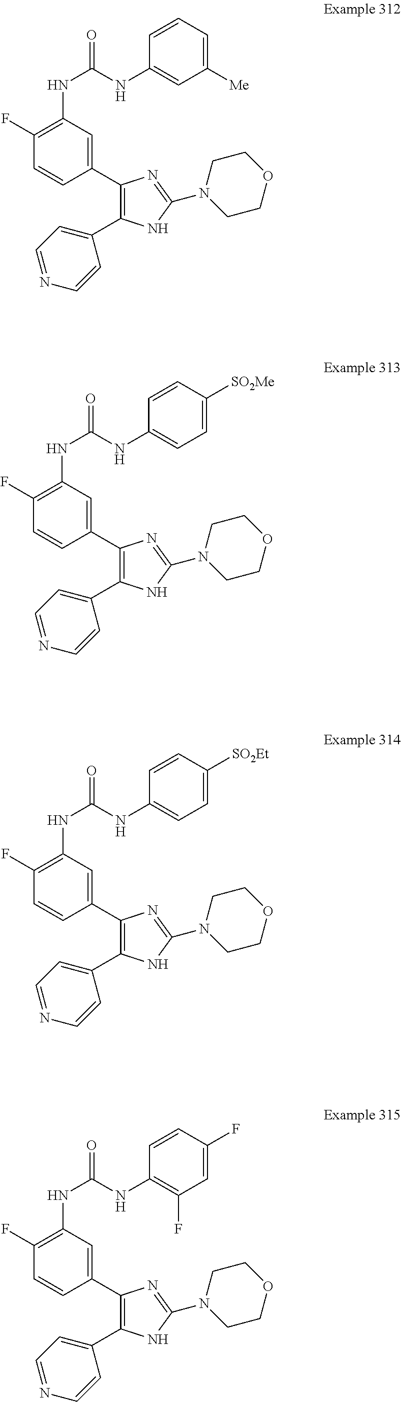

The following examples are synthesized according to the following synthetic scheme.

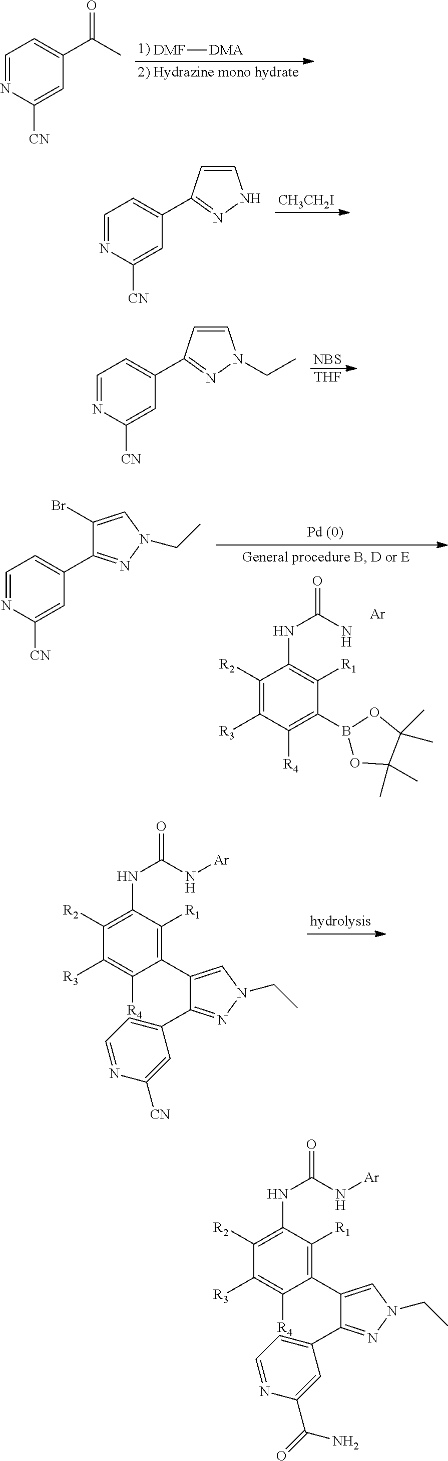

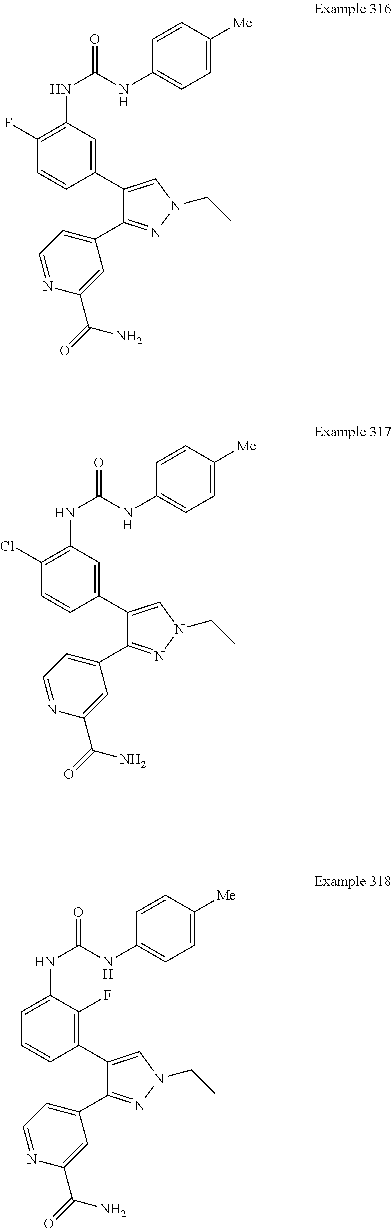 [SMILES: CCn1cc(-c2ccc(F)c(NC(=O)Nc3ccc(C)cc3)c2)c(-c2ccnc(C(N)=O)c2)n1 ; CCn1cc(-c2ccc(Cl)c(NC(=O)Nc3ccc(C)cc3)c2)c(-c2ccnc(C(N)=O)c2)n1 ; CCn1cc(-c2cccc(NC(=O)Nc3ccc(C)cc3)c2F)c(-c2ccnc(C(N)=O)c2)n1]

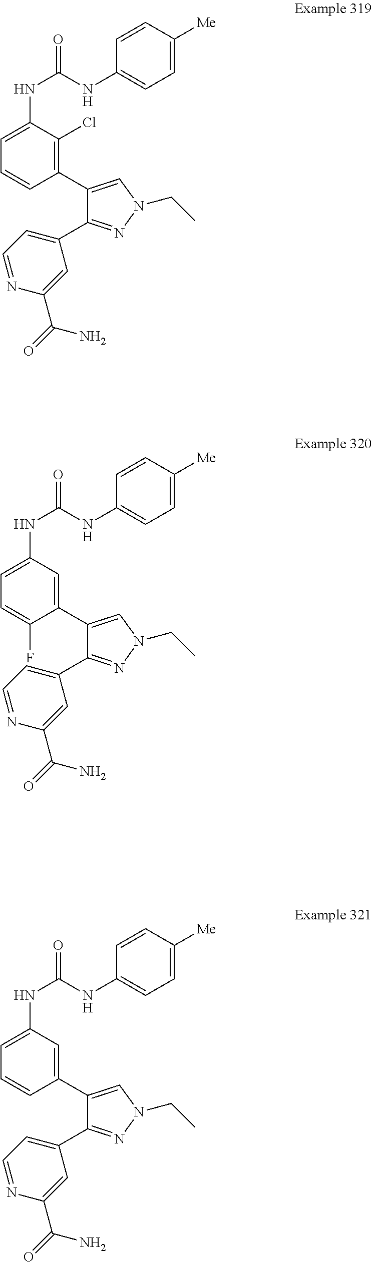 [SMILES: CCn1cc(-c2cccc(NC(=O)Nc3ccc(C)cc3)c2Cl)c(-c2ccnc(C(N)=O)c2)n1 ; CCn1cc(-c2cc(NC(=O)Nc3ccc(C)cc3)ccc2F)c(-c2ccnc(C(N)=O)c2)n1 ; CCn1cc(-c2cccc(NC(=O)Nc3ccc(C)cc3)c2)c(-c2ccnc(C(N)=O)c2)n1]

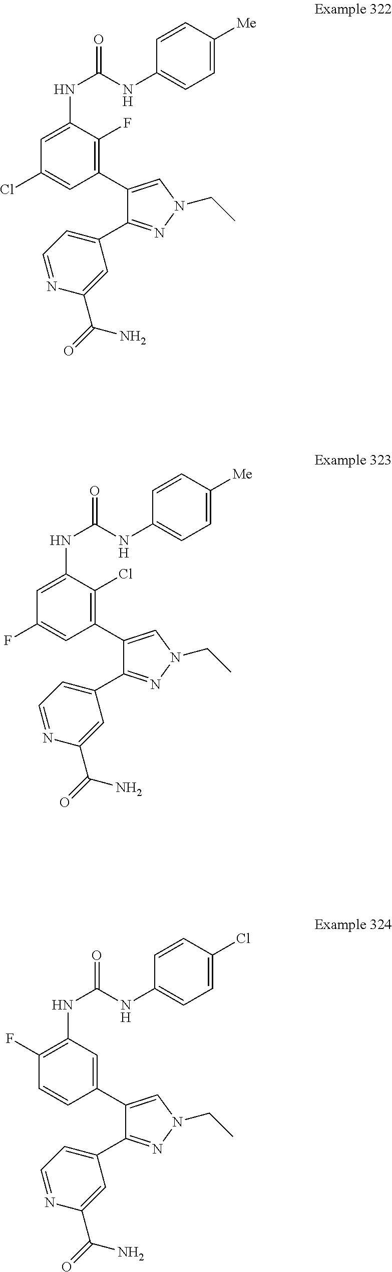 [SMILES: CCn1cc(-c2cc(Cl)cc(NC(=O)Nc3ccc(C)cc3)c2F)c(-c2ccnc(C(N)=O)c2)n1 ; CCn1cc(-c2cc(F)cc(NC(=O)Nc3ccc(C)cc3)c2Cl)c(-c2ccnc(C(N)=O)c2)n1 ; CCn1cc(-c2ccc(F)c(NC(=O)Nc3ccc(Cl)cc3)c2)c(-c2ccnc(C(N)=O)c2)n1]

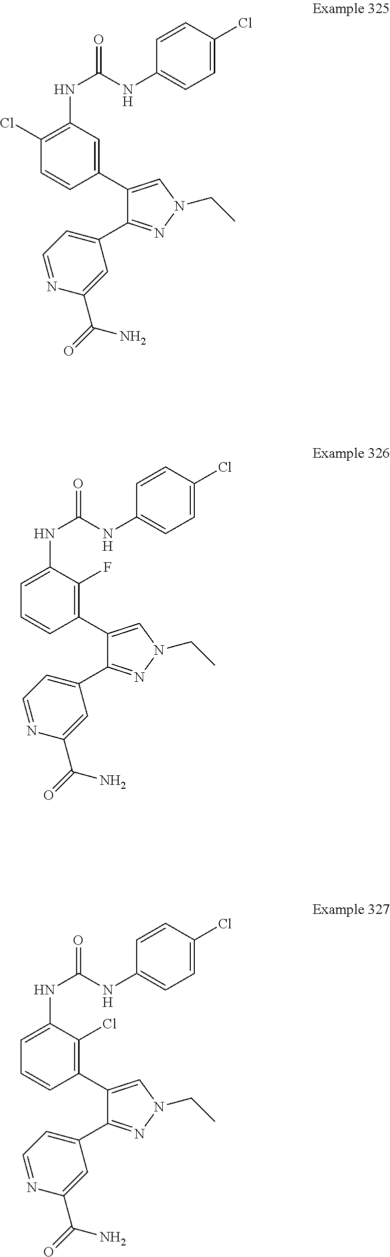 [SMILES: CCn1cc(-c2ccc(Cl)c(NC(=O)Nc3ccc(Cl)cc3)c2)c(-c2ccnc(C(N)=O)c2)n1 ; CCn1cc(-c2cccc(NC(=O)Nc3ccc(Cl)cc3)c2F)c(-c2ccnc(C(N)=O)c2)n1 ; CCn1cc(-c2cccc(NC(=O)Nc3ccc(Cl)cc3)c2Cl)c(-c2ccnc(C(N)=O)c2)n1]

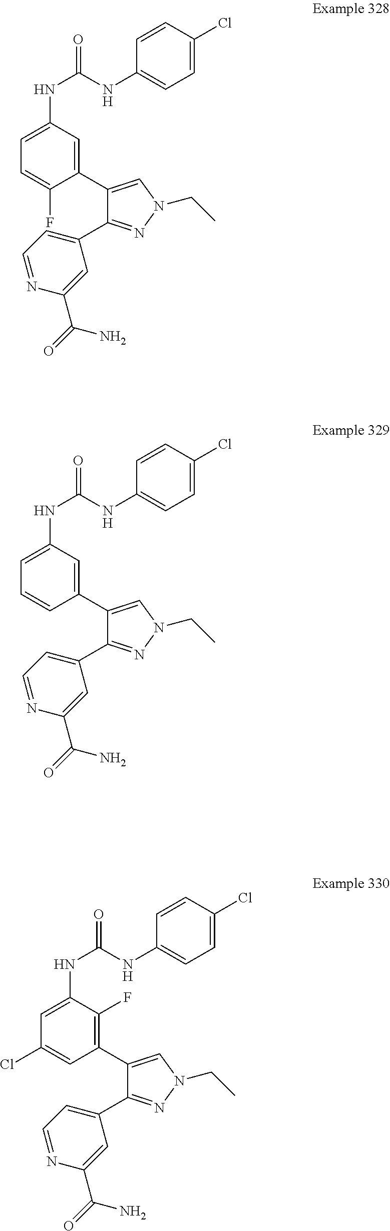 [SMILES: CCn1cc(-c2cc(NC(=O)Nc3ccc(Cl)cc3)ccc2F)c(-c2ccnc(C(N)=O)c2)n1 ; CCn1cc(-c2cccc(NC(=O)Nc3ccc(Cl)cc3)c2)c(-c2ccnc(C(N)=O)c2)n1 ; CCn1cc(-c2cc(Cl)cc(NC(=O)Nc3ccc(Cl)cc3)c2F)c(-c2ccnc(C(N)=O)c2)n1]

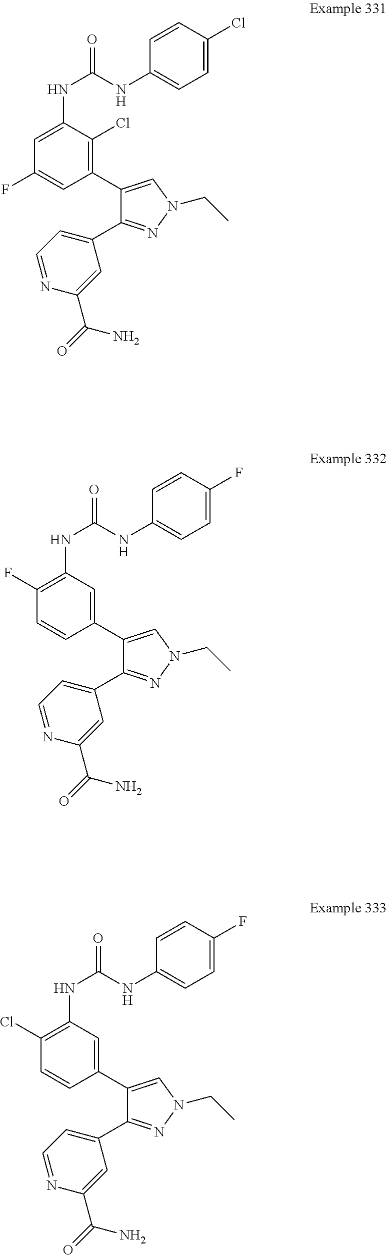 [SMILES: CCn1cc(-c2cc(F)cc(NC(=O)Nc3ccc(Cl)cc3)c2Cl)c(-c2ccnc(C(N)=O)c2)n1 ; CCn1cc(-c2ccc(F)c(NC(=O)Nc3ccc(F)cc3)c2)c(-c2ccnc(C(N)=O)c2)n1 ; CCn1cc(-c2ccc(Cl)c(NC(=O)Nc3ccc(F)cc3)c2)c(-c2ccnc(C(N)=O)c2)n1]

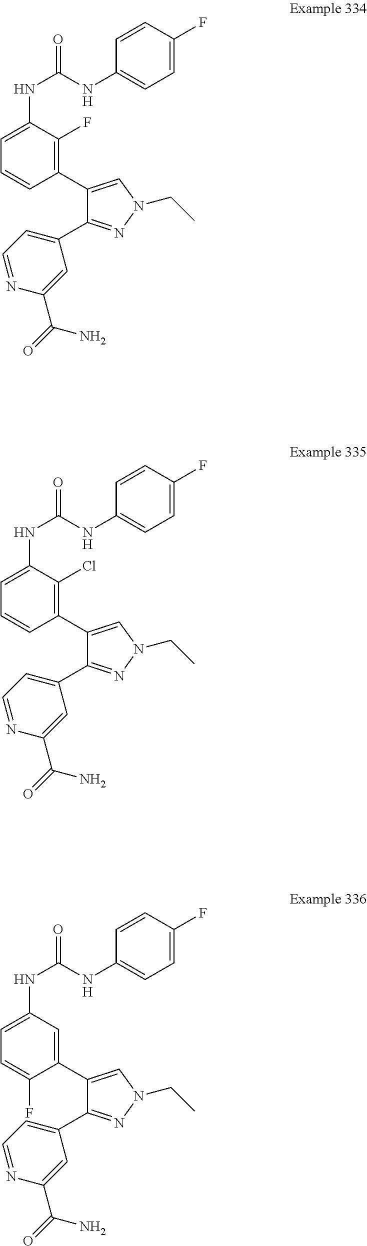 [SMILES: CCn1cc(-c2cccc(NC(=O)Nc3ccc(F)cc3)c2F)c(-c2ccnc(C(N)=O)c2)n1 ; CCn1cc(-c2cccc(NC(=O)Nc3ccc(F)cc3)c2Cl)c(-c2ccnc(C(N)=O)c2)n1 ; CCn1cc(-c2cc(NC(=O)Nc3ccc(F)cc3)ccc2F)c(-c2ccnc(C(N)=O)c2)n1]

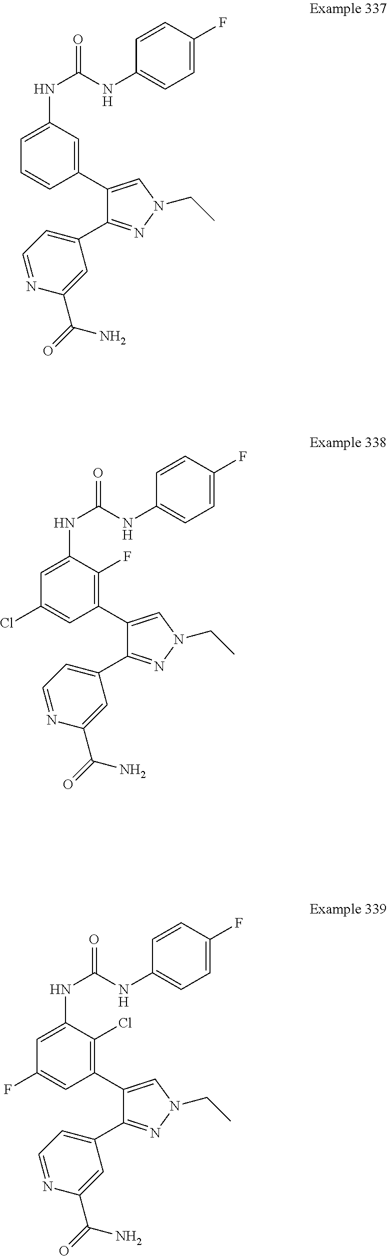 [SMILES: CCn1cc(-c2cccc(NC(=O)Nc3ccc(F)cc3)c2)c(-c2ccnc(C(N)=O)c2)n1 ; CCn1cc(-c2cc(Cl)cc(NC(=O)Nc3ccc(F)cc3)c2F)c(-c2ccnc(C(N)=O)c2)n1 ; CCn1cc(-c2cc(F)cc(NC(=O)Nc3ccc(F)cc3)c2Cl)c(-c2ccnc(C(N)=O)c2)n1]

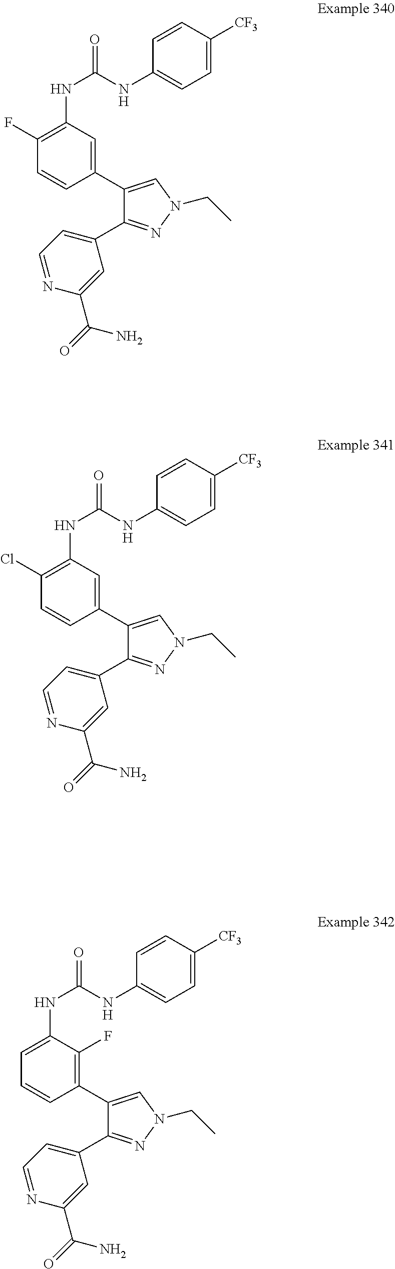 [SMILES: CCn1cc(-c2ccc(F)c(NC(=O)Nc3ccc(C(F)(F)F)cc3)c2)c(-c2ccnc(C(N)=O)c2)n1 ; CCn1cc(-c2ccc(Cl)c(NC(=O)Nc3ccc(C(F)(F)F)cc3)c2)c(-c2ccnc(C(N)=O)c2)n1 ; CCn1cc(-c2cccc(NC(=O)Nc3ccc(C(F)(F)F)cc3)c2F)c(-c2ccnc(C(N)=O)c2)n1]

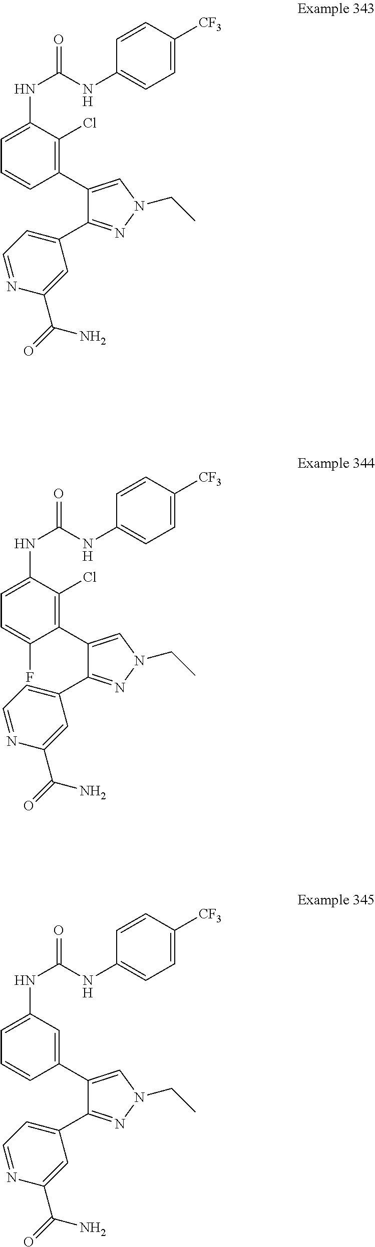 [SMILES: CCn1cc(-c2cccc(NC(=O)Nc3ccc(C(F)(F)F)cc3)c2Cl)c(-c2ccnc(C(N)=O)c2)n1 ; CCn1cc(-c2c(F)ccc(NC(=O)Nc3ccc(C(F)(F)F)cc3)c2Cl)c(-c2ccnc(C(N)=O)c2)n1 ; CCn1cc(-c2cccc(NC(=O)Nc3ccc(C(F)(F)F)cc3)c2)c(-c2ccnc(C(N)=O)c2)n1]

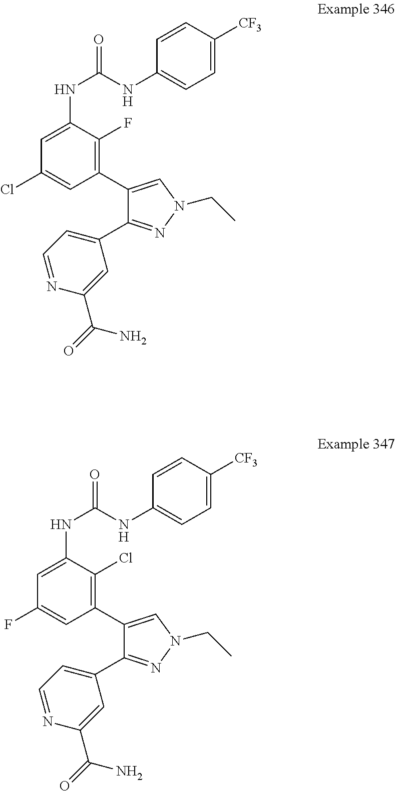 [SMILES: CCn1cc(-c2cc(Cl)cc(NC(=O)Nc3ccc(C(F)(F)F)cc3)c2F)c(-c2ccnc(C(N)=O)c2)n1 ; CCn1cc(-c2cc(F)cc(NC(=O)Nc3ccc(C(F)(F)F)cc3)c2Cl)c(-c2ccnc(C(N)=O)c2)n1]

The following examples are synthesized according to the following synthetic scheme.

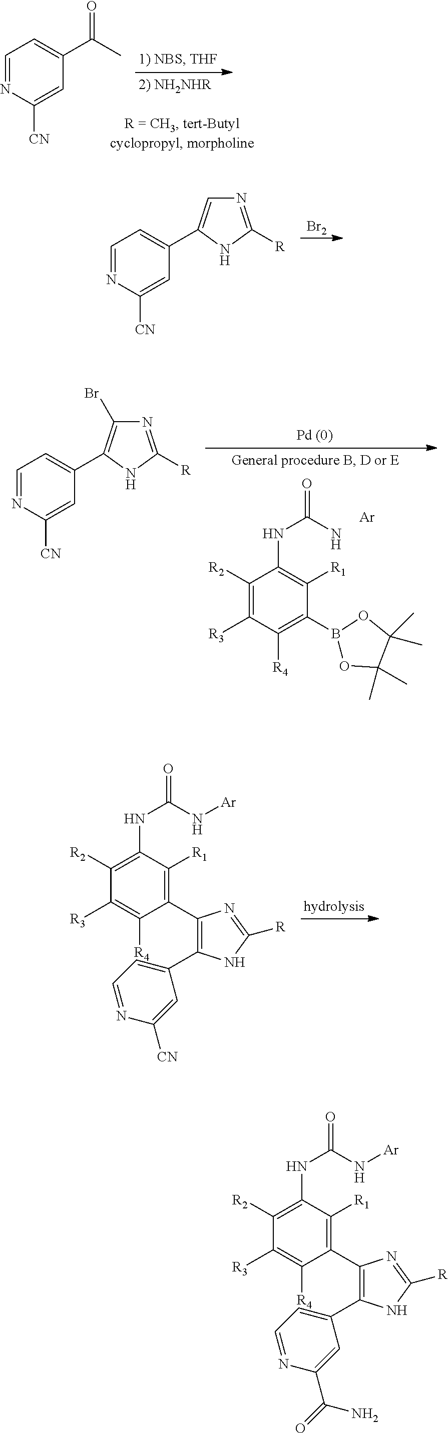

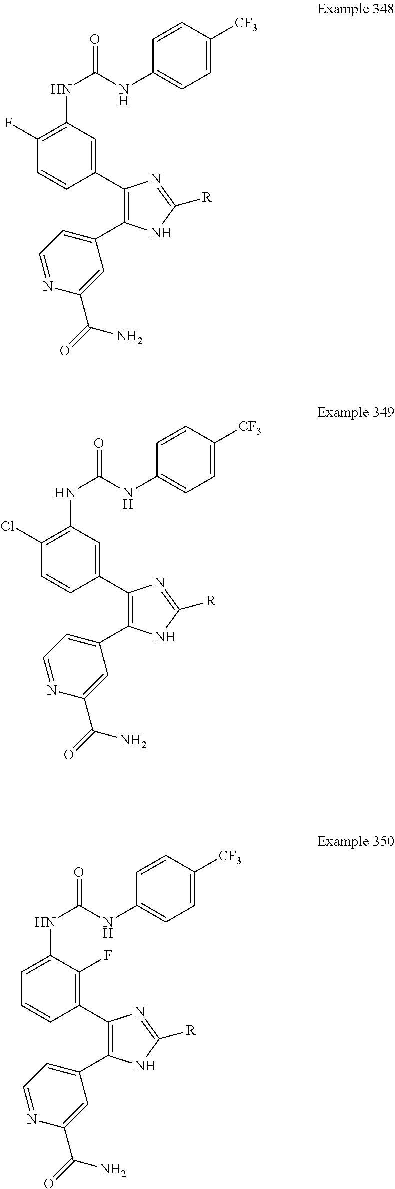

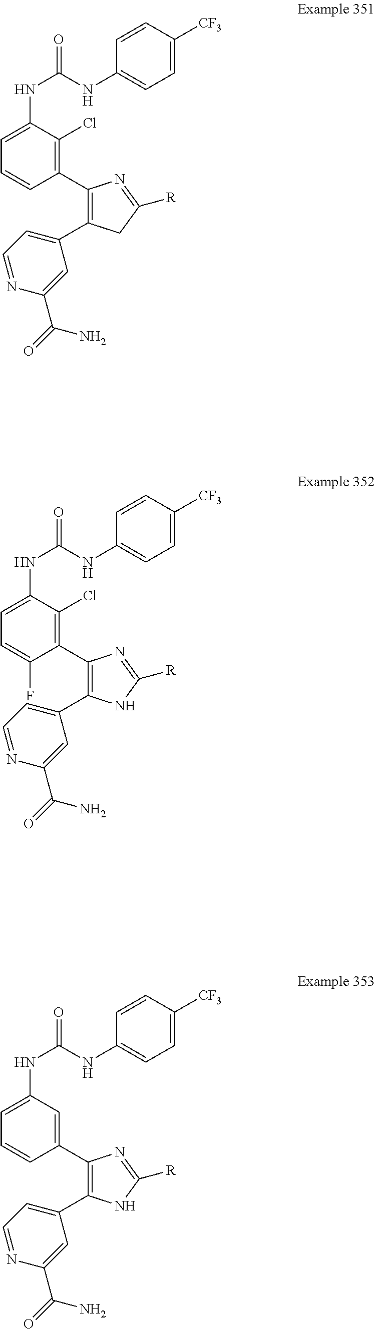

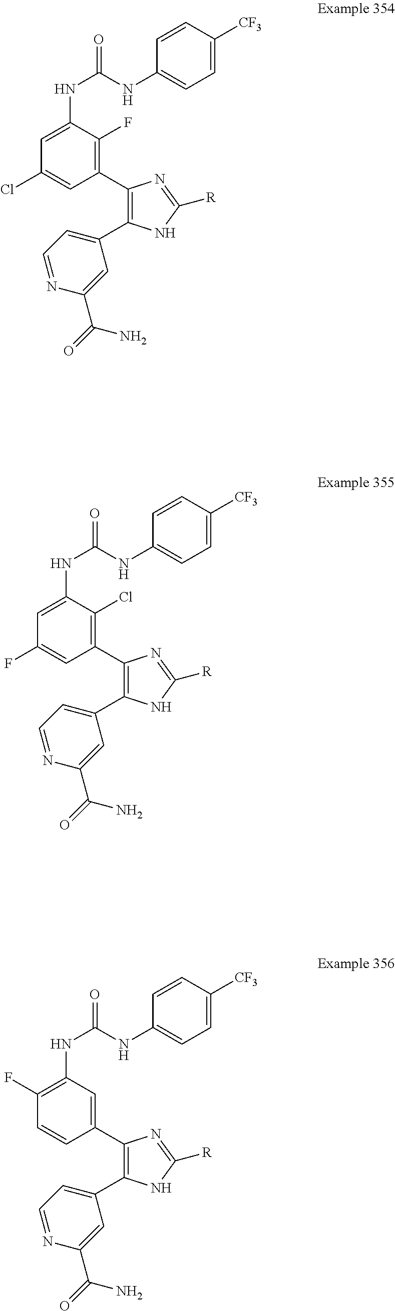

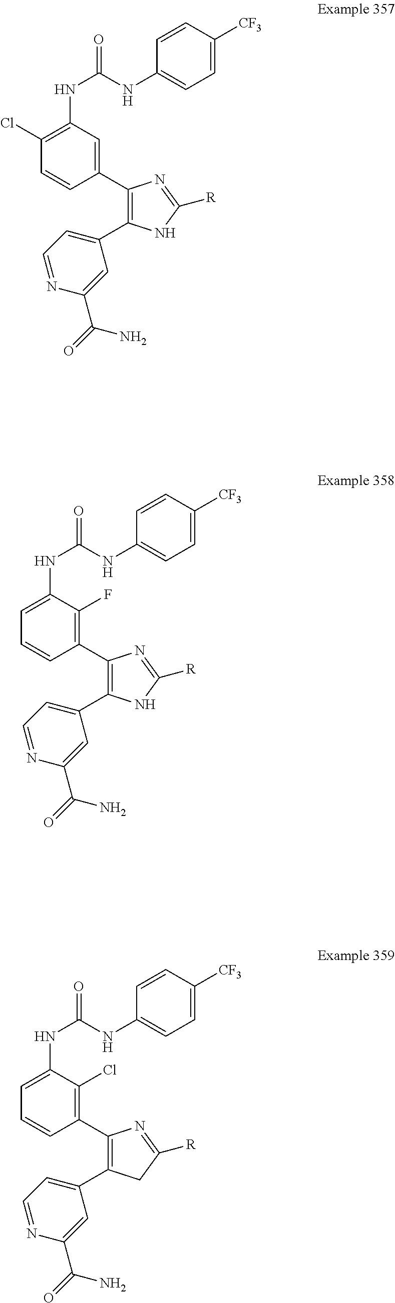

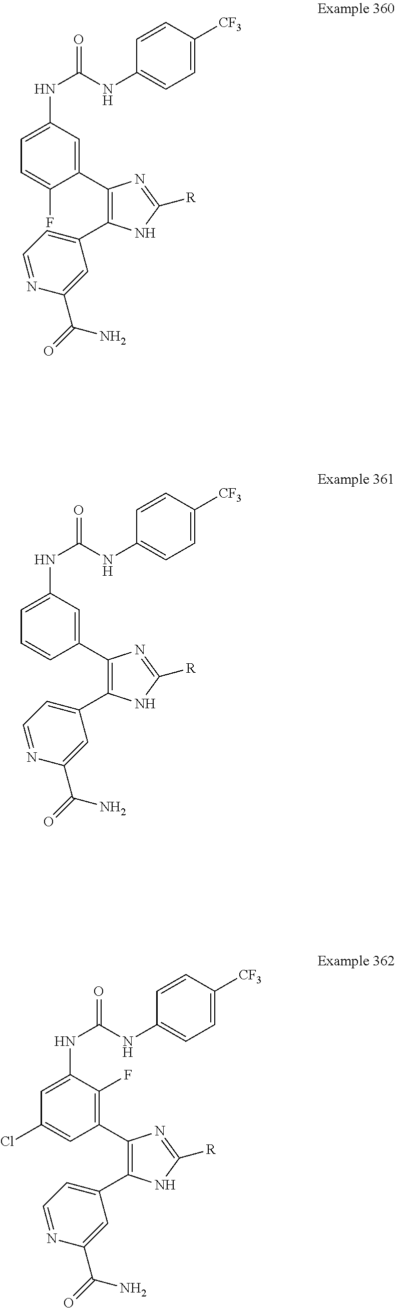

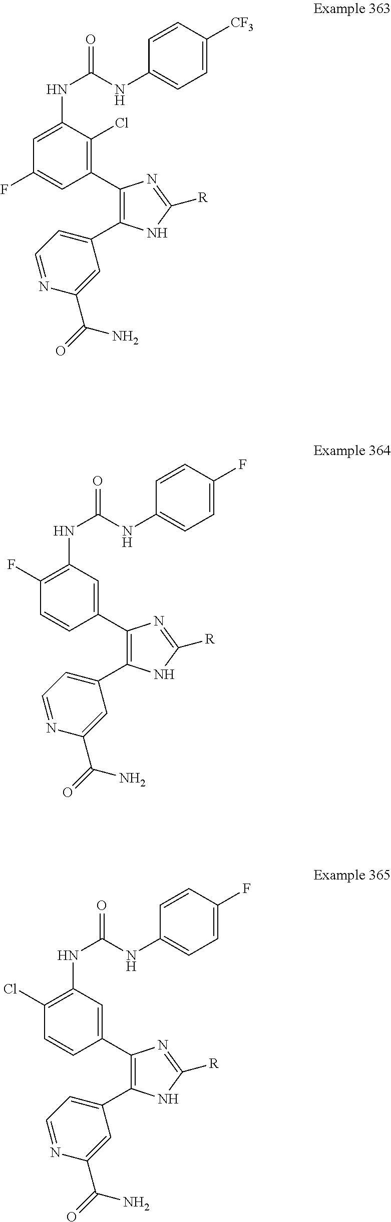

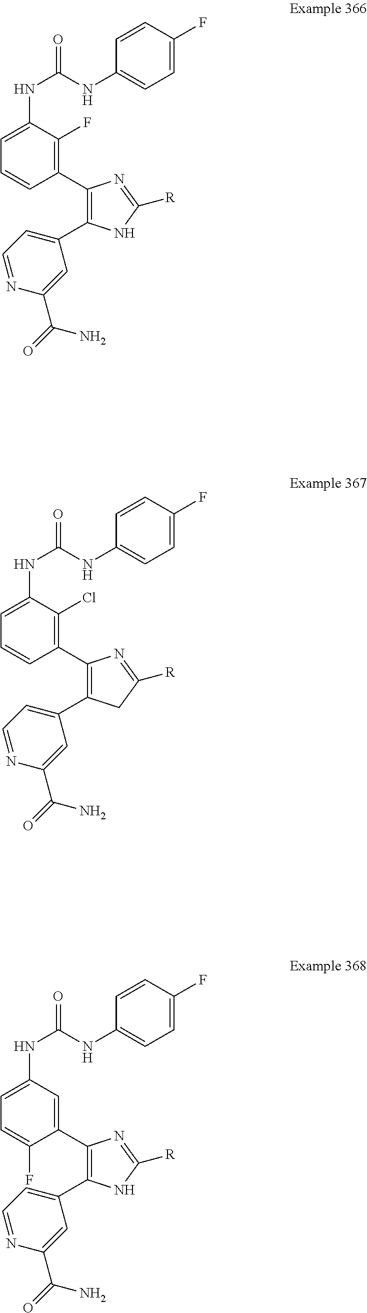

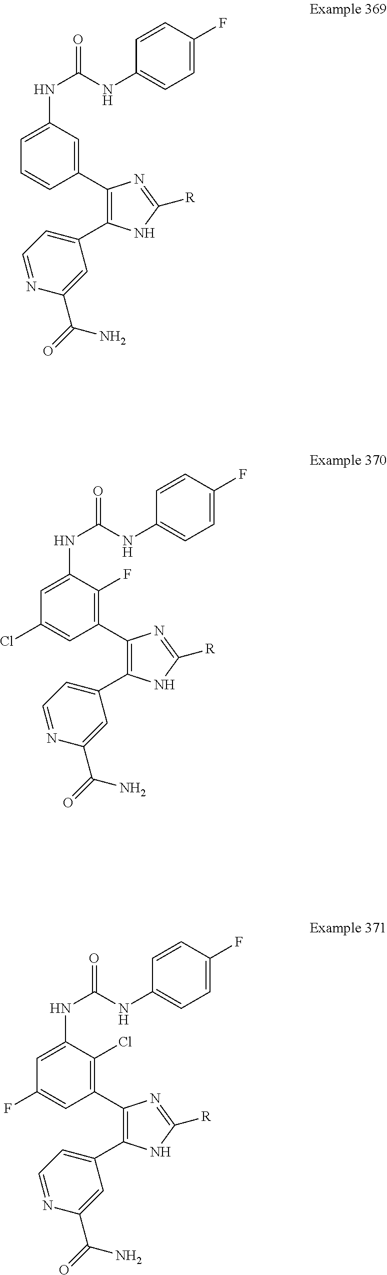

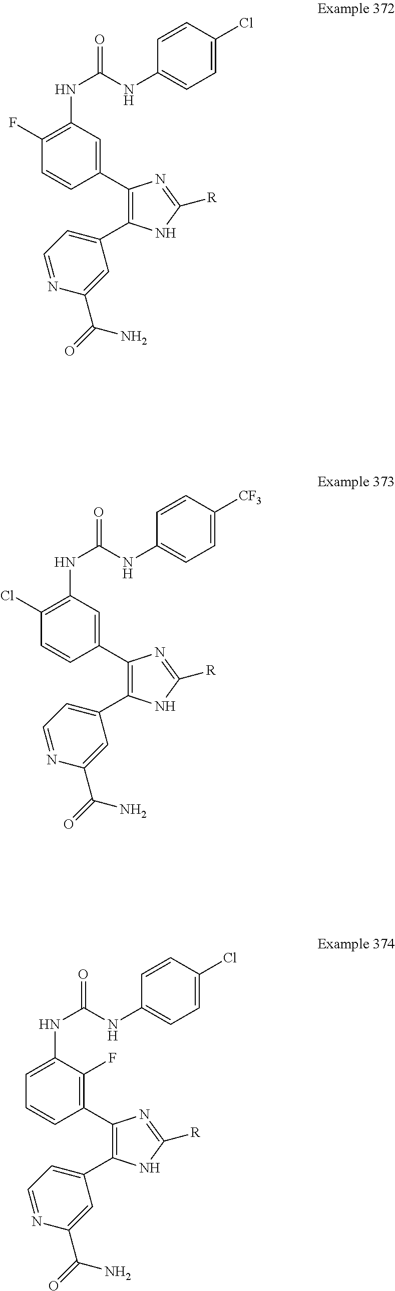

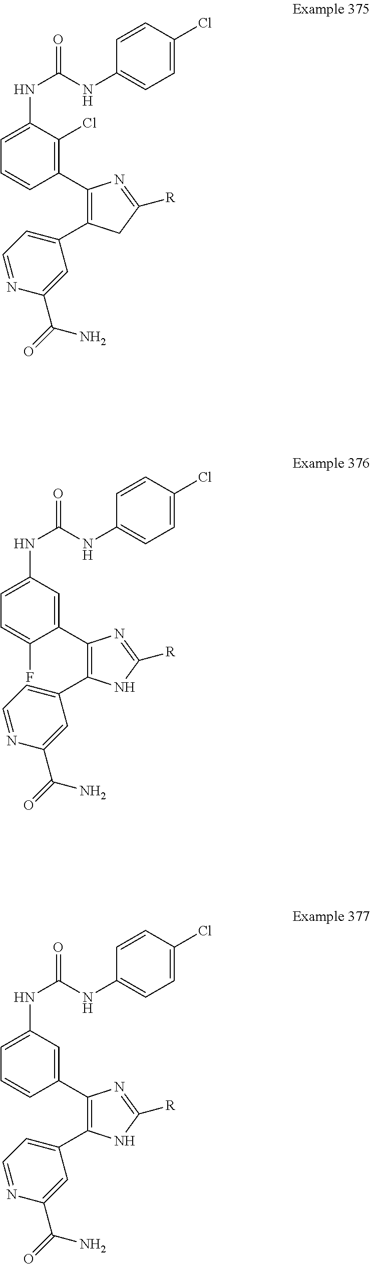

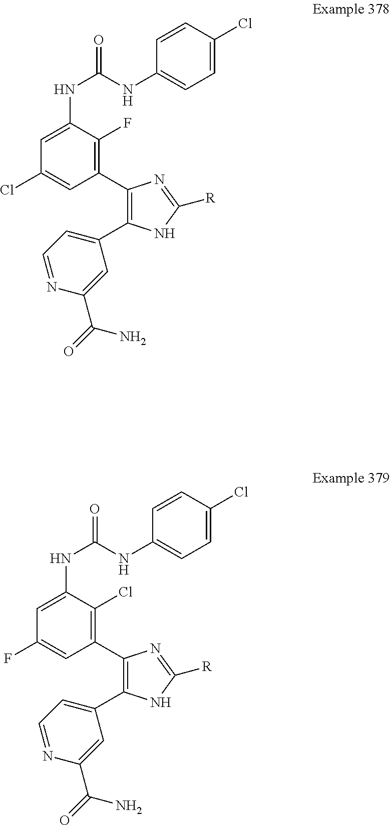

The following examples are synthesized according to example 63 using intermediate 31

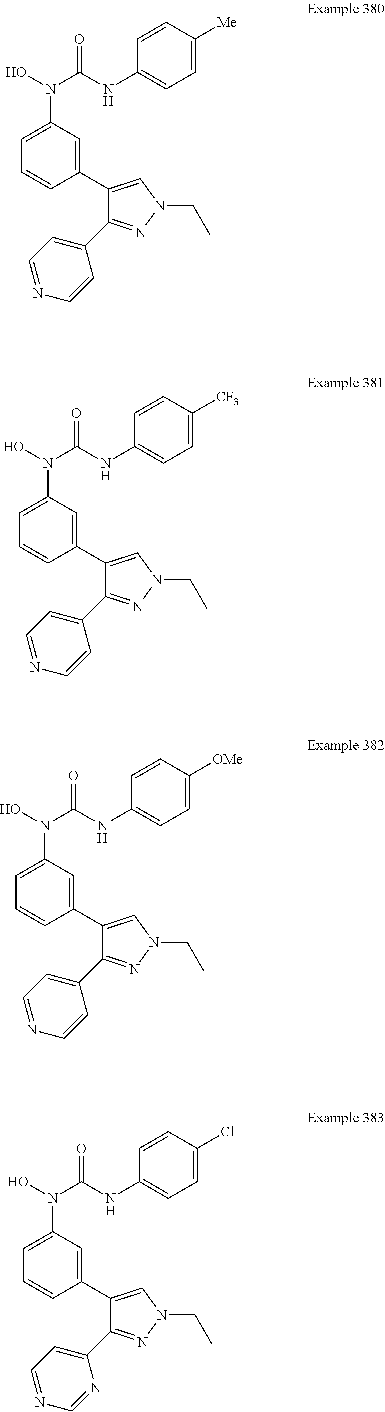 [SMILES: CCn1cc(-c2cccc(N(O)C(=O)Nc3ccc(C)cc3)c2)c(-c2ccncc2)n1 ; CCn1cc(-c2cccc(N(O)C(=O)Nc3ccc(C(F)(F)F)cc3)c2)c(-c2ccncc2)n1 ; CCn1cc(-c2cccc(N(O)C(=O)Nc3ccc(OC)cc3)c2)c(-c2ccncc2)n1 ; CCn1cc(-c2cccc(N(O)C(=O)Nc3ccc(Cl)cc3)c2)c(-c2ccncn2)n1]

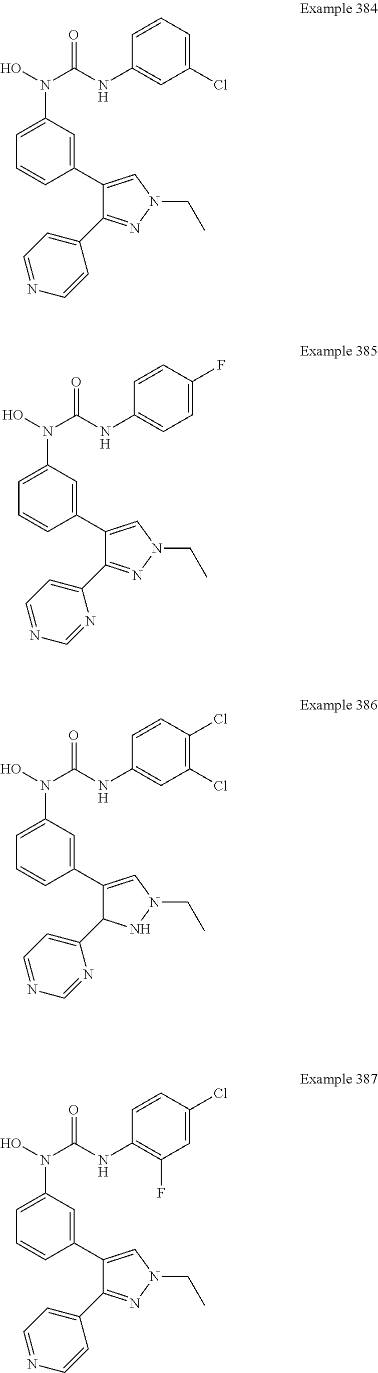 [SMILES: CCn1cc(-c2cccc(N(O)C(=O)Nc3cccc(Cl)c3)c2)c(-c2ccncc2)n1 ; CCn1cc(-c2cccc(N(O)C(=O)Nc3ccc(F)cc3)c2)c(-c2ccncn2)n1 ; CCN1C=C(c2cccc(N(O)C(=O)Nc3ccc(Cl)c(Cl)c3)c2)C(c2ccncn2)N1 ; CCn1cc(-c2cccc(N(O)C(=O)Nc3ccc(Cl)cc3F)c2)c(-c2ccncc2)n1]

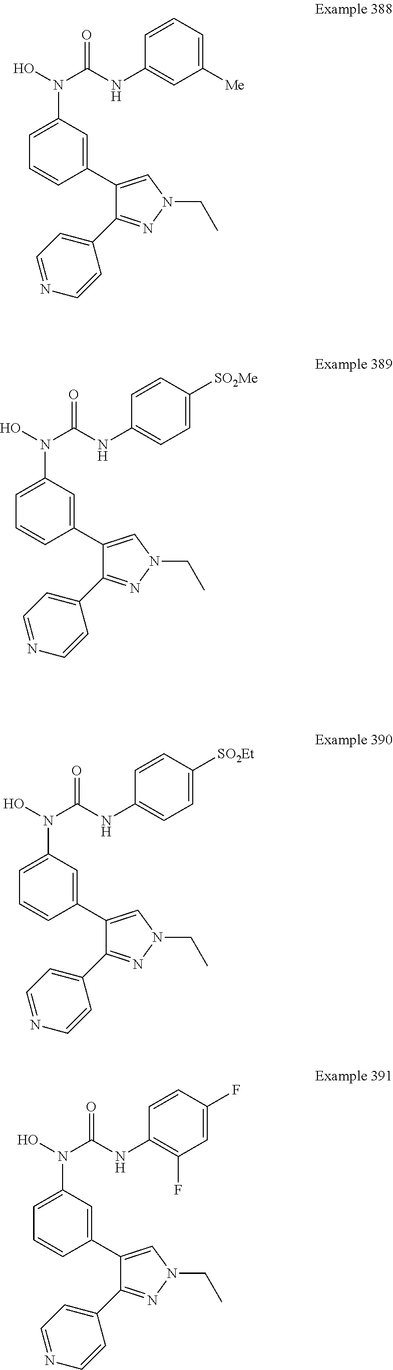

The following examples are synthesized according to example 63 using intermediate 4

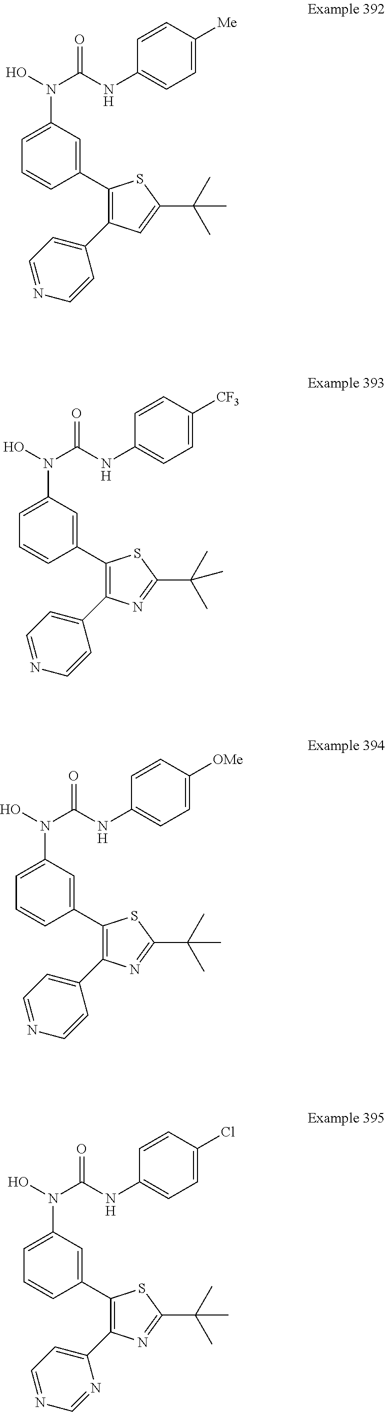 [SMILES: Cc1ccc(NC(=O)N(O)c2cccc(-c3sc(C(C)(C)C)cc3-c3ccncc3)c2)cc1 ; CC(C)(C)c1nc(-c2ccncc2)c(-c2cccc(N(O)C(=O)Nc3ccc(C(F)(F)F)cc3)c2)s1 ; COc1ccc(NC(=O)N(O)c2cccc(-c3sc(C(C)(C)C)nc3-c3ccncc3)c2)cc1 ; CC(C)(C)c1nc(-c2ccncn2)c(-c2cccc(N(O)C(=O)Nc3ccc(Cl)cc3)c2)s1]

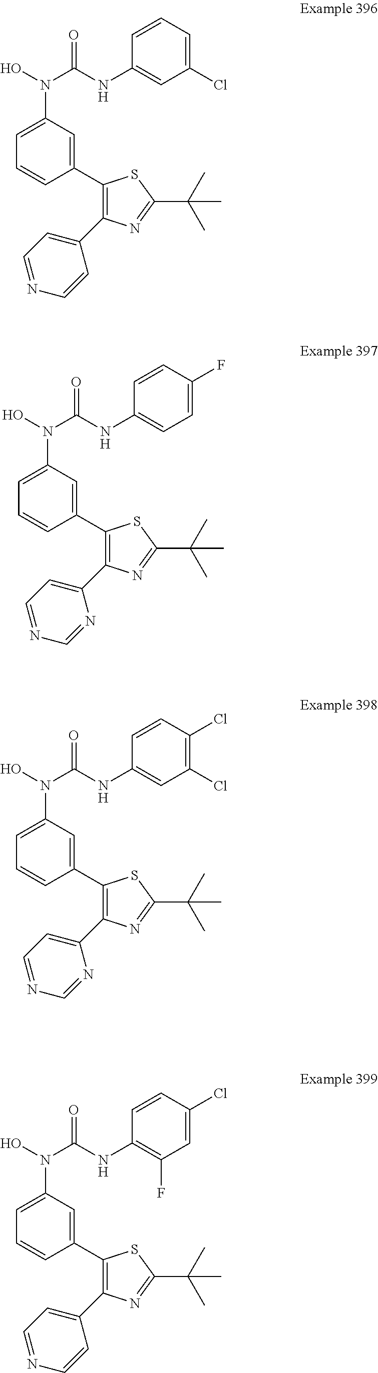 [SMILES: CC(C)(C)c1nc(-c2ccncc2)c(-c2cccc(N(O)C(=O)Nc3cccc(Cl)c3)c2)s1 ; CC(C)(C)c1nc(-c2ccncn2)c(-c2cccc(N(O)C(=O)Nc3ccc(F)cc3)c2)s1 ; CC(C)(C)c1nc(-c2ccncn2)c(-c2cccc(N(O)C(=O)Nc3ccc(Cl)c(Cl)c3)c2)s1 ; CC(C)(C)c1nc(-c2ccncc2)c(-c2cccc(N(O)C(=O)Nc3ccc(Cl)cc3F)c2)s1]

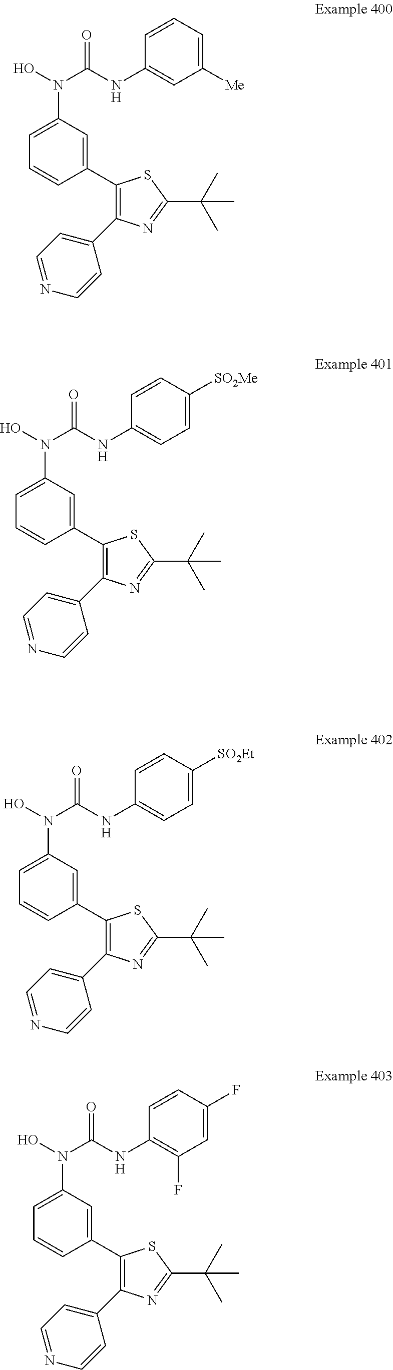

The following examples are synthesized according to example 63 using intermediate 10

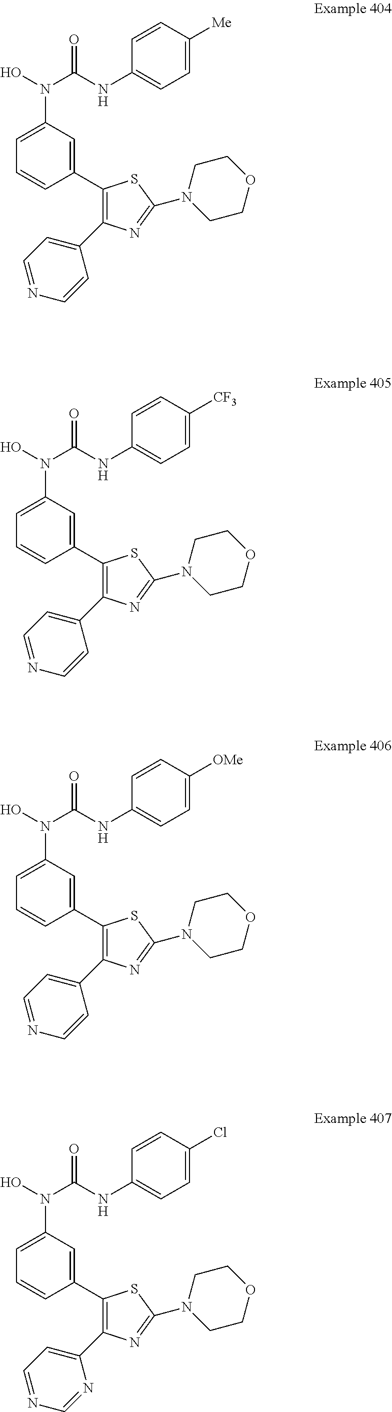 [SMILES: Cc1ccc(NC(=O)N(O)c2cccc(-c3sc(N4CCOCC4)nc3-c3ccncc3)c2)cc1 ; O=C(Nc1ccc(C(F)(F)F)cc1)N(O)c1cccc(-c2sc(N3CCOCC3)nc2-c2ccncc2)c1 ; COc1ccc(NC(=O)N(O)c2cccc(-c3sc(N4CCOCC4)nc3-c3ccncc3)c2)cc1 ; O=C(Nc1ccc(Cl)cc1)N(O)c1cccc(-c2sc(N3CCOCC3)nc2-c2ccncn2)c1]

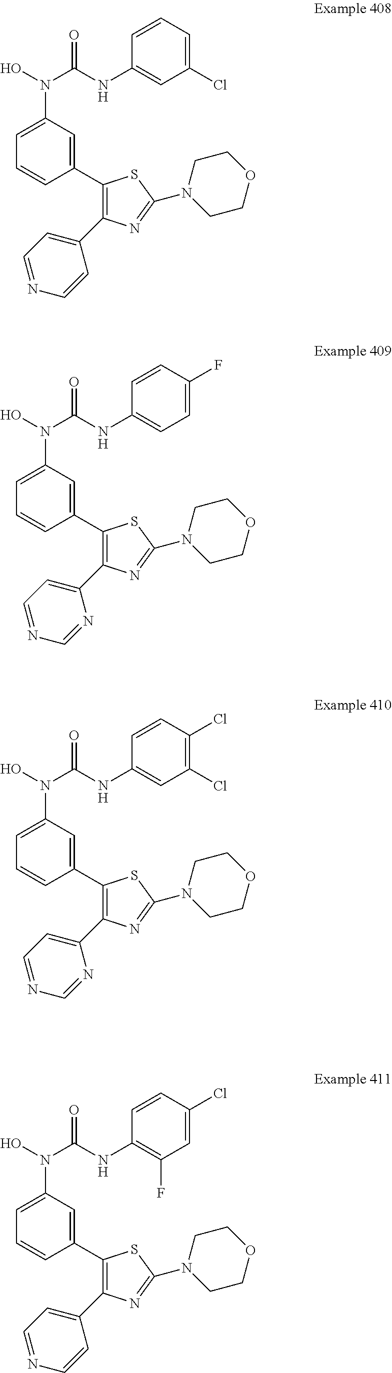 [SMILES: O=C(Nc1cccc(Cl)c1)N(O)c1cccc(-c2sc(N3CCOCC3)nc2-c2ccncc2)c1 ; O=C(Nc1ccc(F)cc1)N(O)c1cccc(-c2sc(N3CCOCC3)nc2-c2ccncn2)c1 ; O=C(Nc1ccc(Cl)c(Cl)c1)N(O)c1cccc(-c2sc(N3CCOCC3)nc2-c2ccncn2)c1 ; O=C(Nc1ccc(Cl)cc1F)N(O)c1cccc(-c2sc(N3CCOCC3)nc2-c2ccncc2)c1]

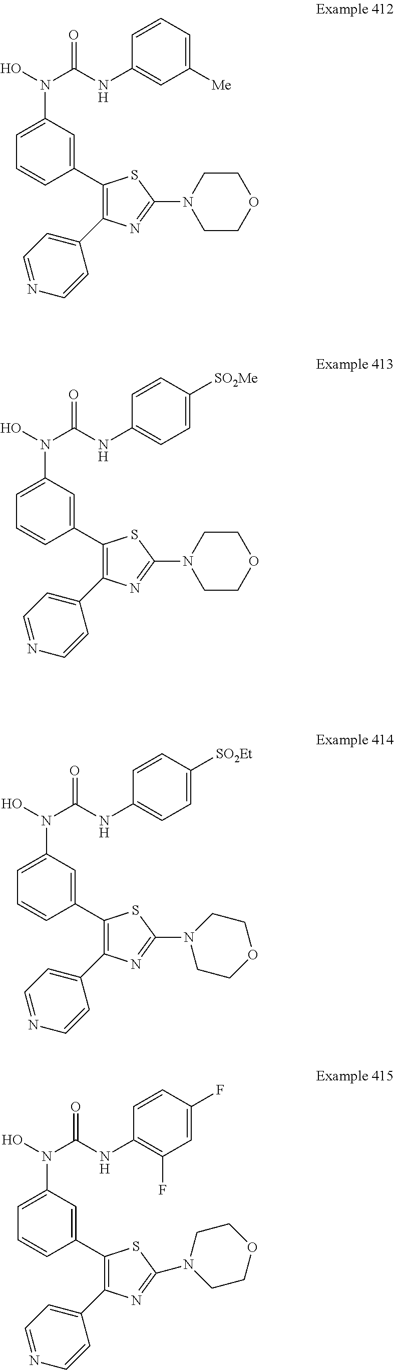

The following examples are synthesized according to example 63 using intermediate 12

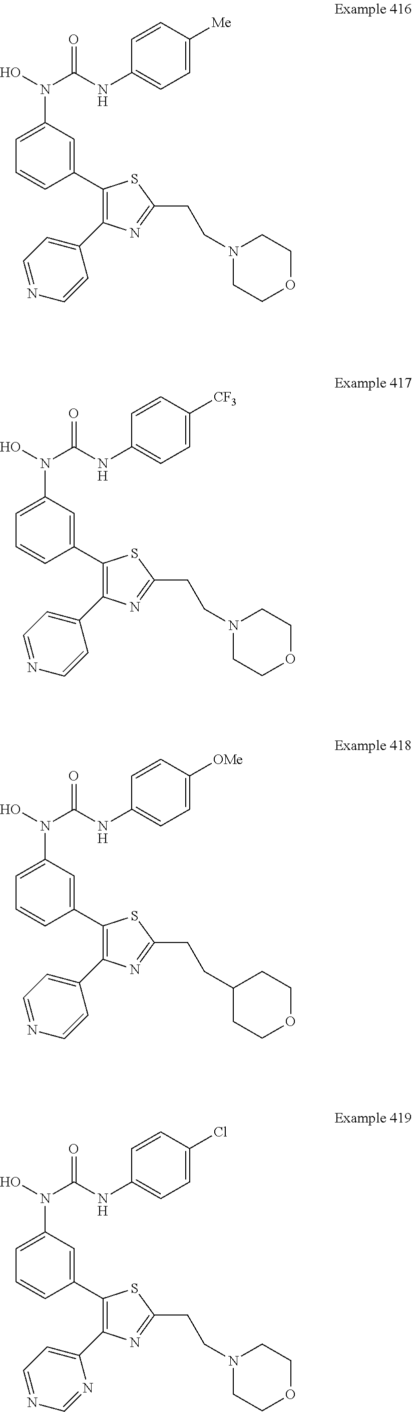 [SMILES: Cc1ccc(NC(=O)N(O)c2cccc(-c3sc(CCN4CCOCC4)nc3-c3ccncc3)c2)cc1 ; O=C(Nc1ccc(C(F)(F)F)cc1)N(O)c1cccc(-c2sc(CCN3CCOCC3)nc2-c2ccncc2)c1 ; COc1ccc(NC(=O)N(O)c2cccc(-c3sc(CCC4CCOCC4)nc3-c3ccncc3)c2)cc1 ; O=C(Nc1ccc(Cl)cc1)N(O)c1cccc(-c2sc(CCN3CCOCC3)nc2-c2ccncn2)c1]

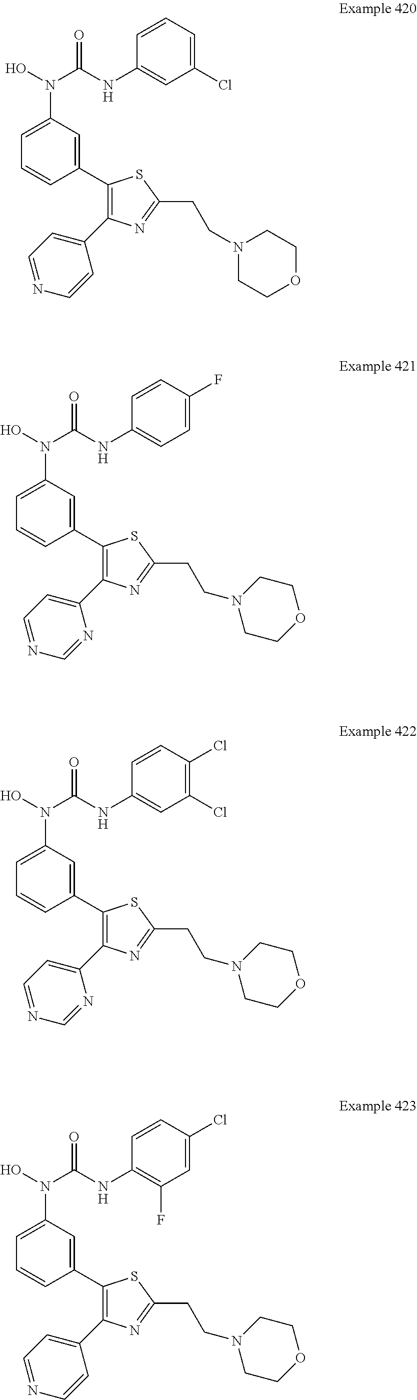 [SMILES: O=C(Nc1cccc(Cl)c1)N(O)c1cccc(-c2sc(CCN3CCOCC3)nc2-c2ccncc2)c1 ; O=C(Nc1ccc(F)cc1)N(O)c1cccc(-c2sc(CCN3CCOCC3)nc2-c2ccncn2)c1 ; O=C(Nc1ccc(Cl)c(Cl)c1)N(O)c1cccc(-c2sc(CCN3CCOCC3)nc2-c2ccncn2)c1 ; O=C(Nc1ccc(Cl)cc1F)N(O)c1cccc(-c2sc(CCN3CCOCC3)nc2-c2ccncc2)c1]

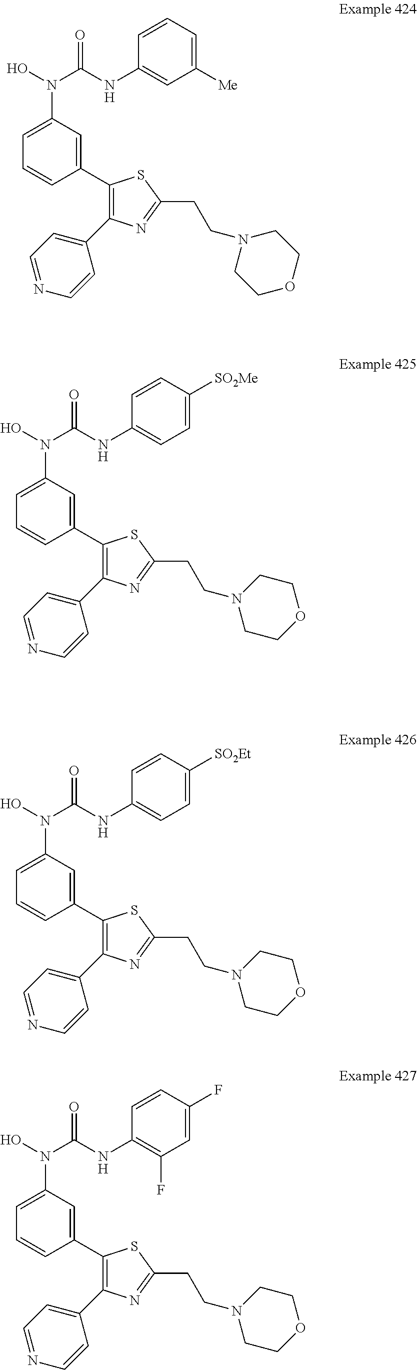

### II. Biological Evaluation

The ability of compounds described herein to inhibit RAF kinase activity were determined from biochemical kinase inhibition assays using recombinant RAF proteins as known in the art. In addition, the ability of compounds described herein to selectively inhibit cell growth of cultured cells containing either V600E activated B-RAF or wild-type B-RAF was performed as described below.

### In Vitro Assay for Determining Inhibition of RAF Kinases

Solutions of varying concentrations of test compounds or vehicle were added to 10 nM recombinant wild-type A-RAF, wild-type B-RAF, or wild-type C-RAF proteins incubated in the presence of different concentrations of ATP and 1 μM MEK (K97R) as substrate, as previously described (Wilhelm, S. M., et al., Cancer Res., 64: 7099-7109, 2004; Mason, C S., et al., EMBO J. 18: 2137-2148, 1999; Marais, R., et al., J. Biol. Chem., 272: 4378-4383, 1997). At least triplicate determinations for each individual test compound concentration were made and data plotted as mean±standard deviation relative to the control vehicle.

### In Vitro Assay for Determining Inhibition of B-RAF Kinase or Mutant B-RAF Kinase

Solutions of varying concentrations of test compounds or vehicle were added to 10 nM recombinant V600E-mutated B-RAF proteins incubated in the presence of different concentrations of ATP and 1 MEK (K97R) as substrate, as previously described (Wilhelm, S. M., et al., Cancer Res., 64: 7099-7109, 2004; Mason, C. S., et al.; EMBO J. 18: 2137-2.148, 1999; Marais, R., et al., J. Biol. Chem., 272: 4378-4383, 1997). At least triplicate determinations for each individual test compound concentration were made and data plotted as mean±standard deviation relative to the control vehicle.

### In Vitro Assays for Tumor Cell Growth

Briefly, growth inhibition of cells containing V600E activated B-RAF (A375, Colo205) versus cell lines with wild-type B-RAF (A431) were measured under anchorage-dependent conditions using MTT (3-(4,5-dimethylthiazol-2-yl)-2,5-diphenyltetrazolium bromide), following 72 hours incubation with either compound or vehicle, as previously described (Haass, N. K., et al., Clinical Cancer Res., 14: 230-239, 2008): Cell lines are obtained from the American Type Tissue Culture Collection (Maryland, USA) and cultured in media containing heat-inactivated 10% fetal bovine serum. Cell cultures were also maintained in 10 U/mL, penicillin, 100 μg/mL streptomycin and 2 mM glutamine. At least triplicate determinations for each individual test compound concentration were made and data plotted as mean±standard deviation relative to the control vehicle.

Table 2 provides biological and chemical characterization data for the compounds of Formula (I)-(IV) as described herein.

TABLE 2

| | IC₅₀ (μM) | | | |
|---|---|---|---|---|
| | B-Raf | IC₅₀ (μM) | IC₅₀ (μM) | ¹H NMR |
| Example | V600E | C-Raf | A375 | (δ, ppm) |
| 1 | A | C | D | (CDCl₃, ppm) 2.34 (s, 3H), 2.77 (s, 3H), |
| | | | | 6.73 (br s, 1H), 7.03 (m, 2H), 7.14 (d, |
| | | | | 2H), 7.20 (d, 2H), 7.31 (m, 2H), 7.45 (d, |
| | | | | 2H), 7.46 (br d, 1H), 8.49 (d, 2H) |
| 2 | A | | | (DMSO-d6) 2.76 (s, 3H), 6.98 (d, 1H), |
| | | | | 7.40 (t, 1H), 7.43 (d, 2H), 7.44 (m, 2H), |
| | | | | 7.63 (s, 2H), 8.10 (s, 1H), 8.53 (d, 2H); |
| | | | | 9.05 (s, 1H), 9.23 (s, 1H) |
| 3 | A | | C | (CDCl₃) 2.77 (s, 3H), 7.04 (d, 1H), 7.20- |
| | | | | 7.26 (m, 5H), 7.32 (t, 1H), 7.41 (br s, |
| | | | | 1H), 7.48 (d, 2H), 7.53 (d, 1H), 7.77 (br |
| | | | | s, 1H), 8.45 (d, 2H) |
| 4 | B | | | (CDCl₃) 2.77 (s, 3H),6.95 (t, 2H) 7.02 (d, |
| | | | | 1H), 7.24-7.25 (m, 4H), 7.30 (t, 1H), 7.47 |
| | | | | (d, 2H), 7.51 (d, 1H), 7.60 (br s, 1H), |
| | | | | 7.46 (d, 2H) |
| 5 | B | | | (CDCl₃) 2.78 (s, 3H), 6.62 (m, 1H), 6.88 |
| | | | | (ddd, 1H), 7.08 (d, 1H), 7.08 (t, 1H), 7.38 |
| | | | | (t, 1H), 7.50 (s and d, 3H), 7.65 (d, 1H), |
| | | | | 8.07 (m, 1H), 8.44 (d, 2H), 8.66 (s, 1H) |
| 6 | B | | | (DMSO-d6) |
| 7 | A | B | C | (DMSO-d6)□2.26 (s, 3H), 2.75 (s, 3H), |
| | | | | 6.98 (m, 1H), 7.09 (d, 2H), 7.33 (m, 3H), |
| | | | | 7.43 (d, 2H), 8.30 (d, 1H), 8.52 (d, 2H), |
| | | | | 8.71 (br s, 1H), 9.05 (br s, 1H) |
| 8 | A | | | (DMSO-d6) 2.25 (s, 3H), 2.79 (s, 3H), |
| | | | | 7.08 (d, 2H), 7.30 (t, 1H), 7.32 (d, 2H), |
| | | | | 7.41 (d, 2H), 7.43 (m, 1H), 7.60 (dd, 1H), |
| | | | | 8.54 (d, 2H), 8.62 (br s, 1H), 8.82 (br s, |
| | | | | 1H) |
| 9 | A | | B | |
| 10 | A | | | (DMSO-d6) 2.76 (s, 3H), 7.00 (m, 1H), |
| | | | | 7.05-7.07 (m, 1H), 7.21 (m, 1H), 7.24 |
| | | | | (m, 1H), 7.33 (m, 2H), 7.70 (t, 1H), 8.26 |
| | | | | (dd, 1H), 8.53 (d, 2H), 8.80 (br d, 1H), |
| | | | | 9.32 brs, 1H). |
| 11 | A | | | (DMSO-d6) 2.75 (s, 3H), 7.01 (m, 1H), |
| | | | | 2,27 (dd, 1H), 7.35 (dd, 1H), 7,42 (d, |
| | | | | 2H), 7.53 (d, 1H), 7.87 (d, 1H), 8.23 (dd, |
| | | | | 1H), 8.53 (d, 2H), 8.84 br d, 1H), 9.41 |
| | | | | (br s, 1H). |
| 12 | A | | | (DMSO-d6) 2.75 (s, 3H), 7.0 (m, 1H), |
| | | | | 7.23 (m, 1H), 7.35 (dd, 1H), 7.42 (d, 2H), |
| | | | | 7.49 (dd, 1H), 8.15 (brt, 1H), 8.30 (dd, |
| | | | | 1H), 8.53 (d, 2H), 9.19 (br d, 1H) 9.24 |
| | | | | (brd, 1H). |
| 13 | A | | C | (DMSO-d6) 2.75 (s, 3h), 6.98 (m, 1h), |
| | | | | 7.32 (m, 1H), 7.34 (d, 2H), 7.42-7.45 (m, |
| | | | | 4H), 8.26 (d, 1H) 8.52 (d, 2H), 8.76 (s, |
| | | | | 1H), 9.25 (s, 1H). |
| 14 | A | | C | (DMSO-d6) 2,76 (s, 3H), 7.01 (m, 1H), |
| | | | | 7.35 (dd, 1H), 7.42 (d, 2H), 7.65 (s, 4H), |
| | | | | 8.27 (dd, 1H), 8.53 (d, 2H), 8.85 (br d, |
| | | | | 1H), 9.52 (br s, 1H) |
| 15 | B | | C | (DMSO-d6) 2.79 (s, 3H), 7.10 (t, 1H), |
| | | | | 7.28 (t, 1H), 7.41 (d, 2H), 7.67 (s, 4H), |
| | | | | 8.29 (t, 1H), 8.55 (d, 2H), 8.76 (s, 1H), |
| | | | | 9.45 (s, 1H). |
| 16 | A | | D | (DMSO-d6) 2.75 (s, 3H), 7.00 (m, 1H), |
| | | | | 7.33 (m, 2H), 7.38 (d, 2H), 7.53 (m, 2H), |
| | | | | 8.01 (br s, 1H), 8.26 (dd, 1H), 8.52 (d, |
| | | | | 2H), 8.83 (br d, 1H), 9.47 (s, 1H). |
| 17 | A | | D | (DMSO-d6) 2.76 (s, 3H), 3.17 (s, 3H), |
| | | | | 6.99 (d, 1H), 7.39 (t, 2H), 7.5 (d, 2H), |
| | | | | 7.54 (m, 2H), 7.68 (d, 2H), 7.82 (d, 2H), |
| | | | | 8.53 (d, 2H), 9.01 (s, 1H), 9.24 (s, 1H). |
| 18 | B | | | (DMSO-d6) 2.80 (s, 3H), 7.09 (t, 1H), |
| | | | | 7.27 (t, 1H), 7.36 (d, 2H), 7.42 (d, 2H), |
| | | | | 7.48 (d, 2H), 8.29 (t, 1H), 8.56 (d, 2H), |
| | | | | 8.67 (s, 1H), 9.19 (s, 1H) |
| 19 | A | | | |
| 20 | A | | C | (DMSO-d6) 2.26 (s, 3H), 2.72 (s, 3H), |
| | | | | 5.95 (s, 2H), 6.37 (d, 1H), 6.75 (s, 1H), |
| | | | | 6.93 (d, 1H), 7.09 (s, 1H), 7.33 (m, 3H), |
| | | | | 7.50 (m, 2H), 7.79 (d, 1H), 8.60 (s, 1H), |
| | | | | 8.80 (s, 1H) |
| 21 | A | | C | (DMSO-d6) 2.73 (s, 3H), 5.96 (s, 2H), |
| | | | | 6.37 (d, 1H), 6.74 (s, 1H), 6.94 (d, 1H), |
| | | | | 7.33 (m, 3H), 7.50 (m, 4H), 7.79 (d, 1H), |
| | | | | 9.14 (broad s, 2H) |
| 22 | A | | C | (DMSO-d6) 2.73 (s, 3H), 5.95 (s, 2H), |
| | | | | 6.38 (d, 1H), 6.71 (s, 1H), 6.96 (m, 1H), |
| | | | | 7.30 (dd, 1H), 7.34 (d, 2H), 7.48 (d, 2H), |
| | | | | 7.80 (d, 1H), 8.26 (dd, 1H), 8.78 (s, 1H), |
| | | | | 9.31 (s, 1H) |
| 23 | B | | | (DMSO-d6) 2.76 (s, 3H), 5.97 (s, 2H), |
| | | | | 6.34 (d, 1H), 6.74 (s, 1H), 7.05 (t, 1H), |
| | | | | 7.24 (t, 1H), 7.36 (d, 2H), 7.49 (d, 2H), |
| | | | | 7.80 (d, 1H), 8.26 (t, 1H), 8.75 (s, 1H), |
| | | | | 9.26 (s, 1H) |
| 24 | B | | B | (DMSO-d6) 2.76 (s, 3H), 5.98 (s, 2H), |
| | | | | 6.34 (d, 1H), 6.74 (s, 1H), 7.08 (t, 1H), |
| | | | | 7.26 (t, 1H), 7.67 (s, 4H), 7.80 (d, 1H), |
| | | | | 8.26 (t, 1H), 8.83 (s, 1H), 9.53 (s, 1H) |
| 25 | A | | C | (DMSO-d6) 2.76 (s, 3H), 5.97 (s, 2H), |
| | | | | 6.38 (d, 1H), 6.71 (s, 1H), 7.52 (t, 1H), |
| | | | | 7.66 (s, 4H), 7.80 (d, 1H), 8.20 (t, 1H), |
| | | | | 8.90 (s, 1H), 9.58 (s, 1H) |
| 26 | A | | D | (DMSO-d6) 2.73 (s, 3H), 5.97 (s, 2H), |
| | | | | 6.38 (d, 1H), 6.75 (s, 1H), 6.98 (d, 1H), |
| | | | | 7.36 (t, 1H), 7.52 (m, 2H), 7.66 (s, 4H), |
| | | | | 7.80 (d, 1H), 8.96 (s, 1H), 9.13 (s, 1H) |
| 27 | B | | | (DMSO-d6) 1.49 (s, 9H), 2.26 (s, 3H), |
| | | | | 6.96 (d, 1H), 7.09 (d, 2H), 7.34 (m, 3H), |
| | | | | 7.46 (m, 3H), 7.61 (s, 1H), 8.54 (d, 2H), |
| | | | | 8.62 (s, 1H), 8.80 (s, 1H) |
| 28 | C | | | (DMSO-d6) 1.47 (s, 9H), 2.26 (s, |
| | | | | 3H), 3.41 (m, 4H), 6.40 (d, 1H), 6.65 (t, |
| | | | | 1H), 6.77 (s, 1H), 6.95 (d, 1H), 7.10 (d, |
| | | | | 2H), 7.33 (m, 3H), 7.46 (d, 1H), 7.56 (s, |
| | | | | 1H), 7.86 (d, 1H), 8.59 (s, 1H), 8.77 (s, |
| | | | | 1H) |
| 29 | A | | | (DMSO-d6) 1.47 (s, 9H), 2.26 (s, 3H), |
| | | | | 3.38 (q, 2H), 3.50 (t, 2H), 6.38 (d, 1H), |
| | | | | 6.60 (t, 1H), 6.77 (s, 1H), 6.95 (d, 1H), |
| | | | | 7.10 (d, 2H), 7.33 (m, 3H), 7.46 (d, 1H), |
| | | | | 7.56 (s, 1H), 7.86 (d, 1H), 8.60 (s, 1H), |
| | | | | 8.78 (s, 1H) |
| 30 | A | | | (DMSO-d6) 1.47 (s, 9H), 2.26 (s, 3H), |
| | | | | 2.45 (s, 6H), 2,74 (m, 2H), 3.36 (m, 2H), |
| | | | | 6.43 (d, 1H), 6.65 (t, 1H), 6.79 (s, 1H), |
| | | | | 6.96 (d, 1H), 7.10 (d, 2H), 7.33 (m, 3H), |
| | | | | 7.43 (d, 1H), 7.60 (s, 1H), 7.89 (d, 1H), |
| | | | | 8.66 (s, 1H), 8.83 (s, 1H) |
| 31 | A | | | (DMSO-d6) 1.47 (s, 9H), 1.80 (m, 4H), |
| | | | | 2.26 (s, 3H), 2.85 (m, 6H), 3.36 (m, 2H), |
| | | | | 6.46 (d, 1H), 6.71 (t, 1H), 6.77 (s, 1H), |
| | | | | 6.95 (d, 1H), 7.10 (d, 2H), 7.33 (m, 3 H), |
| | | | | 7.45 (d, 1H), 7.60 (s, 1H), 7.89 (d, 1H), |
| | | | | 8.71 (s, 1H), 8.89 (s, 1H) |
| 32 | A | | | (DMSO-d6) 1.04 (d, 3H), 1.47 (s, 9H), |
| | | | | 2.26 (s, 3H), 3.22 (m, 2H), 3.51 (s, 3H), |
| | | | | 3.68 (m, 1H), 6.39 (d, 1H), 6.66 (t, 1H), |
| | | | | 6.79 (s, 1H), 6.95 (d, 1H), 7.05 (d, 1H), |
| | | | | 7.10 (d, 2H), 7.33 (m, 3H), 7.47 (d, 1H), |
| | | | | 7.56 (s, 1H), 7.85 (d, 1H), 8.60 (s, 1H), |
| | | | | 8.78 (s, 1H) |
| 33 | A | | C | (DMSO-d6) 3.49 (br t, 4H), 3.77 (br t, |
| | | | | 4H), 6.93 (d, 1H), 7.30-7.52 (m, 8H), |
| | | | | 8.49 (d, 2H), 8.87 (br s, 2H). |
| 34 | A | | C | (DMSO-d6) 2.46 (br s, 2H), 2.56 (m, |
| | | | | 2H), 3.44 (q, 2H), 3.61 (m, 4H), 6.88 (d, |
| | | | | 1H), 7.29-7.49 (m, 9H), 7.80 (t, 1H), 8.47 |
| | | | | (d, 2H), 8.84 (d, 2H). |
| 35 | A | | B | (DMSO-d6) 2.46 (br s, 4H), 2.55 (m, |
| | | | | 2H), 3.44 (m, 2H), 3.60 (m, 4H), 6.92 |
| | | | | (m, 1H), 7.25 (dd, 1H), 7.36 (m, 2H), 7.38 |
| | | | | (m, 2H), 8.18 (d, 1H), 8.47 (d, 2H), 8.73 |
| | | | | (br s, 1H), 9.28 (br s, 1H). |
| 36 | A | | | (DMSO-d6) 2.46 (br s, 4H), 2.55 (m, |
| | | | | 2H), 3.44 (m, 2H), 3.60 (m, 4H), 6.92 |
| | | | | (m, 1H), 7.25 (dd, 1H), 7.36 (m, 2H), 7.38 |
| | | | | (m, 2H), 8.18 (d, 1H), 8.47 (d, 2H), 8.73 |
| | | | | (br s, 1H), 9.28 (br s, 1H). |
| 37 | A | | C | (CDCl₃) 2.54 (br s, 4H), 2.69 (t, 2H), |
| | | | | 3.41 (q, 2H), 3.76 (t, 4H), 5.99 (t, 1H), |
| | | | | 7.03-7.08 (m, 2H), 7.40-7.52 (m, 6H), |
| | | | | 7.57 (d, 1H), 7.77 (s, 1H), 7.91 (s, 1H), |
| | | | | 8.41 (d, 2H). |
| 38 | B | | | (DMSO-d6) 7.04 (t, 1H), 7.22 (t, 1H), |
| | | | | 7.33 (d, 2H), 7.40 (s, 2H), 7.67 (s, 4H), |
| | | | | 8.21 (t, 1H), 8.49 (d, 2H), 8.72 (s, 1H), |
| | | | | 9.45 (s, 1H) |
| 39 | C | | | (DMSO-d6) 7.02 (t, 1H), 7.20 (t, 1H), |
| | | | | 7.33 (m, 6H), 7.49 (d, 2H), 8.20 (t, 1H), |
| | | | | 8.48 (d, 2H), 8.62 (s, 1H), 9.18 (s, 1H) |
| 40 | A | | | (DMSO-d6) 6.90 (m, 1H), 7.23-7.35 (m, |
| | | | | 7H), 7.48 (d, 2H), 8.16 (d, 1H), 8.47 (d, |
| | | | | 2H), 8.84 (s, 1H), 9.41 (s, 1H) |
| 41 | A | | C | (DMSO-d6) 2.74 (s, 1H), 7.05 (d, 1H), |
| | | | | 7.09 (s, 1H), 7.18 (dd, 1H), 7.25 (d, 2H), |
| | | | | 7.26 (t, 1H), 7.37 (m, 3H), 8.46 (d, 2H), |
| | | | | 10.97 (s, 1H) |
| 42 | B | | | (DMSO-d6) 2.74 (s, 3H), 7.05 (s, 1H), |
| | | | | 7.16 (t, 1H), 7.22-7.30 (m, 5H), 7.35 (t, |
| | | | | 1H), 7.73, (m, 1H), 8.44 (d, 2H), 11.11 |
| | | | | (s, 1H) |
| 43 | C | | | (DMSO-d6) 2.71 (s, 3H), 6.03 (s, 2H), |
| | | | | 6.09 (d, 1H), 6.73 (s, 1H), 7.02 (d, 1H), |
| | | | | 7.16 (m, 2H), 7.29 (m, 3H), 7.67 (d, 1H), |
| | | | | 7.74 (m, 1H), 11.1 (broad s, 1H) |
| 44 | B | | | (DMSO-d6) 1.47 (s, 9H), 7.09 (d, 1H), |
| | | | | 7.17 (s, 1H), 7.23 (d, 1H), 7.28 (m, 4H), |
| | | | | 7.35 (t, 1H), 7,74 (m, 1H), 8.46 (d, 2H), |
| | | | | 11.1 (broad s, 1H) |
| 45 | B | | | (DMSO-d6) 2.29 (s, 3H), 2.72 (s, 3H), |
| | | | | 3.59 (s, 2H), 5.95 (s, 2H), 6.34 (d, 1H), |
| | | | | 6.72 (s, 1H), 6.99 (d, 1H), 7.14 (d, 2H), |
| | | | | 7.22 (d, 2H), 7.34 (d, 1H), 7.67 (m, 2H), |
| | | | | 7.77 (d, 1H), 10.3 (s, 1H) |
| 46 | C | | | (DMSO-d6) 1.48 (s, 9H), 7.03 (d, 1H), |
| | | | | 7.14 (d, 2H), 7.21 (d, 2H), 7.37 (t, 1H), |
| | | | | 7.42 (d, 2H), 7.63 (d, 1H), 7.76 (s, 1H), |
| | | | | 8.53 (d, 2H), 10.3 (s, 1H) |
| 47 | A | | D | (DMSO-d6) 2.26 (s, 3H), 2.75 (s, 3H), |
| | | | | 3.65 (s, 2H), 7.11 (d, 2H), 7.25 (m, 1H), |
| | | | | 7.40 (m, 5H), 7.46 (d, 2H), 8.49 (d, 2H), |
| | | | | 10.0 (s, 1H) |
| 48 | A | | C | (DMSO-d6) 1.49 (t, 3H), 4.26 (q, 2H), |
| | | | | 6.87 (m, 1H), 7.26 (dd, 1H), 7.35 (d, 2H), |
| | | | | 7.42 (broad s, 2H), 7.47 (d, 2H), 8.03 (s, |
| | | | | 1H), 8.15 (d, 1H), 8.53 (broad s, 2H), |
| | | | | 8.69 (s, 1H), 9.27 (s, 1H) |
| 49 | A | | B | (DMSO-d6) 1.50 (t, 3H), 4.29 (q, 2H), |
| | | | | 6.99 (t, 1H), 7.20 (t, 1H), 7.37 (m, 4H), |
| | | | | 7.50 (d, 2H), 8.10 (s, 1H), 8.17 (t, 1H), |
| | | | | 8.53 (d, 2H), 8.70 (s, 1H), 9.32 (s, 1H) |
| 50 | A | C | B | (DMSO-d6) 1.50 (t, 3H), 4.29 (q, 2H), |
| | | | | 7.00 (t, 1H), 7.21 (t, 1H), 7.39 (d, 2H), |
| | | | | 7.67 (m, 4H), 8.10 (s, 1H), 8.15 (t, 1H), |
| | | | | 8.53 (d, 2H), 8.94 (s, 1H), 9.74 (s, 1H) |
| 51 | A | | C | (DMSO-d6) 1.49 (t, 3H), 3.17 (s, 3H), |
| | | | | 4.26 (q, 2H), 6.94 (t, 1H), 7.42-7.46 (m, |
| | | | | 4H), 7.68 (d, 2H), 7.82 (d, 2H), 8.06 (s, |
| | | | | 1H), 8.53 (d, 2H), 8.89 (s, 1H), 9.20 (s, |
| | | | | 1H) |
| 52 | A | B | A | (CDCl₃) 1.59 (t, 3H), 4.26 (q, 2H), 7.02 |
| | | | | (d, 1H), 7.12 (s, 1H), 7.32 (t, 1H), 7.41 |
| | | | | (d, 2H), 7.47 (s, 1H), 7.49-7.52 (m, 3H), |
| | | | | 7.54 (dd, 1H), 7.85 (s, 1H), 8.10 (s, 1H), |
| | | | | 8.45 (d 2H). |
| 53 | A | | C | (DMSO-d6) 1.50 (t, 3H), 4.01 (m, 4H), |
| | | | | 6.99 (d, 1H), 7.40 (t, 1H), 7.44 (d, 2H), |
| | | | | 7.62 (m, 2H), 7.74 (d, 2H), 7.86 (d, 2H), |
| | | | | 8.09 (s, 1H), 8.53 (d, 2H) |
| 54 | A | | C | (DMSO-d6) 1.09 (t, 3H), 1,44 (t, 3H), |
| | | | | 2.53 (q, 2H), 4.22 (q, 2H), 7.31 (d, 1H), |
| | | | | 7.43 (t, 1H), 7.47 (m, 4H), 7.68 (d, 2H), |
| | | | | 7.70 (d, 2H), 8.05 (s, 1H), 8.05 (d, 2H), |
| | | | | 8.89 (s, 1H), 9.22 (s, 1H), |
| 55 | A | | D | (DMSO-d6) 1.47 (t, 3H), 3.21 (s, 3H), |
| | | | | 4.25 (q, 2H), 6.91 (d, 1H) 7.32 (t, 1H), |
| | | | | 7.45 m(4H), 7.52 (m, 2H), 7.54 (d, 1H), |
| | | | | 8.05 (s, 1H), 8.53 (d, 2H), 8.83 (s, 1H), |
| | | | | 9.11 (s, 1H). |
| 56 | A | | D | (DMSO-d6).49 (t, 3H), 2.40 (d, 3H), 4.25 |
| | | | | (q, 2H), 6.91 (d, 1H), 7.30-7.34 (m, 2H), |
| | | | | 7.41-7.63 (m, 4H), 7.45 (dd, 4H), 8.05 (s, |
| | | | | 1H), 8.52 (d, 2H), 8.86 (s, 1H), 9.12 (s, |
| | | | | 1H). |
| 57 | A | | A | (DMSO-d6) 1.49 (t, 3 H), 3.73 (s, 3H), |
| | | | | 4.25 (q, 2H), 6.86 (d, 3H), 7.29 (t, 1H), |
| | | | | 7.34 (d, 2H), 7.36 (m, 2H), 7.42 (d, 2H), |
| | | | | 8.03, (s, 1H), 8.47 (, s 1H), 8.53 (d, 2H), |
| | | | | 8.62 (s, 1H). |
| 58 | D | | | |
| 59 | A | | D | |
| 60 | | | | (CDCl₃) 1.31 (t, 3H), 4.0 (q, 2H), 4.98 |
| | | | | (brs, 2H), 6.34 (s, 1 H), 6.42 (d, 1H), 6.94 |
| | | | | (d, 1H), 7.30 (t, 1H), 7.33 (d, 1.H), 7.45 |
| | | | | (d, 2H), 7.59 (d, 2H), 8.06 (d, 1H), 8.11 |
| | | | | (d, 1H), 8.88 (brs, 1H), 9.19 (brs, 1H) |
| 61 | | | | (CD₃OD) 1.58 (t, 3H), 4.340 (q, 2H), |
| | | | | 6.85 (d, 1H), 7.05 (t, 1H), 7.14 (t, 1H), |
| | | | | 7.58 (d, 2H), 7.64 (d, 2H), 7.88 (s, 1H), |
| | | | | 8.05 (t, 1H), 8.08(8, 1H) |
| 62 | | | | |
| 63 | A | | C | (DMSO-d6) 1.49 (t, 3H), 4.25 (q, 2H), |
| | | | | 7.02 (d, 1H), 7.37 (t, 1H), 7.43 (d, 2H), |
| | | | | 7.59 (m, 2H), 7.60 (d, 2H), 7.92 (d, 2H), |
| | | | | 8.08. (s, 1H), 8.52 (d, 2H), 9.77 (s, 1H), |
| | | | | 10.80 (s, 1H). |

Note:
Biochemical and cell-based activity is designated within the following ranges:
A: ≦0.10 μM
B: >0.10 μM to ≦1.0 μM
C: >1.0 μM to ≦10 μM
D: >10 μM

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It intended that the following claims define the scope of the invention and that Methods and structures within the scope of these claims and their equivalents be covered thereby.

Various embodiments of the present invention are provided below.
1. A compound of Formula (I), or a tautomer, steroisomer, geometric isomer, a pharmaceutically acceptable salt, solvate, or hydrate thereof;

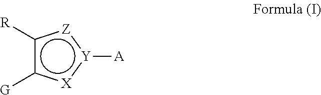

wherein
Z is N, Y is C, and X is NH;
or
Z is CH, Y is N, and X is N;
or
Z is N, Y is N, and X is N;
R is

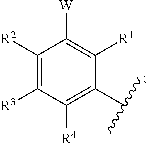

G is selected from

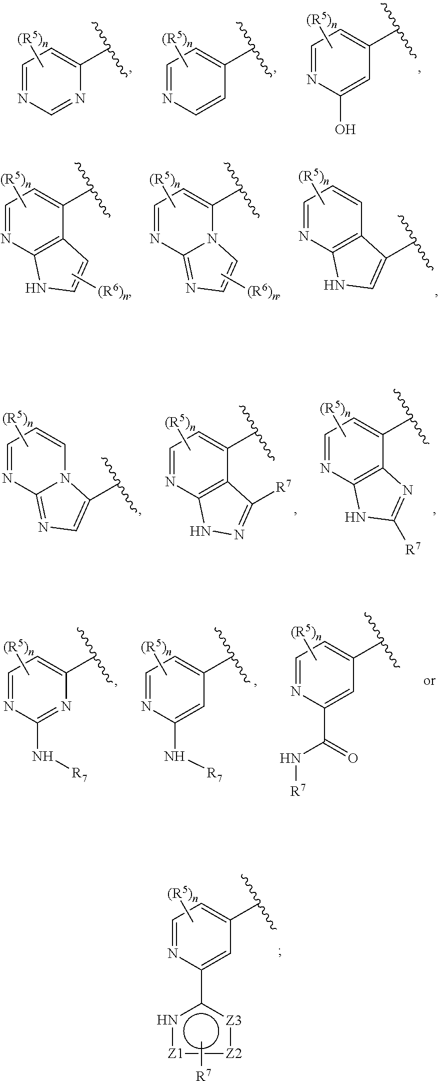

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;

R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;

R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHSO₂Ar, —NHCOAr, —NHSO₂NHAr, —NHSO₂N(Ar)₂, —NHCONHAr, —N(OH)CONHAr, —NHCON(Ar)₂, —NHCSNHAr, —NHCSN(Ar)₂, NHCOC(R¹¹)(R¹²)Ar, —C(R¹¹)(R¹²)CONHAr;
Ar is:

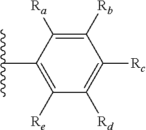

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently, selected from H, or C₁-C₆ alkyl; or for the instance wherein R¹¹ and R¹² are attached geminal carbon substituents, R¹¹ and R¹² together with the carbon atom to which they are attached are joined to form a C₃-C₆ cycloalkyl; and
n is 0, 1, or 2.
2. The compound of embodiment 1, wherein Z is CH, Y is N, and X is N.
3. The compound of embodiment 1, wherein Z is N, Y is C, and X is NH.
4. The compound of embodiment 1, wherein Z is N, Y is N, and X is N.
5. The compound of embodiment 1, wherein W is NHSO₂Ar.
6. The compound of embodiment 1, wherein W is NHCONHAr.
7. The compound of embodiment 1 wherein G

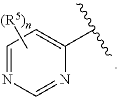

8. The compound of embodiment 1, wherein is

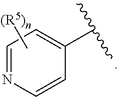

9. The compound of embodiment 1, wherein G is

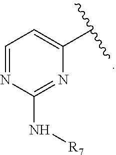

10. The compound of embodiment 1, wherein G is

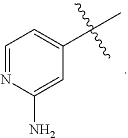 [SMILES: *c1ccnc(N)c1]

11. The compound of embodiment 1, wherein A is an optionally substituted alkyl or optionally substituted cycloalkyl.
12. The compound of embodiment 11, wherein A is an optionally substituted group selected from methyl, ethyl, trifluoromethyl, 2,2,2-trifluoroethyl, n-propyl, n-butyl, s-butyl, i-butyl, t-butyl, cyclopropyl or cyclobutyl.
13. The compound of embodiment 1, wherein. A is —NR⁹R¹⁰.
14. The compound of embodiment 1, wherein A is an optionally substituted C-attached heterocycloalkyl.
15. The compound of embodiment 1, wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl.
16. The compound of embodiment 15, wherein R³ and R⁴ are hydrogen.
17. The compound of embodiment 16, wherein R¹ and R² are each independently selected from hydrogen, F, Cl, CN, OH, CH—, F, CHF₂, CF₃, or C₂F₅.
18. The compound of embodiment 6, wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).
19: A pharmaceutical composition comprising a compound of embodiment 1, or a stereoisomer, tautomer, hydrate, solvate or pharmaceutically acceptable salt thereof, and at least one pharmaceutically acceptable excipient.
20. A method of inhibiting a protein kinase comprising contacting the protein kinase with an inhibitory concentration of a compound of embodiment 1.
21. The compound of embodiment 1 selected from the following:

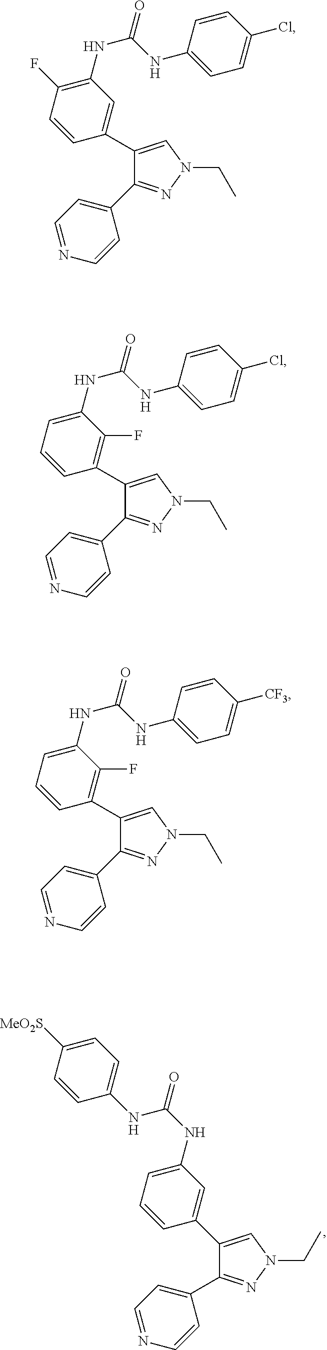 [SMILES: CCn1cc(-c2ccc(F)c(NC(=O)Nc3ccc(Cl)cc3)c2)c(-c2ccncc2)n1 ; CCn1cc(-c2cccc(NC(=O)Nc3ccc(Cl)cc3)c2F)c(-c2ccncc2)n1 ; CCn1cc(-c2cccc(NC(=O)Nc3ccc(C(F)(F)F)cc3)c2F)c(-c2ccncc2)n1 ; CCn1cc(-c2cccc(NC(=O)Nc3ccc(S(C)(=O)=O)cc3)c2)c(-c2ccncc2)n1]

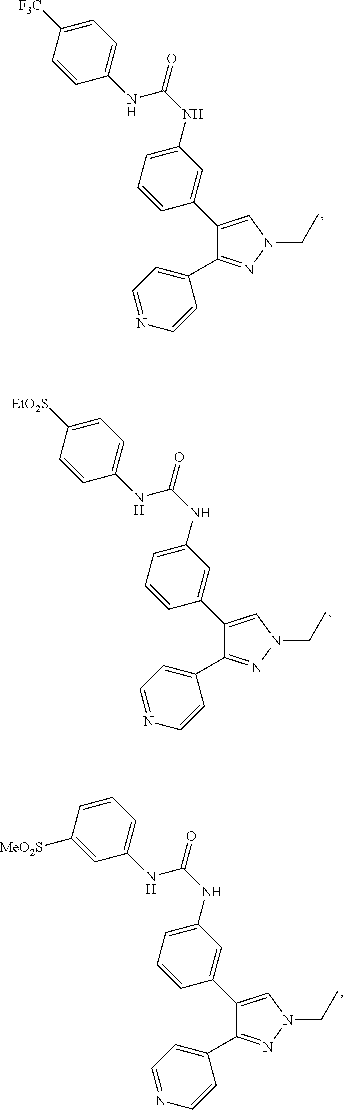

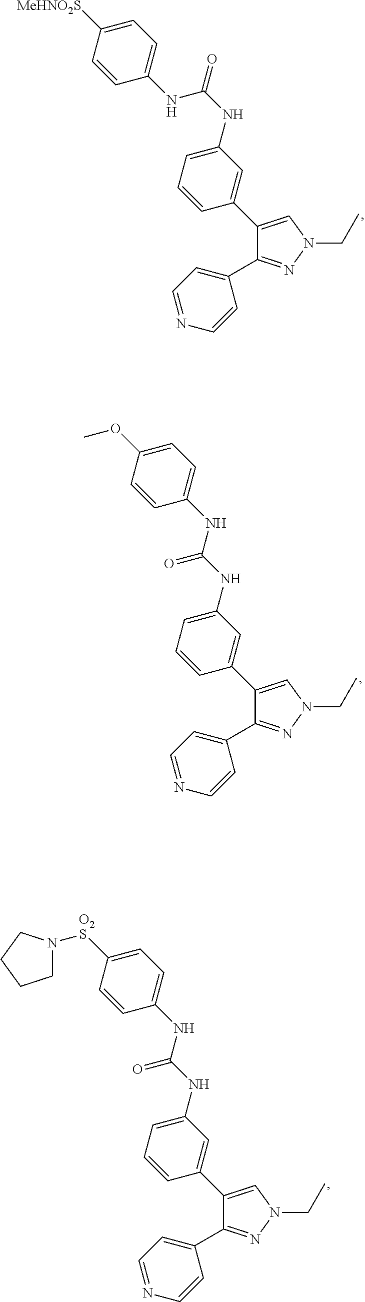

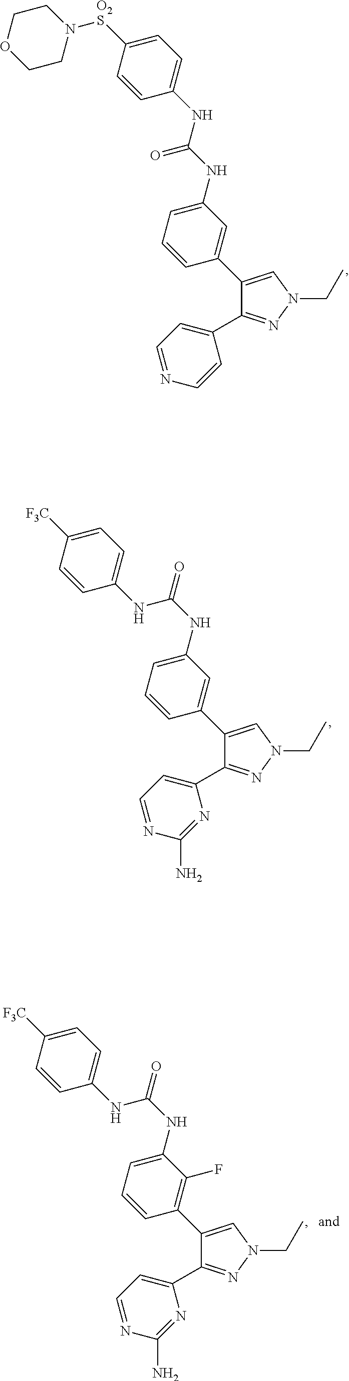 [SMILES: CCn1cc(-c2cccc(NC(=O)Nc3ccc(S(=O)(=O)N4CCOCC4)cc3)c2)c(-c2ccncc2)n1 ; CCn1cc(-c2cccc(NC(=O)Nc3ccc(C(F)(F)F)cc3)c2)c(-c2ccnc(N)n2)n1 ; CCn1cc(-c2cccc(NC(=O)Nc3ccc(C(F)(F)F)cc3)c2F)c(-c2ccnc(N)n2)n1]

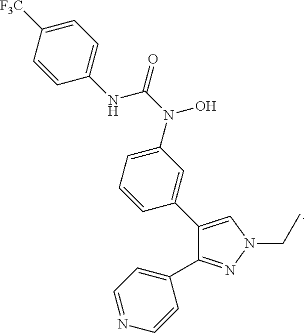 [SMILES: CCn1cc(-c2cccc(N(O)C(=O)Nc3ccc(C(F)(F)F)cc3)c2)c(-c2ccncc2)n1]

22. The method of embodiment 20, wherein the protein kinase is selected from A-RAF, B-RAF and C-RAF.
23. The method of embodiment 22, wherein the protein kinase is B-RAF.
24. The method of embodiment 23, wherein the protein kinase is a B-RAF mutant.
25. The method of embodiment 24, Wherein the protein kinase is the B-RAF V600E mutant.
26. A method of inhibiting RAF kinase mediated signalling in a cell comprising contacting the cell with an inhibitory concentration of a compound of embodiment 1.
27. The method of embodiment 26, wherein the cell is characterized by increased activity of the RAS-RAF-MEK-ERK pathway compared to a non-transformed cell.
28. The method of embodiment 26, wherein the cell is characterized by a B-RAF gain-of-function mutation.
29. The method of embodiment 26, wherein the cell is characterized by the presence of the B-RAF V600E mutant.
30. A method of treating a human disease or disorder mediated by RAF kinase signalling comprising administering to a patient a therapeutically effective amount of a composition comprising a compound of embodiment 1.
31. The method of embodiment 30, wherein the RAF kinase is B-RAF kinase.
32. The method of embodiment 30 or 31, wherein the disease or disorder is a proliferative disease.
33. The method of embodiment 32, wherein the proliferative disease is selected from melanoma, ovarian cancer, colorectal cancer, thyroid cancer, cholangiocarcinoma, or lung adenocarcinoma.
34. The method of embodiment 30, wherein the RAF kinase is selected from human A-RAF, B-RAF and C-RAF, or a homolog or an ortholog thereof
35. A compound of Formula (IT), or a tautomer, steroisomer, geometric isomer, a pharmaceutically acceptable salt, solvate, or hydrate thereof:

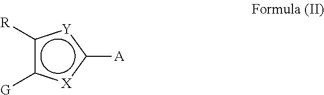

wherein
X is S and Y is N; or
X is N and Y is S; or
X is O and Y is N; or
X is N and Y is O;
R is

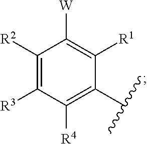

G is selected from:

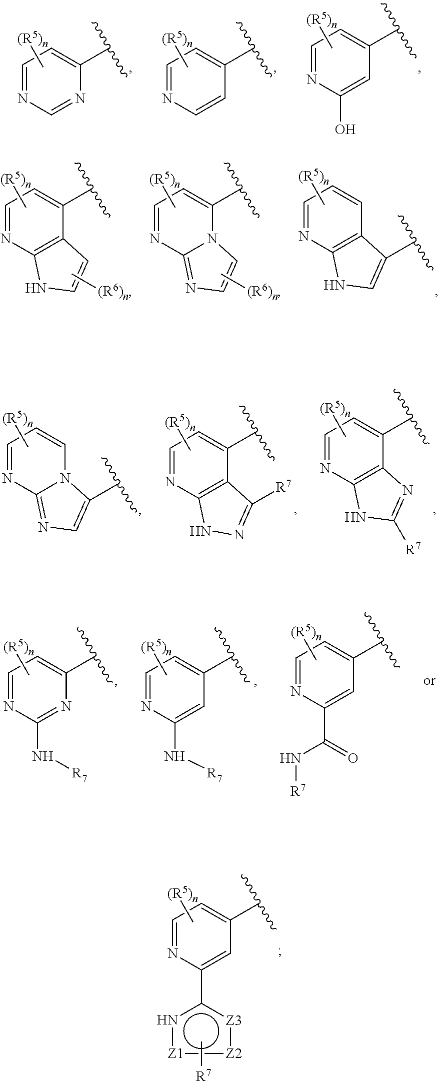

R⁵ and R⁶ are each independently selected from H, optionally substituted alkyl, optionally substituted cycloalkyl optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl or optionally substituted heteroalkyl;
R¹, R², R³ and R⁴ are each independently selected from hydrogen, F, Cl, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHCOAr, —NHSO₂NHAr, —N(OH)CONHAr, —NHSO₂N(Ar)₂, —NHCONHAr, —NHCON(Ar)₂, —NHCOC(R¹¹)(R¹²)Ar, —C(R¹¹)(R¹²)CONHAr, NHCSNHAr, NHCSN(Ar)₂ or —N(R¹¹)CON(R¹²)Ar;
Ar is:

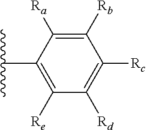

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl), NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
each R¹¹ and R¹² is independently selected from H, or C₁-C₆ alkyl; or for the instance wherein R¹¹ and R¹² are attached germinal carbon substituents, R¹¹ and R¹² together with the carbon atom to which they are attached are joined to form a C₃-C₆ cycloalkyl; and n is 0, 1, or 2.
36. The compound of embodiment 35, wherein X is S and Y is N.
37. The compound of embodiment 35, wherein X is N and Y is S.
38. The compound of embodiment 35, wherein W is NHSO₂Ar,
39. The compound of embodiment 35, wherein W is NHCONHAr.
40. The compound of embodiment 35, wherein G is

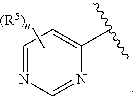

41. The compound of embodiment 35, wherein G is

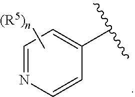

42. The compound of embodiment 35, wherein G is

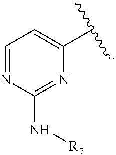

43. The compound of embodiment 35, wherein G is

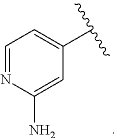 [SMILES: *c1ccnc(N)c1]

44. The compound of embodiment 35, wherein A is an optionally substituted alkyl or optionally substituted cycloalkyl.
45. The compound of embodiment 44, wherein A is an optionally substituted group selected from methyl, ethyl, trifluoromethyl, 2,2,2-trifluoroethyl, n-propyl, n-butyl, s-butyl, i-butyl, t-butyl, cyclopropyl or cyclobutyl.
46. The compound of embodiment 35, wherein A is NR⁹R¹⁰.
47. The compound of embodiment 35, wherein A is an optionally C-attached heterocycloalkyl.
48. The compound of embodiment 35, wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl.
49. The compound of embodiment 48, wherein R³ and R⁴ are hydrogen.
50. The compound of embodiment 49, wherein R¹ and R² are each independently selected from hydrogen, F, Cl, CN, OH, CH₂F, CHF₂, CF₃, or C₂F₅.
51. The compound of embodiment 39, wherein Ra, Rb, Rc, Rd and Re are each independently selected from hydrogen, F Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or SO₂(C₁-C₅ optionally substituted alkyl).
52. A pharmaceutical composition comprising a compound of embodiment 35, or a stereoisomer, tautomer, hydrate, solvate or pharmaceutically acceptable salt thereof, and at least one pharmaceutically acceptable excipient.
53. A method of inhibiting a protein kinase comprising contacting the protein kinase with an inhibitory concentration of a compound of embodiment 35.
54. The compound of embodiment 35 selected from the following:

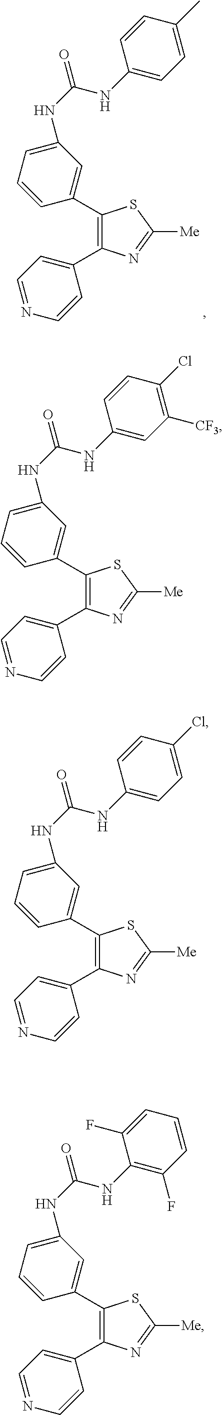 [SMILES: Cc1ccc(NC(=O)Nc2cccc(-c3sc(C)nc3-c3ccncc3)c2)cc1 ; Cc1nc(-c2ccncc2)c(-c2cccc(NC(=O)Nc3ccc(Cl)c(C(F)(F)F)c3)c2)s1 ; Cc1nc(-c2ccncc2)c(-c2cccc(NC(=O)Nc3ccc(Cl)cc3)c2)s1 ; Cc1nc(-c2ccncc2)c(-c2cccc(NC(=O)Nc3c(F)cccc3F)c2)s1]

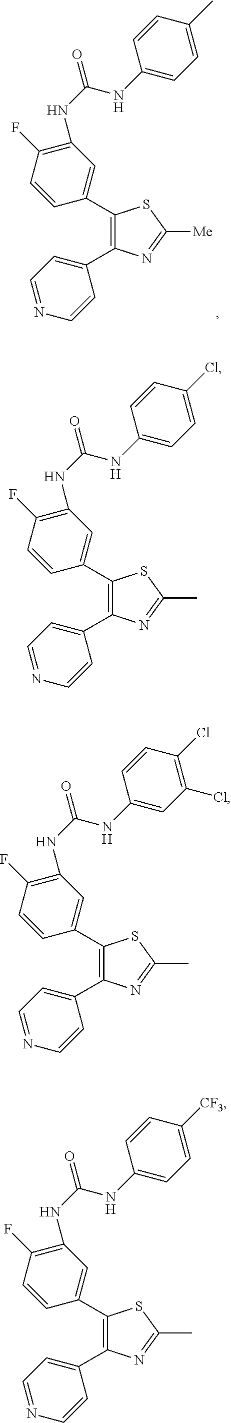 [SMILES: Cc1ccc(NC(=O)Nc2cc(-c3sc(C)nc3-c3ccncc3)ccc2F)cc1 ; Cc1nc(-c2ccncc2)c(-c2ccc(F)c(NC(=O)Nc3ccc(Cl)cc3)c2)s1 ; Cc1nc(-c2ccncc2)c(-c2ccc(F)c(NC(=O)Nc3ccc(Cl)c(Cl)c3)c2)s1 ; Cc1nc(-c2ccncc2)c(-c2ccc(F)c(NC(=O)Nc3ccc(C(F)(F)F)cc3)c2)s1]

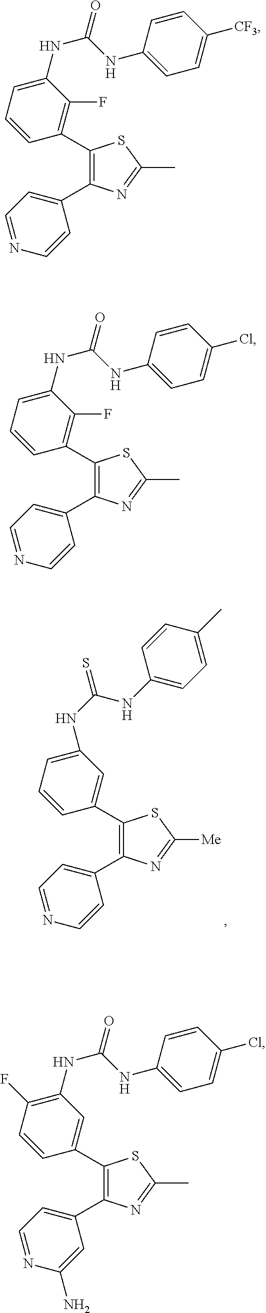 [SMILES: Cc1nc(-c2ccncc2)c(-c2cccc(NC(=O)Nc3ccc(C(F)(F)F)cc3)c2F)s1 ; Cc1nc(-c2ccncc2)c(-c2cccc(NC(=O)Nc3ccc(Cl)cc3)c2F)s1 ; Cc1ccc(NC(=S)Nc2cccc(-c3sc(C)nc3-c3ccncc3)c2)cc1 ; Cc1nc(-c2ccnc(N)c2)c(-c2ccc(F)c(NC(=O)Nc3ccc(Cl)cc3)c2)s1]

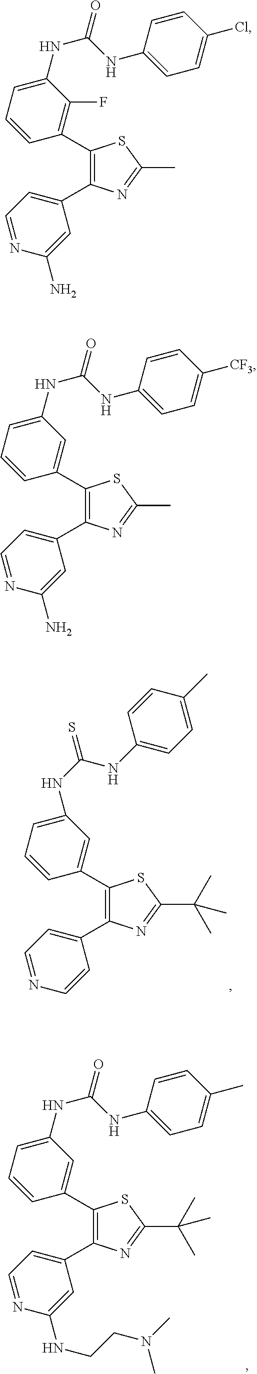 [SMILES: Cc1nc(-c2ccnc(N)c2)c(-c2cccc(NC(=O)Nc3ccc(Cl)cc3)c2F)s1 ; Cc1nc(-c2ccnc(N)c2)c(-c2cccc(NC(=O)Nc3ccc(C(F)(F)F)cc3)c2)s1 ; Cc1ccc(NC(=S)Nc2cccc(-c3sc(C(C)(C)C)nc3-c3ccncc3)c2)cc1 ; Cc1ccc(NC(=O)Nc2cccc(-c3sc(C(C)(C)C)nc3-c3ccnc(NCCN(C)C)c3)c2)cc1]

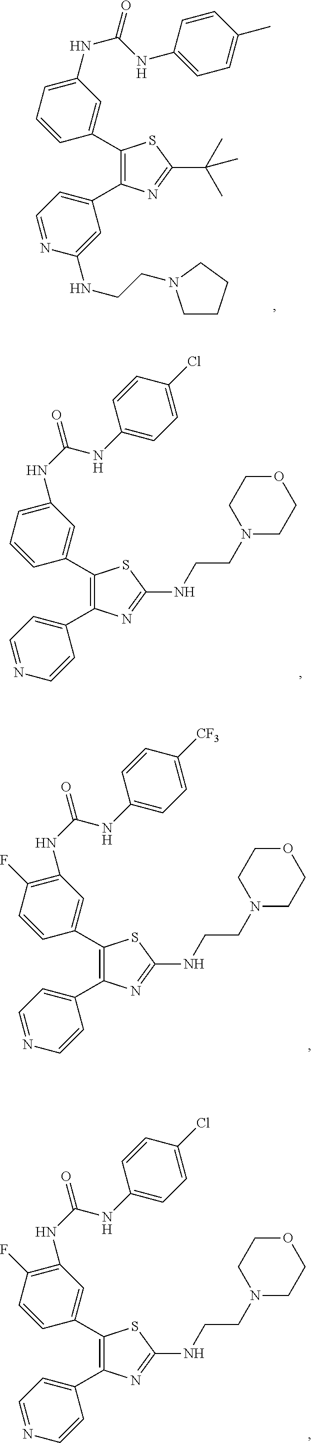 [SMILES: Cc1ccc(NC(=O)Nc2cccc(-c3sc(C(C)(C)C)nc3-c3ccnc(NCCN4CCCC4)c3)c2)cc1 ; O=C(Nc1ccc(Cl)cc1)Nc1cccc(-c2sc(NCCN3CCOCC3)nc2-c2ccncc2)c1 ; O=C(Nc1ccc(C(F)(F)F)cc1)Nc1cc(-c2sc(NCCN3CCOCC3)nc2-c2ccncc2)ccc1F ; O=C(Nc1ccc(Cl)cc1)Nc1cc(-c2sc(NCCN3CCOCC3)nc2-c2ccncc2)ccc1F]

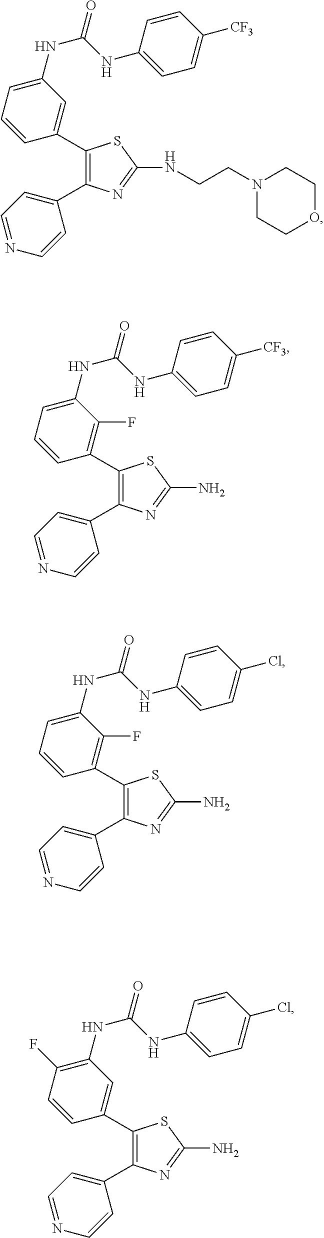 [SMILES: O=C(Nc1ccc(C(F)(F)F)cc1)Nc1cccc(-c2sc(NCCN3CCOCC3)nc2-c2ccncc2)c1 ; Nc1nc(-c2ccncc2)c(-c2cccc(NC(=O)Nc3ccc(C(F)(F)F)cc3)c2F)s1 ; Nc1nc(-c2ccncc2)c(-c2cccc(NC(=O)Nc3ccc(Cl)cc3)c2F)s1 ; Nc1nc(-c2ccncc2)c(-c2ccc(F)c(NC(=O)Nc3ccc(Cl)cc3)c2)s1]

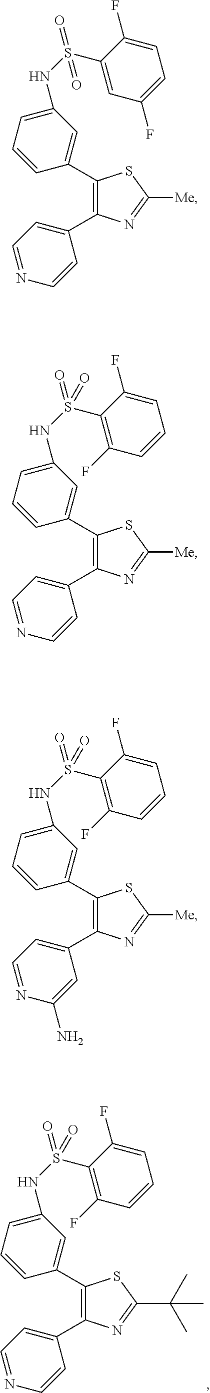 [SMILES: Cc1nc(-c2ccncc2)c(-c2cccc(NS(=O)(=O)c3cc(F)ccc3F)c2)s1 ; Cc1nc(-c2ccncc2)c(-c2cccc(NS(=O)(=O)c3c(F)cccc3F)c2)s1 ; Cc1nc(-c2ccnc(N)c2)c(-c2cccc(NS(=O)(=O)c3c(F)cccc3F)c2)s1 ; CC(C)(C)c1nc(-c2ccncc2)c(-c2cccc(NS(=O)(=O)c3c(F)cccc3F)c2)s1]

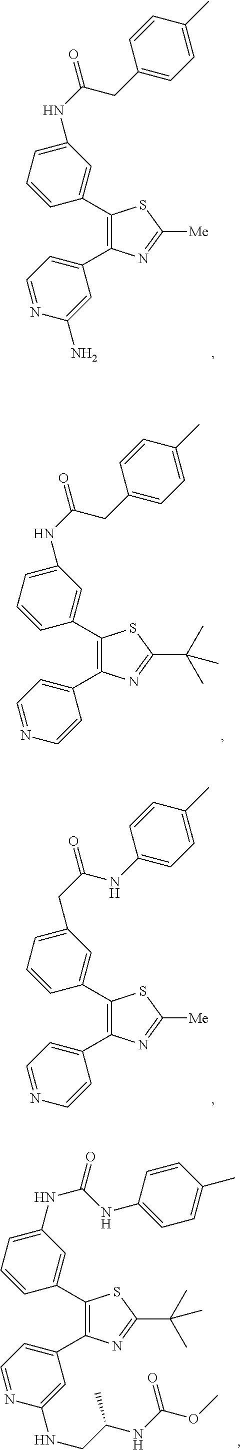 [SMILES: Cc1ccc(CC(=O)Nc2cccc(-c3sc(C)nc3-c3ccnc(N)c3)c2)cc1 ; Cc1ccc(CC(=O)Nc2cccc(-c3sc(C(C)(C)C)nc3-c3ccncc3)c2)cc1 ; Cc1ccc(NC(=O)Cc2cccc(-c3sc(C)nc3-c3ccncc3)c2)cc1 ; COC(=O)N[C@@H](C)CNc1cc(-c2nc(C(C)(C)C)sc2-c2cccc(NC(=O)Nc3ccc(C)cc3)c2)ccn1]

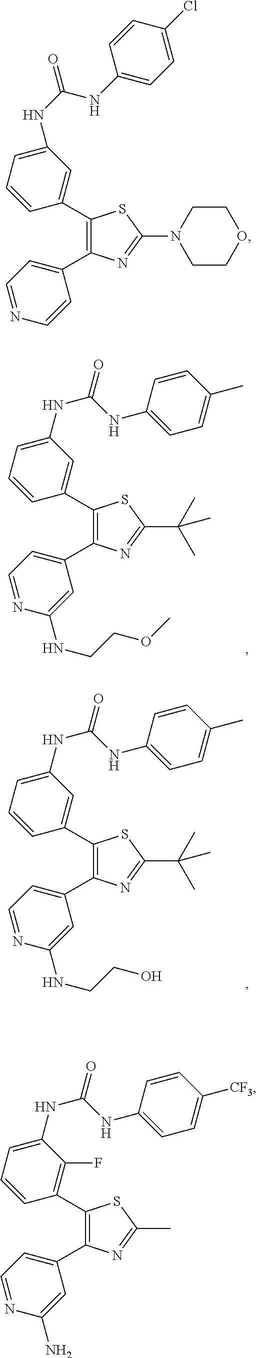

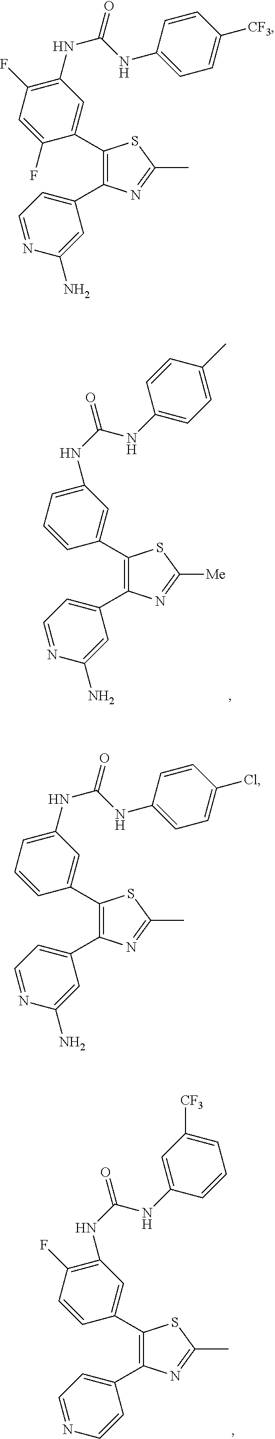 [SMILES: Cc1nc(-c2ccnc(N)c2)c(-c2cc(NC(=O)Nc3ccc(C(F)(F)F)cc3)c(F)cc2F)s1 ; Cc1ccc(NC(=O)Nc2cccc(-c3sc(C)nc3-c3ccnc(N)c3)c2)cc1 ; Cc1nc(-c2ccnc(N)c2)c(-c2cccc(NC(=O)Nc3ccc(Cl)cc3)c2)s1 ; Cc1nc(-c2ccncc2)c(-c2ccc(F)c(NC(=O)Nc3cccc(C(F)(F)F)c3)c2)s1]

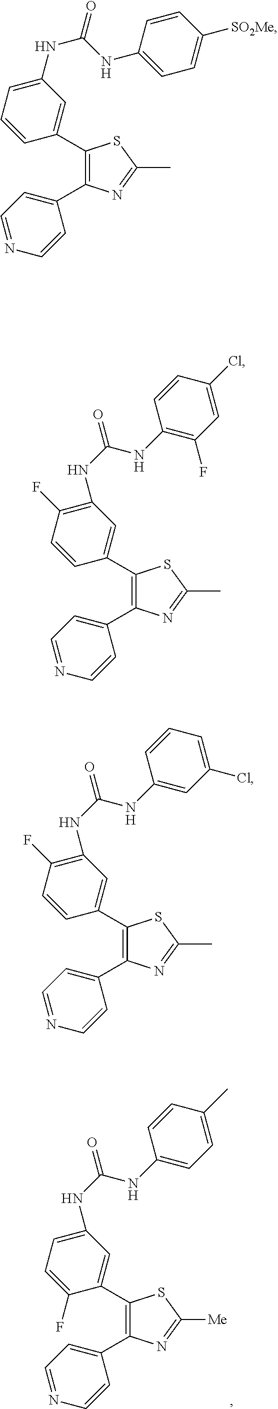 [SMILES: Cc1nc(-c2ccncc2)c(-c2cccc(NC(=O)Nc3ccc(S(C)(=O)=O)cc3)c2)s1 ; Cc1nc(-c2ccncc2)c(-c2ccc(F)c(NC(=O)Nc3ccc(Cl)cc3F)c2)s1 ; Cc1nc(-c2ccncc2)c(-c2ccc(F)c(NC(=O)Nc3cccc(Cl)c3)c2)s1 ; Cc1ccc(NC(=O)Nc2ccc(F)c(-c3sc(C)nc3-c3ccncc3)c2)cc1]

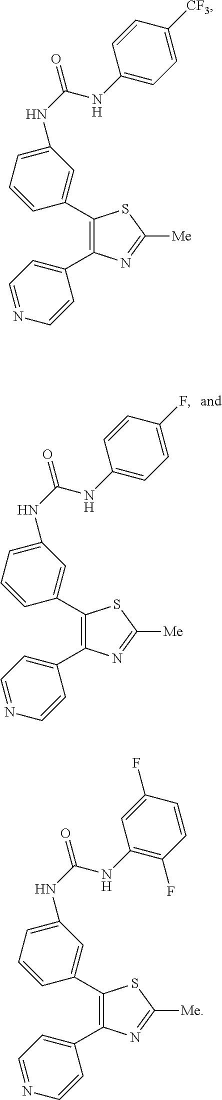 [SMILES: Cc1nc(-c2ccncc2)c(-c2cccc(NC(=O)Nc3ccc(C(F)(F)F)cc3)c2)s1 ; Cc1nc(-c2ccncc2)c(-c2cccc(NC(=O)Nc3ccc(F)cc3)c2)s1 ; Cc1nc(-c2ccncc2)c(-c2cccc(NC(=O)Nc3cc(F)ccc3F)c2)s1]

55. The method of embodiment 53, wherein the protein kinase is selected from A-RAF, B-RAF and C-RAF.
56. The method of embodiment 55, wherein the protein kinase is B-RAF.
57. The method of embodiment 56, wherein the protein kinase is a B-RAF mutant.
58. The method of embodiment 57, wherein the protein kinase is the B-RAF V600E mutant.
59. A method of inhibiting RAF kinase mediated signalling in a cell comprising contacting the cell with an inhibitory concentration of a compound of embodiment 35.
60. The method of embodiment 59, wherein the cell is characterized by increased activity of the RAS-RAF-MEK-ERK pathway compared to a non-transformed cell.
61. The method of embodiment 59, wherein the cell is characterized by a B-RAF gain-of-function mutation.
62. The method of embodiment 59, wherein the cell is characterized by the presence of the B-RAF V600E mutant
63. A method of treating a human disease or disorder mediated by RAF kinase signalling comprising administering to a patient a therapeutically effective amount of a composition comprising a compound of embodiment 35.
64. The method of embodiment 63, wherein the RAF kinase is B-RAF kinase.
65. The method of embodiment 63 or 64, wherein the disease or disorder is a proliferative disease.
66. The method of embodiment 65, wherein the proliferative disease is selected from melanoma, ovarian cancer, colorectal cancer, thyroid cancer, cholangiocarcinoma, or lung adenocarcinoma.
67. The method of embodiment 63, wherein the RAF kinase is selected from human A-RAF, B-RAF and C-RAF, or a homolog or an ortholog thereof
68. A compound of Formula (III), or a tautomer, steroisomer, geometric isomer, a pharmaceutically acceptable salt, solvate, or hydrate thereof:

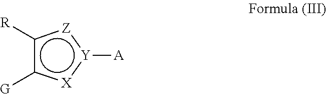

wherein
Z is N, Y is C, and X is NH;
or
Z is N, Y is N, and X is CH;
or
Z is CH, Y is N, and X is N;
or
Z is N, Y is N, and X is N;
R is

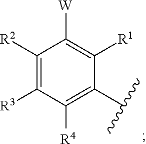

G is selected from:

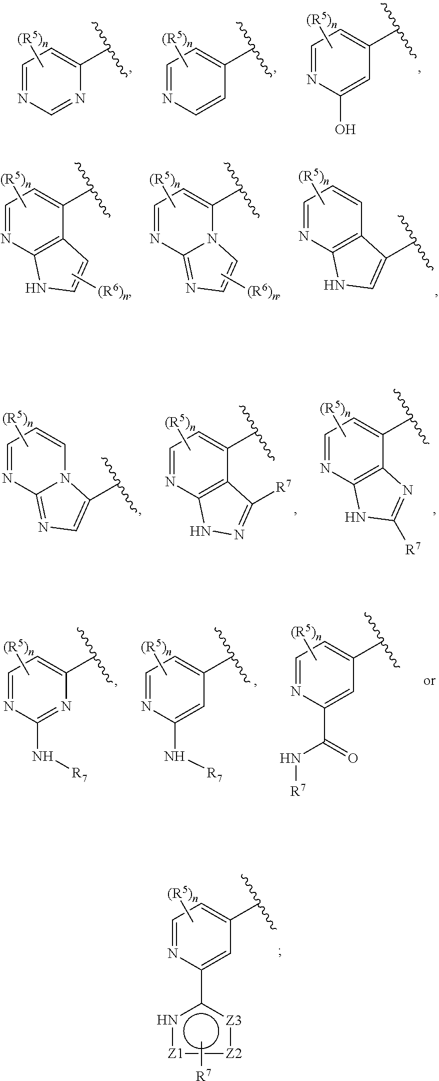

R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, Optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

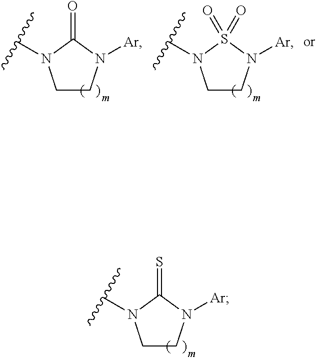

Ar is:

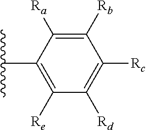

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl), CONH(C₁-C₅ optionally substituted alkyl), —S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3; and
n is 0, 1, or 2.
69. The compound of embodiment 68, wherein Z is N, Y is C, and X is NH.
70. The compound of embodiment 68, wherein Z is N, Y is N, and X is CH.
71. The compound of embodiment 68, wherein Z is CH, Y is N, and X is N.
72. The compound of embodiment 68, wherein Z is N, Y is N, and X is N.
73. The compound of embodiment 68, wherein W is

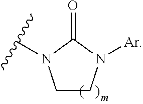

74. The compound of embodiment 68, wherein G is

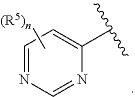

75. The compound of embodiment 68, wherein G is

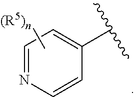

76. The compound of embodiment 68, wherein G is

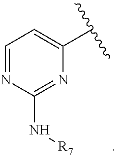

77. The compound of embodiment 68, wherein G is

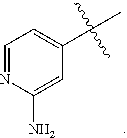 [SMILES: *c1ccnc(N)c1]

78. The compound of embodiment 68, wherein A is an optionally substituted alkyl or optionally substituted cycloalkyl.

79. The compound of embodiment 78, wherein A is an optionally substituted group selected from methyl, ethyl, trifluoromethyl, 2,2,2-trifluoroethyl, n-propyl, i-propyl, n-butyl, s-butyl, i-butyl, t-butyl, cyclopropyl or cyclobutyl.
80. The compound of embodiment 68, wherein A is —NR⁹R¹⁰.
81. The compound of embodiment 68, wherein A is an optionally substituted C-attached heterocycloalkyl.
82. The compound of embodiment 68, wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl.
83. The compound of embodiment 82, wherein R³ and R⁴ are hydrogen.
84. The compound of embodiment 83, wherein R¹ and R² are each independently selected from hydrogen, F, Cl, CN, OH, CH₂F, CHF₂, CF₃, or C₂F₅.
85. The compound of embodiment 73, wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).
86. A pharmaceutical composition comprising a compound of embodiment 68, or a stereoisomer, tautomer, hydrate, solvate or pharmaceutically acceptable salt thereof, and at least one pharmaceutically acceptable excipient.
87. A method of inhibiting a protein kinase comprising contacting the protein kinase with an inhibitory concentration of a compound of embodiment 68.
88. The compound of embodiment 68 selected from the following:

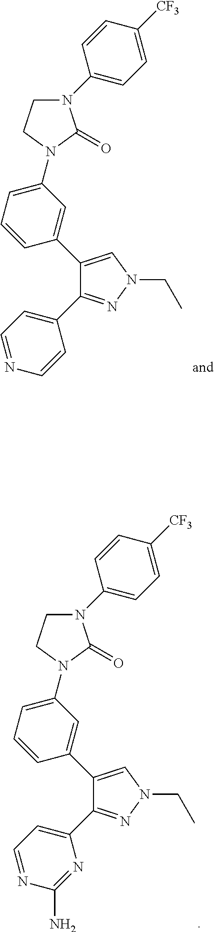 [SMILES: CCn1cc(-c2cccc(N3CCN(c4ccc(C(F)(F)F)cc4)C3=O)c2)c(-c2ccncc2)n1 ; CCn1cc(-c2cccc(N3CCN(c4ccc(C(F)(F)F)cc4)C3=O)c2)c(-c2ccnc(N)n2)n1]

89. The method of embodiment 87, wherein the protein kinase is selected from A-RAF, B-RAF and C-RAF.
90. The method of embodiment 89, wherein the protein kinase is B-RAF.
91. The method of embodiment 90, wherein the protein kinase is a B-RAF mutant.
92. The method of embodiment 91, wherein the protein kinase is the B-RAF V600E mutant.
93. A method of inhibiting RAF kinase mediated signalling in a cell comprising contacting the cell with an inhibitory concentration of a compound of embodiment 68.
94. The method of embodiment 93, wherein the cell is characterized by increased activity of the RAS-RAF-MEK-ERK pathway compared to a non-transformed cell.
95. The method of embodiment 93, wherein the cell is characterized by a B-RAF gain-of-function mutation.
96. The method of embodiment 93, wherein the cell is characterized by the presence of the B-RAF V600E mutant.
97. A method of treating a human disease or disorder mediated by RAF kinase signalling comprising administering to a patient a therapeutically effective amount of a composition comprising a compound of embodiment 68.
98. The method of embodiment 97, wherein the RAF kinase is B-RAF kinase.
99. The method of embodiment 97 or 98, wherein the disease or disorder is a proliferative disease.
100. The method of embodiment 99, wherein the proliferative disease is selected from melanoma, ovarian cancer, colorectal cancer, thyroid cancer, cholangiocarcinoma, or lung adenocarcinoma.
101. The method of embodiment 97, wherein the RAF kinase is selected from human A-RAF, B-RAF and C-RAF, or a homolog or an ortholog thereof.
102. A compound of Formula (IV), or a tautomer, steroisomer, geometric isomer, a pharmaceutically acceptable salt, solvate, or hydrate thereof:

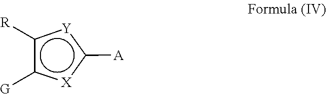

wherein.
X is S and Y is N; or
X is N and Y is S; or
X is O and Y is N; or
X is N and Y is O;
R is

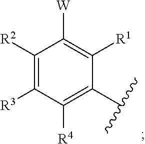

G is selected from:

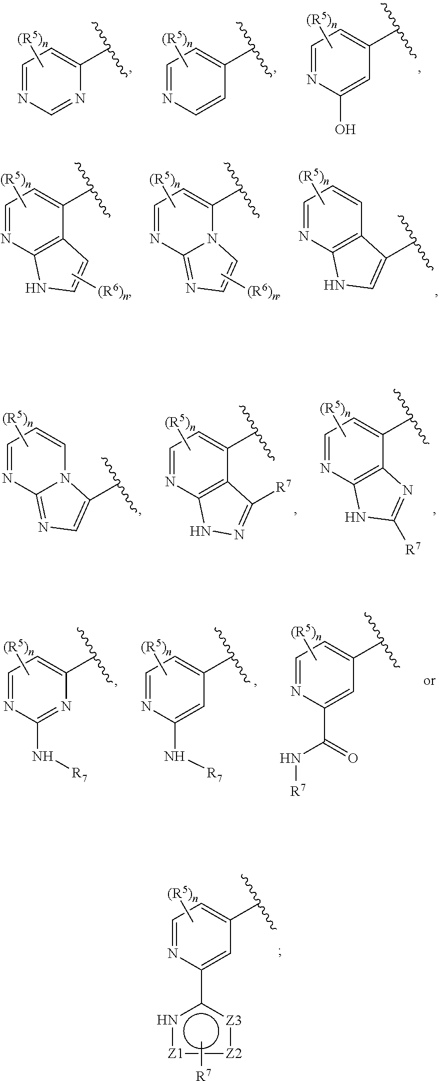

R⁵ and R⁶ are each independently selected from H, optionally substituted alkyl, optionally substituted cycloalkyl, optionally substituted aryl, optionally substituted heteroaryl optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected froth H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z₁ is N or C(R⁵);
Z₂ is N or C(R⁵);
Z₃ is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, CN, CF₃, CH₂F, CHF₂, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from

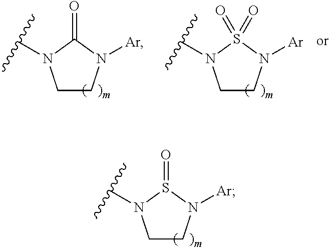

Ar is:

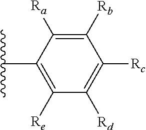

Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), NHSO₂(C₁-C₅ optionally substituted alkyl), NHCO(C₁-C₅ optionally substituted alkyl), CONH(C₁-C₅ optionally substituted alkyl)-S(C₁-C₅ optionally substituted alkyl), or optionally substituted heterocycloalkyl;
each R⁹ and R¹⁰ is independently selected from H, optionally substituted alkyl or optionally substituted cycloalkyl;
m is 1, 2, or 3;
n is 0, 1, or 2.
103. The compound of embodiment 102, wherein X is S and Y is N.
104. The compound of embodiment 102, wherein X is N and Y is S.
105. The compound of embodiment 102, wherein W is

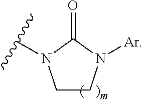

106. The compound of embodiment 102, wherein G is

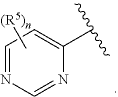

107. The compound of embodiment 102, wherein G is

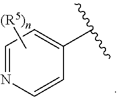

108. The compound of embodiment 102, wherein G is

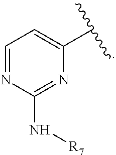

109. The compound of embodiment 102, wherein G is

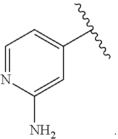 [SMILES: *c1ccnc(N)c1]

110. The compound of embodiment 102, wherein A is an optionally substituted alkyl or optionally substituted cycloalkyl.
111. The compound of embodiment 110, wherein A is an optionally substituted group selected from methyl, ethyl, trifluoromethyl, 2,2,2-trifluoroethyl, n-propyl, propyl, n-butyl, s-butyl, i-butyl, t-butyl, cyclopropyl or cyclobutyl.
112. The compound of embodiment 102, wherein A is NR⁹R¹⁰.
113. The compound of embodiment 102, wherein A is an optionally C-attached heterocycloalkyl.
114. The compound of embodiment 102, wherein R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, or C₁-C₅ optionally substituted alkyl.
115. The compound of embodiment 114, wherein R³ and R⁴ are hydrogen.
116. The compound of embodiment 115, wherein R¹ and R² are each independently selected froth hydrogen, F, Cl, CN, OH, CH₂F, CHF₂, CF₃, or C₂F₅.
117. The compound of embodiment 103, wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).
118. A pharmaceutical composition comprising a compound of embodiment 102, or a stereoisomer, tautomer, hydrate, solvate or pharmaceutically acceptable salt thereof, and at least one pharmaceutically acceptable excipient.
119. A method of inhibiting a protein kinase comprising contacting the protein kinase with an inhibitory concentration of a compound of embodiment 102.
120. The method of embodiment 119, wherein the protein kinase is selected from A-RAF, B-RAF and C-RAF.
121. The method of embodiment 120, wherein the protein kinase is B-RAF.
122. The method of embodiment 121, wherein the protein kinase is a B-RAF mutant.
123. The method of embodiment 122, wherein the protein kinase is the B-RAF V600E mutant.
124. A method of inhibiting RAF kinase mediated signalling in a cell comprising contacting, the cell with an inhibitory concentration of a compound of embodiment 102.
125. The method of embodiment 124, wherein the cell is characterized by increased activity of the RAS-RAF-MEK-ERK pathway compared to a non-transformed cell.
126. The method of embodiment 124, wherein the cell is characterized by a B-RAF gain-of-function mutation.
127. The method of embodiment 124, wherein the cell is characterized by the presence of the B-RAF V600E mutant.
128. A method of treating a human disease or disorder mediated by RAF kinase signalling comprising administering to a patient a therapeutically effective amount of a composition comprising a compound of embodiment 102.
129. The method of embodiment 128, wherein the RAF kinase is B-RAF kinase.
130. The method of embodiment 128 or 129, wherein the disease or disorder is a proliferative disease.
131. The method of embodiment 130, wherein the proliferative disease is selected from melanoma, ovarian cancer, colorectal cancer, thyroid cancer, cholangiocarcinoma, or lung adenocarcinoma.
132. The method of embodiment 129, wherein the RAF kinase is selected from human A-RAF, B-RAF and C-RAF, or a homolog or an ortholog thereof.

1. A compound of Formula (I), or a tautomer, steroisomer, geometric isomer, a pharmaceutically acceptable salt, solvate, or hydrate thereof: 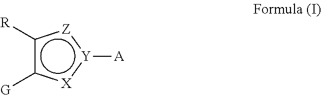
wherein
Z is N, Y is C, and X is NH;
or
Z is CH, Y is N, and X is N;
or
Z is N, Y is N, and X is N;
R is 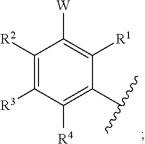
G is selected from: 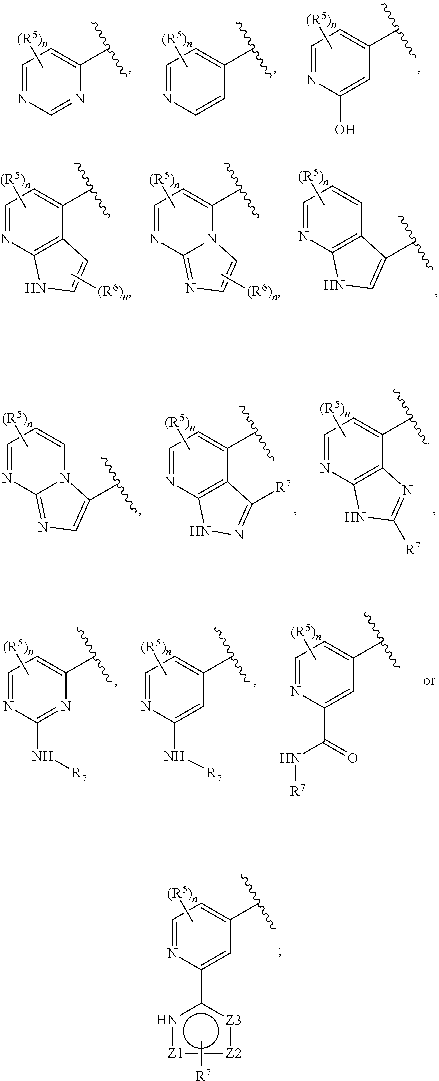
s R⁵ and R⁶ are each independently selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), F, Cl, Br, CF₃, CN, or OH;
R⁷ is selected from H, optionally substituted alkyl, optionally substituted cycloalkyl, optionally substituted aryl, optionally substituted heteroaryl, optionally substituted heteroalkyl, optionally substituted heterocycloalkyl, -(optionally substituted alkylene)-(optionally substituted heterocycloalkyl), -(optionally substituted alkylene)-(optionally substituted alkoxy), -(optionally substituted alkylene)-(NHCO₂H), or —SO₂NH(C₁-C₅ optionally substituted alkyl);
Z1 is N or C(R⁵);
Z2 is N or C(R⁵);
Z3 is N or C(R⁵);
A is selected from H, alkyl, optionally substituted alkyl, —NR⁹R¹⁰, optionally substituted N-attached heterocycloalkyl, optionally substituted C-attached heterocycloalkyl, optionally substituted cycloalkyl, or optionally substituted heteroalkyl;
R¹, R², R³ and R⁴ are each independently selected from hydrogen, F, Cl, CN, OH, CH₂F, CHF₂, CF₃, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), —S(C₁-C₅ optionally substituted alkyl), or optionally substituted heterocycloalkyl;
W is selected from —NHSO₂Ar, —NHCOAr, —NHSO₂NHAr, —NHSO₂N(Ar)₂, —NHCONHAr, —N(OH)CONHAr, —NHCON(Ar)₂, —NHCSNHAr, —NHCSN(Ar)₂, —NHCOC(R¹¹)(R¹²)Ar, —C(R¹¹)(R¹²)CONHAr;
Ar is: 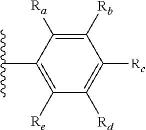
Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), —SO₂(C₁-C₅ optionally substituted alkyl), SO₂NH(C₁-C₅ optionally substituted alkyl), SO₂N(C₁-C₅ optionally substituted alkyl)₂, SO₂—(N-attached heterocycloalkyl), NHSO₂(C₁-C₅ optionally substituted alkyl), NHCO(C₁-C₅ optionally substituted alkyl); CONH(C₁-C₅— optionally substituted alkyl), —S(C₁-C₅ optionally substituted alkyl), or -optionally substituted heterocycloalkyl;
each R⁹ and R¹⁰ is independently selected from H, optionally substituted alkyl or optionally substituted cycloalkyl;
each R¹¹ and R¹² is independently selected from H, or C₁-C₆ alkyl; or for the instance wherein R¹¹ and R¹² are attached geminal carbon substituents, R¹¹ and R¹² together with the carbon atom to which they are attached are joined to form a C₃-C₆cycloalkyl; and
n is 0, 1, or 2.

2. The compound of claim 1, wherein Z is CH, Y is N, and X is N.

3. The compound of claim 1, wherein Z is N, Y is C, and X is NH.

4. The compound of claim 1, wherein Z is N, Y is N, and X is N.

5. The compound of claim 1, wherein W is NHSO₂Ar.

6. The compound of claim 1, wherein W is NHCONHAr.

7. The compound of claim 1, wherein G is

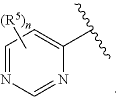

8. The compound of claim 1, wherein G is

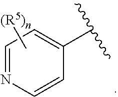

9. The compound of claim 1, wherein G is

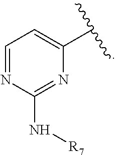

10. The compound of claim 1, wherein G is

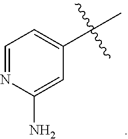 [SMILES: *c1ccnc(N)c1]

11. The compound of claim 1, wherein A is an optionally substituted alkyl or optionally substituted cycloalkyl.

12. The compound of claim 11, wherein A is an optionally substituted group selected from methyl, ethyl; trifluoromethyl, 2,2,2-trifluoroethyl, n-propyl, i-propyl, n-butyl, s-butyl, t-butyl, cyclopropyl or cyclobutyl.

13. The compound of claim 1, wherein A is NR⁹R¹⁰.

14. The compound of claim 1, wherein A is an optionally substituted C-attached heterocycloallyl.

15. The compound of claim 1, wherein R¹, R², R³ and R⁴ are each independently selected from hydrogen, F, Cl, CN, OH, CH₂F, CHF₂, CF₃, C₂F₅, NO₂, NH₂, NH(C₁-C₅ optionally substituted alkyl), —N(C₁-C₅ optionally substituted alkyl)₂, or C₁-C₅ optionally substituted alkyl.

16. The compound of claim 15, wherein R³ and R⁴ are hydrogen.

17. The compound of claim 16, wherein R¹ and R² are each independently selected from hydrogen, F, Cl, CN, —OH, CH₂F, CHF₂, CF₃, or C₂F₅.

18. The compound of claim 6, wherein Ra, Rb, Rc, Rd and Re are each independently selected from hydrogen, F, Cl, CN, CF₃, OH, C₂F₅, NO₂, NH₂, —NH(C₁-C₅ optionally substituted alkyl), —N(C₁-C₅ optionally substituted alkyl)₂, C₁-C₅ optionally substituted alkyl, —O(C₁-C₅ optionally substituted alkyl), or —SO₂(C₁-C₅ optionally substituted alkyl).

19. A pharmaceutical composition comprising a compound of claim 1, or a stereoisomer, tautomer, hydrate; solvate or pharmaceutically acceptable salt thereof, and at least one pharmaceutically acceptable excipient.

20. A method of inhibiting a protein kinase comprising contacting the protein kinase with an inhibitory concentration of a compound of claim 1.
